(19) United States
(12) Patent Application Publication
(10) Pub. No.: US 2013/0035326 A1
(43) Pub. Date: Feb. 7, 2013

(54) BIARYL COMPOUNDS AND METHODS OF USE THEREOF

(71) Applicants: Sunny Abraham, San Diego, CA (US); Mark W. Holladay, San Diego, CA (US); Gang Liu, San Diego, CA (US); Shimin Xu, San Diego, CA (US)

(72) Inventors: Sunny Abraham, San Diego, CA (US); Mark W. Holladay, San Diego, CA (US); Gang Liu, San Diego, CA (US); Shimin Xu, San Diego, CA (US)

(73) Assignee: AMBIT BIOSCIENCES CORPORATION, San Diego, CA (US)

(21) Appl. No.: 13/390,911

(22) PCT Filed: Aug. 18, 2010
(86) PCT No.: PCT/US10/45876

§ 371 Date: Oct. 5, 2012

Related U.S. Application Data
(60) Provisional application No. 61/235,316, filed on Aug. 19, 2009.

(51) Int. Cl. C07D 413/12; C07D 498/04; A61K 31/5383; A61K 31/4375; C07D 471/04; C07D 413/14; A61K 31/444; A61K 31/553; A61K 31/4545; A61K 31/506; A61K 31/5377; A61K 31/505; C07D 401/12; A61K 31/497; A61K 31/437; A61K 31/5513; C07D 239/42; C07D 401/04; A61P 29/00; A61P 37/00; A61P 35/00; A61P 35/02; A61P 37/06; A61P 17/02; A61P 13/12; A61P 17/06; A61P 37/08; A61P 11/00; A61P 19/02; A61P 25/00; A61P 19/10; A61P 11/06; A61P 37/02; A61P 31/00; A61P 3/10; A61P 1/00; A61P 1/04; A61P 19/04; A61K 31/4439

(52) U.S. Cl. 514/211.1; 546/272.1; 514/340; 544/105; 514/230.5; 544/131; 514/300; 546/122; 546/256; 514/333; 540/552; 546/194; 514/318; 514/343; 544/331; 514/272; 544/122; 514/235.8; 514/236.8; 544/123; 546/275.4; 514/341; 544/405; 514/255.05; 546/116; 514/302; 514/303; 546/118; 546/119; 540/502; 514/221; 544/332

(57) ABSTRACT

Provided herein are compounds for treatment of KIT, CSF-1R and/or FLT3 kinase mediated diseases. Also provided are pharmaceutical compositions comprising the compounds and methods of using the compounds and compositions.

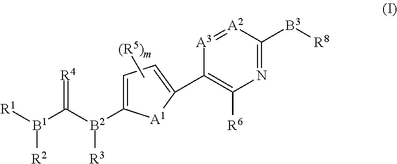

### RELATED APPLICATIONS

This application claims priority to U.S. provisional application No. 61/235,316, filed Aug. 19, 2009. The disclosure of the above referenced application is incorporated by reference herein in its entirety.

### FIELD

Provided herein are biaryl compounds. In certain embodiments, the compounds are modulators of type III receptor tyrosine kinase family. In other embodiments, the compounds are modulators of FLT3 and/or CSF-1R kinases. Also provided are compositions comprising the compounds and methods of use thereof. The compounds provided are useful in the treatment, prevention, or amelioration of diseases or disorders related to FLT3 and/or CSF-1R kinase activity or one or more symptoms associated with such diseases or disorders.

### BACKGROUND

Protein kinases (PKs) are enzymes that catalyze the phosphorylation of hydroxy groups on tyrosine, serine and threonine residues of proteins. Receptor tyrosine kinases (RTK's) are a sub-family of protein kinases that play a critical role in cell signalling and are involved in a variety of cancer related processes including cell proliferation, survival, angiogenesis, invasion and metastasis. A class of RTK known as the type III receptor tyrosine kinase family, which includes the receptors PDGFR α, PDGFR β, FLT3, Kit, VEGFR and CSF1R, has been implicated in various proliferative and inflammatory diseases.

CSF-1R (also known as macrophage colony stimulating factor receptor (M-CSFR) or fms) is a receptor for the macrophage colony stimulating factor (M-CSF or CSF-1). Binding of the CSF-1 ligand to its receptor results in dimerization and auto-phosphorylation of the receptor and leads to activation of downstream signal transduction pathways including the PI3K/Akt and the mitogen activating protein kinase MAPK pathways. Activation of CSF-1R leads to the proliferation, survival, motility and differentiation of cells of the monocyte/macrophage lineage and hence plays a role in normal tissue development and immune defense. Activation of CSF-1R also leads to the proliferation and differentiation of osteoclast precursors and impacts the process of bone resorption.

Aberrant expression and activation of CSF-1R and/or its ligand have been found in human myeloid leukaemia, prostate, breast, ovarian, endometrial and a variety of other cancers. A number of studies have demonstrated that the overexpression of CSF-1R is associated with poor prognosis in several of these cancers. In addition, the CSF-1/CSF-1R signaling plays a key role in the regulation of tumour-associated macrophage, which have been postulated to play a significant role in tumour angiogenesis, invasion and progression (E. Sapi, Exp Biol Med, 2004, 229:1-11).

Another member of the PDGFR family, Flt3 (also called Flk2), plays an important role in the proliferation and differentiation of hematopoietic stem cells and activating mutation or overexpression of this receptor is found in AML (See, Heinrich Mini-Reviews in Medicinal Chemistry (2004) 4(3):255-271, Kiyoi et al. Int J Hematol (2005) 82:85-92). More than a dozen known Flt3 inhibitors are being developed and some have shown promising clinical effects against AML (See Levis et al. Int J Hematol. (2005) 82:100-107). The Flt3 receptor is also expressed in a large portion of dendritic cell progenitors and stimulation of the receptor causes the proliferation and differentiation of these progenitors into dendritic cells (DC). Since dendritic cells are the main initiators of the T-cell mediated immune response, including the autoreactive immune response, Flt3 inhibition is a mechanism for downregulating DC-mediated inflammatory and autoimmune responses. One study shows the Flt3 inhibtor CEP-701 to be effective in reducing myelin loss in experimental autoimmune encephalomyelitis (EAE), a mouse model for multiple sclerosis (See Whartenby et al. PNAS (2005) 102: 16741-16746). A high level of the Flt3 ligand is found in the serum of patients with Langerhans cell histiocytosis and systemic lupus erythematosus, which further implicates Flt3 signaling in the disregulation of dendritic cell progenitors in those autoimmune diseases (See Rolland et al. J Immunol. (2005) 174:3067-3071).

There continues to be a need for identification of small molecules that inhibit CSF-1R and/or FLT-3 kinases, particularly compounds useful for the treatment of CSF-1R and/or FLT-3 mediated diseases.

### SUMMARY

Provided herein are compounds of formula (I) or pharmaceutically acceptable salts, solvates, hydrates or clathrates thereof. In certain embodiment, the compounds have activity as KIT, CSF-1R and/or FLT3 kinase modulators. The compounds are useful in medical treatments, pharmaceutical compositions and methods for modulating the activity of KIT, CSF-1R and/or FLT3 kinases, including wildtype and/or mutated forms of KIT, CSF-1R and/or FLT3 kinases. In certain embodiments, the compounds provided herein have activity as KIT, CSF-1R and/or FLT3 kinase modulators. In one embodiment, the compounds for use in the compositions and methods provided herein have formula (I).

In certain embodiments, provided herein are compounds of Formula I:

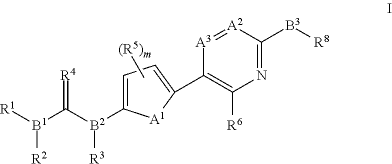

or pharmaceutically acceptable salts, solvates, hydrates or clathrates thereof, wherein:

R¹ is optionally substituted aryl, optionally substituted heteroaryl or optionally substituted heterocyclyl; where the substituents when present are selected from one, two or three R⁹ groups, wherein each R⁹ is independently selected from halo, alkyl, alkenyl, alkynyl, alkoxy, hydroxyl, haloalkoxy, cycloalkyl, cycloalkylalkyl, hydroxyalkyl, haloalkyl, aryl, arylalkyl, heterocyclyl, heterocyclylalkyl, heteroaryl, and heteroarylalkyl, where the alkyl, alkenyl, alkynyl, alkoxy, haloalkoxy, cycloalkyl, cycloalkylalkyl, hydroxyalkyl, haloalkyl, aryl, heterocyclyl, and heteroaryl groups are optionally substituted with 1 to 5 groups selected from halo, alkyl, alkenyl, alkynyl, haloalkyl, alkoxyalkyl, aryl, hydroxy, alkoxy, cycloalkyl, cyano, —R^{u}N(R^{y})(R^{z}), —R^{u}S(O)ₙR^{x}, —R^{u}C(O)OR^{x} and —R^{u}OC(O)R^{x};

R² and R³ are each independently hydrogen, halo, haloalkyl, hydroxy, alkyl, alkenyl, alkynyl, alkoxy or amino;

R⁴ is O, S, N—CN, or N—NO₂;

B¹ is N or CR^{2a};

B² is N or CR^{3a};

R^{2a} and R^{3a} are each independently hydrogen, halo, haloalkyl, hydroxy, alkyl, alkenyl, alkynyl, alkoxy or amino;

R⁵ is halo, alkyl, alkenyl, alkynyl, cycloalkyl, haloalkyl, hydroxyalkyl, alkoxyalkyl, cycloalkylalkyl, cyano, amino, hydroxy, alkoxy, —R^{u}N(R^{y})(R^{z}), aryl, heterocyclyl, or heteroaryl;

R⁶ is hydrogen, halo, alkyl, alkenyl, alkynyl, cycloalkyl, haloalkyl, hydroxyalkyl, alkoxyalkyl, cycloalkylalkyl, cyano, amino, hydroxy, alkoxy, hydroxyalkoxyalkyl, —R^{u}N(R^{y})(R^{z}), aryl, heterocyclyl, or heteroaryl;

B³ is O, NR⁷ or CR^{7a}R^{7a};

R⁷ is hydrogen, alkyl, alkenyl or alkynyl;

each R^{7a} is independently hydrogen, alkyl, alkenyl or alkynyl;

A² and R⁸ are selected as follows:

a) R⁸ is hydrogen, alkyl, alkenyl, alkynyl, cycloalkyl, —R^{u}OR^{x}, —R^{u}N(R^{y})(R^{z}), —R^{u}S(O)ₙN(R^{y})(R^{z}), —R^{u}S(O)ₙR^{x}, heterocyclyl, aryl, or heteroaryl; and A² is N, CH or CR¹⁰; or

b) A² is C; and R⁸ together with A² forms a 5-7 membered substituted or unsubstituted heterocycle, where the substituents when present are one, two or three Q groups, each independently selected from oxo, halo, hydroxyl, alkoxy, alkyl, alkenyl, alkynyl, cycloalkyl, —R^{u}N(R^{y})(R^{z}), —R^{u}S(O)ₙR^{x}, aryl, heterocyclyl, heteroaryl, hydroxyalkyl, haloalkyl and alkoxyalkyl;

R⁷ and R⁸ are each optionally substituted with 1-6, 1-3, one, two or three Q¹ groups, each independently selected from halo, hydroxyl, alkoxy, cycloalkyl, alkyl, alkenyl, alkynyl, haloalkyl, —R^{u}N(R^{y})(R^{z}), —R^{u}S(O)ₙR^{x}, aryl, heterocyclyl and heteroaryl;

Q and Q¹ groups are each optionally substituted with 1-8, 1-6, 1-5, 1-3, one, two or three Q² groups are each independently selected from halo, alkyl, alkenyl, alkynyl, cycloalkyl, haloalkyl, aryl, amino, hydroxyl and alkoxy;

each R^{u} is independently alkylene, alkenylene or alkynylene or a direct bond;

each R^{x} is independently hydrogen, haloalkyl, alkyl, alkenyl or alkynyl;

each R^{y} and R^{z} is independently selected from (i) or (ii) below:
(i) R^{y} and R^{z} are each independently hydrogen, alkyl, alkenyl, alkynyl, cycloalkyl, alkoxyalkyl, or haloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five halo, haloalkyl, alkyl, alkenyl or alkynyl groups;

A¹ is N═CR^{9a}, NR^{9a}, S, O, CR^{9a}═CR^{9a}, CR^{9a}═N; or N═N;

A³ is N, CH or C¹⁰;

each R^{9a} is independently hydrogen, halo, alkyl, alkenyl, alkynyl, cycloalkyl, haloalkyl, hydroxyalkyl, alkoxyalkyl, cycloalkylalkyl, cyano, amino, hydroxyl, aryl, —R^{u}N(R^{y})(R^{z}), —R^{u}S(O)ₙR^{x} or alkoxy;

R¹⁰ is halo, alkyl, alkenyl, alkynyl, cycloalkyl, haloalkyl, hydroxyalkyl, alkoxyalkyl, cycloalkylalkyl, cyano, amino, hydroxyl, alkoxy, —R^{u}N(R^{a})(R^{b}), —R^{u}OR^{x}, —R^{u}OR^{x}OR^{x}, —C(O)N(R^{y})(R^{z}), —R^{u}S(O)ₙR^{x}, aryl, heterocyclyl, or heteroaryl;

R^{a} and R^{b} are each independently hydrogen, alkyl, alkenyl or alkynyl; or R^{a} and R^{b}, together with the nitrogen atom to which they are attached, form an optionally substituted heterocyclyl or heteroaryl, wherein the substituents when present are selected from halo, alkyl, hydroxy and haloalkyl;

each R^{9a} is optionally substituted with 1-8, 1-6, 1-5, one, two or three Q¹ groups, each independently selected from halo, hydroxyl, alkoxy, cycloalkyl, alkyl, alkenyl, alkynyl, haloalkyl, aryl, heterocyclyl and heteroaryl;

each R¹⁰ is optionally substituted with 1-8, 1-6, 1-5, one, two or three Q¹ groups, each independently selected from halo, hydroxyl, alkoxy, cycloalkyl, alkyl, alkenyl, alkynyl or haloalkyl;

n is 0-2;

m is 0-2; and

wherein the compound is selected such that:

a) when A² is N, B³ is NH, R¹ is phenyl, A¹ is CH═CH and R⁸ is H, then R⁶ is not amino;

b) when R¹ thienyl, B¹ is CH, A² is N, B³ is NH, A¹ is CH═CH and R⁸ is H, then R⁶ is not amino; and

c) when R¹ is pyrazol-3-yl; 1,4,5,6-tetrahydrocyclopenta[c]pyrazol-3-yl; or pyridinyl, then B² is not CH, and

d) when R¹ is piperazinyl, then B¹ is not CH.

In certain embodiments, provided herein are compounds of Formula I:

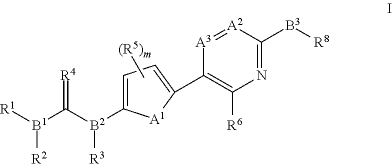

or pharmaceutically acceptable salts, solvates, hydrates or clathrates thereof, wherein:

R¹ is optionally substituted aryl, optionally substituted heteroaryl or optionally substituted heterocyclyl; where the substituents when present are selected from one, two or three R⁹ groups, wherein each R⁹ is independently selected from halo, alkyl, alkenyl, alkynyl, alkoxy, hydroxyl, haloalkoxy, cycloalkyl, cycloalkylalkyl, hydroxyalkyl, haloalkyl, aryl, arylalkyl, heterocyclyl, heterocyclylalkyl, heteroaryl, and heteroarylalkyl, where the alkyl, alkenyl, alkynyl, alkoxy, haloalkoxy, cycloalkyl, cycloalkylalkyl, hydroxyalkyl, haloalkyl, aryl, heterocyclyl, and heteroaryl groups are optionally substituted with 1 to 5 groups selected from halo, alkyl, alkenyl, alkynyl, haloalkyl, alkoxyalkyl, aryl, hydroxy, alkoxy, cycloalkyl, cyano, —R^{u}N(R^{y})(R^{z}), —R^{u}S(O)ₙR^{x}, —R^{u}C(O)OR^{x} and —R^{u}OC(O)R^{x};

R² and R³ are each independently hydrogen, halo, haloalkyl, hydroxy, alkyl, alkenyl, alkynyl, alkoxy or amino;

R⁴ is O, S, N—CN, or N—NO₂;

B¹ is N or CR^{2a};

B² is N or CR^{3a};

R^{2a} and R^{3a} are each independently hydrogen, halo, haloalkyl, hydroxy, alkyl, alkenyl, alkynyl, alkoxy or amino

R⁵ is halo, alkyl, alkenyl, alkynyl, cycloalkyl, haloalkyl, hydroxyalkyl, alkoxyalkyl, cycloalkylalkyl, cyano, amino, hydroxy, alkoxy, —R^{u}N(R^{y})(R^{z}), aryl, heterocyclyl, or heteroaryl;

R⁶ is hydrogen, halo, alkyl, alkenyl, alkynyl, cycloalkyl, haloalkyl, hydroxyalkyl, alkoxyalkyl, cycloalkylalkyl, cyano, amino, hydroxy, alkoxy, hydroxyalkoxyalkyl, —R^{u}N(R^{y})(R^{z}), aryl, heterocyclyl, or heteroaryl;

B³ is O, NR⁷ or CR^{7a}R^{7a};

R⁷ is hydrogen, alkyl, alkenyl or alkynyl;

each R^{7a} is independently hydrogen, alkyl, alkenyl or alkynyl;

A² and R⁸ are selected as follows:

a) R⁸ is hydrogen, alkyl, alkenyl, alkynyl, cycloalkyl, —R^{u}OR^{x}, —R^{u}N(R^{y})(R^{z}), —R^{u}S(O)ₙN(R^{y})(R^{z}), —R^{u}S(O)ₙR^{x}, heterocyclyl, aryl, or heteroaryl; and A² is N, CH or CR¹⁰; or

b) A² is C; and R⁸ together with A² forms a 5-7 membered substituted or unsubstituted heterocycle, where the substituents when present are one, two or three Q groups, each independently selected from oxo, halo, hydroxyl, alkoxy, alkyl, alkenyl, alkynyl, cycloalkyl, —R^{u}N(R^{y})(R^{z}), —R^{u}S(O)ₙR^{x}, aryl, heterocyclyl, heteroaryl, hydroxyalkyl, haloalkyl and alkoxyalkyl;

R⁷ and R⁸ are each optionally substituted with 1-6, 1-3, one, two or three Q¹ groups, each independently selected from halo, hydroxyl, alkoxy, cycloalkyl, alkyl, alkenyl, alkynyl, haloalkyl, —R^{u}N(R^{y})(R^{z}), —R^{u}S(O)ₙR^{x}, aryl, heterocyclyl and heteroaryl;

Q and Q¹ groups are each optionally substituted with 1-8, 1-6, 1-5, 1-3, one, two or three Q² groups each independently selected from halo, alkyl, alkenyl, alkynyl, cycloalkyl, haloalkyl, aryl, amino, hydroxyl and alkoxy;

each R^{u} is independently alkylene, alkenylene or alkynylene or a direct bond;

each R^{x} is independently hydrogen, haloalkyl, alkyl, alkenyl or alkynyl;

each R^{y} and R^{z} is independently selected from (i) or (ii) below:
(i) R^{y} and R^{z} are each independently hydrogen, alkyl, alkenyl, alkynyl, cycloalkyl, alkoxyalkyl, or haloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five halo, haloalkyl, alkyl, alkenyl or alkynyl groups;

A¹ is N═CR^{9a}, NR^{9a}, S, O, CR^{9a}═CR^{9a}, CR^{9a}═N; or N═N;

A³ is N, CH or CR¹⁰;

each R^{9a} is independently hydrogen, halo, alkyl, alkenyl, alkynyl, cycloalkyl, haloalkyl, hydroxyalkyl, alkoxyalkyl, cycloalkylalkyl, cyano, amino, hydroxyl, aryl, —R^{u}N(R^{y})(R^{z}), —R^{u}S(O)ₙR^{x} or alkoxy;

R¹⁰ is halo, alkyl, alkenyl, alkynyl, cycloalkyl, haloalkyl, hydroxyalkyl, alkoxyalkyl, cycloalkylalkyl, cyano, amino, hydroxyl, alkoxy, —R^{u}N(R^{a})(R^{b}), —R^{u}OR^{x}, —R^{u}OR^{x}OR^{x}, —C(O)N(R^{y})(R^{z}), —R^{u}S(O)ₙR^{x}, aryl, heterocyclyl, or non-azole heteroaryl;

R^{a} and R^{b} are each independently hydrogen, alkyl, alkenyl or alkynyl; or R^{a} and R^{b}, together with the nitrogen atom to which they are attached, form an optionally substituted heterocyclyl or heteroaryl, wherein the substituents when present are selected from halo, alkyl, hydroxy and haloalkyl;

R^{9a} and R¹⁰ are each optionally substituted with 1-8, 1-6, 1-5, one, two or three Q¹ groups, each independently selected from halo, hydroxyl, alkoxy, cycloalkyl, alkyl, alkenyl, alkynyl, haloalkyl, aryl, heterocyclyl and heteroaryl;

n is 0-2;

m is 0-2; and

wherein the compound is selected such that:

a) when A² is N, B³ is NH, R¹ is phenyl, A¹ is CH═CH and R⁸ is H, then R⁶ is not amino;

b) when R¹ thienyl, B¹ is CH, A² is N, B³ is NH, A¹ is CH═CH and R⁸ is H, then R⁶ is not amino; and

c) when R¹ is pyrazol-3-yl; 1,4,5,6-tetrahydrocyclopenta[c]pyrazol-3-yl; or pyridinyl, then B² is not CH, and

d) when R¹ is piperazinyl, then B¹ is not CH.

In one embodiment, the compound provided herein is a compound of formula (I). In one embodiment, the compound provided herein is a pharmaceutically acceptable salt of the compound of formula (I). In one embodiment, the compound provided herein is a solvate of the compound of formula (I). In one embodiment, the compound provided herein is a hydrate of compound of formula (I). In one embodiment, the compound provided herein is a prodrug of the compound of formula (I). In one embodiment, the compound provided herein is a clathrate of the compound of formula (I).

Also provided are pharmaceutical compositions formulated for administration by an appropriate route and means containing effective concentrations of one or more of the compounds provided herein, or pharmaceutically acceptable salts, solvates, hydrates and prodrugs thereof, and optionally comprising at least one pharmaceutical carrier.

Such pharmaceutical compositions deliver amounts effective for the treatment, prevention, or amelioration of diseases or disorders that are modulated or otherwise affected by KIT, CSF-1R and/or FLT3 kinases, or one or more symptoms or causes thereof. Such diseases or disorders include without limitation, cancers, nonmalignant proliferation diseases, atherosclerosis, restenosis following vascular angioplasty, fibroproliferative disorders, inflammatory diseases or disorders related to immune dysfunction, infectious diseases, and/or diseases or disorders that can be treated, prevented or managed by modulating the activity, binding or sub-cellular distribution of kinases, wherein such methods comprise administering to a subject, e.g., a human, in need of such treatment, prevention or management a therapeutically and prophylactically effective amount of a compound provided herein. Such diseases or disorders are further described herein.

Also provided herein are combination therapies using one or more compounds or compositions provided herein, or pharmaceutically acceptable derivatives thereof, in combination with other pharmaceutically active agents for the treatment of the diseases and disorders described herein.

In one embodiment, such additional pharmaceutical agents include one or more chemotherapeutic agents, anti-proliferative agents, anti-inflammatory agents, immunomodulatory agents or immunosuppressive agents.

The compounds or compositions provided herein, or pharmaceutically acceptable derivatives thereof, may be administered simultaneously with, prior to, or after administration of one or more of the above agents. Pharmaceutical compositions containing a compound provided herein and one or more of the above agents are also provided.

In certain embodiments, provided herein are methods of treating, preventing or ameliorating a disease or disorder that is modulated or otherwise affected by KIT, CSF-1R and/or FLT3 kinase such as wild type and/or mutant KIT, CSF-1R and/or FLT3 kinase, or one or more symptoms or causes thereof.

In practicing the methods, effective amounts of the compounds or compositions containing therapeutically effective concentrations of the compounds, which are formulated for systemic delivery, including parenteral, oral, or intravenous delivery, or for local or topical application are administered to an individual exhibiting the symptoms of the disease or disorder to be treated. The amounts are effective to ameliorate or eliminate one or more symptoms of the disease or disorder.

Further provided is a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like.

These and other aspects of the subject matter described herein will become evident upon reference to the following detailed description.

### DETAILED DESCRIPTION

Provided herein are compounds of formula I that have activity as KIT, CSF-1R and/or FLT3 kinase modulators. Further provided are methods of treating, preventing or ameliorating diseases that are modulated by KIT, CSF-1R and/or FLT3 kinase, and pharmaceutical compositions and dosage forms useful for such methods. The methods and compositions are described in detail in the sections below.

### A. DEFINITIONS

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

“Alkyl” refers to a straight or branched hydrocarbon chain group consisting solely of carbon and hydrogen atoms, containing no unsaturation, having from one to ten, one to eight, one to six or one to four carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like.

The term “branched alkyl” refers to hydrocarbon chain containing at least one forked carbon in the chain, with the smallest branched alkyl being an isopropyl group. Examples of branched alkyl groups include but is not limited to —CH(CH₃)₂, —C(CH₃)₃, —CH(CH₃)(CH₂CH₃), —CH(CH₂CH₃)₂, —C(CH₃)(CH₂CH₃)₂, —C(CH₃)₂(CH₂CH₃), —C(CH₂CH₃)₃, —C(CH₃)₂(CH(CH₃)₂) and —C(CH₃)₂(C(CH₃)₃).

“Alkenyl” refers to a straight or branched hydrocarbon chain group consisting solely of carbon and hydrogen atoms, containing at least one double bond, having from two to ten carbon atoms, and which is attached to the rest of the molecule by a single bond or a double bond, e.g., ethenyl, prop-1-enyl, but-1-enyl, pent-1-enyl, penta-1,4-dienyl, and the like.

“Alkynyl” refers to a straight or branched hydrocarbon chain group consisting solely of carbon and hydrogen atoms, containing at least one triple bond, having from two to ten carbon atoms, and which is attached to the rest of the molecule by a single bond or a triple bond, e.g., ethynyl, prop-1-ynyl, but-1-ynyl, pent-1-ynyl, pent-3-ynyl and the like.

“Alkoxy” refers to the group having the formula —OR wherein R is alkyl or haloalkyl. An “optionally substituted alkoxy” refers to the group having the formula —OR wherein R is an optionally substituted alkyl as defined herein.

“Amine” or “amino” refers to a group having the formula —NR′R″ wherein R′ and R″ are each independently hydrogen, alkyl, haloalkyl, hydroxyalkyl or alkoxyalkyl.

“Aryl” refers to a group of carbocylic ring system, including monocyclic, bicyclic, tricyclic, tetracyclic C₆-C₁₈ ring systems, wherein at least one of the rings is aromatic. The aryl may be fully aromatic, examples of which are phenyl, naphthyl, anthracenyl, acenaphthylenyl, azulenyl, fluorenyl, indenyl and pyrenyl. The aryl may also contain an aromatic ring in combination with a non-aromatic ring, examples of which are acenaphene, indene, and fluorene.

“Cycloalkyl” refers to a stable monovalent monocyclic or bicyclic hydrocarbon group consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl, norbornane, norbornene, adamantyl, bicyclo[2.2.2]octane and the like.

“Azolyl” refers to a 5-membered heterocyclic or heteroaryl ring system containing at least one nitrogen atom. Exemplary azolyl rings include pyrazole, thiazole, oxazole, diathiazole, thiadiazole, diazole, and triazole.

“Alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl, S(═O)₂R′ or COR′, where R′ is alkyl, aryl, aralkyl, heteroaryl, heteroaralkyl, —OY or —NYY′, where Y and Y′ are each independently hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—) The term “lower alkylene” refers to alkylene groups having 1 to 6 carbons. In certain embodiments, alkylene groups are lower alkylene, including alkylene of 1 to 3 carbon atoms.

“Alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

“Alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C—C≡CC—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

“Halo, “halogen” or “halide” refers to F, Cl, Br or I.

“Haloalkyl” refers to an alkyl group, in certain embodiments, C₁₋₆alkyl group in which one or more of the hydrogen atoms are replaced by halogen. Such groups include, but are not limited to, chloromethyl, trifluoromethyl 1-chloro-2-fluoroethyl, 2,2-difluoroethyl, 2-fluoropropyl, 2-fluoropropan-2-yl, 2,2,2-trifluoroethyl, 1,1-difluoroethyl, 1,3-difluoro-2-methylpropyl, 2,2-difluorocyclopropyl, (trifluoromethyl)cyclopropyl, 4,4-difluorocyclohexyl and 2,2,2-trifluoro-1,1-dimethyl-ethyl.

“Heterocyclyl” refers to a stable 3- to 15-membered non-aromatic ring radical which consists of carbon atoms and from one to five heteroatoms selected from a group consisting of nitrogen, oxygen and sulfur. In one embodiment, the heterocyclic ring system radical may be a monocyclic, bicyclic or tricyclic ring or tetracyclic ring system, which may include fused or bridged ring systems; and the nitrogen or sulfur atoms in the heterocyclic ring system radical may be optionally oxidized; the nitrogen atom may be optionally quaternized; and the heterocyclyl radical may be partially or fully saturated. The heterocyclic ring system may be attached to the main structure at any heteroatom or carbon atom which results in the creation of a stable compound. Exemplary heterocylic radicals include, morpholinyl, piperidinyl, piperazinyl, pyranyl, pyrrolidinyl and others.

“Heteroaryl” refers to a heterocyclyl group as defined above which is aromatic. The heteroaryl group may be attached to the main structure at any heteroatom or carbon atom which results in the creation of a stable compound. Examples of such heteroaryl groups include, but are not limited to: furanyl, imidazolyl, oxazolyl, isoxazolyl, pyrimidinyl, pyridinyl, thiazolyl, thienyl and others.

“Heterocyclylalkyl” refers to a group of the formula —RₐRₑ wherein Rₐ is an alkyl group as defined above and Rₑ is a heterocyclyl group as defined herein, where the alkyl group Rₐ may attach at either the carbon atom or the heteroatom of the heterocyclyl group Rₑ. The alkyl group and the heterocyclyl group may be optionally substituted as defined herein.

As used herein, “substituted alkyl,” “substituted aryl,” “substituted heteroaryl” and “substituted heterocyclyl” refer to alkyl, aryl, heteroaryl and heterocyclyl groups, respectively, that are substituted with one or more substituents, in certain embodiments one to three or four substituents, where the substituents are as defined herein, generally selected from Q¹.

“IC₅₀” refers to an amount, concentration or dosage of a particular test compound that achieves a 50% inhibition of a maximal response, such as cell growth or proliferation measured via any the in vitro or cell based assay described herein.

“Oxo” refers to the group ═O attached to a carbon atom.

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

As used herein and unless otherwise indicated, the term “hydrate” means a compound provided herein or a salt thereof, that further includes a stoichiometric or non-stoichiometeric amount of water bound by non-covalent intermolecular forces.

As used herein and unless otherwise indicated, the term “solvate” means a solvate formed from the association of one or more solvent molecules to a compound provided herein. The term “solvate” includes hydrates (e.g., mono-hydrate, dihydrate, trihydrate, tetrahydrate and the like).

As used herein, “substantially pure” means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

Unless stated otherwise specifically described in the specification, it is understood that the substitution can occur on any atom of the alkyl, alkenyl, alkynyl, cycloalkyl, heterocyclyl, aryl or heteroaryl group.

Unless specifically stated otherwise, where a compound may assume alternative tautomeric, regioisomeric and/or stereoisomeric forms, all alternative isomers are intended to be encompassed within the scope of the claimed subject matter. For example, where a compound is described as having one of two tautomeric forms, it is intended that the both tautomers be encompassed herein.

Thus, the compounds provided herein may be enantiomerically pure, or be stereoisomeric or diastereomeric mixtures.

It is to be understood that the compounds provided herein may contain chiral centers. Such chiral centers may be of either the (R) or (S) configuration, or may be a mixture thereof. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

Optically active (+) and (−), (R)- and (S)-, or (D)- and (L)-isomers may be prepared using chiral synthons or chiral reagents, or resolved using conventional techniques, such as reverse phase HPLC.

As used herein, “isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen”, the position is understood to have hydrogen at its natural isotopic composition.

As used herein, “isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

Where the number of any given substituent is not specified (e.g., haloalkyl), there may be one or more substituents present. For example, “haloalkyl” may include one or more of the same or different halogens.

In the description herein, if there is any discrepancy between a chemical name and chemical structure, the structure preferably controls.

“Anti-cancer agents” refers to anti-metabolites (e.g., 5-fluoro-uracil, methotrexate, fludarabine), antimicrotubule agents (e.g., vinca alkaloids such as vincristine, vinblastine; taxanes such as paclitaxel, docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, JM-216 or satraplatin, CI-973), anthracyclines (e.g., doxrubicin, daunorubicin), antitumor antibiotics (e.g., mitomycin, idarubicin, adriamycin, daunomycin), topoisomerase inhibitors (e.g., etoposide, camptothecins), anti-angiogenesis agents (e.g. Sutent® and Bevacizumab) or any other cytotoxic agents, (estramustine phosphate, prednimustine), hormones or hormone agonists, antagonists, partial agonists or partial antagonists, kinase inhibitors, and radiation treatment.

“Anti-inflammatory agents” refers to matrix metalloproteinase inhibitors, inhibitors of pro-inflammatory cytokines (e.g., anti-TNF molecules, TNF soluble receptors, and IL1) non-steroidal anti-inflammatory drugs (NSAIDs) such as prostaglandin synthase inhibitors (e.g., choline magnesium salicylate, salicylsalicyclic acid), COX-1 or COX-2 inhibitors), or glucocorticoid receptor agonists such as corticosteroids, methylprednisone, prednisone, or cortisone.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. 1972, 11:942-944).

### B. COMPOUNDS

In certain embodiments, provided herein are compounds of Formula I:

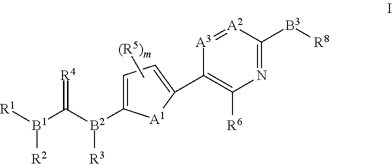

or pharmaceutically acceptable salts, solvates, hydrates or clathrates thereof, wherein:

R¹ is optionally substituted aryl, optionally substituted heteroaryl or optionally substituted heterocyclyl; where the substituents when present are selected from one, two or three R⁹ groups, wherein each R⁹ is independently selected from halo, alkyl, alkenyl, alkynyl, alkoxy, hydroxyl, haloalkoxy, cycloalkyl, cycloalkylalkyl, hydroxyalkyl, haloalkyl, aryl, arylalkyl, heterocyclyl, heterocyclylalkyl, heteroaryl, and heteroarylalkyl, where the alkyl, alkenyl, alkynyl, alkoxy, haloalkoxy, cycloalkyl, cycloalkylalkyl, hydroxyalkyl, haloalkyl, aryl, heterocyclyl, and heteroaryl groups are each optionally substituted with 1 to 5 groups selected from halo, hydroxy, alkoxy,

cycloalkyl, cyano, and —R^{u}N(R^{y})(R^{z});

R² and R³ are each independently hydrogen or alkyl;

R⁴ is O, S, N—CN, or N—NO₂;

B¹ and B² are each independently selected from N and CH;

R⁵ is halo, alkyl, alkenyl, alkynyl, cycloalkyl, haloalkyl, hydroxyalkyl, alkoxyalkyl, cycloalkylalkyl, cyano, amino, hydroxy, alkoxy, —R^{u}N(R^{y})(R^{z}), aryl, heterocyclyl, or heteroaryl;

R⁶ is hydrogen, halo, alkyl, alkenyl, alkynyl, cycloalkyl, haloalkyl, hydroxyalkyl, alkoxyalkyl, cycloalkylalkyl, cyano, amino, hydroxy, alkoxy, hydroxyalkoxyalkyl, —R^{u}N(R^{y})(R^{z}), aryl, heterocyclyl, or heteroaryl;

B³ is O, NR⁷ or CR^{7a}R^{7a};

R⁷ is hydrogen or alkyl;

each R^{7a} is independently hydrogen or alkyl;

A² and R⁸ are selected as follows:

a) R⁸ is hydrogen, alkyl, cycloalkyl, —R^{u}OR^{x}, —R^{u}N(R^{y})(R^{z}), heterocyclyl, aryl, or heteroaryl; and A² is N, CH or CR¹⁰; or

b) A² is C; and R⁸ together with A² forms a 5-7 membered substituted or unsubstituted heterocycle, where the substituents when present are one, two or three Q groups, each independently selected from oxo, halo, hydroxyl, alkoxy, alkyl, alkenyl, alkynyl, cycloalkyl, —R^{u}N(R^{y})(R^{z}), aryl, heterocyclyl, heteroaryl, hydroxyalkyl, haloalkyl and alkoxyalkyl;

R⁷ and R⁸ are optionally substituted with one, two or three Q¹ groups, each independently selected from halo, hydroxyl, alkoxy, cycloalkyl, alkyl, alkenyl, alkynyl, haloalkyl, heterocyclyl and heteroaryl;

Q and Q¹ groups are each optionally substituted with one, two or three Q² groups each independently selected from halo, alkyl, alkenyl, alkynyl, cycloalkyl, haloalkyl, hydroxyl and alkoxy;

each R^{u} is independently alkylene or a direct bond;

each R^{x} is independently hydrogen or alkyl;

each R^{y} and R^{z} is independently selected from (i) or (ii) below:
(i) R^{y} and R^{z} are each independently hydrogen, alkyl, alkenyl, alkynyl, cycloalkyl, alkoxyalkyl, or haloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five alkyl groups;

A¹ is N═CR^{9a}, NR^{9a}, S, O, CR^{9a}═CR^{9a}, CR^{9a}═N; or N═N;

A³ is N, CH or CR¹⁰;

each R^{9a} is independently hydrogen, halo, alkyl, alkenyl, alkynyl, cycloalkyl, haloalkyl, hydroxyalkyl, alkoxyalkyl, cycloalkylalkyl, cyano, amino, hydroxyl, aryl, —R^{u}N(R^{y})(R^{z}), or alkoxy;

R¹⁰ is halo, alkyl, alkenyl, alkynyl, cycloalkyl, haloalkyl, hydroxyalkyl, alkoxyalkyl, cycloalkylalkyl, cyano, amino, hydroxyl, alkoxy, —R^{u}N(R^{y})(R^{z}), —C(O)N(R^{y})(R^{z}), —R^{u}S(O)ₙR^{x}, aryl, heterocyclyl, or non-azole heteroaryl;

m is 0-2; and

wherein the compound is selected with a proviso that when A² is N, B³ is NH, R¹ is phenyl, A¹ is CH═CH and R⁸ is H, then R⁶ is not amino.

In certain embodiments, provided herein are compounds of Formula I, wherein R¹ is optionally substituted aryl, optionally substituted heteroaryl or optionally substituted heterocyclyl; where the substituents when present are selected from one, two or three R⁹ groups, wherein each R⁹ is independently selected from halo, alkyl, alkenyl, alkynyl, alkoxy, hydroxyl, haloalkoxy, cycloalkyl, cycloalkylalkyl, hydroxyalkyl, haloalkyl, aryl, arylalkyl, heterocyclyl, heterocyclylalkyl, heteroaryl, and heteroarylalkyl, where the alkyl, alkenyl, alkynyl, alkoxy, haloalkoxy, cycloalkyl, cycloalkylalkyl, hydroxyalkyl, haloalkyl, aryl, heterocyclyl, and heteroaryl groups are optionally substituted with 1 to 5 groups selected from halo, alkyl, alkenyl, alkynyl, aryl, hydroxy, alkoxy, cycloalkyl, cyano, —R^{u}C(O)OR^{x}, —R^{u}N(R^{y})(R^{z}) and —R^{u}OC(O)R^{x};

R² and R³ are each independently hydrogen, halo, hydroxy, alkyl or amino;

R⁴ is O, S, N—CN, or N—NO₂;

B¹ is selected from N and CR^{2a};

B² is N or CR^{3a};

R^{2a} and R^{3a} are each independently hydrogen, alkyl or halo;

R⁵ is halo, alkyl, alkenyl, alkynyl, cycloalkyl, haloalkyl, hydroxyalkyl, alkoxyalkyl, cycloalkylalkyl, cyano, amino, hydroxy, alkoxy, —R^{u}N(R^{y})(R^{z}), aryl, heterocyclyl, or heteroaryl;

R⁶ is hydrogen, halo, alkyl, alkenyl, alkynyl, cycloalkyl, haloalkyl, hydroxyalkyl, alkoxyalkyl, cycloalkylalkyl, cyano, amino, hydroxy, alkoxy, hydroxyalkoxyalkyl, —R^{u}N(R^{y})(R^{z}), aryl, heterocyclyl, or heteroaryl;

B³ is O, NR⁷, CH₂, or CR^{7a}R^{7a};

R⁷ is hydrogen or alkyl;

each R^{7a} is independently hydrogen or alkyl;

A² and R⁸ are selected as follows:

a) R⁸ is hydrogen, alkyl, alkenyl, alkynyl, cycloalkyl, —R^{u}OR^{x}, —R^{u}N(R^{y})(R^{z}), —R^{u}S(O)ₙR^{x}, heterocyclyl, aryl, or heteroaryl; and A² is N, CH or CR¹⁰; or

b) A² is C; and R⁸ together with A² forms a 5-7 membered substituted or unsubstituted heterocycle, where the substituents when present are one, two or three Q groups, each independently selected from oxo, halo, hydroxyl, alkoxy, alkyl, alkenyl, alkynyl, cycloalkyl, —R^{u}N(R^{y})(R^{z}), aryl, heterocyclyl, heteroaryl, hydroxyalkyl, haloalkyl and alkoxyalkyl;

R⁷ and R⁸ are each optionally substituted with one, two or three Q¹ groups, each independently selected from halo, hydroxyl, alkoxy, cycloalkyl, alkyl, alkenyl, alkynyl, haloalkyl, heterocyclyl and heteroaryl;

Q and Q¹ groups are each optionally substituted with 1-6, 1-5, 1-3, one, two or three Q² groups each independently selected from halo, alkyl, alkenyl, alkynyl, cycloalkyl, haloalkyl, amino, hydroxyl and alkoxy;

each R^{u} is independently alkylene, alkenylene or alkynylene or a direct bond;

each R^{x} is independently hydrogen, alkyl, alkenyl or alkynyl;

each R^{y} and R^{z} is independently selected from (i) or (ii) below:
(i) R^{y} and R^{z} are each independently hydrogen, alkyl, alkenyl, alkynyl, cycloalkyl, alkoxyalkyl or haloalkyl; or
(ii) R^{y} and R^{z}, together with the nitrogen atom to which they are attached, form a heterocyclyl or heteroaryl, optionally substituted with an alkyl;

A¹ is N═CR^{9a}, NR^{9a}, S, CR^{9a}═CR^{9a}, CR^{9a}═N;

A³ is N, CH or CR¹⁰;

each R^{9a} is independently hydrogen, halo, alkyl, alkenyl, alkynyl, cycloalkyl, haloalkyl, hydroxyalkyl, alkoxyalkyl, cycloalkylalkyl, cyano, amino, hydroxyl, aryl, —R^{u}N(R^{y})(R^{z}), or alkoxy;

R¹⁰ is halo, alkyl, alkenyl, alkynyl, cycloalkyl, haloalkyl, hydroxyalkyl, alkoxyalkyl, cycloalkylalkyl, cyano, amino, hydroxyl, alkoxy, —R^{u}N(R^{y})(R^{z}), —R^{u}S(O)ₙR^{x}, —C(O)N(R^{y})(R^{z}), aryl, heterocyclyl, or non-azole heteroaryl;

R^{9a} and R¹⁰ are each optionally substituted with 1-8, 1-6, 1-5, one, two or three Q¹ groups, each independently selected from halo, hydroxyl, alkoxy, cycloalkyl, alkyl, alkenyl, alkynyl, haloalkyl, aryl, heterocyclyl and heteroaryl;

n is 0-2;

m is 0-2; and

wherein the compound is selected such that

a) when A² is N, B³ is NH, R¹ is phenyl, A¹ is CH═CH and R⁸ is H, then R⁶ is not amino;

b) when R¹ thienyl, B¹ is CH, A² is N, B³ is NH, A¹ is CH═CH and R⁸ is H, then R⁶ is not amino; and

c) when R¹ is pyrazol-3-yl; 1,4,5,6-tetrahydrocyclopenta[c]pyrazol-3-yl; or pyridinyl, then B² is not CH, and

d) when R¹ is piperazinyl, then B¹ is not CH.

In certain embodiments, provided herein are compounds of Formula I or pharmaceutically acceptable salts, solvates, hydrates or clathrates thereof, wherein R¹ is substituted aryl, substituted heteroaryl or substituted heterocyclyl; where the substituents are selected from one, two or three R⁹ groups, wherein at least one R⁹ is a branched alkyl, haloalkyl, heterocyclyl or cycloalkyl, and wherein the second and third optional R⁹ groups is selected from halo, alkyl, haloalkyl, cycloalkyl and cycloalkylalkyl, where the alkyl, branched alkyl, haloalkyl, cycloalkyl or cycloalkylalkyl groups are each optionally substituted with 1 to 5 halo, alkyl, cycloalkyl or —R^{u}OC(O)R^{x} groups;

R² and R³ are each independently hydrogen, halo, hydroxy, amino or alkyl;

R⁴ is O, S, N—CN, or N—NO₂;

B¹ is selected from N and CR^{2a};

B² is N or CR^{3a};

R^{2a} and R^{3a} are each independently hydrogen, halo, or alkyl;

R⁵ is halo, alkyl, alkenyl, alkynyl, cycloalkyl, haloalkyl, hydroxyalkyl, alkoxyalkyl, cycloalkylalkyl, cyano, amino, hydroxy, alkoxy, —R^{u}N(R^{y})(R^{z}), aryl, heterocyclyl, or heteroaryl;

R⁶ is hydrogen, halo, alkyl, alkenyl, alkynyl, cycloalkyl, haloalkyl, hydroxyalkyl, alkoxyalkyl, cycloalkylalkyl, cyano, amino, hydroxy, alkoxy, hydroxyalkoxyalkyl, —R^{u}N(R^{y})(R^{z}), aryl, heterocyclyl, or heteroaryl;

B³ is O, NR⁷ or CR^{7a}R^{7a};

R⁷ is hydrogen or alkyl;

each R^{7a} is independently hydrogen or alkyl;

A² and R⁸ are selected as follows:

a) R⁸ is hydrogen, alkyl, alkenyl, alkynyl, cycloalkyl, —R^{u}OR^{x}, —R^{u}N(R^{y})(R^{z}), —R^{u}S(O)ₙR^{x}, heterocyclyl, aryl, or heteroaryl; and A² is N, CH or CR¹⁰; or

b) A² is C; and R⁸ together with A² forms a 5-7 membered substituted or unsubstituted heterocycle, where the substituents when present are one, two or three Q groups, each independently selected from oxo, halo, hydroxyl, alkoxy, alkyl, alkenyl, alkynyl, cycloalkyl, —R^{u}N(R^{y})(R^{z}), aryl, heterocyclyl, heteroaryl, hydroxyalkyl, haloalkyl and alkoxyalkyl;

R⁷ and R⁸ are optionally substituted with one, two or three Q¹ groups, each independently selected from halo, hydroxyl, alkoxy, cycloalkyl, alkyl, alkenyl, alkynyl, haloalkyl, heterocyclyl and heteroaryl;

Q and Q¹ groups are each optionally substituted with 1-6, 1-5, 1-3, one, two or three Q² groups each independently selected from halo, alkyl, alkenyl, alkynyl, cycloalkyl, haloalkyl, amino, hydroxyl and alkoxy;

each R^{u} is independently alkylene or a direct bond;

each R^{x} is independently hydrogen or alkyl;

each R^{y} and R^{z} is independently selected from (i) or (ii) below:

(i) R^{y} and R^{z} are each independently hydrogen, alkyl, alkenyl, alkynyl, cycloalkyl, alkoxyalkyl, or haloalkyl; or

(ii) R^{y} and R^{z}, together with the nitrogen atom to which they are attached, form a heterocyclyl or heteroaryl, optionally substituted with one or more, in one embodiment, one to six, in another embodiment, one, two, three, four or five alkyl groups;

A¹ is N═CR^{9a}, NR^{9a}, S, O, CR^{9a}═CR^{9a}, CR^{9a}═N or N═N;

A³ is N, CH or CR¹⁰;

each R^{9a} is independently hydrogen, halo, alkyl, alkenyl, alkynyl, cycloalkyl, haloalkyl, hydroxyalkyl, alkoxyalkyl, cycloalkylalkyl, cyano, amino, hydroxyl, aryl, —R^{u}N(R^{y})(R^{z}), or alkoxy;

R¹⁰ is halo, alkyl, alkenyl, alkynyl, cycloalkyl, haloalkyl, hydroxyalkyl, alkoxyalkyl, cycloalkylalkyl, cyano, amino, hydroxyl, alkoxy, —R^{u}N(R^{a})(R^{b}), —R^{u}OR^{x}, —R^{u}OR^{x}OR^{x}, —C(O)N(R^{y})(R^{z}), —R^{u}S(O)ₙR^{x}, aryl, heterocyclyl, or non-azole heteroaryl;

R^{9a} and R¹⁰ are each optionally substituted with one, two or three Q¹ groups, each independently selected from halo, hydroxyl, alkoxy, alkyl, alkenyl, alkynyl, or haloalkyl;

n is 0-2; and

m is 0-2.

In certain embodiments, provided herein are compounds of Formula I or pharmaceutically acceptable salts, solvates, hydrates or clathrates thereof, wherein:

R¹ is optionally substituted aryl, optionally substituted heteroaryl or optionally substituted heterocyclyl; where the substituents when present are selected from one, two or three R⁹ groups, wherein each R⁹ is independently selected from halo, alkyl, alkenyl, alkynyl, alkoxy, hydroxyl, haloalkoxy, cycloalkyl, cycloalkylalkyl, hydroxyalkyl, haloalkyl, aryl, arylalkyl, heterocyclyl, heterocyclylalkyl, heteroaryl, and heteroarylalkyl, where the alkyl, alkenyl, alkynyl, alkoxy, haloalkoxy, cycloalkyl, cycloalkylalkyl, hydroxyalkyl, haloalkyl, aryl, heterocyclyl, and heteroaryl groups are optionally substituted with 1 to 5 groups selected from halo, alkyl, alkenyl, alkynyl, aryl, hydroxy, alkoxy, cycloalkyl, cyano, —R^{u}N(R^{y})(R^{z}), —R^{u}S(O)ₙR^{x}—R^{u}C(O)OR^{x} and —R^{u}OC(O)R^{x};

R² and R³ are each independently hydrogen, halo, alkyl, alkenyl, alkynyl or haloalkyl;

R⁴ is O, S, N—CN, or N—NO₂;

B¹ is selected from N and CR^{2a};

B² is selected from N and CR^{3a};

R^{2a} and R^{3a} are each independently hydrogen, halo, alkyl, alkenyl, alkynyl or haloalkyl;

R⁵ is halo, alkyl, alkenyl, alkynyl, cycloalkyl, haloalkyl, hydroxyalkyl, alkoxyalkyl, cycloalkylalkyl, cyano, amino, hydroxy, alkoxy, —R^{u}N(R^{y})(R^{z}), aryl, heterocyclyl, or heteroaryl;

R⁶ is hydrogen, halo or cyano;

B³ is O, NR⁷ or CR^{7a}R^{7a};

R⁷ is hydrogen, alkyl, alkenyl or alkynyl;

each R^{7a} is independently hydrogen, alkyl, alkenyl or alkynyl;

A² and R⁸ are selected as follows:

a) R⁸ is hydrogen, alkyl, alkenyl, alkynyl, cycloalkyl, —OR^{x}, —C₂₋₆alkylene-N(R^{y})(R^{z}), —R^{u}S(O)ₙN(R^{y})(R^{z}), —R^{u}S(O)ₙR^{x}, heterocyclyl, aryl, or heteroaryl; and A² is N, CH or CR¹⁰; or

b) A² is C; and R⁸ together with A² form a 5-7 membered substituted or unsubstituted heterocycle containing one additional heteroatom, where the substituents when present are one, two or three Q groups, each independently selected from oxo, halo, hydroxyl, alkoxy, alkyl, alkenyl, alkynyl, cycloalkyl, —R^{u}N(R^{y})(R^{z}), aryl, heterocyclyl, heteroaryl, hydroxyalkyl, haloalkyl and alkoxyalkyl;

R⁷ and R⁸ are each optionally substituted with 1-6, 1-3, one, two or three Q¹ groups, each independently selected from halo, hydroxyl, alkoxy, cycloalkyl, alkyl, alkenyl, alkynyl, haloalkyl, —R^{u}N(R^{y})(R^{z}), aryl, heterocyclyl and heteroaryl;

Q and Q¹ groups are each optionally substituted with 1-6, 1-5, 1-3, one, two or three Q² groups each independently selected from halo, alkyl, alkenyl, alkynyl, cycloalkyl, haloalkyl, aryl, amino, hydroxyl and alkoxy;

each R^{u} is independently alkylene, alkenylene or alkynylene or a direct bond;

each R^{x} is independently hydrogen, alkyl, alkenyl or alkynyl;

each R^{y} and R^{z} is independently selected from (i) or (ii) below:
(i) R^{y} and R^{z} are each independently hydrogen, alkyl, alkenyl, alkynyl, cycloalkyl, alkoxyalkyl, or haloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five halo, alkyl, haloalkyl, alkenyl or alkynyl groups;

A¹ is N═CR^{9a}, CR^{9a}═CR^{9a} or CR^{9a}═N;

A³ is N, CH or CR¹⁰;

each R^{9a} is independently hydrogen, halo, alkyl or haloalkyl;

R¹⁰ is halo, alkyl, alkenyl, alkynyl, cycloalkyl, haloalkyl, hydroxyalkyl, alkoxyalkyl, cycloalkylalkyl, cyano, amino, hydroxyl, alkoxy, —R^{u}N(R^{a})(R^{b}), —R^{u}OR^{x}, —R^{u}OR^{x}OR^{x}, —C(O)N(R^{y})(R^{z}), —R^{u}S(O)ₙR^{x}, aryl, heterocyclyl, or non-azole heteroaryl;

R^{a} and R^{b} are each independently hydrogen, alkyl, alkenyl, alkynyl, cycloalkyl, alkoxyalkyl, or haloalkyl;

R^{9a} and R¹⁰ are each optionally substituted with 1-8, 1-6, 1-5, one, two or three Q¹ groups, each independently selected from halo, hydroxyl, alkoxy, cycloalkyl, alkyl, alkenyl, alkynyl, haloalkyl, aryl, heterocyclyl and heteroaryl;

n is 0-2; and

m is 0-2.

In certain embodiments, provided herein are compounds of Formula I or pharmaceutically acceptable salts, solvates, hydrates or clathrates thereof, wherein when R¹ is phenyl, then B³—R⁸ is not NH₂. In certain embodiments, provided herein are compounds of Formula I or pharmaceutically acceptable salts, solvates, hydrates or clathrates thereof, wherein when R¹ is phenyl, then R⁶ is not NH₂. In certain embodiments, provided herein are compounds of Formula I or pharmaceutically acceptable salts, solvates, hydrates or clathrates thereof, wherein when R¹ is phenyl, then at least one of B³—R⁸ and R⁶ is not NH₂.

In certain embodiments, provided herein are compounds of Formula I or pharmaceutically acceptable salts, solvates, hydrates or clathrates thereof, wherein when R¹ is phenyl, then —B¹C(R⁴)B²— is not —CHC(O)N—.

In certain embodiments, provided herein are compounds of Formula I or pharmaceutically acceptable salts, solvates, hydrates or clathrates thereof, wherein when R¹ thienyl, A² is N, B³ is NH, A¹ is CH═CH and R⁸ is H, then R⁶ is not amino. In certain embodiments, provided herein are compounds of Formula I or pharmaceutically acceptable salts, solvates, hydrates or clathrates thereof, wherein when R¹ is thienyl, then B³—R⁸ is not NH₂. In certain embodiments, provided herein are compounds of Formula I or pharmaceutically acceptable salts, solvates, hydrates or clathrates thereof, wherein when R¹ is thienyl, then R⁶ is not NH₂. In certain embodiments, provided herein are compounds of Formula I or pharmaceutically acceptable salts, solvates, hydrates or clathrates thereof, wherein when R¹ is thienyl, then at least one of B³—R⁸ and R⁶ is not NH₂.

In certain embodiments, provided herein are compounds of Formula I or pharmaceutically acceptable salts, solvates, hydrates or clathrates thereof, wherein when R¹ is thienyl, then —B¹C(R⁴)B²— is not —CHC(O)N—.

In certain embodiments, provided herein are compounds of Formula I or pharmaceutically acceptable salts, solvates, hydrates or clathrates thereof, wherein when R¹ is pyrazol-3-yl, then B² is not CH. In certain embodiments, provided herein are compounds of Formula I or pharmaceutically acceptable salts, solvates, hydrates or clathrates thereof, wherein when R¹ is pyrazolyl, then B² is not CH. In certain embodiments, provided herein are compounds of Formula I or pharmaceutically acceptable salts, solvates, hydrates or clathrates thereof, wherein when R¹ is pyrazolyl, then —B¹C(R⁴)B²— is not —NC(O)CH—.

In certain embodiments, provided herein are compounds of Formula I or pharmaceutically acceptable salts, solvates, hydrates or clathrates thereof, wherein when R¹ is 1,4,5,6-tetrahydrocyclopenta[c]pyrazol-3-yl, then B² is not CH. In certain embodiments, provided herein are compounds of Formula I or pharmaceutically acceptable salts, solvates, hydrates or clathrates thereof, wherein when R¹ is 1,4,5,6-tetrahydrocyclopenta[c]pyrazol-3-yl, then —B¹C(R⁴)B²— is not —NC(O)CH—.

In certain embodiments, provided herein are compounds of Formula I or pharmaceutically acceptable salts, solvates, hydrates or clathrates thereof, wherein when R¹ is pyridinyl, then B² is not CH. In certain embodiments, provided herein are compounds of Formula I or pharmaceutically acceptable salts, solvates, hydrates or clathrates thereof, wherein when R¹ is pyridinyl, then —B¹C(R⁴)B²— is not —NC(O)CH—.

In certain embodiments, provided herein are compounds of Formula I or pharmaceutically acceptable salts, solvates, hydrates or clathrates thereof, wherein when R¹ is piperazinyl, then B¹ is not CH. In certain embodiments, provided herein are compounds of Formula I or pharmaceutically acceptable salts, solvates, hydrates or clathrates thereof, wherein when R¹ is piperazinyl, then —B¹C(R⁴)B²— is not —CHC(O)N—.

In certain embodiments, provided herein are compounds of Formula I or pharmaceutically acceptable salts, solvates, hydrates or clathrates thereof, wherein R¹ is substituted aryl, substituted heteroaryl or substituted heterocyclyl; where the substituents are selected from one, two or three R⁹ groups, wherein at least one R⁹ is a branched alkyl, cycloalkyl, haloalkyl or heterocyclyl, and wherein the second and third optional R⁹ groups is selected from halo, alkyl, haloalkyl, cycloalkyl and cycloalkylalkyl, where the alkyl, branched alkyl, haloalkyl, cycloalkyl or cycloalkylalkyl groups are each optionally substituted with 1 to 5 groups selected from halo, alkyl, cycloalkyl and —R^{u}OC(O)R^{x}.

In certain embodiments, provided herein are compounds of Formula I wherein, when R⁹ is a branched alkyl, hydroxyalkyl, haloalkyl, heterocyclyl or cycloalkyl, R⁹ is selected from —CH(CH₃)₂, —C(CH₃)₂CH₂OH, —CF₃, —C(CH₃)₃, —CF₂(CH₃), —C(CH₃)(CH₂F)₂, —C(CH₃)₂CF₃, —C(CH₃)₂CH₂F, —CF(CH₃)₂, cyclopropyl, cyclobutyl, cyclopentyl, cyclohexyl,

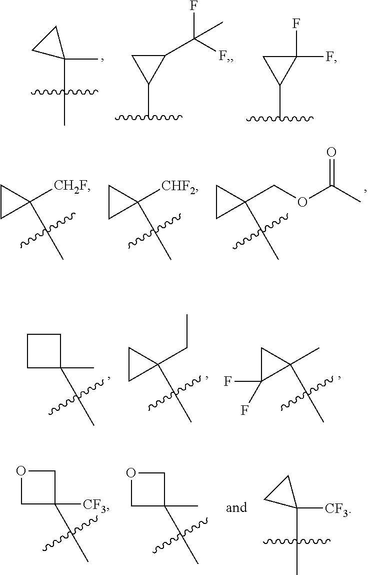

In certain embodiments, provided herein are compounds of Formula I wherein R¹ is substituted aryl or substituted heteroaryl. In certain embodiments, provided herein are compounds of Formula I wherein R¹ is substituted azolyl. In certain embodiments, provided herein are compounds of Formula I, wherein R¹ is substituted phenyl or substituted isoxazolyl. In certain embodiments, provided herein are compounds of Formula I, wherein A¹ is N═CR^{9a} or CR^{9a}═CR^{9a} and A² and A³ are each CH or CR¹⁰. The compound of Formula I, wherein B³ is NH or CR^{7a}R^{7a}. In certain embodiments, provided herein are compounds of Formula I, wherein B³ is NH. In yet other certain embodiments, provided herein are compounds of Formula I wherein R¹ is substituted aryl or substituted heteroaryl, A¹ is N═CR^{9a}, S or CR^{9a}═CR^{9a} and A² and A³ are each CH or CR¹⁰. In yet other embodiments, provided herein are compounds of Formula I wherein R¹ is substituted aryl or substituted heteroaryl, A¹ is N═CR^{9a}, S or CR^{9a}═CR^{9a} and A² and A³ are each CH or CR¹⁰ and when R⁹ is a branched alkyl, hydroxyalkyl, haloalkyl, heterocyclyl or cycloalkyl, R⁹ is selected from —CH(CH₃)₂, —C(CH₃)₂CH₂OH, —CF₃, —C(CH₃)₃, —CF₂(CH₃), —C(CH₃)(CH₂F)₂, —C(CH₃)₂CF₃, —C(CH₃)₂CH₂F, —CF(CH₃)₂, cyclopropyl, cyclobutyl, cyclopentyl, cyclohexyl,

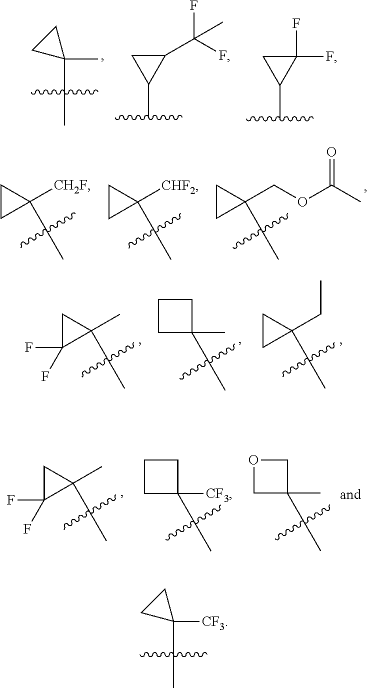

In yet other certain embodiments, provided herein are compounds of Formula I wherein R¹ is substituted aryl or substituted heteroaryl, A¹ is N═CR^{9a}, S or CR^{9a}═CR^{9a} and A² and A³ are each CH or CR¹⁰ and when R⁹ is a branched alkyl, hydroxyalkyl, haloalkyl, heterocyclyl or cycloalkyl, R⁹ is selected from —CH(CH₃)₂, —C(CH₃)₂CH₂OH, —CF₃, —C(CH₃)₃, —CF₂(CH₃), —C(CH₃)(CH₂F)₂, —C(CH₃)₂CF₃, —C(CH₃)₂CH₂F, —CF(CH₃)₂, cyclopropyl, cyclobutyl, cyclopentyl, cyclohexyl,

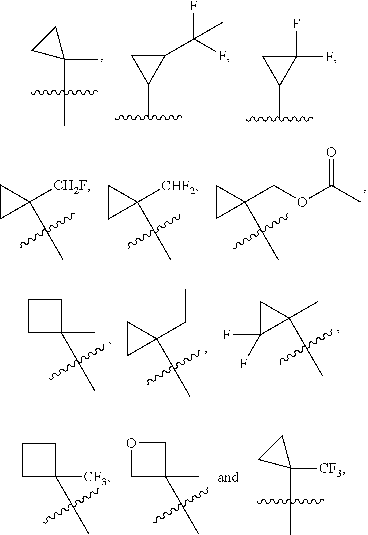

and the optional second or third R⁹ is halo, alkyl, haloalkyl, alkoxy or haloalkoxy.

In certain embodiments, provided herein are compounds of Formula II:

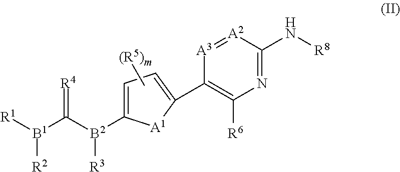

or pharmaceutically acceptable salts, solvates, hydrates or clathrates thereof, wherein:

R¹ is optionally substituted aryl, optionally substituted heteroaryl or optionally substituted heterocyclyl; where the substituents when present are selected from one, two or three R⁹ groups, wherein each R⁹ is independently selected from halo, alkyl, alkenyl, alkynyl, alkoxy, hydroxyl, haloalkoxy, heterocyclyl and cycloalkyl, where the alkyl, alkenyl, alkynyl, alkoxy, haloalkoxy, heterocyclyl and cycloalkyl groups are optionally substituted with 1 to 5 groups selected from halo, alkyl, haloalkyl, alkoxyalkyl, hydroxy, alkoxy, cycloalkyl and —R^{u}OC(O)R^{x};

R² and R³ are each independently hydrogen, halo, hydroxy, haloalkyl or alkyl;

R⁴ is O or S;

B¹ is selected from N and CR^{2a};

B² is N or CR^{3a};

R^{2a} and R^{3a} are each independently hydrogen, halo, or alkyl;

A¹ is N═CR^{9a}, S or CR^{9a}═CR^{9a};

R⁵ is halo, alkyl, alkenyl, alkynyl, cycloalkyl, haloalkyl, hydroxyalkyl, alkoxyalkyl, cycloalkylalkyl, cyano, amino, hydroxyl or alkoxy;

R⁶ is hydrogen, halo, alkyl, alkenyl, alkynyl, cycloalkyl, haloalkyl, hydroxyalkyl, alkoxyalkyl, hydroxyalkoxyalkyl, heterocyclylalkyl, cycloalkylalkyl, cyano, amino, hydroxyl or alkoxy;

A² and R⁸ are selected as follows:

a) R⁸ is hydrogen, alkyl, alkenyl, alkynyl, cycloalkyl, —R^{u}OR^{x}, —R^{u}N(R^{y})(R^{z}), —R^{u}S(O)ₙR^{x}, heterocyclyl, aryl, or heteroaryl; and A² is N, CH or CR¹⁰; or

b) A² is C; and R⁸ together with A² forms a 5-7 membered substituted or unsubstituted heterocyclyl, where the substituents when present are one, two or three Q groups, each independently selected from oxo, halo, hydroxyl, alkoxy, alkyl, alkenyl, alkynyl, cycloalkyl, heterocyclyl, heteroaryl, hydroxyalkyl, haloalkyl and alkoxyalkyl;

R⁸ is optionally substituted with one, two or three Q¹ groups, each independently selected from halo, hydroxyl, alkoxy, cycloalkyl, alkyl, alkenyl, alkynyl, haloalkyl, heterocyclyl and heteroaryl;

Q and Q¹ groups are each optionally substituted with 1-6, 1-5, one, two or three Q² groups each independently selected from halo, alkyl, alkenyl, alkynyl, cycloalkyl, haloalkyl, amino, hydroxyl and alkoxy;

each R^{u} is independently alkylene or a direct bond;

each R^{x} is independently hydrogen or alkyl;

each R^{y} and R^{z} is independently selected from (i) or (ii) below:
(i) R^{y} and R^{z} are each independently hydrogen, alkyl, alkenyl, alkynyl, or cycloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five alkyl groups;

A³ is N, CH or CR¹⁰;

R^{9a} is hydrogen, halo or alkyl;

each R¹⁰ is independently alkyl, hydroxyalkyl, cyano, —R^{u}N(R^{a})(R^{b}), —R^{u}S(O)ₙR^{x} or —C(O)N(R^{y})(R^{z});

n is 0-2;

m is 0-2; and

wherein the compound is selected with a proviso that when A² is N, R¹ is phenyl, A¹ is CH═CH and R⁸ is H, then R⁶ is not amino.

In certain embodiments, provided herein are compounds of Formula II, or pharmaceutically acceptable salts, solvates, hydrates or clathrates thereof, wherein:

R¹ is substituted aryl, substituted heteroaryl or substituted heterocyclyl; where the substituents are selected from one, two or three R⁹ groups, wherein at least one R⁹ is a branched alkyl, haloalkyl, heterocyclyl or cycloalkyl, and wherein the second and third optional R⁹ groups is selected from halo, alkyl, haloalkyl, cycloalkyl and cycloalkylalkyl, where the alkyl, branched alkyl, haloalkyl, cycloalkyl or cycloalkylalkyl groups are each optionally substituted with 1 to 5 groups selected from halo, hydroxy, alkyl, cycloalkyl and —R^{u}OC(O)R^{x};

R² and R³ are each independently hydrogen, halo, haloalkyl, hydroxy, amino or alkyl;

R⁴ is O, S, N—CN, or N—NO₂;

A¹ is N═CR^{9a}, NR^{9a}, S, O, CR^{9a}═CR^{9a}, CR^{9a}═N or N═N;

B¹ is N or CR^{2a};

B² is N or CR^{3a};

R^{2a} and R^{3a} are each independently hydrogen, halo, or alkyl;

R⁵ is halo, alkyl, alkenyl, alkynyl, cycloalkyl, haloalkyl, hydroxyalkyl, alkoxyalkyl, cycloalkylalkyl, cyano, amino, hydroxy, alkoxy, —R^{u}N(R^{y})(R^{z}), aryl, heterocyclyl, or heteroaryl;

R⁶ is hydrogen, halo, alkyl, alkenyl, alkynyl, cycloalkyl, haloalkyl, hydroxyalkyl, alkoxyalkyl, cycloalkylalkyl, cyano, amino, hydroxy, alkoxy, —R^{u}N(R^{y})(R^{z}), aryl, heterocyclyl, or heteroaryl;

A² and R⁸ are selected as follows:

a) R⁸ is hydrogen, alkyl, alkenyl, alkynyl, cycloalkyl, —R^{u}OR^{x}, —R^{u}N(R^{y})(R^{z}), —R^{u}S(O)ₙR^{x}, heterocyclyl, aryl, or heteroaryl; and A² is N, CH or CR¹⁰; or

b) A² is C; and R⁸ together with A² forms a 5-7 membered substituted or unsubstituted heterocyclyl, where the substituents when present are one, two or three Q groups, each independently selected from oxo, halo, hydroxyl, alkoxy, alkyl, alkenyl, alkynyl, cycloalkyl, heterocyclyl, heteroaryl, hydroxyalkyl, haloalkyl and alkoxyalkyl;

R⁸ is optionally substituted with 1-6, 1-4, one, two or three Q¹ groups, each independently selected from halo, hydroxyl, alkoxy, cycloalkyl, alkyl, alkenyl, alkynyl, haloalkyl, heterocyclyl and heteroaryl;

Q and Q¹ groups are each optionally substituted with 1-6, 1-5, 1-4, one, two or three Q² groups each independently selected from halo, alkyl, alkenyl, alkynyl, cycloalkyl, haloalkyl, amino, hydroxyl and alkoxy;

each R^{u} is independently alkylene or a direct bond;

each R^{x} is independently hydrogen or alkyl;

each R^{y} and R^{z} is independently selected from (i) or (ii) below:
(i) R^{y} and R^{z} are each independently hydrogen, alkyl, alkenyl, alkynyl, or cycloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five halo, haloalkyl or alkyl groups;

A³ is N, CH or CR¹⁰;

each R^{9a} is independently hydrogen, halo, alkyl, alkenyl, alkynyl, cycloalkyl, haloalkyl, hydroxyalkyl, alkoxyalkyl, cycloalkylalkyl, cyano, amino, hydroxyl, aryl, —R^{u}N(R^{y})(R^{z}), or alkoxy;

R¹⁰ is halo, alkyl, alkenyl, alkynyl, cycloalkyl, haloalkyl, hydroxyalkyl, alkoxyalkyl, cycloalkylalkyl, cyano, amino, hydroxyl, alkoxy, —R^{u}N(R^{a})(R^{b}), —R^{u}OR^{x}, —R^{u}OR^{x}OR^{x}, —C(O)NR^{y}R^{z}, —R^{u}S(O)ₙR^{x}, aryl, heterocyclyl, or heteroaryl;

n is 0-2; and

m is 0-2.

In one embodiment, provided herein are compounds of Formula II, wherein R¹ is substituted phenyl, substituted isoxazolyl or substituted pyrazolyl. In one embodiment, R¹ is substituted isoxazolyl. In certain embodiments, provided herein are compounds of Formula II wherein R¹ is optionally substituted phenyl, optionally substituted isoxazolyl, optionally substituted 1-pyrazolyl or optionally substituted 5-pyrazolyl.

In certain embodiments, provided herein are compounds of Formula II wherein when R⁹ is a branched alkyl, hydroxyalkyl, haloalkyl, heterocyclyl or cycloalkyl, R⁹ is selected from —CH(CH₃)₂, —C(CH₃)₂CH₂OH, —CF₃, —C(CH₃)₃, —CF₂(CH₃), —C(CH₃)(CH₂F)₂, —C(CH₃)₂CF₃, —C(CH₃)₂CH₂F, —CF(CH₃)₂, cyclopropyl, cyclobutyl, cyclopentyl, cyclohexyl,

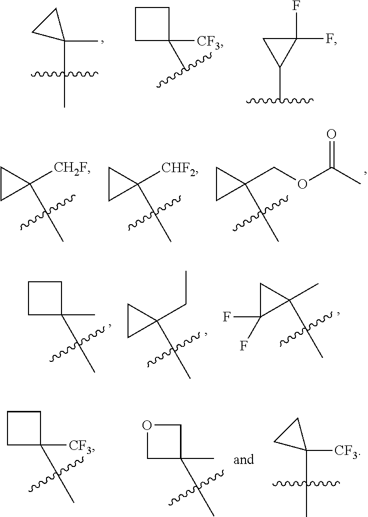

In certain embodiments, provided herein are compounds of Formula II wherein R¹ is substituted aryl or substituted heteroaryl. In certain embodiments, provided herein are compounds of Formula II wherein R¹ is substituted azolyl. In certain embodiments, provided herein are compounds of Formula II, wherein R¹ is substituted phenyl or substituted isoxazolyl. In certain embodiments, provided herein are compounds of Formula II, wherein A¹ is N═CR^{9a}, S or CR^{9a}═CR^{9a} and A² and A³ are each CH or CR¹⁰. In certain embodiments, provided herein are compounds of Formula II wherein A¹ is N═CR^{9a} or CR^{9a}═CR^{9a} and A² and A³ are each CH or CR¹⁰. In yet other certain embodiments, provided herein are compounds of Formula I, wherein R¹ is substituted aryl or substituted heteroaryl, A¹ is N═CR^{9a}, S or CR^{9a}═CR^{9a} and A² and A³ are each CH or CR¹⁰. In yet other certain embodiments, provided herein are compounds of Formula I, wherein R¹ is substituted aryl or substituted heteroaryl, A¹ is N═CR^{9a}, S or CR^{9a}═CR^{9a} and A² and A³ are each CH or CR¹⁰ and R⁹ is selected from —CH(CH₃)₂, —C(CH₃)₂CH₂OH, —CF₃, —C(CH₃)₃, —CF₂(CH₃), —C(CH₃)(CH₂F)₂, —C(CH₃)₂CF₃, —C(CH₃)₂CH₂F, —CF(CH₃)₂, cyclopropyl, cyclobutyl, cyclopentyl, cyclohexyl,

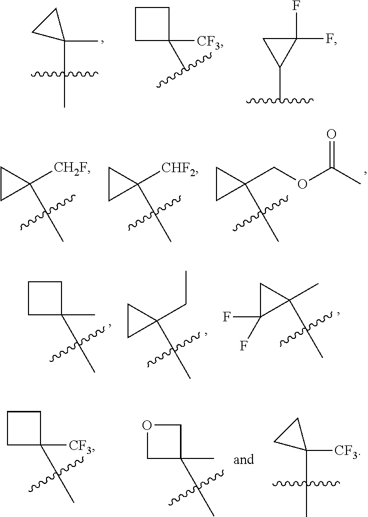

In certain embodiments, provided herein are compounds of Formula II, or pharmaceutically acceptable salts, solvates, hydrates or clathrates thereof, wherein:

R¹ is optionally substituted azolyl; where the substituents when present are selected from one, two or three R⁹ groups, wherein each R⁹ is independently selected from halo, cycloalkyl and alkyl, where alkyl and cycloalkyl are each optionally substituted with 1 to 5 groups selected from halo, alkyl, hydroxy, heterocyclyl and cycloalkyl;

R² and R³ are each independently hydrogen, halo, hydroxy, amino or alkyl;

B¹ is N or CR^{2a};

B² is N or CR^{3a};

R^{2a} and R^{3a} are each independently hydrogen, halo, or alkyl;

R⁴ is O;

A¹ is N═CR^{9a}, S or CR^{9a}═CR^{9a};

R⁵ is halo, alkyl, haloalkyl, or alkoxy;

R⁶ is hydrogen, halo, alkyl or alkoxy;

A² and R⁸ are selected as follows:

a) R⁸ is hydrogen, alkyl, alkenyl, alkynyl, cycloalkyl, —R^{u}OR^{x}, —R^{u}N(R^{y})(R^{z}), —R^{u}S(O)ₙR^{x}, heterocyclyl, aryl, or heteroaryl; and A² is N, CH or CR¹⁰; or

b) A² is C; and R⁸ together with A² forms a 5-7 membered substituted or unsubstituted heterocyclyl, where the substituents when present are one, two or three Q groups, each independently selected from oxo, halo, hydroxyl, alkoxy, alkyl, alkenyl, alkynyl, cycloalkyl, heterocyclyl, heteroaryl, hydroxyalkyl, haloalkyl and alkoxyalkyl;

R⁸ is optionally substituted with 1-6, 1-5, one, two or three Q¹ groups, each independently selected from halo, hydroxyl, alkoxy, cycloalkyl, alkyl, alkenyl, alkynyl, haloalkyl, heterocyclyl and heteroaryl;

Q and Q¹ groups are each optionally substituted with 1-6, 1-5, 1-4, one, two or three Q² groups each independently selected from halo, alkyl, alkenyl, alkynyl, cycloalkyl, haloalkyl, amino, hydroxyl and alkoxy;

each R^{u} is independently alkylene or a direct bond;

each R^{x} is independently hydrogen or alkyl;

each R^{y} and R^{z} is independently selected from (i) or (ii) below:
(i) R^{y} and R^{z} are each independently hydrogen, alkyl, alkenyl, alkynyl, or cycloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five alkyl groups;

A³ is CH or CR¹⁰;

R^{9a} is hydrogen, halo or alkyl;

each R¹⁰ is independently alkyl, hydroxyalkyl, cyano, —R^{u}N(R^{a})(R^{b}), —R^{u}OR^{x}, —R^{u}OR^{x}OR^{x}, —R^{u}S(O)ₙR^{x}, —C(O)N(R^{y})(R^{z});

R^{a} and R^{b} are each independently hydrogen or alkyl;

n is 0-2; and

m is 0 or 1.

In certain embodiments, provided herein are compounds of Formula II, wherein:

R¹ is optionally substituted aryl; where the substituents when present are selected from one, two or three R⁹ groups, wherein each R⁹ is independently selected from halo, cycloalkyl and alkyl, where the alkyl and cycloalkyl are optionally substituted with 1 to 5 groups selected from halo, alkyl and cycloalkyl;

R² and R³ are each independently hydrogen, halo, hydroxy, amino or alkyl;

B¹ is N or CR^{2a};

B² is N or CR^{3a};

R^{2a} and R^{3a} are each independently hydrogen, halo, or alkyl;

R⁴ is O;

A¹ is N═CR^{9a}, S or CR^{9a}═CR^{9a};

R⁵ is halo, alkyl, haloalkyl or alkoxy;

R⁶ is hydrogen, halo, alkyl or alkoxy;

R⁸ is hydrogen, alkyl, alkenyl, alkynyl, cycloalkyl, —R^{u}OR^{x}, —R^{u}N(R^{y})(R^{z}), —R^{u}S(O)ₙR^{x}, heterocyclyl, aryl, or heteroaryl; R⁸ is optionally substituted with 1-6, 1-5, 1-4, one, two or three Q¹ groups, each independently selected from halo, hydroxyl, alkoxy, cycloalkyl, alkyl, alkenyl, alkynyl, haloalkyl, heterocyclyl and heteroaryl;

Q and Q¹ groups are each optionally substituted with 1-6, 1-5, 1-4, one, two or three Q² groups each independently selected from halo, alkyl, alkenyl, alkynyl, cycloalkyl, haloalkyl, amino, hydroxyl and alkoxy;

each R^{u} is independently alkylene or a direct bond;

each R^{x} is independently hydrogen or alkyl;

each R^{y} and R^{z} is independently selected from (i) or (ii) below:
(i) R^{y} and R^{z} are each independently hydrogen, alkyl, alkenyl, alkynyl, or cycloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five alkyl groups;

A² is N;

A³ is CH or CR¹⁰;

R^{9a} is hydrogen, halo or alkyl;

R¹⁰ is alkyl, hydroxyalkyl, cyano, —R^{u}N(R^{a})(R^{b}), —R^{u}OR^{x}, —R^{u}OR^{x}OR^{x}, —R^{u}S(O)ₙR^{x}, or —C(O)N(R^{y})(R^{z}) where R^{u} is alkylene, and R^{a} and R^{b} are each hydrogen;

n is 0-2; and

m is 0 or 1.

In certain embodiments, provided herein are compounds of Formula I, II or III wherein A² is C; and R⁸ together with A² form a 5-7 membered substituted or unsubstituted heterocycle with one additional heteroatom, where the substituents when present are one, two or three Q groups, each independently selected from oxo, halo, hydroxyl, alkoxy, alkyl, alkenyl, alkynyl, cycloalkyl, —R^{u}N(R^{y})(R^{z}), —R^{u}S(O)ₙR^{x}, aryl, heterocyclyl, heteroaryl, hydroxyalkyl, haloalkyl and alkoxyalkyl. In certain embodiments, the one additional heteroatom is O, S(O) or S(O)₂. In certain embodiments, provided herein are compounds of Formula II wherein A² is C; and R⁸ together with A² form a 5-7 membered substituted or unsubstituted heterocycle with one additional heteroatom, where the substituents when present are one, two or three Q groups, each independently selected from oxo, halo, hydroxyl, alkoxy, alkyl, alkenyl, alkynyl, cycloalkyl, —R^{u}N(R^{y})(R^{z}), —R^{u}S(O)ₙR^{x}, aryl, heterocyclyl, heteroaryl, hydroxyalkyl, haloalkyl and alkoxyalkyl. In certain embodiments, the one additional heteroatom is O, S(O) or S(O)₂. In certain embodiments, provided herein are compounds of Formula II wherein A² is C; and R⁸ together with A² form a 5-7 membered substituted or unsubstituted heterocycle with one additional oxygen heteroatom, where the substituents when present are one, two or three Q groups, each independently selected from oxo, halo, hydroxyl, alkoxy, alkyl, alkenyl, alkynyl, cycloalkyl, —R^{u}N(R^{y})(R^{z}), —R^{u}S(O)ₙR^{x}, aryl, heterocyclyl, heteroaryl, hydroxyalkyl, haloalkyl and alkoxyalkyl.

In certain embodiment, provided herein are compounds of Formula I, II or III, wherein R¹ is

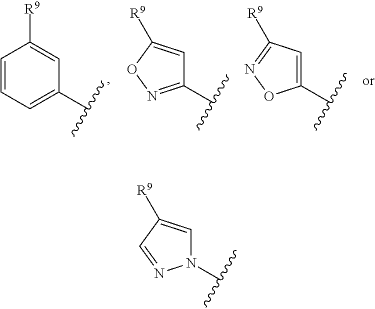

wherein R⁹ is alkyl, haloalkyl, cycloalkyl, cycloalkylalkyl, heterocyclyl or heterocyclylalkyl optionally substituted with 1 to 5 groups selected from halo, alkyl, alkenyl, alkynyl, haloalkyl, alkoxyalkyl, hydroxy, alkoxy, cycloalkyl, cyano, —R^{u}N(R^{y})(R^{z}), —R^{u}S(O)ₙR^{x}, —R^{u}C(O)OR^{x} and —R^{u}OC(O)R^{x};

B¹ is N and B² is selected from N and CR^{3a};

R² is H;

R³ is hydrogen, halo, haloalkyl, hydroxy, alkyl, alkenyl, alkynyl, alkoxy or amino; R^{3a} is hydrogen, halo, haloalkyl, hydroxy, alkyl, alkenyl, alkynyl, alkoxy or amino and the other variables are as described elsewhere herein.

In certain embodiment, provided herein are compounds of Formula I, wherein R¹ is

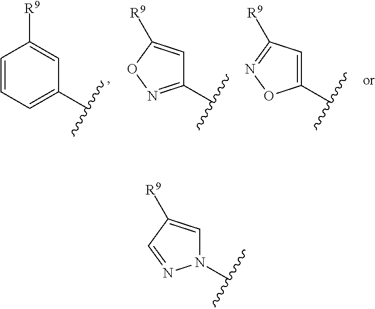

wherein R⁹ is alkyl, haloalkyl, cycloalkyl, cycloalkylalkyl, heterocyclyl or heterocyclylalkyl optionally substituted with 1 to 5 groups selected from halo, alkyl, alkenyl, alkynyl, haloalkyl, alkoxyalkyl, hydroxy, alkoxy, cycloalkyl, cyano, —R^{u}N(R^{y})(R^{z}), —R^{u}S(O)ₙR^{x}, —R^{u}C(O)OR^{x} and —R^{u}OC(O)R^{x};

B¹ is N and B² is selected from N and CR^{3a};

R² is H;

R³ is independently hydrogen, halo, alkyl, alkenyl, alkynyl or haloalkyl; R^{3a} is independently hydrogen, halo, alkyl, and the other variables are as described elsewhere herein.

In one embodiment, provided herein are compounds of Formula III:

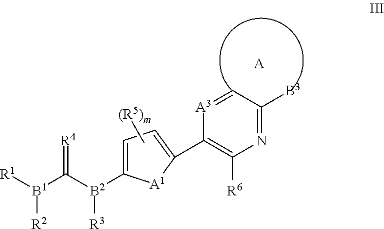

or pharmaceutically acceptable salts, solvates, hydrates or clathrates thereof, wherein the variables are as described elsewhere herein. In one embodiment, provided herein are compounds of Formula III or pharmaceutically acceptable salts, solvates, hydrates or clathrates thereof, wherein:

R¹ is optionally substituted aryl, heteroaryl or heterocyclyl; where the substituents when present are selected from one, two or three R⁹ groups, wherein each R⁹ is independently selected from halo, alkyl, alkenyl, alkynyl, alkoxy, hydroxyl, haloalkoxy, heterocyclyl and cycloalkyl, where the alkyl, alkenyl, alkynyl, alkoxy, haloalkoxy, and cycloalkyl groups are optionally substituted with 1 to 5 groups selected from halo, haloalkyl, alkoxyalkyl, hydroxy, alkoxy and cycloalkyl;

R² and R³ are each independently hydrogen, halo, hydroxy, amino or alkyl;

B¹ is N or CR^{2a};

B² is N or CR^{3a};

R^{2a} and R^{3a} are each independently hydrogen, halo, or alkyl;

R⁴ is O or S;

A¹ is N═CR^{9a}, S, CR^{9a}═CR^{9a} or CR^{9a}═N;

R⁵ is halo, alkyl, alkenyl, alkynyl, cycloalkyl, haloalkyl, hydroxyalkyl, alkoxyalkyl, cycloalkylalkyl, cyano, amino, hydroxyl or alkoxy;

R⁶ is hydrogen, halo, alkyl, alkenyl, alkynyl, cycloalkyl, haloalkyl, hydroxyalkyl, alkoxyalkyl, hydroxyalkoxyalkyl, heterocyclylalkyl, cycloalkylalkyl, cyano, amino, hydroxyl or alkoxy;

B³ is NR⁷;

R⁷ is hydrogen or alkyl;

ring A is a 5-7 membered heterocyclyl optionally substituted with one, two or three Q groups, each independently selected from oxo, halo, hydroxyl, alkoxy, alkyl, alkenyl, alkynyl, cycloalkyl, heterocyclyl, heteroaryl, hydroxyalkyl, haloalkyl and alkoxyalkyl;

each Q is optionally substituted with one, two or three Q² groups each independently selected from halo, alkyl, alkenyl, alkynyl, cycloalkyl, haloalkyl, hydroxyl and alkoxy;

A³ is N, CH or CR¹⁰;

R^{9a} is hydrogen, halo or alkyl;

R¹⁰ is alkyl, hydroxyalkyl, cyano, —R^{u}N(R^{a})(R^{b}), —R^{u}OR^{x}, —R^{u}OR^{x}OR^{x}, —R^{u}S(O)ₙR^{x}, or —C(O)N(R^{y})(R^{z}) where R^{u} is direct bond or alkylene, and R^{a} and R^{b} are each hydrogen;

each R^{x} is independently hydrogen, alkyl, alkenyl or alkynyl;

each R^{y} and R^{z} is independently selected from (i) or (ii) below:
(i) R^{y} and R^{z} are each independently hydrogen, alkyl, alkenyl, alkynyl, cycloalkyl, alkoxyalkyl, or haloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five halo, alkyl, haloalkyl, alkenyl or alkynyl groups;

n is 0-2; and

m is 0-2.

In certain embodiments, provided herein are compounds of Formula III wherein R¹⁰ is alkyl, hydroxyalkyl, cyano, —R^{u}N(R^{y})(R^{z}), where R^{u} is alkylene and R^{y} and R^{z} together form a heteroaryl or heterocyclyl ring. In certain embodiments, provided herein are compounds of Formula III wherein A¹ is N═CR^{9a}, S or CR^{9a}═CR^{9a}. In certain embodiments, provided herein are compounds of Formula III wherein R¹ is isoxazolyl, phenyl or pyrazolyl. In certain embodiments, provided herein are compounds of Formula III wherein R¹ is isoxazolyl, phenyl, 1-pyrazolyl or 5-pyrazolyl.

In certain embodiments, the compounds provided herein are selected such that when A² is N, B³ is NH, R¹ is aryl, A¹ is CH═CH and R⁸ is H, then R⁶ is not amino. In certain embodiments, the compounds provided herein are selected such that when A² is N, B³ is NH, R¹ is aryl, A¹ is CR^{9a}═CR^{9a} and R⁸ is H, then R⁶ is not amino. In certain embodiments, the compounds provided herein are selected such that when A² is N, B³ is NH, and R⁸ is H, then R⁶ is not amino. In certain embodiments, the compounds provided herein are selected such that when A² is N, B³ is NH, then R⁶ is not amino. In certain embodiments, the compounds provided herein are selected such that when A² is N, R⁸ is H, then R⁶ is not amino. In certain embodiments, the compounds provided herein are selected such that when A² is N, then R⁶ is not amino.

In certain embodiments, provided herein are compounds of Formula IVA, IVB, IVC or IVD:

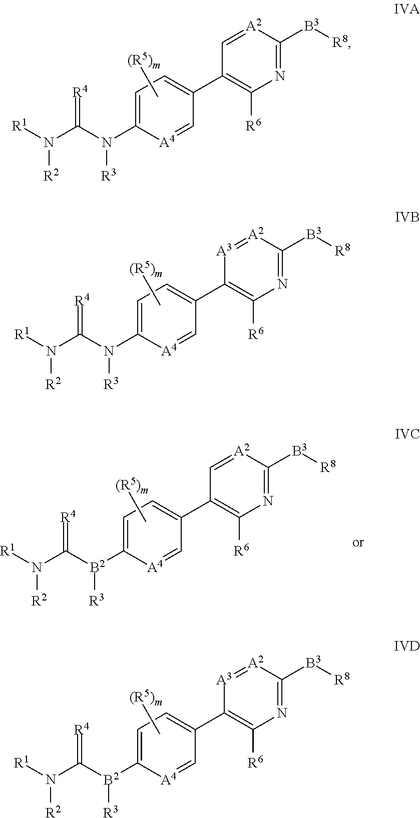

or pharmaceutically acceptable salts, solvates, hydrates or clathrates thereof, wherein the variables are as described elsewhere herein. In certain embodiments, provided herein are compounds of Formula IVA, IVB, IVC or IVD or pharmaceutically acceptable salts, solvates, hydrates or clathrates thereof, wherein R¹ is isoxazolyl, 1-pyrazolyl or 5-pyrazolyl, and the other variables are as described elsewhere herein

In one embodiment, provided herein are compounds of Formula IVA, IVB, IVC or IVD or pharmaceutically acceptable salts, solvates, hydrates or clathrates thereof, wherein:

R¹ is optionally substituted 5 to 6 membered aryl or heteroaryl; where the substituents when present are selected from one, two or three R⁹ groups, wherein each R⁹ is independently selected from halo, alkyl and cycloalkyl, where the alkyl and cycloalkyl is optionally substituted with 1 to 5 groups selected from halo, hydroxy and cycloalkyl;

A⁴ is N, or CR^{9a};

R² and R³ are each independently hydrogen, halo, hydroxy, amino or alkyl;

B² is CR^{3a};

R^{3a} is hydrogen, halo, or alkyl;

R⁴ is O or S;

R⁵ is halo, alkyl, haloalkyl or alkoxy;

R⁶ is hydrogen, halo, alkyl or alkoxy;

B³ is O, NH, or CH₂;

A² and R⁸ are selected as follows:

a) R⁸ is hydrogen, alkyl, alkenyl, alkynyl, cycloalkyl, —R^{u}OR^{x}, —R^{u}N(R^{y})(R^{z}), —R^{u}S(O)ₙR^{x}, heterocyclyl, aryl, or heteroaryl; and A² is N, CH or CR¹⁰; or

b) A² is C; and R⁸ together with A² forms a 5-7 membered substituted or unsubstituted heterocyclyl, where the substituents when present are one, two or three Q groups, each independently selected from oxo, halo, hydroxyl, alkoxy, alkyl, alkenyl, alkynyl, cycloalkyl, heterocyclyl, heteroaryl, hydroxyalkyl, haloalkyl and alkoxyalkyl;

R⁸ is optionally substituted with one, two or three Q¹ groups, each independently selected from halo, hydroxyl, alkoxy, cycloalkyl, alkyl, alkenyl, alkynyl, haloalkyl, heterocyclyl and heteroaryl;

Q and Q¹ groups are each optionally substituted with 1-6, 1-5, 1-3, one, two or three Q² groups each independently selected from halo, alkyl, alkenyl, alkynyl, cycloalkyl, haloalkyl, amino, hydroxyl and alkoxy;

each R^{u} is independently alkylene or a direct bond;

each R^{x} is independently hydrogen or alkyl;

each R^{y} and R^{z} is independently selected from (i) or (ii) below:
(i) R^{y} and R^{z} are each independently hydrogen, alkyl, alkenyl, alkynyl, or cycloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five alkyl or halo groups;

A³ is N, CH or CR^{10a};

R^{9a} is hydrogen, halo or alkyl;

R¹⁰ is alkyl, hydroxyalkyl, cyano, —R^{u}N(R^{a})(R^{b}), —R^{u}OR^{x}, —R^{u}OR^{x}OR^{x}, —R^{u}S(O)ₙR^{x}, or —R^{u}N(R^{y})(R^{z});

R^{a} and R^{b} are each independently hydrogen or alkyl;

R^{10a} is alkyl, haloalkyl, alkoxy or halo;

n is 0-2;

m is 0 or 1 and other variables are as described elsewhere herein.

In certain embodiments, provided herein are compounds of Formula IVA, IVB, IVC or IVD or pharmaceutically acceptable salts, solvates, hydrates or clathrates thereof, wherein

R¹ is substituted 5- to 6-membered aryl or substituted 5- to 6-membered heteroaryl where the substituents are selected from one, two or three R⁹ groups, wherein at least one R⁹ is a branched alkyl, haloalkyl, heterocyclyl or cycloalkyl and wherein the second and third optional R⁹ groups is selected from halo, alkyl, haloalkyl, cycloalkyl and cycloalkylalkyl, where the alkyl, branched alkyl, haloalkyl, cycloalkyl or cycloalkylalkyl groups are each optionally substituted with 1 to 5 groups selected from halo, hydroxy, alkyl, heterocyclyl or cycloalkyl;

A⁴ is N, or CR^{9a};

R² and R³ are each independently hydrogen, halo, hydroxy, amino or alkyl;

B² is CR^{3a};

R^{3a} is hydrogen, halo, or alkyl;

R⁴ is O or S;

R⁵ is halo, alkyl, haloalkyl or alkoxy;

R⁶ is hydrogen, halo, alkyl or alkoxy;

B³ is O, NH, or CH₂;

A² and R⁸ are selected as follows:

a) R⁸ is hydrogen, alkyl, alkenyl, alkynyl, cycloalkyl, —R^{u}OR^{x}, —R^{u}N(R^{y})(R^{z}), —R^{u}S(O)ₙR^{x}, heterocyclyl, aryl, or heteroaryl; and A² is N, CH or CR¹⁰; or

b) A² is C; and R⁸ together with A² forms a 5-7 membered substituted or unsubstituted heterocyclyl, where the substituents when present are one, two or three Q groups, each independently selected from oxo, halo, hydroxyl, alkoxy, alkyl, alkenyl, alkynyl, cycloalkyl, heterocyclyl, heteroaryl, hydroxyalkyl, haloalkyl and alkoxyalkyl;

R⁸ is optionally substituted with one, two or three Q¹ groups, each independently selected from halo, hydroxyl, alkoxy, cycloalkyl, alkyl, alkenyl, alkynyl, haloalkyl, heterocyclyl and heteroaryl;

Q and Q¹ groups are each optionally substituted with 1-6, 1-5, 1-3, one, two or three Q² groups each independently selected from halo, alkyl, alkenyl, alkynyl, cycloalkyl, haloalkyl, amino, hydroxyl and alkoxy;

each R^{u} is independently alkylene or a direct bond;

each R^{x} is independently hydrogen or alkyl;

each R^{y} and R^{z} is independently selected from (i) or (ii) below:
(i) R^{y} and R^{z} are each independently hydrogen, alkyl, alkenyl, alkynyl, or cycloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five halo or alkyl groups;

A³ is N, CH or CR^{10a};

R^{10a} is alkyl, haloalkyl, alkoxy or halo;

R^{9a} is hydrogen, halo or alkyl;

R¹⁰ is alkyl, hydroxyalkyl, cyano, —R^{u}N(R^{a})(R^{b}), —R^{u}OR^{x}, —R^{u}OR^{x}OR^{x}, —R^{u}S(O)ₙR^{x}, —R^{u}N(R^{y})(R^{z}), or —C(O)N(R^{y})(R^{z});

R^{a} and R^{b} are each independently hydrogen or alkyl;

n is 0-2; and

m is 0 or 1.

In certain embodiments, provided herein are compounds of Formula IVA, IVB, IVC or IVD or pharmaceutically acceptable salts, solvates, hydrates or clathrates thereof, wherein

R¹ is substituted 5- to 6-membered aryl or substituted 5- to 6-membered heteroaryl where the substituents are selected from one, two or three R⁹ groups, wherein at least one R⁹ is a branched alkyl, haloalkyl, heterocyclyl or cycloalkyl, and wherein the second and third optional R⁹ groups is selected from halo, alkyl, haloalkyl, cycloalkyl and cycloalkylalkyl, where the alkyl, branched alkyl, haloalkyl, cycloalkyl, heterocyclyl or cycloalkylalkyl groups are each optionally substituted with 1 to 5 halo or hydroxy groups;

A⁴ is N or CR^{9a};

R² and R³ are each independently hydrogen, halo, hydroxy, amino or alkyl;

B² is CR^{3a};

R^{3a} is hydrogen, halo, or alkyl;

R⁴ is O or S;

R⁵ is halo, alkyl, haloalkyl or alkoxy;

R⁶ is hydrogen, halo, alkyl or alkoxy;

B³ is O, NH, or CH₂;

A² and R⁸ are selected as follows:

a) R⁸ is hydrogen, alkyl, alkenyl, alkynyl, cycloalkyl, —R^{u}OR^{x}, —R^{u}N(R^{y})(R^{z}), —R^{u}S(O)ₙR^{x}, heterocyclyl, aryl, or heteroaryl; and A² is N, CH or CR¹⁰; or

b) A² is C; and R⁸ together with A² forms a 5-7 membered substituted or unsubstituted heterocyclyl, where the substituents when present are one, two or three Q groups, each independently selected from oxo, halo, hydroxyl, alkoxy, alkyl, alkenyl, alkynyl, cycloalkyl, heterocyclyl, heteroaryl, hydroxyalkyl, haloalkyl and alkoxyalkyl;

R⁸ is optionally substituted with one, two or three Q¹ groups, each independently selected from halo, hydroxyl, alkoxy, cycloalkyl, alkyl, alkenyl, alkynyl, haloalkyl, heterocyclyl and heteroaryl;

Q and Q¹ groups are each optionally substituted with 1-6, 1-5, 1-3, one, two or three Q² groups each independently selected from halo, alkyl, alkenyl, alkynyl, cycloalkyl, haloalkyl, hydroxyl, amino and alkoxy;

each R^{u} is independently alkylene or a direct bond;

each R^{x} is independently hydrogen or alkyl;

each R^{y} and R^{z} is independently selected from (i) or (ii) below:
(i) R^{y} and R^{z} are each independently hydrogen, alkyl, alkenyl, alkynyl, or cycloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five halo or alkyl groups;

A³ is N, CH or CR^{10a};

R^{10a} is alkyl, haloalkyl, alkoxy or halo;

R^{9a} is hydrogen, halo or alkyl;

R¹⁰ is alkyl, hydroxyalkyl, cyano, —R^{u}N(R^{a})(R^{b}), —R^{u}OR^{x}, —R^{u}OR^{x}OR^{x}, —R^{u}S(O)ₙR^{x} or —C(O)N(R^{y})(R^{z});

R^{a} and R^{b} are each independently hydrogen or alkyl;

n is 0-2; and

m is 0 or 1.

In certain embodiments, R¹ is optionally substituted 5 to 6 membered heteroaryl; where substituents when present are selected from one, two or three R⁹ groups, wherein R⁹ is halo, cycloalkyl, heterocyclyl or alkyl, where cycloalkyl, heterocyclyl and alkyl are each optionally substituted with 1 to 5 groups selected from halo, alkyl and cycloalkyl. In certain embodiments, R¹ is a substituted 5 to 6 membered heteroaryl substituted with one, two or three R⁹ groups, wherein at least one R⁹ is a branched alkyl, heterocyclyl or cycloalkyl, and wherein the second and third optional R⁹ groups is selected from halo and alkyl, wherein the alkyl, cycloalkyl and branched alkyl may be optionally substituted with 1 to 5 groups selected from halo, hydroxy, and alkyl.

In certain embodiments, R¹ is optionally substituted azolyl; where substituents when present are selected from one, two or three R⁹ groups, wherein R⁹ is halo or alkyl, where alkyl is optionally substituted with 1 to 5 groups selected from halo, hydroxy and cycloalkyl. In certain embodiments, R¹ is substituted azolyl substituted with one, two or three R⁹ groups, wherein at least one R⁹ is a branched alkyl, heterocyclyl or cycloalkyl, and wherein the second and third optional R⁹ groups is selected from halo and alkyl, wherein the alkyl, branched alkyl, heterocyclyl and cycloalkyl are each optionally substituted with 1 to 5 groups selected from halo, hydroxy, haloalkyl, alkoxyalkyl, and alkyl.

In certain embodiments, R¹ is optionally substituted aryl; where substituents when present are selected from one, two or three R⁹ groups, wherein R⁹ is halo or alkyl, where alkyl is optionally substituted with 1 to 5 groups selected from halo and cycloalkyl. In certain embodiments, R¹ is optionally substituted phenyl; where substituents when present are selected from one, two or three R⁹ groups, wherein R⁹ is halo or alkyl, where alkyl is optionally substituted with 1 to 5 groups selected from halo, hydroxy and cycloalkyl.

In one embodiment, R¹ is:

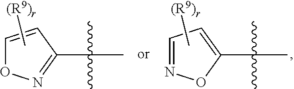

where r is 0, 1 or 2, and R⁹ is as described elsewhere herein. In one embodiment, R⁹ is alkyl, cycloalkyl or haloalkyl where the alkyl, cycloalkyl or haloalkyl is optionally substituted with 1 to 5 groups selected from halo, hydroxyl, haloalkyl, alkoxyalkyl and cycloalkyl. In one embodiment, R⁹ is alkyl, cycloalkyl or haloalkyl where the alkyl, cycloalkyl or haloalkyl is optionally substituted with 1 to 5 groups selected from halo, haloalkyl, alkoxyalkyl, hydroxy, alkoxy, alkoxyalkyl and cycloalkyl. In one embodiment, R⁹ is alkyl, cycloalkyl or haloalkyl where the alkyl, cycloalkyl and haloalkyl is optionally substituted with 1 to 5 groups selected from halo, hydroxy and cycloalkyl. In one embodiment, R⁹ is alkyl, where alkyl is optionally substituted with 1 to 5 groups selected from halo, hydroxy and cycloalkyl.

In one embodiment, R¹ is:

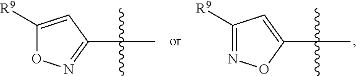

where R⁹ is as described elsewhere herein. In one embodiment, R⁹ is alkyl, cycloalkyl or haloalkyl where the alkyl, cycloalkyl and haloalkyl is optionally substituted with 1 to 5 groups selected from halo, hydroxy and cycloalkyl. In one embodiment, R⁹ is alkyl, cycloalkyl or haloalkyl where the alkyl, cycloalkyl or haloalkyl is optionally substituted with 1 to 5 groups selected from halo, hydroxyl, haloalkyl, alkoxyalkyl and cycloalkyl. In one embodiment, R⁹ is alkyl, cycloalkyl or haloalkyl where the alkyl, cycloalkyl or haloalkyl is optionally substituted with 1 to 5 groups selected from halo, haloalkyl, alkoxyalkyl, hydroxy, alkoxy, alkoxyalkyl and cycloalkyl. In one embodiment, R⁹ is alkyl, where alkyl is optionally substituted with 1 to 5 groups selected from halo and cycloalkyl. In one embodiment, R⁹ is —CF₃, —C(CH₃)₃, —C(CH₃)(CH₂F)₂, —C(CH₃)₂CF₃, —C(CH₃)₂CH₂F, —CF(CH₃)₂, or

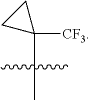 [SMILES: *C1(C(F)(F)F)CC1]

In another embodiment, R⁹ is —CH(CH₃)₂, —CF₃, —C(CH₃)₃, —CF₂(CH₃), —C(CH₃)(CH₂F)₂, —C(CH₃)₂CF₃, —CF(CH₃)₂, cyclopropyl, cyclobutyl, cyclopentyl, cyclohexyl,

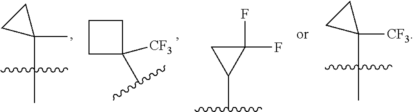

In another embodiment, R⁹ is selected from —CH(CH₃)₂, —C(CH₃)CH₂OH, —CF₃, —C(CH₃)₃, —CF₂(CH₃), —C(CH₃)(CH₂F)₂, —C(CH₃)₂CF₃, —C(CH₃)₂CH₂F, —CF(CH₃)₂, cyclopropyl, cyclobutyl, cyclopentyl, cyclohexyl,

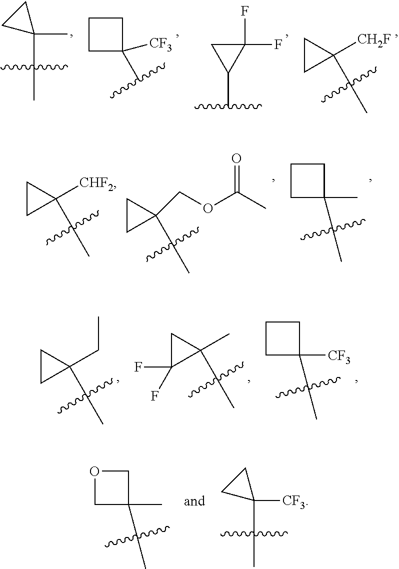

In one embodiment, R¹ is phenyl, optionally substituted with one or two R⁹ groups, where each R⁹ is independently halo or alkyl, where alkyl is optionally substituted with 1 to 5 groups selected from halo, hydroxy and cycloalkyl. In one embodiment, R⁹ is alkyl, cycloalkyl or haloalkyl where the alkyl, cycloalkyl and haloalkyl is optionally substituted with 1 to 5 groups selected from halo, hydroxy and cycloalkyl. In one embodiment, R⁹ is fluoro, —CF₃, —C(CH₃)₃, —C(CH₃)(CH₂F)₂, —C(CH₃)₂CF₃, —C(CH₃)₂CH₂F, —CF(CH₃)₂, or

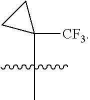 [SMILES: *C1(C(F)(F)F)CC1]

In another embodiment, R⁹ is —CH(CH₃)₂, —CF₃, —C(CH₃)₃, —CF₂(CH₃), —C(CH₃)(CH₂F)₂, —C(CH₃)₂CF₃, —CF(CH₃)₂, cyclopropyl, cyclobutyl, cyclopentyl, cyclohexyl,

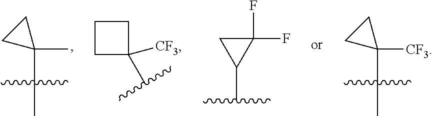

In another embodiment, R⁹ is selected from —CH(CH₃)₂, —C(CH₃)₂CH₂OH, —CF₃, —C(CH₃)₃, —CF₂(CH₃), —C(CH₃)(CH₂F)₂, —C(CH₃)₂CF₃, —C(CH₃)₂CH₂F, —CF(CH₃)₂, cyclopropyl, cyclobutyl, cyclopentyl, cyclohexyl,

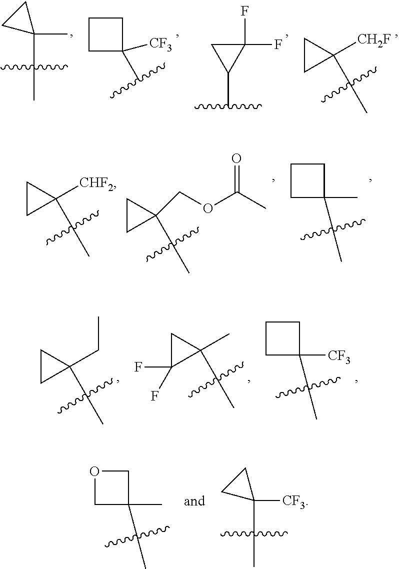

In one embodiment, R¹ is:

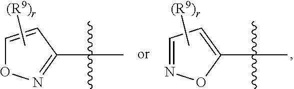

where r is 0, 1 or 2, and R⁹ is as described elsewhere herein. In one embodiment, R⁹ is alkyl, cycloalkyl or haloalkyl where the alkyl, cycloalkyl and haloalkyl is optionally substituted with 1 to 5 groups selected from halo and cycloalkyl. In one embodiment, R⁹ is alkyl, cycloalkyl or haloalkyl where the alkyl, cycloalkyl and haloalkyl is optionally substituted with 1 to 5 groups selected from halo, hydroxy and cycloalkyl. In one embodiment, R⁹ is alkyl, where alkyl is optionally substituted with 1 to 5 groups selected from halo, hydroxy and cycloalkyl.

In one embodiment, R¹ is:

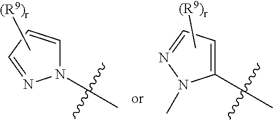

where r is 0, 1 or 2, and R⁹ is as described elsewhere herein. In one embodiment, R⁹ is alkyl, cycloalkyl or haloalkyl where the alkyl, cycloalkyl and haloalkyl is optionally substituted with 1 to 5 groups selected from halo, hydroxy and cycloalkyl. In one embodiment, R⁹ is alkyl, cycloalkyl or haloalkyl where the alkyl, cycloalkyl or haloalkyl is optionally substituted with 1 to 5 groups selected from halo, haloalkyl, alkoxyalkyl, hydroxy, alkoxy, alkoxyalkyl and cycloalkyl. In one embodiment, R⁹ is —CF₃, —C(CH₃)₃, —C(CH₃)(CH₂F)₂, —C(CH₃)₂CF₃, —C(CH₃)₂CH₂F, —CF(CH₃)₂, or

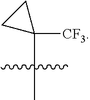 [SMILES: *C1(C(F)(F)F)CC1]

In another embodiment, R⁹ is —CH(CH₃)₂, —CF₃, —C(CH₃)₃, —CF₂(CH₃), —C(CH₃)(CH₂F)₂, —C(CH₃)₂CF₃, —CF(CH₃)₂, cyclopropyl, cyclobutyl, cyclopentyl, cyclohexyl,

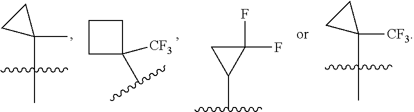

In another embodiment, R⁹ is selected from —CH(CH₃)₂, —C(CH₃)₂CH₂OH, —CF₃, —C(CH₃)₃, —CF₂(CH₃), —C(CH₃)(CH₂F)₂, —C(CH₃)₂CF₃, —C(CH₃)₂CH₂F, —CF(CH₃)₂, cyclopropyl, cyclobutyl, cyclopentyl, cyclohexyl,

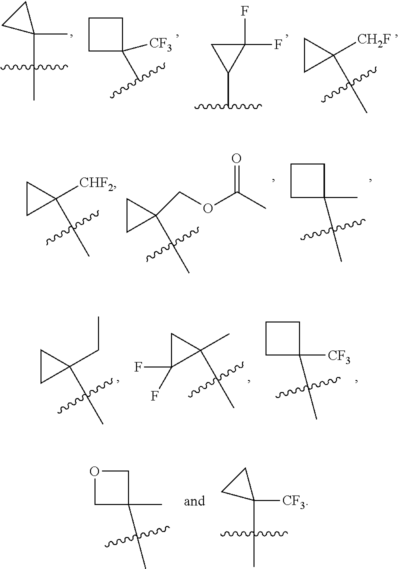

In certain embodiments, R² and R³ are each hydrogen.

In certain embodiments, R² is hydrogen, alkyl, halo or amino. In certain embodiments, R³ is hydrogen, alkyl, halo, hydroxy or amino. In certain embodiments, R² is hydrogen and R³ is hydrogen, alkyl, halo, hydroxy or amino. In certain embodiments, R³ is hydrogen. In certain embodiments, R³ is halo. In certain embodiments, R³ is alkyl. In certain embodiments, R³ is amino. In certain embodiments, R³ is hydroxy.

In certain embodiments, B¹ is CH or CR^{2a}, where R^{2a} is halo or alkyl. In certain embodiments, B¹ is CH.

In certain embodiments, B² is CH or CR^{3a}, where R^{3a} is hydrogen or alkyl. In certain embodiments, B² is CH. In certain embodiments, B² is CR^{3a}, where R^{3a} is hydrogen, halo or alkyl.

In certain embodiments, R⁴ is O or S. In certain embodiments, R⁴ is O.

In certain embodiments, R⁵ is halo. In one embodiment, R⁵ is fluoro or chloro.

In certain embodiments, R⁶ is hydrogen, halo, alkyl or alkoxy. In certain embodiments, R⁶ is hydrogen, fluoro, methyl or methoxy.

In certain embodiments, R⁸ is selected as follows:

a) R⁸ is hydrogen, alkyl, alkenyl, alkynyl, cycloalkyl, alkoxyalkyl, —R^{u}S(O)ₙR^{x}, —R^{u}N(R^{y})(R^{z}), or heterocyclyl; or

b) R⁸ together with A² forms a 5-7 membered heterocyclyl, optionally substituted with oxo;

R⁸ is optionally substituted with 1-6, 1-5, 1-4, one, two or three Q¹ groups, each independently selected from halo, hydroxyl, alkoxy, carboxy, cycloalkyl, alkyl, alkenyl, alkynyl, haloalkyl, heterocyclyl and heteroaryl; and

R^{y} and R^{z} are each independently selected from (i) or (ii) below:
(i) R^{y} and R^{z} are each independently hydrogen, alkyl, or cycloalkyl, or
(ii) R^{y} and R^{z}, together with the nitrogen atom to which they are attached, form a 5 or 6 membered heterocyclyl, optionally substituted with one or more, in one embodiment, one to six, in another embodiment, one, two, three, four or five alkyl or halo groups; and

Q and Q¹ groups are each optionally substituted with 1-6, 1-5, 1-4, one, two or three Q² groups each independently selected from halo, alkyl, alkenyl, alkynyl, cycloalkyl, haloalkyl, amino, hydroxyl and alkoxy;

In certain embodiments, R⁸ is hydrogen, alkyl, alkenyl, alkynyl, cycloalkyl, heterocyclyl, heterocyclylalkyl, heterocyclylalkenyl where the alkyl, alkenyl, alkynyl, cycloalkyl, heterocyclyl and heterocyclylalkyl, heterocyclylalkenyl are optionally substituted with 1-6, 1-5, 1-4, one or two alkyl, hydroxy, alkoxy, amino, alkylsulfonyl or halo groups. In one embodiment, R⁸ is hydrogen, methyl, tert-butyl, isopropyl, cyclopropyl, morpholinylmethyl, morpholinylethyl, piperidinylethyl, morpholinylpropyl, pyrrolidinylethyl, pyrrolidinylmethyl, tetrahydropyranyl, dimethylaminoethyl, methoxyethyl, or methylsulfonylethyl, where R⁸ is optionally substituted with one or two fluoro, methyl, hydroxy, amino or ethyl groups. In certain embodiments, R⁸ is hydrogen. In certain embodiments, B³ is NH and R⁸ is hydrogen.

In certain embodiments, R⁸ together with A² forms a 5-7 membered heterocyclyl, optionally substituted with alkyl, hydroxyalkyl or oxo.

In certain embodiments, R¹⁰ is halo, alkyl, hydroxyalkyl, alkoxyalkyl, cyano, —R^{u}N(R^{a})(R^{b}), —R^{u}S(O)ₙR^{x}, or —C(O)N(R^{y})(R^{z}), where R^{u} is alkylene, R^{x} is hydrogen or alkyl, n is 0-2, R^{a} and R^{b} are each independently hydrogen or alkyl, and R^{y} and R^{z} are each independently hydrogen or alkyl or R^{y} and R^{z} together form a heterocyclyl or heteroaryl ring. In certain embodiments, R¹⁰ is alkyl, hydroxyalkyl, cyano, CONH₂, —S(O)₀₋₂CH₃—, or -amino.

In certain embodiments, A¹ is N═CR^{9a}, CR^{9a}═CR^{9a} or CR^{9a}═N, where R^{9a} is hydrogen, halo or alkyl. In certain embodiments, A¹ is N═CH, CH═CH or CH═N. In certain embodiments, A¹ is CR^{9a}═CR^{9a} or N═CR^{9a}, where R^{9a} is hydrogen, halo or alkyl. In certain embodiments, A¹ is CH═CH or N═CH.

In certain embodiments, A¹ is N═CH; and A² and A³ are each CH. In certain embodiments, A¹ is CH═CH; and; and A² and A³ are each CH. In certain embodiments, A¹ is CH═CH; and; A³ is CH and A² is N.

In certain embodiments, A³ is CH, CR^{10a} or N, where R^{10a} is alkyl, halo or alkoxy.

In certain embodiments, A⁴ is N, or CR^{9a}, where R^{9a} is hydrogen, halo or alkyl.

In certain embodiments, m is 0 or 1. In certain embodiments, m is 0. In certain embodiments, m is 1.

In certain embodiments, n is 0, 1 or 2. In certain embodiments, n is 0. In certain embodiments, n is 1. In certain embodiments, n is 2.

In one embodiment, provided herein are compounds of formula VA, VB, VC or VD:

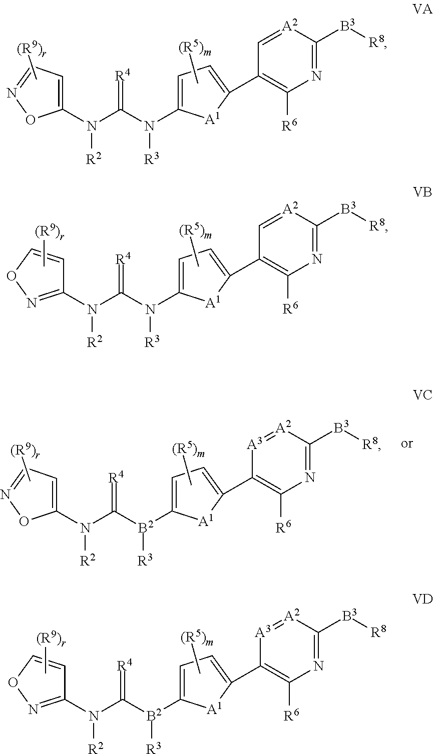

or a pharmaceutically acceptable salt, solvate, clathrate or hydrate thereof, wherein the variables are as described elsewhere herein. In certain embodiments, B³ is NH and R⁸ is hydrogen. In certain embodiments, B² is CR^{3a}; R^{3a} is hydrogen, alkyl, or halo; R³ is hydrogen, alkyl, amino or halo; and R⁸ is hydrogen. In one embodiment, provided herein is a compound of formula VA, VB, VC or VD, or a pharmaceutically acceptable salt, solvate, clathrate or hydrate thereof, wherein A¹ is N═CR^{9a}, S, CR^{9a}═N or CR^{9a}═CR^{9a};

R² is hydrogen or alkyl;

B² is N or CR^{3a};

R^{3a} is hydrogen, halo or alkyl;

R³ is hydrogen, halo, hydroxy, amino or alkyl;

R⁴ is O or S;

R⁵ is halo, alkyl, haloalkyl or alkoxy;

R⁶ is hydrogen, halo, alkyl, or alkoxy;

B³ is O, NH, or CH₂;

A² and R⁸ are selected as follows:

a) R⁸ is selected from hydrogen, alkyl, alkenyl, alkynyl, cycloalkylalkyl, cycloalkyl, heterocyclyl, heterocyclylalkyl and heterocyclylalkenyl, where the alkyl, alkenyl, alkynyl, cycloalkyl, heterocyclyl, heterocyclylalkyl and heterocyclylalkenyl are optionally substituted with 1-6, 1-5, one or two alkyl, hydroxy, alkoxy, amino, alkylsulfonyl, or halo groups; and A² is N, CH or CR¹⁰; or

b) A² is C; and R⁸ together with A² forms a 5-7 membered substituted or unsubstituted heterocyclyl, optionally substituted with alkyl, hydroxyalkyl or oxo;

R⁹ is alkyl, where alkyl is optionally substituted with 1 to 5 groups selected from halo, hydroxy and cycloalkyl;

R^{9a} is hydrogen, halo, alkyl, or alkoxy;

R¹⁰ is alkyl, hydroxyalkyl, amido, cyano, —R^{u}S(O)ₙR^{x}, —C(O)N(R^{y})(R^{z}), —R^{u}N(R^{a})(R^{b}), —R^{u}OR^{x}, or —R^{u}OR^{x}OR^{x},

R^{u} is alkylene,

R^{a} and R^{b} are each independently hydrogen or alkyl

R^{y} and R^{z} are each independently hydrogen or alkyl or a heterocyclyl or heteroaryl, optionally substituted with one or more, in one embodiment, one to six, in another embodiment, one, two, three, four or five halo or alkyl;

A³ is N, CH or CR^{10a};

R^{10a} is halo, alkyl, or alkoxy;

n is 0-2;

m is 0 or 1; and

r is 1 or 2.

In one embodiment, provided herein are compounds of formula VA, VB, VC or VD or a pharmaceutically acceptable salt, solvate, clathrate or hydrate thereof, wherein

at least one R⁹ is branched alkyl or cycloalkyl and the second optional R⁹ is selected from halo, alkyl, haloalkyl, cycloalkyl and cycloalkylalkyl, where the alkyl, branched alkyl, haloalkyl, cycloalkyl or cycloalkylalkyl groups are each optionally substituted with 1 to 5 groups selected from halo, hydroxy or cycloalkyl;

R² is hydrogen or alkyl;

B² is N or CR^{3a};

R^{3a} is hydrogen, halo or alkyl;

R³ is hydrogen, halo, hydroxy, amino or alkyl;

R⁴ is O or S;

B³ is O, NH, or CH₂;

A¹ is N═CR^{9a}, NR^{9a}, S, O, CR^{9a}═CR^{9a} or CR^{9a}═N;

A² and R⁸ are selected as follows:

a) R⁸ is selected from hydrogen, alkyl, alkenyl, alkynyl, cycloalkylalkyl, cycloalkyl, heterocyclyl, heterocyclylalkyl and heterocyclylalkenyl, where the alkyl, alkenyl, alkynyl, cycloalkyl, heterocyclyl, heterocyclylalkyl and heterocyclylalkenyl are optionally substituted with 1-6, 1-5, one or two alkyl, hydroxy, alkoxy, amino, alkylsulfonyl, or halo groups; and A² is N, CH or CR¹⁰; or

b) A² is C; and R⁸ together with A² forms a 5-7 membered substituted or unsubstituted heterocyclyl, optionally substituted with alkyl, hydroxyalkyl or oxo;

R^{9a} is hydrogen, halo, alkyl, or alkoxy;

R⁵ is halo, alkyl, alkenyl, alkynyl, cycloalkyl, haloalkyl, hydroxyalkyl, alkoxyalkyl, cycloalkylalkyl, cyano, amino, hydroxy, alkoxy, —R^{u}N(R^{y})(R^{z}), aryl, heterocyclyl, or heteroaryl;

R⁶ is hydrogen, halo, alkyl, or alkoxy;

R¹⁰ is alkyl, hydroxyalkyl, cyano, amido, —R^{u}SR^{x}, —R^{u}SOR^{x}, —R^{u}S(O)₂R^{x}, —R^{u}N(R^{a})(R^{b}), —R^{u}OR^{x}, or —R^{u}OR^{x}OR^{x}, where R^{x} is hydrogen or alkyl, R^{u} is alkylene, R^{a} and R^{b} are each independently hydrogen or alkyl;

A³ is N, CH or CR^{10a};

R^{10a} is halo, alkyl, or alkoxy;

m is 0 or 1;

r is 1 or 2 and other variables are as described elsewhere herein.

In one embodiment, provided herein are compounds of formula VIA, VIB, VIC or VID:

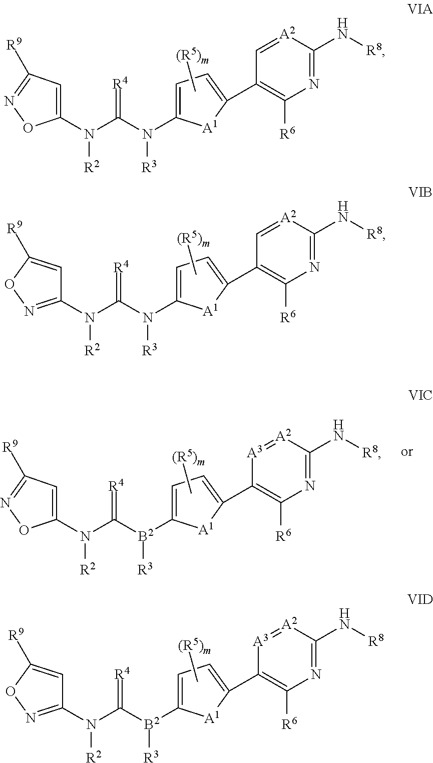

or a pharmaceutically acceptable salt, solvate, clathrate or hydrate thereof, wherein the variables are as described elsewhere herein. In certain embodiments, R⁸ is hydrogen. In one embodiment, provided herein is a compound of formula VIA, VIB, VIC or VID or a pharmaceutically acceptable salt, solvate, clathrate or hydrate thereof, wherein A¹ is N═CR^{9a}, S or CR^{9a}═CR^{9a};

R² is hydrogen or alkyl;

B² is N or CR^{3a};

R^{3a} is hydrogen, halo or alkyl;

R³ is hydrogen, halo, hydroxy, amino or alkyl;

R⁴ is O or S;

R⁵ is halo, alkyl, haloalkyl or alkoxy;

R⁶ is hydrogen, halo, alkyl, or alkoxy;

A² and R⁸ are selected as follows:

a) R⁸ is selected from hydrogen, alkyl, alkenyl, alkynyl, cycloalkylalkyl, cycloalkyl, heterocyclyl, heterocyclylalkyl and heterocyclylalkenyl, where the alkyl, alkenyl, alkynyl, cycloalkyl, heterocyclyl, heterocyclylalkyl and heterocyclylalkenyl are optionally substituted with 1-6, 1-5, one or two alkyl, hydroxy, alkoxy, amino, alkylsulfonyl, or halo groups; one or two alkyl or halo groups; and A² is N, CH or CR¹⁰; or

b) A² is C; and R⁸ together with A² forms a 5-7 membered substituted or unsubstituted heterocyclyl, optionally substituted with alkyl, hydroxyalkyl or oxo;

A³ is N, CH or CR^{10a};

R^{10a} is halo, alkyl, or alkoxy;

R⁹ is alkyl, where alkyl is optionally substituted with 1 to 5 groups selected from halo and cycloalkyl;

R^{9a} is hydrogen, halo, alkyl, or alkoxy;

R¹⁰ is alkyl, hydroxyalkyl, cyano, amido, —R^{u}SR^{x}, —R^{u}SOR^{x}, —R^{u}S(O)₂R^{x}, —R^{u}N(R^{a})(R^{b}), —R^{u}OR^{x}, or —R^{u}OR^{x}OR^{x}, where R^{x} is hydrogen or alkyl, R^{u} is alkylene, R^{a} and R^{b} are each independently hydrogen or alkyl or R^{a} and R^{b} together form a heterocyclyl ring; and

m is 0 or 1.

In one embodiment, provided herein are compounds of formula VIIA VIIB, VIIC or VIID:

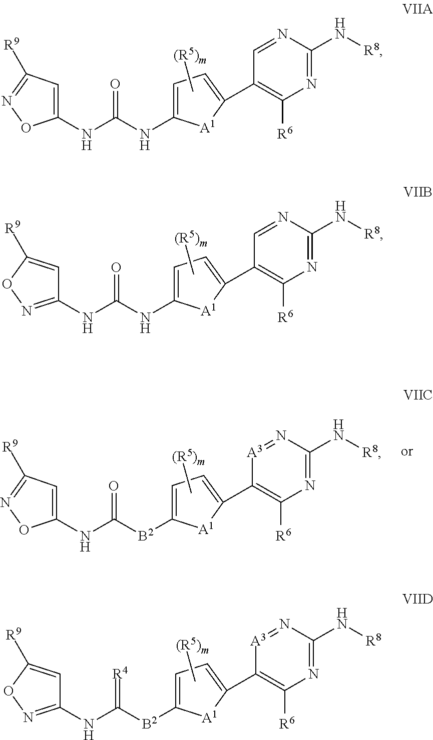

or a pharmaceutically acceptable salt, solvate, clathrate or hydrate thereof, wherein the variables are as described elsewhere herein. In one embodiment, A¹ is N═CH, S or CH═CH, and the other variables are as described elsewhere herein. In one embodiment, A¹ is N═CH, S, CH═CH or CH═N, and the other variables are as described elsewhere herein. In one embodiment, A¹ is N═CH, and other variables are as described elsewhere herein. In one embodiment, A¹ is S, and other variables are as described elsewhere herein. In one embodiment, A¹ is CH═CH, and the other variables are as described elsewhere herein. In one embodiment, A³ is CH or CR^{10a}, where R^{10a} is alkyl, halo or alkoxy; B² is CR^{3a} or NH, and the other variables are as described elsewhere herein. In certain embodiments, R⁸ is hydrogen and the other variables are as described elsewhere herein.

In one embodiment, provided herein are compounds of formula VIIIA, VIIIB, VIIIC or VIIID:

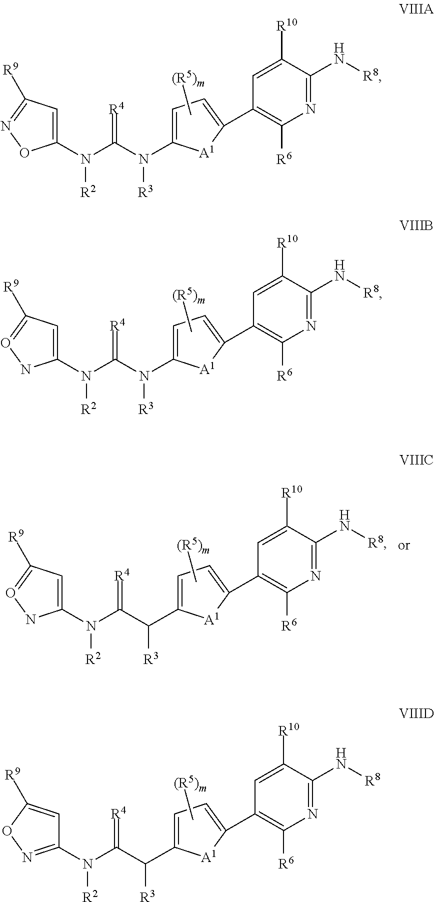

or a pharmaceutically acceptable salt, solvate, clathrate or hydrate thereof, wherein the variables are as described elsewhere herein. In one embodiment, A¹ is N═CH, S, CH═CH or N═CH. In one embodiment, A¹ is N═CH, S or CH═CH. In one embodiment, A¹ is N═CH or CH═CH. In one embodiment, A¹ is N═CH. In one embodiment, A¹ is S. In one embodiment, A¹ is CH═CH. In certain embodiments, R⁸ is hydrogen and the other variables are as described elsewhere herein.

In one embodiment, provided herein are compounds of formula IXA or IXB:

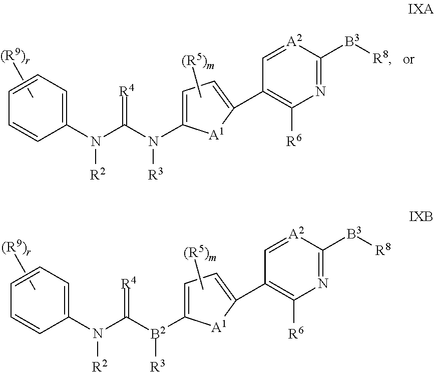

or a pharmaceutically acceptable salt, solvate, clathrate or hydrate thereof, wherein the variables are as described elsewhere herein. In certain embodiments, B³ is NH, R⁸ is hydrogen and the other variables are as described elsewhere herein. In one embodiment, provided herein is a compound of formula IXA or IXB, wherein

at least one R⁹ is branched alkyl or cycloalkyl and the second optional R⁹ is selected from halo, alkyl, haloalkyl, cycloalkyl and cycloalkylalkyl, where the alkyl, branched alkyl, haloalkyl, cycloalkyl or cycloalkylalkyl groups are each optionally substituted with 1 to 5 groups selected from halo, haloalkyl, alkoxyalkyl, hydroxyl, alkoxy or cycloalkyl;

R² is hydrogen or alkyl;

B² is N or CR^{3a};

R^{3a} is hydrogen, halo or alkyl;

R³ is hydrogen, halo, hydroxy, amino or alkyl;

R⁴ is O or S;

R⁵ is halo, alkyl, haloalkyl or alkoxy;

R⁶ is hydrogen, halo, alkyl, or alkoxy;

B³ is O, NH, or CH₂;

A² and R⁸ are selected as follows:

a) R⁸ is selected from hydrogen, alkyl, alkenyl, alkynyl, cycloalkylalkyl, cycloalkyl, heterocyclyl, heterocyclylalkyl and heterocyclylalkenyl, where the alkyl, alkenyl, alkynyl, cycloalkyl, heterocyclyl, heterocyclylalkyl and heterocyclylalkenyl are optionally substituted with 1-6, 1-5, one or two alkyl, hydroxy, alkoxy, amino, alkylsulfonyl, or halo groups; and A² is N, CH or CR¹⁰; or

b) A² is C; and R⁸ together with A² forms a 5-7 membered substituted or unsubstituted heterocyclyl, optionally substituted with alkyl, hydroxyalkyl or oxo;

A¹ is N═CR^{9a}, S or CR^{9a}═CR^{9a};

R^{9a} is hydrogen, halo, alkyl, or alkoxy;

R¹⁰ is alkyl, hydroxyalkyl, cyano, amido, —R^{u} SR^{x}, —R^{u} SOR^{x}, —R^{u} S(O)₂R^{x}, or —R^{u}N(R^{a})(R^{b}), where R^{x} is hydrogen or alkyl, R^{u} is alkylene, R^{a} and R^{b} are each independently hydrogen or alkyl or R^{a} and R^{b} together form a heterocyclyl ring;

m is 0 or 1; and

r is 1 or 2.

In one embodiment, provided herein are compounds of formula XA or XB:

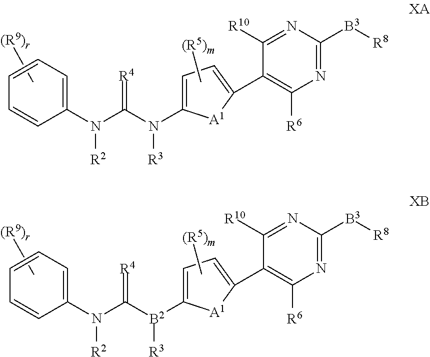

or a pharmaceutically acceptable salt, solvate, clathrate or hydrate thereof, wherein the variables are as described elsewhere herein. In certain embodiments, B³ is NH, R⁸ is hydrogen and the other variables are as described elsewhere herein. In one embodiment, provided herein is a compound of formula XA or XB, wherein

at least one R⁹ is branched alkyl or cycloalkyl and the second optional R⁹ is selected from halo, alkyl, haloalkyl, cycloalkyl and cycloalkylalkyl, where the alkyl, branched alkyl, haloalkyl, cycloalkyl or cycloalkylalkyl groups are each optionally substituted with 1 to 5 groups selected from halo, haloalkyl, alkoxyalkyl, hydroxyl, alkoxy or cycloalkyl;

R² is hydrogen or alkyl;

B² is N or CR^{3a};

R^{3a} is hydrogen, halo or alkyl;

R³ is hydrogen, halo, hydroxy, amino or alkyl;

R⁴ is O or S;

R⁵ is halo, alkyl, haloalkyl or alkoxy;

R⁶ is hydrogen, halo, alkyl, or alkoxy;

B³ is O, NH, or CH₂;

A² and R⁸ are selected as follows:

a) R⁸ is selected from hydrogen, alkyl, alkenyl, alkynyl, cycloalkylalkyl, cycloalkyl, heterocyclyl, heterocyclylalkyl and heterocyclylalkenyl, where the alkyl, alkenyl, alkynyl, cycloalkyl, heterocyclyl, heterocyclylalkyl and heterocyclylalkenyl are optionally substituted with 1-6, 1-5, one or two alkyl, hydroxy, alkoxy, amino, alkylsulfonyl, or halo groups; and A² is N, CH or CR¹⁰; or

b) A² is C; and R⁸ together with A² forms a 5-7 membered substituted or unsubstituted heterocyclyl, optionally substituted with alkyl, hydroxyalkyl, or oxo;

A¹ is N═CR^{9a} or CR^{9a}═CR^{9a};

R^{9a} is hydrogen, halo, alkyl, or alkoxy;

R¹⁰ is alkyl, hydroxyalkyl, cyano, amido, —R^{u}SR^{x}, —R^{u} SOR^{x}, —R^{u} S(O)₂R^{x}, or —R^{u}N(R^{a})(R^{b}), where R^{x} is hydrogen or alkyl, R^{u} is direct bond or alkylene, R^{a} and R^{b} are each independently hydrogen or alkyl, or R^{a} and R^{b} together form a heterocyclyl ring;

m is 0 or 1; and

r is 1 or 2.

In one embodiment, provided herein is a compound of formula XI:

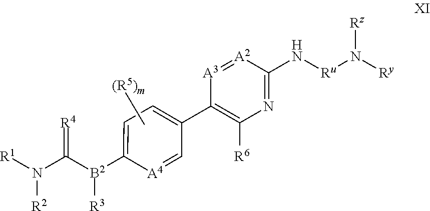

or a pharmaceutically acceptable salt, solvate, clathrate or hydrate thereof, wherein the variables are as described elsewhere herein. In one embodiment, provided herein is a compound of formula XI, wherein

R¹ is substituted 5- to 6-membered aryl or substituted 5- to 6-membered heteroaryl where the substituents are selected from one, two or three R⁹ groups, wherein at least one R⁹ is a branched alkyl, heterocyclyl or cycloalkyl, and wherein the second and third optional R⁹ groups is selected from halo, alkyl, haloalkyl, cycloalkyl and cycloalkylalkyl, where the alkyl, branched alkyl, haloalkyl, cycloalkyl or cycloalkylalkyl groups are each optionally substituted with 1 to 5 groups selected from halo, alkyl, haloalkyl, alkoxyalkyl, hydroxyl, alkoxy and cycloalkyl;

R² is hydrogen or alkyl;

A⁴ is N, or CR^{9a};

R⁴ is O or S;

R⁵ is halo, alkyl, haloalkyl or alkoxy;

R⁶ is hydrogen, halo, alkyl, or alkoxy;

B² is N or CR^{3a};

R^{3a} is hydrogen, halo or alkyl;

R³ is hydrogen, halo, hydroxy, amino or alkyl;

A² is N, CH or CR¹⁰;

A³ is N, CH or CR¹⁰;

R^{9a} is hydrogen, halo, alkyl, or alkoxy;

R^{10a} is halo, alkyl, or alkoxy;

m is 0 or 1;

R¹⁰ is alkyl, hydroxyalkyl, cyano, amido, —R^{u}S(O)₀₋₂R^{x} or —R^{u}N(R^{a})(R^{b}),

R^{a} and R^{b} are each independently hydrogen, or alkyl;

each R^{u} is independently alkylene, alkenylene or alkynylene or a direct bond; and

each R^{y} and R^{z} is independently selected from (i) or (ii) below:
(i) R^{y} and R^{z} are each independently hydrogen, alkyl, alkenyl, alkynyl, cycloalkyl, alkoxyalkyl, or haloalkyl; or
(ii) R^{y} and R^{z}, together with the nitrogen atom to which they are attached, form a heterocyclyl or heteroaryl, optionally substituted with one or more, in one embodiment, one to six, in another embodiment, one, two, three, four or five halo, haloalkyl, alkyl, alkenyl or alkynyl groups.

In one embodiment, provided herein is a compound of formula XII:

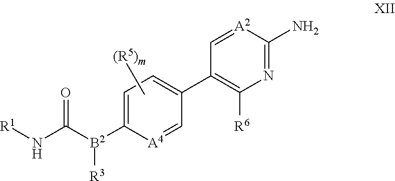

or a pharmaceutically acceptable salt, solvate, clathrate or hydrate thereof, wherein the variables are as described elsewhere herein. In one embodiment, provided herein is a compound of formula XII, wherein

R¹ is substituted isoxazolyl where the substituents are selected from one or two R⁹ groups, wherein at least one R⁹ is a branched alkyl, heterocyclyl or cycloalkyl, and wherein the second optional R⁹ group is selected from halo, alkyl, haloalkyl, cycloalkyl and cycloalkylalkyl, where the alkyl, branched alkyl, haloalkyl, cycloalkyl or cycloalkylalkyl groups are each optionally substituted with one or two groups selected from halo, alkyl, haloalkyl, alkoxyalkyl, hydroxy, alkoxy and cycloalkyl;

B² is N or CR^{3a};

R^{3a} is hydrogen, halo or alkyl;

R³ is hydrogen, halo, hydroxy, amino or alkyl;

A⁴ is N, or CR^{9a};

R⁵ is halo, alkyl, haloalkyl or alkoxy;

A² is N, CH or CR¹⁰;

R^{9a} is hydrogen, halo, alkyl, or alkoxy;

m is 0 or 1;

R¹⁰ is alkyl, hydroxyalkyl, cyano, or amido.

In one embodiment, provided herein is a compound of formula XIII:

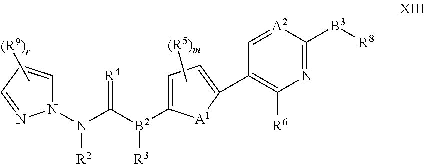

or a pharmaceutically acceptable salt, solvate, clathrate or hydrate thereof, wherein the variables are as described elsewhere herein. In certain embodiments, B³ is NH, R⁸ is hydrogen and the other variables are as described elsewhere herein. In one embodiment, provided herein is a compound of formula XII, wherein

at least one R⁹ is branched alkyl or cycloalkyl and the second optional R⁹ is selected from halo, alkyl, haloalkyl, cycloalkyl and cycloalkylalkyl, where the alkyl, branched alkyl, haloalkyl, cycloalkyl or cycloalkylalkyl groups are each optionally substituted with 1 to 5 groups selected from halo, alkyl, haloalkyl, alkoxyalkyl, hydroxyl, alkoxy and cycloalkyl;

R² and R³ are each independently hydrogen, halo, hydroxy, amino or alkyl;

R⁴ is O or S;

R⁵ is halo, alkyl, haloalkyl or alkoxy;

R⁶ is hydrogen, halo, alkyl, or alkoxy;

B³ is O, NH, or CH₂;

A² and R⁸ are selected as follows:

a) R⁸ is selected from hydrogen, alkyl, alkenyl, alkynyl, cycloalkylalkyl, cycloalkyl, heterocyclyl, heterocyclylalkyl and heterocyclylalkenyl, where the alkyl, alkenyl, alkynyl, cycloalkyl, heterocyclyl, heterocyclylalkyl and heterocyclylalkenyl are optionally substituted with 1-6, 1-5, one or two alkyl, hydroxy, alkoxy, amino, alkylsulfonyl, or halo groups; and A² is N, CH or CR¹⁰; or

b) A² is C; and R⁸ together with A² forms a 5-7 membered substituted or unsubstituted heterocyclyl, optionally substituted with alkyl, hydroxyalkyl or oxo;

A¹ is N═CR^{9a}, S or CR^{9a}═CR^{9a};

R^{9a} is hydrogen, halo, alkyl, or alkoxy;

R¹⁰ is alkyl, hydroxyalkyl, cyano, amido, —R^{u} SR^{x}, —R^{u}SOR^{x}, —R^{u} S(O)₂R^{x}, —R^{u}N(R^{a})(R^{b}), —R^{u}OR^{x}, or —R^{u}OR^{x}OR^{x}, where W is hydrogen or alkyl, R^{u} is direct bond or alkylene, R^{a} and R^{b} are each independently hydrogen or alkyl; or R^{a} and R^{b} together form a heterocyclyl ring;

m is 0 or 1; and

r is 1 or 2.

In another embodiment, R⁹ is substituted 1 to 5 groups selected from halo, alkyl, hydroxy and cycloalkyl. In another embodiment, R⁹ is substituted with 1 to 5 groups selected from halo, hydroxyl and cycloalkyl. In another embodiment, R⁹ is substituted 1 to 5 groups selected from halo, alkyl and cycloalkyl.

In another embodiment, the compound is selected from Tables 1, 2 and 3.

In another embodiment, the compound provided is selected from
1-(4-(6-aminopyridin-3-yl)phenyl)-3-(5-tert-butylisoxazol-3-yl)urea,
1-[4-(6-amino-5-cyanopyridin-3-yl)-phenyl]-3-(5-tert-butylisoxazol-3-yl)urea,
1-(5-tert-butylisoxazol-3-yl)-3-(4-(3-oxo-3,4-dihydro-2H-pyrido[3,2-b][1,4]oxazin-7-yl)phenyl)urea,
1-(5-tert-butylisoxazol-3-yl)-3-(4-(3,4-dihydro-2H-pyrido[3,2-b][1,4]oxazin-7-yl)phenyl)urea,
1-(5-tert-butylisoxazol-3-yl)-3-(4-(3-(2-hydroxyethyl)-3,4-dihydro-2H-pyrido[3,2-b][1,4]oxazin-7-yl)phenyl)urea,
1-(5-tert-butylisoxazol-3-yl)-3-(4-(5-cyano-6-(2-morpholinoethylamino)pyridin-3-yl)phenyl)urea,
1-(5-tert-butyl-isoxazol-3-yl)-3-{4-[6-(2-morpholin-4-yl-ethylamino)-pyridin-3-yl]-phenyl}urea,
1-(4-(6-amino-5-(hydroxymethyl)pyridin-3-yl)phenyl)-3-(5-tert-butylisoxazol-3-yl)urea,
1-(5-tert-butylisoxazol-3-yl)-3-(4-(7-oxo-5,6,7,8-tetrahydro-1,8-naphthyridin-3-yl)phenyl)urea,
1-(4-(6-amino-2,4-dimethylpyridin-3-yl)phenyl)-3-(5-tert-butylisoxazol-3-yl)urea,
1-(4-(6-aminopyridin-3-yl)-3-fluorophenyl)-3-(5-tert-butylisoxazol-3-yl)urea,
1-(5-tert-butylisoxazol-3-yl)-3-(4-(5,6,7,8-tetrahydro-1,8-naphthyridin-3-yl)phenyl)urea,
1-(4-(6-amino-5-(morpholinomethyl)pyridin-3-yl)phenyl)-3-(5-tert-butylisoxazol-3-yl)urea,
1-[4-(6-aminopyridin-3-yl)-2-fluorophenyl]-3-(5-tert-butylisoxazol-3-yl)-urea,
1-(4-(6-aminopyridin-3-yl)-2-chlorophenyl)-3-(5-tert-butylisoxazol-3-yl)urea,
1-(4-(6-aminopyridin-3-yl)-3-chlorophenyl)-3-(5-tert-butylisoxazol-3-yl)urea,
1-(6′-amino-[3,3′]bipyridinyl-6-yl)-3-(5-tert-butylisoxazol-3-yl)urea,
2-(4-(6-aminopyridin-3-yl)phenyl)-N-(5-tert-butylisoxazol-3-yl)acetamide,
1-(5-(6-aminopyridin-3-yl)thiophen-2-yl)-3-(5-tert-butylisoxazol-3-yl)urea,
1-(4-(6-aminopyridin-3-yl)-2,5-difluorophenyl)-3-(5-tert-butylisoxazol-3-yl)urea,
1-(5-tert-butylisoxazol-3-yl)-3-(4-(1,2,3,5-tetrahydropyrido[2,3-e][1,4]oxazepin-7-yl)phenyl)urea,
1-(4-(6-amino-5-((2-hydroxyethoxy)methyl)pyridin-3-yl)phenyl)-3-(5-tert-butylisoxazol-3-yl)urea,
1-(4-(6-amino-2-methylpyridin-3-yl)phenyl)-3-(5-tert-butylisoxazol-3-yl)urea,
1-(4-(6-amino-4-methylpyridin-3-yl)phenyl)-3-(5-tert-butylisoxazol-3-yl)urea,
1-(6′-amino-2′-methyl-3,3′-bipyridin-6-yl)-3-(5-tert-butylisoxazol-3-yl)urea,
1-(6′-amino-4′-methyl-3,3′-bipyridin-6-yl)-3-(5-tert-butylisoxazol-3-yl)urea,
1-(5-tert-butylisoxazol-3-yl)-3-(2-fluoro-4-(6-(2-(piperidin-1-yl)ethylamino)pyridin-3-yl)phenyl)urea,
1-(5-tert-butylisoxazol-3-yl)-3-(4-(6-(3-morpholinopropylamino)pyridin-3-yl)phenyl)urea,
1-(5-tert-butylisoxazol-3-yl)-3-(4-(6-(2-(1-methylpyrrolidin-2-yl)ethylamino)pyridin-3-yl)phenyl)urea,
1-(5-tert-butylisoxazol-3-yl)-3-(4-(6-((1-ethylpyrrolidin-2-yl)methylamino)pyridin-3-yl)phenyl)urea,
1-(4-(6-aminopyridin-3-yl)phenyl)-3-(5-tert-butylisoxazol-3-yl)-1-methylurea

5-(4-(3-(3-(2-fluoropropan-2-yl)isoxazol-5-yl)ureido)phenyl)pyridin-2-aminium methanesulfonate,
1-(4-(6-aminopyridin-3-yl)phenyl)-3-(3-(2-fluoropropan-2-yl)isoxazol-5-yl)urea

5-(4-(3-(5-(1-(trifluoromethyl)cyclopropyl)isoxazol-3-yl)ureido)phenyl)pyridin-2-aminium methanesulfonate,
1-(4-(6-aminopyridin-3-yl)phenyl)-3-(5-(1-(trifluoromethyl)cyclopropyl)isoxazol-3-yl)urea,
5-(4-(3-(5-(1,3-difluoro-2-methylpropan-2-yl)isoxazol-3-yl)ureido)phenyl)pyridin-2-aminium methanesulfonate,
1-(4-(6-aminopyridin-3-yl)phenyl)-3-(5-(1,3-difluoro-2-methylpropan-2-yl)isoxazol-3-yl)urea,
5-(4-(3-(3-(2-fluoropropan-2-yl)isoxazol-5-yl)ureido)phenyl)pyridin-2-aminium methanesulfonate,
1-(4-(6-aminopyridin-3-yl)phenyl)-3-(5-(1,1,1-trifluoro-2-methylpropan-2-yl)isoxazol-3-yl)urea,
4-(2-(5-(4-(3-(5-(1-(trifluoromethyl)cyclopropyl)isoxazol-3-yl)ureido)phenyl)pyridin-2-ylamino)ethyl)morpholin-4-ium methanesulfonate,
1-(4-(6-(2-morpholinoethylamino)pyridin-3-yl)phenyl)-3-(5-(1-(trifluoromethyl)cyclopropyl)isoxazol-3-yl)urea,
4-(2-(5-(4-(3-(5-(1,3-difluoro-2-methylpropan-2-yl)isoxazol-3-yl)ureido)phenyl)pyridin-2-ylamino)ethyl)morpholin-4-ium methanesulfonate,
1-(5-(1,3-difluoro-2-methylpropan-2-yl)isoxazol-3-yl)-3-(4-(6-(2-morpholinoethylamino)pyridin-3-yl)phenyl)urea,
4,4-(2-(5-(4-(3-(3-(2-fluoropropan-2-yl)isoxazol-5-yl)ureido)phenyl)pyridin-2-ylamino)ethyl)morpholin-4-ium methanesulfonate,
1-(3-(2-fluoropropan-2-yl)isoxazol-5-yl)-3-(4-(6-(2-morpholinoethylamino)pyridin-3-yl)phenyl)urea,
4-(2-(5-(4-(3-(5-(1,1,1-trifluoro-2-methylpropan-2-yl)isoxazol-3-yl)ureido)phenyl)pyridin-2-ylamino)ethyl)morpholin-4-ium methanesulfonate,
1-(4-(6-(2-morpholinoethylamino)pyridin-3-yl)phenyl)-3-(5-(1,1,1-trifluoro-2-methylpropan-2-yl)isoxazol-3-yl)urea,
1-(6′-amino-3,3′-bipyridin-6-yl)-3-(5-(1-(trifluoromethyl)cyclopropyl)isoxazol-3-yl)urea,
1-(6′-amino-3,3′-bipyridin-6-yl)-3-(5-(1,3-difluoro-2-methylpropan-2-yl)isoxazol-3-yl)urea,
1-(6′-amino-3,3′-bipyridin-6-yl)-3-(3-(2-fluoropropan-2-yl)isoxazol-5-yl)urea,
1-(6′-amino-3,3′-bipyridin-6-yl)-3-(5-(1,1,1-trifluoro-2-methylpropan-2-yl)isoxazol-3-yl)urea,
1-(4-(2-aminopyrimidin-5-yl)phenyl)-3-(5-tert-butylisoxazol-3-yl)urea,
1-(5-tert-butylisoxazol-3-yl)-3-{4-[2-(2-morpholin-4-yl-ethylamino)-pyrimidin-5-yl]-phenyl}urea,
N-(4-(2-aminopyrimidin-5-yl)phenyl)-2-(3-(trifluoromethyl)phenyl)acetamide,
1-(5-tert-butyl-isoxazol-3-yl)-3-{4-[2-(2-morpholin-4-yl-ethoxy)-pyrimidin-5-yl]-phenyl}-urea,
1-[4-(2-aminopyrimidin-5-yl)-2-methoxy-phenyl]-3-(5-tert-butylisoxazol-3-yl)-urea,
1-(4-(2-amino-4-methylpyrimidin-5-yl)phenyl)-3-(5-tert-butylisoxazol-3-yl)urea,
1-[4-(2-amino-4-methoxypyrimidin-5-yl)-phenyl]-3-(5-tert-butylisoxazol-3-yl)-urea,
1-(5-tert-butylisoxazol-3-yl)-3-(4-(2-(morpholinomethyl)pyrimidin-5-yl)phenyl)urea,
1-[5-(2-fluoro-1-fluoromethyl-1-methylethyl)isoxazol-3-yl]-3-{4-[2-(2-morpholin-4-yl-ethylamino)pyrimidin-5-yl]phenyl}urea,
1-{4-[2-(2-morpholin-4-yl-ethylamino)-pyrimidin-5-yl]-phenyl}-3-[5-(1-trifluoromethyl-cyclopropyl)-isoxazol-3-yl]urea,
1-(4-(2-(2-morpholinoethylamino)pyrimidin-5-yl)phenyl)-3-(3-(trifluoromethyl)phenyl)urea,
1-(2-fluoro-5-methylphenyl)-3-(4-(2-(2-morpholinoethylamino)pyrimidin-5-yl)phenyl)urea,
1-(5-tert-butylisoxazol-3-yl)-3-(4-(2-(3-morpholinopropyl)pyrimidin-5-yl)phenyl)urea,
1-(5-tert-butylisoxazol-3-yl)-3-(4-(2-(2-(dimethylamino)ethylamino)pyrimidin-5-yl)phenyl)urea,
1-(5-tert-butylisoxazol-3-yl)-3-{4-[2-(2-methoxyethylamino)pyrimidin-5-yl]-phenyl}urea,
1-[4-(6-aminopyridin-3-yl)-2-fluorophenyl]-3-(5-tert-butylisoxazol-3-yl)-urea,
1-(5-tert-butylisoxazol-3-yl)-3-(4-(2-(2-(piperidin-1-yl)ethylamino)pyrimidin-5-yl)phenyl)urea,
1-(5-tert-butylisoxazol-3-yl)-3-{5-[2-(2-morpholin-4-yl-ethylamino)pyrimidin-5-yl]-pyridin-2-yl}urea,
1-(5-(2-(tert-butylamino)pyrimidin-5-yl)pyridin-2-yl)-3-(5-tert-butylisoxazol-3-yl)urea,
1-(5-tert-butylisoxazol-3-yl)-3-(5-(2-(tetrahydro-2H-pyran-4-ylamino)pyrimidin-5-yl)pyridin-2-yl)urea,
1-(5-tert-butylisoxazol-3-yl)-3-[5-(2-cyclopropylaminopyrimidin-5-yl)-pyridin-2-yl]-urea,
1-(5-tert-butylisoxazol-3-yl)-3-(5-(2-(isopropylamino)pyrimidin-5-yl)pyridin-2-yl)urea,
N-(5-(2-(cyclopropylamino)pyrimidin-5-yl)pyridine-2-yl)-2-(3-(trifluoromethyl)phenyl)acetamide,
N-(5-(2-(isopropylamino)pyrimidin-5-yl)pyridin-2-yl)-2-(3-(trifluoromethyl)phenyl)acetamide,
1-(4-(6-aminopyridin-3-yl)-2-methoxyphenyl)-3-(5-(1,3-difluoro-2-methylpropan-2-yl)isoxazol-3-yl)urea,
1-(4-(6-aminopyridin-3-yl)-2-methoxyphenyl)-3-(5-(tert-butyl)isoxazol-3-yl)urea,
1-(4-(6-aminopyridin-3-yl)phenyl)-3-(3-(tert-butyl)isoxazol-5-yl)urea,
2-(4-(6-aminopyridin-3-yl)phenyl)-N-(5-(tert-butyl)isoxazol-3-yl)propanamide,
2-(4-(6-aminopyridin-3-yl)phenyl)-N-(5-(1,1,1-trifluoro-2-methylpropan-2-yl)isoxazol-3-yl)acetamide,
2-(4-(6-aminopyridin-3-yl)phenyl)-N-(5-(1-(trifluoromethyl)cyclopropyl)isoxazol-3-yl)acetamide,
2-(4-(6-aminopyridin-3-yl)phenyl)-N-(5-(1-(trifluoromethyl)cyclopropyl)isoxazol-3-yl)acetamide,
2-(4-(2-aminopyrimidin-5-yl)phenyl)-N-(5-(tert-butyl)isoxazol-3-yl)acetamide,
N-(5-(tert-butyl)isoxazol-3-yl)-2-(4-(2-(2-morpholinoethyl)amino)pyrimidin-5-yl)phenyl)acetamide,
2-(6′-amino-[3,3′-bipyridin]-6-yl)-N-(5-(tert-butyl)isoxazol-3-yl)acetamide,
2-(5-(2-aminopyrimidin-5-yl)pyridin-2-yl)-N-(5-(tert-butyl)isoxazol-3-yl)acetamide,
2-(4-(6-aminopyridin-3-yl)-2-fluorophenyl)-N-(5-(tert-butyl)isoxazol-3-yl)acetamide,
2-(4-(6-aminopyridin-3-yl)-2-fluorophenyl)-N-(5-(tert-butyl)isoxazol-3-yl)acetamide,
2-(4-(2-aminopyrimidin-5-yl)-2-fluorophenyl)-N-(5-(tert-butyl)isoxazol-3-yl)acetamide,
1-(4-(6-aminopyridin-3-yl)phenyl)-3-(5-(1-hydroxy-2-methylpropan-2-yl)isoxazol-3-yl)urea,
1-(4-(6-aminopyridin-3-yl)phenyl)-3-(3-(1-hydroxy-2-methylpropan-2-yl)isoxazol-5-yl)urea,
1-(4-(6-aminopyridin-3-yl)phenyl)-3-(3-(1-hydroxy-2-methylpropan-2-yl)-1-methyl-1H-pyrazol-5-yl)urea,
2-(4-(6-aminopyridin-3-yl)phenyl)-N-(3-(1-hydroxy-2-methylpropan-2-yl)isoxazol-5-yl)acetamide,
2-(4-(6-aminopyridin-3-yl)phenyl)-N-(3-(tert-butyl)isoxazol-5-yl)acetamide,
2-(4-(6-amino-5-methylpyridin-3-yl)phenyl)-N-(5-(tert-butyl)isoxazol-3-yl)acetamide,
2-(4-(6-amino-4-methylpyridin-3-yl)phenyl)-N-(5-(tert-butyl)isoxazol-3-yl)acetamide,
2-(4-(6-amino-2-methylpyridin-3-yl)phenyl)-N-(5-(tert-butyl)isoxazol-3-yl)acetamide,
2-(4-(6-aminopyridin-3-yl)phenyl)-N-(5-(1-hydroxy-2-methylpropan-2-yl)isoxazol-3-yl)acetamide,
N-(5-(tert-butyl)isoxazol-3-yl)-2-(4-(6-((2-methoxyethyl)amino)pyridin-3-yl)phenyl)acetamide,
1-(4-(6-aminopyridin-3-yl)phenyl)-3-(3-(1-fluoro-2-methylpropan-2-yl)-1-methyl-1H-pyrazol-5-yl)urea,
2-(4-(6-aminopyridin-3-yl)phenyl)-N-(3-(tert-butyl)-1H-pyrazol-1-yl)acetamide compound with 2-(4-(6-aminopyridin-3-yl)phenyl)-N-(5-(tert-butyl)-1H-pyrazol-1-yl)acetamide (1:1),
N-(5-(tert-butyl)isoxazol-3-yl)-2-(4-(6-((2-(methylsulfonyl)ethyl)amino)pyridin-3-yl)phenyl)acetamide,
2-(4-(6-amino-5-cyanopyridin-3-yl)phenyl)-N-(5-(tert-butyl)isoxazol-3-yl)acetamide,
2-(4-(6-amino-5-fluoropyridin-3-yl)phenyl)-N-(5-(tert-butyl)isoxazol-3-yl)acetamide,
2-(4-(6-amino-2-fluoropyridin-3-yl)phenyl)-N-(5-(tert-butyl)isoxazol-3-yl)acetamide,
N-(3-(tert-butyl)isoxazol-5-yl)-2-(4-(6-((2-morpholinoethyl)amino)pyridin-3-yl)phenyl)acetamide,
1-(4-(6-aminopyridin-3-yl)phenyl)-3-(3-(1-fluoro-2-methylpropan-2-yl)isoxazol-5-yl)urea,
1-(4-(6-aminopyridin-3-yl)phenyl)-3-(5-(3-methyloxetan-3-yl)isoxazol-3-yl)urea,
2-(4-(6-aminopyridin-3-yl)phenyl)-N-(5-(3-methyloxetan-3-yl)isoxazol-3-yl)acetamide,
2-(4-(6-aminopyridin-3-yl)phenyl)-N-(5-(1-methylcyclopropyl)isoxazol-3-yl)acetamide,
2-(4-(6-aminopyridin-3-yl)phenyl)-N-(3-(2-fluoropropan-2-yl)isoxazol-5-yl)acetamide,
2-(4-(6-aminopyridin-3-yl)phenyl)-N-(5-(2,2-difluoro-1-methylcyclopropyl)isoxazol-3-yl)acetamide,
2-(4-(6-aminopyridin-3-yl)phenyl)-N-(5-(1-(fluoromethyl)cyclopropyl)isoxazol-3-yl)acetamide,
1-(6′-amino-[3,3′-bipyridin]-6-yl)-3-(5-(1-methylcyclopropyl)isoxazol-3-yl)urea,
N-(5-(tert-butyl)isoxazol-3-yl)-2-(4-(6-((2-morpholinoethyl)amino)pyridin-3-yl)phenyl)acetamide,
1-(4-(6-aminopyridin-3-yl)phenyl)-3-(5-(1-methylcyclopropyl)isoxazol-3-yl)urea,
1-(5-(tert-butyl)isoxazol-3-yl)-3-(4-(6-((2-(4,4-difluoropiperidin-1-yl)ethyl)amino)pyridin-3-yl)phenyl)urea,
N-(5-(tert-butyl)isoxazol-3-yl)-2-(4-(6-((2-(4,4-difluoropiperidin-1-yl)ethyl)amino)pyridin-3-yl)phenyl)acetamide,
2-(4-(6-((2-morpholinoethyl)amino)pyridin-3-yl)phenyl)-N-(5-(1,1,1-trifluoro-2-methylpropan-2-yl)isoxazol-3-yl)acetamide,
N-(5-(tert-butyl)isoxazol-3-yl)-2-(4-(6-(methylamino)pyridin-3-yl)phenyl)acetamide,

N-(5-(tert-butyl)isoxazol-3-yl)-2-(4-(6-(ethylamino)pyridin-3-yl)phenyl)acetamide,
1-(4-(6-aminopyridin-3-yl)phenyl)-3-(5-(2,2-difluoro-1-methylcyclopropyl)isoxazol-3-yl)urea,
2-(4-(5-amino-6-methylpyrazin-2-yl)phenyl)-N-(5-(tert-butyl)isoxazol-3-yl)acetamide,
3-amino-6-(4-(2-((5-(tert-butyl)isoxazol-3-yl)amino)-2-oxoethyl)phenyl)pyrazine-2-carboxamide,
2-(4-(6-amino-5-chloropyridin-3-yl)phenyl)-N-(5-(tert-butyl)isoxazol-3-yl)acetamide,
2-(4-(6-amino-5-(trifluoromethyl)pyridin-3-yl)phenyl)-N-(5-(tert-butyl)isoxazol-3-yl)acetamide,
N-(5-(tert-butyl)isoxazol-3-yl)-2-(4-(6-((2-(1,2,2,6,6-pentamethylpiperidin-4-ylidene)ethyl)amino)pyridin-3-yl)phenyl)acetamide,
N-(3-(2-fluoropropan-2-yl)isoxazol-5-yl)-2-(4-(6-((2-morpholinoethyl)amino)pyridin-3-yl)phenyl)acetamide,
2-(4-(6-amino-2-fluoropyridin-3-yl)phenyl)-N-(3-(tert-butyl)isoxazol-5-yl)acetamide,
N-(5-(tert-butyl)isoxazol-3-yl)-2-(4-(5,6,7,8-tetrahydro-1,8-naphthyridin-3-yl)phenyl)acetamide,
1-(4-(6-aminopyridin-3-yl)phenyl)-3-(5-(1-(trifluoromethyl)cyclobutyl)isoxazol-3-yl)urea,
N-(5-(tert-butyl)isoxazol-3-yl)-2-(4-(6-((2-(3-methyloxetan-3-yl)ethyl)amino)pyridin-3-yl)phenyl)acetamide,
2-(4-(6-aminopyridin-3-yl)-2,6-difluorophenyl)-N-(5-(tert-butyl)isoxazol-3-yl)acetamide,
2-(4-(6-aminopyridin-3-yl)phenyl)-N-(5-(1-(trifluoromethyl)cyclobutyl)isoxazol-3-yl)acetamide,
2-(4-(6-aminopyridin-3-yl)-3-fluorophenyl)-N-(5-(tert-butyl)isoxazol-3-yl)acetamide,
2-(4-(6-aminopyridin-3-yl)phenyl)-N-(4-(trifluoromethyl)-1H-pyrazol-1-yl)acetamide,
2-(4-(6-amino-5-fluoropyridin-3-yl)phenyl)-N-(5-(1-(trifluoromethyl)cyclopropyl)isoxazol-3-yl)acetamide,
2-(4-(6-amino-5-chloropyridin-3-yl)phenyl)-N-(5-(3-methyloxetan-3-yl)isoxazol-3-yl)acetamide,
2-(4-(6-amino-2-(trifluoromethyl)pyridin-3-yl)phenyl)-N-(5-(tert-butyl)isoxazol-3-yl)acetamide,
2-(4-(6-amino-5-methylpyridin-3-yl)phenyl)-N-(5-(1-(trifluoromethyl)cyclopropyl)isoxazol-3-yl)acetamide,
2-(4-(6-amino-2-methoxypyridin-3-yl)phenyl)-N-(5-(tert-butyl)isoxazol-3-yl)acetamide,
2-(4-(6-amino-2-chloropyridin-3-yl)phenyl)-N-(5-(tert-butyl)isoxazol-3-yl)acetamide,
2-(4-(6-amino-5-chloropyridin-3-yl)phenyl)-N-(5-(1-(trifluoromethyl)cyclopropyl)isoxazol-3-yl)acetamide,
2-(4-(6-amino-2-fluoropyridin-3-yl)phenyl)-N-(5-(1-methylcyclopropyl)isoxazol-3-yl)acetamide,
2-(4-(6-amino-2-fluoropyridin-3-yl)phenyl)-N-(5-(1-methylcyclopropyl)isoxazol-3-yl)acetamide,
2-(4-(6-amino-5-(trifluoromethyl)pyridin-3-yl)phenyl)-N-(5-(3-methyloxetan-3-yl)isoxazol-3-yl)acetamide,
2-(4-(6-amino-5-methoxypyridin-3-yl)phenyl)-N-(5-(tert-butyl)isoxazol-3-yl)acetamide,
2-(4-(6-amino-2-fluoropyridin-3-yl)phenyl)-N-(5-(1-(trifluoromethyl)cyclopropyl)isoxazol-3-yl)acetamide,
2-(4-(6-amino-5-fluoropyridin-3-yl)phenyl)-N-(5-(1-methylcyclopropyl)isoxazol-3-yl)acetamide,
2-(4-(6-aminopyridin-3-yl)-2-fluorophenyl)-N-(5-(1-(trifluoromethyl)cyclopropyl)isoxazol-3-yl)acetamide,
2-(4-(6-amino-2-fluoropyridin-3-yl)phenyl)-N-(5-(3-methyloxetan-3-yl)isoxazol-3-yl)acetamide,
N-(5-(tert-butyl)isoxazol-3-yl)-2-(4-(2-oxo-2,3-dihydrooxazolo[4,5-b]pyridin-6-yl)phenyl)acetamide,
2-(4-(6-amino-2-fluoropyridin-3-yl)phenyl)-N-(5-(1,1,1-trifluoro-2-methylpropan-2-yl)isoxazol-3-yl)acetamide,
2-(4-(6-aminopyridin-3-yl)phenyl)-N-(3-cyclobutylisoxazol-5-yl)acetamide;
2-(4-(6-aminopyridin-3-yl)phenyl)-N-(5-(1-methylcyclobutyl)isoxazol-3-yl)acetamide,
2-(4-(6-amino-5-methylpyridin-3-yl)phenyl)-N-(5-(3-methyloxetan-3-yl)isoxazol-3-yl)acetamide,
2-(4-(6-amino-5-fluoropyridin-3-yl)phenyl)-N-(5-(3-methyloxetan-3-yl)isoxazol-3-yl)acetamide,

N-(5-(tert-butyl)isoxazol-3-yl)-2-(4-(2-oxo-2,3-dihydro-1H-imidazo[4,5-b]pyridin-6-yl)phenyl)acetamide,
2-(6′-amino-5-fluoro-[3,3′-bipyridin]-6-yl)-N-(5-(tert-butyl)isoxazol-3-yl)acetamide,
2-(4-(6-aminopyridin-3-yl)phenyl)-N-(5-(1-(difluoromethyl)cyclopropyl)isoxazol-3-yl)acetamide,
2-(4-(6-amino-4-chloropyridin-3-yl)phenyl)-N-(5-(tert-butyl)isoxazol-3-yl)acetamide,
2-(4-(6-amino-4-fluoropyridin-3-yl)phenyl)-N-(5-(tert-butyl)isoxazol-3-yl)acetamide,
2-(4-(6-amino-2,5-difluoropyridin-3-yl)phenyl)-N-(5-(tert-butyl)isoxazol-3-yl)acetamide,
2-(4-(6-aminopyridin-3-yl)phenyl)-N-(5-(1-(1,1-difluoroethyl)cyclopropyl)isoxazol-3-yl)acetamide,
2-(4-(6-amino-5-(1-methyl-1H-pyrazol-4-yl)pyridin-3-yl)phenyl)-N-(5-(tert-butyl)isoxazol-3-yl)acetamide,
2-(6′-amino-[2,3′-bipyridin]-5-yl)-N-(5-(tert-butyl)isoxazol-3-yl)acetamide,
2-(4-(6-amino-4-(trifluoromethyl)pyridin-3-yl)phenyl)-N-(5-(tert-butyl)isoxazol-3-yl)acetamide,
2-(4-(6-amino-4-methoxypyridin-3-yl)phenyl)-N-(5-(tert-butyl)isoxazol-3-yl)acetamide,
2-(4-(6-amino-5-methylpyridin-3-yl)phenyl)-N-(5-(1-methylcyclopropyl)isoxazol-3-yl)acetamide,
2-(4-(6-amino-2-fluoropyridin-3-yl)phenyl)-N-(5-(1-(1,1-difluoroethyl)cyclopropyl)isoxazol-3-yl)acetamide,
2-(4-(6-amino-5-chloropyridin-3-yl)phenyl)-N-(5-(1-methylcyclopropyl)isoxazol-3-yl)acetamide,
2-(4-(6-amino-5-(trifluoromethyl)pyridin-3-yl)phenyl)-N-(5-(1-methylcyclopropyl)isoxazol-3-yl)acetamide,
2-(4-(6-amino-2-methylpyridin-3-yl)phenyl)-N-(5-(1-methylcyclopropyl)isoxazol-3-yl)acetamide,
2-(4-(6-amino-5-(difluoromethyl)pyridin-3-yl)phenyl)-N-(5-(tert-butyl)isoxazol-3-yl)acetamide,
2-(4-(6-amino-5-methoxypyridin-3-yl)phenyl)-N-(5-(1-methylcyclopropyl)isoxazol-3-yl)acetamide,
2-(4-(5-amino-3-methylpyrazin-2-yl)phenyl)-N-(5-(tert-butyl)isoxazol-3-yl)acetamide,
2-(4-(6-amino-2-cyanopyridin-3-yl)phenyl)-N-(5-(tert-butyl)isoxazol-3-yl)acetamide,
2-(4-(6-amino-2-fluoropyridin-3-yl)phenyl)-N-(5-(1-(trifluoromethyl)cyclobutyl)isoxazol-3-yl)acetamide,
(1-(3-(2-(4-(6-aminopyridin-3-yl)phenyl)acetamido)isoxazol-5-yl)cyclopropyl)methyl acetate,
2-(4-(6-amino-4-fluoropyridin-3-yl)phenyl)-N-(5-(1-methylcyclopropyl)isoxazol-3-yl)acetamide,
2-(4-(6-aminopyridin-3-yl)phenyl)-N-(5-(1-ethylcyclopropyl)isoxazol-3-yl)acetamide,
2-(4-(6-amino-4-chloropyridin-3-yl)phenyl)-N-(5-(1-methylcyclopropyl)isoxazol-3-yl)acetamide,
2-amino-2-(4-(6-aminopyridin-3-yl)phenyl)-N-(5-(tert-butyl)isoxazol-3-yl)acetamide,
2-(4-(5-amino-3,6-dimethylpyrazin-2-yl)phenyl)-N-(5-(tert-butyl)isoxazol-3-yl)acetamide,
2-(4-(6-amino-5-(tert-butylthio)pyridin-3-yl)phenyl)-N-(5-(tert-butyl)isoxazol-3-yl)acetamide,
2-(4-(6-amino-5-(tert-butylsulfonyl)pyridin-3-yl)phenyl)-N-(5-(tert-butyl)isoxazol-3-yl)acetamide,
2-(4-(6-amino-2,5-difluoropyridin-3-yl)phenyl)-N-(5-(1-methylcyclopropyl)isoxazol-3-yl)acetamide,
2-(4-(6-aminopyridin-3-yl)phenyl)-N-(5-(tert-butyl)isoxazol-3-yl)-2,2-difluoroacetamide,
2-(4-(6-amino-5-(tert-butylsulfinyl)pyridin-3-yl)phenyl)-N-(5-(tert-butyl)isoxazol-3-yl)acetamide,
2-(4-(6-aminopyridin-3-yl)-3-(trifluoromethyl)phenyl)-N-(5-(tert-butyl)isoxazol-3-yl)acetamide,
N-(5-(tert-butyl)isoxazol-3-yl)-2-(4-(2-methyl-3-oxo-2,3-dihydro-1H-pyrazolo[3,4-b]pyridin-5-yl)phenyl)acetamide,
2-(4-(6-aminopyridin-3-yl)phenyl)-N-(5-(tert-butyl)isoxazol-3-yl)-2-hydroxyacetamide,
2-(4-(6-amino-4-fluoropyridin-3-yl)phenyl)-N-(3-(tert-butyl)isoxazol-5-yl)acetamide,
2-(4-(6-amino-4-fluoropyridin-3-yl)phenyl)-N-(5-(1-(1,1-difluoroethyl)cyclopropyl)isoxazol-3-yl)acetamide,
N-(5-(1-methylcyclopropyl)isoxazol-3-yl)-2-(4-(6-((2-(methylsulfonyl)ethyl)amino)pyridin-3-yl)phenyl)acetamide,
2-(4-(6-aminopyridin-3-yl)phenyl)-N-(5-isopropylisoxazol-3-yl)acetamide,
N-(5-(tert-butyl)isoxazol-3-yl)-2-(4-(2-oxo-2,3,4,5-tetrahydro-1H-pyrido[2,3-e][1,4]diazepin-7-yl)phenyl)acetamide,
2-(4-(6-((2-(methylsulfonyl)ethyl)amino)pyridin-3-yl)phenyl)-N-(5-(1-(trifluoromethyl)cyclopropyl)isoxazol-3-yl)acetamide,
N-(5-(tert-butyl)isoxazol-3-yl)-2-(4-(6-oxo-5,6-dihydro-1,5-naphthyridin-3-yl)phenyl)acetamide,
2-(4-(6-amino-4-fluoropyridin-3-yl)phenyl)-N-(5-(1-(trifluoromethyl)cyclopropyl)isoxazol-3-yl)acetamide, and
N-(5-(tert-butyl)isoxazol-3-yl)-2-(4-(4-methyl-2-oxo-2,3,4,5-tetrahydro-1H-pyrido[2,3-e][1,4]diazepin-7-yl)phenyl)acetamide, or a pharmaceutically acceptable salt thereof.

Also provided herein are isotopically enriched analogs of the compounds provided herein. Isotopic enrichment (for example, deuteration) of pharmaceuticals to improve pharmacokinetics (“PK”), pharmacodynamics (“PD”), and toxicity profiles, has been demonstrated previously with some classes of drugs. See, for example, Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade D, Chem. Biol. Interact. 117: 191 (1999).

Isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). (See, e.g., Foster et al., Adv. Drug Res., vol. 14, pp. 1-36 (1985); Kushner et al., Can. J. Physiol. Pharmacol., vol. 77, pp. 79-88 (1999)).

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, will provide a similar kinetic isotope effects.

In another embodiment, provided herein are methods of using the disclosed compounds and compositions, or pharmaceutically acceptable salts, solvates, or hydrates thereof, for the local or systemic treatment or prophylaxis of human and veterinary diseases, disorders and conditions modulated or otherwise affected mediated via CSF-1R and/or FLT3 kinase activity.

### C. FORMULATION OF PHARMACEUTICAL COMPOSITIONS

The pharmaceutical compositions provided herein contain therapeutically effective amounts of one or more of compounds provided herein that are useful in the prevention, treatment, or amelioration of CSF-1R and/or FLT3 kinase mediated diseases or one or more of the symptoms thereof.

The compositions contain one or more compounds provided herein.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the compounds described above are formulated into pharmaceutical compositions using techniques and procedures well known in the art.

In the compositions, effective concentrations of one or more compounds or pharmaceutically acceptable salt, solvate, hydrate or prodrug is (are) mixed with a suitable pharmaceutical carrier or vehicle. The concentrations of the compounds in the compositions are effective for delivery of an amount, upon administration, that treats, prevents, or ameliorates one or more of the symptoms of CSF-1R and/or FLT3 kinase mediated diseases.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of CSF-1R and/or FLT3 kinase mediated diseases.

Typically a therapeutically effective dosage should produce a serum concentration of active ingredient of from about 1 ng/ml to about 50-100 μg/ml. The pharmaceutical compositions typically should provide a dosage of from about 10 mg to about 4000 mg of compound per kilogram of body weight per day. Pharmaceutical dosage unit forms are prepared to provide from about 10 mg to about 1000 mg and in certain embodiments, from about 10 mg to about 500 mg, from about 20 mg to about 250 mg or from about 25 mg to about 100 mg of the essential active ingredient or a combination of essential ingredients per dosage unit form. In certain embodiments, the pharmaceutical dosage unit forms are prepared to provide about 10 mg, 20 mg, 25 mg, 50 mg, 100 mg, 250 mg, 500 mg, 1000 mg or 2000 mg of the essential active ingredient.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing CSF-1R and/or FLT3 kinase mediated diseases. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including, but not limited to, orally, parenterally, rectally, topically and locally. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. In one embodiment, the effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refer to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001%-100% active ingredient, in certain embodiments, about 0.1-85%, typically about 75-95%.

The active compounds or pharmaceutically acceptable derivatives may be prepared with carriers that protect the compound against rapid elimination from the body, such as time release formulations or coatings.

The compositions may include other active compounds to obtain desired combinations of properties. The compounds provided herein, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as CSF-1R and/or FLT3 kinase mediated diseases. It is to be understood that such combination therapy constitutes a further aspect of the compositions and methods of treatment provided herein.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

2. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. In one embodiment, the composition is administered as an aqueous solution with hydroxypropyl-beta-cyclodextrin (HPBCD) as an excipient. In one embodiment, the aqueous solution contains about 1% to about 50% HPBCD. In one embodiment, the aqueous solution contains about 1%, 3%, 5%, 10% or about 20% HPBCD.

Implantation of a slow-release or sustained-release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose, hydroxypropyl-beta-cyclodextrin (HPBCD) or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg, 100-500 mg, 10-500 mg, 50-250 mg or 25-100 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation. These formulations for administration to the respiratory tract can be in the form of an aerosol or solution for a nebulizer, or as a microtine powder for insufflation, alone or in combination with an inert carrier such as lactose. In such a case, the particles of the formulation will typically have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

6. Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used. In another embodiment, polymeric materials can be used. In yet another embodiment, a controlled release system can be placed in proximity of the therapeutic target, i.e., thus requiring only a fraction of the systemic dose. In some embodiments, a controlled release device is introduced into a subject in proximity of the site of inappropriate immune activation or a tumor. The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

7. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### D. EVALUATION OF THE ACTIVITY OF THE COMPOUNDS

Standard physiological, pharmacological and biochemical procedures are available for testing the compounds to identify those that possess biological activities that modulate the activity of FLT3 and/or CSF-1R kinases.

Such assays include, for example, biochemical assays such as binding assays, radioactivity incorporation assays, as well as a variety of cell based assays.

In certain embodiments, the compounds disclosed herein are tested in an M-NFS-60 cell proliferation assay to determine their cellular potency against CSF-1R. M-NFS-60s are mouse monocytic cells that depend on the binding of the ligand M-CSF to its receptor, CSF-1R, to proliferate Inhibition of CSF-1R kinase activity will cause reduced growth and/or cell death. This assay assesses the potency of compounds as CSF-1R inhibitors by measuring the reduction of Alamar Blue reagent by viable cells. An exemplary assay is described in the Examples section.

In certain embodiments, competition binding assays were performed as described in Fabian et al., Nature Biotechnology 2005, 23,329-336.

### E. METHODS OF USE OF THE COMPOUNDS AND COMPOSITIONS

Also provided herein are methods of using the disclosed compounds and compositions, or pharmaceutically acceptable salts, solvates, hydrates or prodrugs thereof, for the treatment, prevention, or amelioration of a disease or disorder that is mediated or otherwise affected via protein kinase activiy or one or more symptoms of diseases or disorders that are mediated or otherwise affected via protein kinase activity (see, Krause and Van Etten, N Engl J Med (2005) 353(2):172-187, Blume-Jensen and Hunter, Nature (2001) 411(17): 355-365 and Plowman et al., DN&P, 7:334-339 (1994)).

In certain embodiments, provided herein are methods of treating the following diseases or disorders:

1) carcinomas include Kit-mediated and/or CSF-1R-mediated carcinomas, adenocarcinoma, squamous cell carcinoma, adenosquamous carcinoma, teratocarcinoma, head and neck cancer, brain cancer, intracranial carcinoma, glioblastoma including PDGFR-mediated glioblastoma, glioblastoma multiforme including PDGFR-mediated glioblastoma multiforme, neuroblastoma, cancer of the larynx, multiple endocrine neoplasias 2A and 2B (MENS 2A and MENS 2B) including RET-mediated MENS, thyroid cancer, including sporadic and familial medullary thyroid carcinoma, papillary thyroid carcinoma, parathyroid carcinoma including any RET-mediated thyroid carcinoma, follicular thyroid cancer, anaplastic thyroid cancer, bronchial carcinoid, oat cell carcinoma, lung cancer, small-cell lung cancer including flt-3 and/or Kit-mediated small cell lung cancer, stomach/gastric cancer, gastrointestinal cancer, gastrointestinal stromal tumors (GIST) including Kit-mediated GIST and PDGFRα-mediated GIST, colon cancer, colorectal cancer, pancreatic cancer, islet cell carcinoma, hepatic/liver cancer, metastases to the liver, bladder cancer, renal cell cancer including PDGFR-mediated renal cell cancer, cancers of the genitourinary tract, ovarian cancer including Kit-mediated and/or PDGFR-mediated and/or CSF-1R-mediated ovarian cancer, endometrial cancer including CSF-1R-mediated endometrial cancer, cervical cancer, breast cancer including Flt-3-mediated and/or PDGFR-mediated and/or CSF-1R-mediated breast cancer, prostate cancer including Kit-mediated prostate cancer, germ cell tumors including Kit-mediated germ cell tumors, seminomas including Kit-mediated seminomas, dysgerminomas, including Kit-mediated dysgerminomas, melanoma including PDGFR-mediated melanoma, metastases to the bone including CSF-1R-mediated bone metastases, metastatic tumors including VEGFR-mediated and/or CSF-1R metastatic tumors, stromal tumors, neuroendocrine tumors, tumor angiogenesis including VEGFR-mediated and/or CSF-1R-mediated tumor angiogenesis, mixed mesodermal tumors;

2) sarcomas including PDGFR-mediated sarcomas, osteosarcoma, osteogenic sarcoma, bone cancer, glioma including PDGFR-mediated and/or CSF-1R-mediated glioma, astrocytoma, vascular tumors including VEGFR-mediated vascular tumors, Kaposi's sarcoma, carcinosarcoma, hemangiosarcomas including VEGFR3-mediated hemangiosarcomas, lymphangiosarcoma including VEGFR3-mediated lymphangiosarcoma;

3) myeloma, leukemia, myeloproliferative diseases, acute myeloid leukemia (AML) including flt-3 mediated and/or KIT-mediated and/or CSF1R-mediated acute myeloid leukemia, chronic myeloid leukemias (CML) including Flt-3-mediated and/or PDGFR-mediated chronic myeloid leukemia, myelodysplastic leukemias including Flt-3-mediated myelodysplastic leukemia, acute megakaryoblastic leukemia CSF1R-mediated acute megakaryoblastic leukemia, myelodysplastic syndrome, including Flt-3 mediated and/or Kit-mediated myelodysplastic syndrome, idiopathic hypereosinophilic syndrome (HES) including PDGFR-mediated HES, chronic eosinophilic leukemia (CEL) including PDGFR-mediated CEL, chronic myelomonocytic leukemia (CMML), mast cell leukemia including Kit-mediated mast cell leukemia, or systemic mastocytosis including Kit-mediated systemic mastocytosis; and

4) lymphoma, lymphoproliferative diseases, acute lymphoblastic leukemia (ALL), B-cell acute lymphoblastic leukemias, T-cell acute lymphoblastic leukemias, natural killer (NK) cell leukemia, B-cell lymphoma, T-cell lymphoma, and natural killer (NK) cell lymphoma, any of which may be Flt-3 mediated and/or PDGFR-mediated, Langerhans cell histiocytosis including CSF-1R-mediated and flt-3-mediated Langerhans cell histiocytosis, mast cell tumors and mastocytosis; 2) Nonmalignant proliferation diseases; atherosclerosis including PDGFR-mediated atherosclerosis, restenosis following vascular angioplasty including PDGFR-mediated restenosis, and fibroproliferative disorders such as obliterative bronchiolitis and idiopathic myelofibrosis, both of which may be PDGFR-mediated, pulmonary fibrosis and obesity;

5) Inflammatory diseases or immune disorders including autoimmune diseases, which include, but is not limited to, tissue transplant rejection, graft-versus-host disease, wound healing, kidney disease, multiple sclerosis, thyroiditis, type 1 diabetes, sarcoidosis, allergic rhinitis, nephritis, Alzheimer's disease, inflammatory bowel disease including Crohn's disease and ulcerative colitis (UC), systemic lupus erythematosis (SLE), arthritis, osteoarthritis, rheumatoid arthritis, psoriatic arthritis, inflammatory arthritis, osteoporosis, asthma and chronic obstructive pulmonary disease (COPD), including any of the aforementioned diseases which are flt-3-mediated and/or CSF-1R-mediated and/or KIT-mediated;

6) Bone diseases including disorders relating to the mineralization, formation and resorption of the bone, including but not limited to osteoporosis, glucocorticoid-induced osteoporosis, periodontitis, bone loss due to cancer therapy, periprosthetic osteolysis, Paget's disease, hypercalcemia, osteomyelitis, and bone pain; and

7) Infectious diseases mediated either via viral or bacterial pathogens and sepsis, including KIT-mediated and/or CSF-1R-mediated sepsis.

Also provided are methods of modulating the activity, or subcellular distribution, of kinases in a cell, tissue or whole organism, using the compounds and compositions provided herein, or pharmaceutically acceptable derivatives thereof. In one embodiment, provided herein are methods of modulating the activity of Flt3 activity in a cell, tissue or whole organism using the compounds and compositions provided herein, or pharmaceutically acceptable derivatives thereof. In one embodiment, provided herein are methods of modulating the activity of CSF-1R activity in a cell, tissue or whole organism using the compounds and compositions provided herein, or pharmaceutically acceptable derivatives thereof. In one embodiment, provided herein are methods of modulating the activity of KIT activity in a cell, tissue or whole organism using the compounds and compositions provided herein, or pharmaceutically acceptable derivatives thereof.

In one embodiment, the methods provided herein are for treating tumor-associated osteolysis, osteoporosis including ovariectomy-induced bone loss, orthopedic implant failure, renal inflammation and glomerulonephritis, transplant rejection including renal and bone marrow allografts and skin xenograft, obesity, Alzheimer's Disease and Langerhans cell histiocytosis. In one embodiment, the methods provided herein are for treating chronic skin disorders including psoriasis.

In another embodiment, a method for treating periodontitis, Langerhans cell histiocytosis, osteoporosis, Paget's disease of bone (PDB), bone loss due to cancer therapy, periprosthetic osteolysis, glucocorticoid-induced osteoporosis, rheumatoid arthritis, psoriatic arthritis, osteoarthritis, and/or inflammatory arthritis is provided herein.

In one embodiment, the methods provided herein are for treating bone diseases including disorders relating to the mineralization, formation and resorption of the bone, including but not limited to osteoporosis, Paget's disease, hypercalcemia, osteolysis, osteomyelitis, and bone pain.

In one embodiment, the methods provided herein are for treating cancers, including, but not limited to head and neck cancer, (originating in lip, oral cavity, oropharynx, hypopharynx, larynx, nasopharynx, nasal cavity and paranasal sinuses or salivary glands); lung cancer, including small cell lung cancer, non-small cell lung cancer; gastrointestinal tract cancers, including esophageal cancer, gastric cancer, colorectal cancer, anal cancer, pancreatic cancer, liver cancer, gallbladder cancer, extrahepatic bile duct cancer, cancer of the ampulla of vater; breast cancer; gynecologic cancers, including, cancer of uterine cervix, cancer of the uterine body, vaginal cancer, vulvar cancer, ovarian cancer, gestational trophoblastic cancer neoplasia; testicular cancer; urinary tract cancers, including, renal cancer, urinary bladder cancer, prostate cancer, penile cancer, urethral cancer; neurologic tumors; endocrine neoplasms, including carcinoid and islet cell tumors, pheochromocytoma, adrenal cortical carcinoma, parathyroid carcinoma and metastases to endocrine glands. In another embodiment, the methods provided herein are for treating carcinoma, breast cancer, ovarian cancer, bone metastases, osteoporosis, Paget's disease, hypercalcemia, osteolysis, osteomyelitis, bone pain, inflammatory bowel disease (IBD), Crohn's disease, ulcerative colitis (UC), systemic lupus erythematosis (SLE), arthritis, osteoarthritis, rheumatoid arthritis, osteoporosis, asthma, chronic obstructive pulmonary disease (COPD), psoriasis and multiple sclerosis.

Further examples of cancers are basal cell carcinoma; squamous cell carcinoma; chondrosarcoma (a cancer arising in cartilage cells); mesenchymal-chondrosarcoma; soft tissue sarcomas, including, malignant tumours that may arise in any of the mesodermal tissues (muscles, tendons, vessels that carry blood or lymph, joints and fat); soft tissue sarcomas include; alveolar soft-part sarcoma, angiosarcoma, fibrosarcoma, leiomyosarcoma, liposarcoma, malignant fibrous histiocytoma, hemangiopericytoma, mesenchymoma, schwannoma, peripheral neuroectodermal tumours, rhabdomyosarcoma, synovial sarcoma; gestational trophoblastic tumour (malignancy in which the tissues formed in the uterus following conception become cancerous); Hodgkin's lymphoma and laryngeal cancer.

In one embodiment, the cancer is a leukemia. In one embodiment, the leukemia is chronic lymphocytic leukemia, chronic myelocytic leukemia, acute lymphoblastic leukemia, acute myeloid leukemia, and acute myeloblastic leukemia.

In another embodiment, the leukemia is acute leukemia. In one embodiment, the acute leukemia is acute myeloid leukemia (AML). In one embodiment, acute myeloid leukemia is undifferentiated AML (MO), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), or megakaryoblastic leukemia (M7). In another embodiment, the acute myeloid leukemia is undifferentiated AML (M0). In yet another embodiment, the acute myeloid leukemia is myeloblastic leukemia (M1). In yet another embodiment, the acute myeloid leukemia is myeloblastic leukemia (M2). In yet another embodiment, the acute myeloid leukemia is promyelocytic leukemia (M3 or M3 variant [M3V]). In yet another embodiment, the acute myeloid leukemia is myelomonocytic leukemia (M4 or M4 variant with eosinophilia [M4E]). In yet another embodiment, the acute myeloid leukemia is monocytic leukemia (M5). In yet another embodiment, the acute myeloid leukemia is erythroleukemia (M6). In yet another embodiment, the acute myeloid leukemia is megakaryoblastic leukemia (M7). In yet another embodiment, the acute myeloid leukemia is promyelocytic leukemia. In yet another embodiment, the leukemia is attributable to a FLT3 internal tandem duplication (ITD) mutation. In yet another embodiment, the leukemia is attributable to a FLT3 point mutation. In yet another embodiment, the leukemia is attributable to a FLT3 point mutation occurring in the juxtamembrane domain. In still another embodiment, the FLT3 point mutation is a point mutation at amino acid D835.

In another embodiment, the acute leukemia is acute lymphocytic leukemia (ALL). In one embodiment, the acute lymphocytic leukemia is leukemia that originates in the blast cells of the bone marrow (B-cells), thymus (T-cells), or lymph nodes. The acute lymphocytic leukemia is categorized according to the French-American-British (FAB) Morphological Classification Scheme as L1-Mature-appearing lymphoblasts (T-cells or pre-B-cells), L2-Immature and pleomorphic (variously shaped) lymphoblasts (T-cells or pre-B-cells), and L3-Lymphoblasts (B-cells; Burkitt's cells). In another embodiment, the acute lymphocytic leukemia originates in the blast cells of the bone marrow (B-cells). In yet another embodiment, the acute lymphocytic leukemia originates in the thymus (T-cells). In yet another embodiment, the acute lymphocytic leukemia originates in the lymph nodes. In yet another embodiment, the acute lymphocytic leukemia is L1 type characterized by mature-appearing lymphoblasts (T-cells or pre-B-cells). In yet another embodiment, the acute lymphocytic leukemia is L2 type characterized by immature and pleomorphic (variously shaped) lymphoblasts (T-cells or pre-B-cells). In yet another embodiment, the acute lymphocytic leukemia is L3 type characterized by lymphoblasts (B-cells; Burkitt's cells).

In yet another embodiment, the leukemia is T-cell leukemia. In one embodiment, the T-cell leukemia is peripheral T-cell leukemia, T-cell lymphoblastic leukemia, cutaneous T-cell leukemia, and adult T-cell leukemia. In another embodiment, the T-cell leukemia is peripheral T-cell leukemia. In yet another embodiment, the T-cell leukemia is T-cell lymphoblastic leukemia. In yet another embodiment, the T-cell leukemia is cutaneous T-cell leukemia. In still another embodiment, the T-cell leukemia is adult T-cell leukemia.

In yet another embodiment, the leukemia is Philadelphia positive. In one embodiment, the Philadelphia positive leukemia is Philadelphia positive AML, including, but not limited to, undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), or megakaryoblastic leukemia (M7). In another embodiment, the Philadelphia positive leukemia is Philadelphia positive ALL.

In still another embodiment, the leukemia is drug resistant. In still another embodiment, the gastrointestinal stromal tumor (GIST) is drug resistant. In still another embodiment, the melanoma is drug resistant. In one embodiment, the subject has developed drug resistance to the anticancer therapy. In another embodiment, the subject has developed drug resistance to a FLT3 kinase inhibitor. In yet another embodiment, the subject has been treated with PKC 412, MLN 578, CEP-701, CT 53518, CT-53608, CT-52923, D-64406, D-65476, AGL-2033, AG1295, AG1296, KN-1022, PKC-412, SU5416, SU5614, SU11248, L-00021649, or CHIR-258. In still another embodiment, the subject has a constitutively activating FLT3 mutation.

The cancers to be treated herein may be primary or metastatic. In one embodiment, the cancer is a solid or blood born metastatic tumor. In another embodiment, the cancer is metastatic cancer of bone.

Also provided are methods of modulating the activity, or subcellular distribution, of FLT3 and/or CSF-1R kinases and/or KIT in a cell, tissue or whole organism, using the compounds and compositions provided herein, or a pharmaceutically acceptable salt, solvate or hydrate thereof thereof.

The active ingredient(s) in one embodiment are administered in an amount sufficient to deliver to a patient a therapeutically effective amount of the active compound in order to e.g., treat the diseases described herein, without causing serious toxic effects in a treated subject.

A typical dose of the compound may be in the range of from about 1 to about 50 mg/kg, from about 1 to about 20 mg/kg, from about 0.1 to about 10 mg/kg, from about 0.5 mg/kg to about 10 mg/kg, of body weight per day, more generally from about 0.1 to about 100 mg/kg body weight of the recipient per day. Lower dosages may be used, for example, doses of about 0.5-100 mg, 0.5-10 mg, or 0.5-5 mg per kilogram body weight per day. Even lower doses may be useful, and thus ranges can include from about 0.1-0.5 mg/kg body weight of the recipient per day. The effective dosage range of the pharmaceutically acceptable derivatives is calculated based on the weight of the parent indole derivative compound to be delivered. If the derivative compound itself exhibits activity, then the effective dosage can be estimated as above using the weight of the derivative, or by other means known to those of skill in the art.

The compounds are conveniently administered in units of any suitable dosage form, including but not limited to one containing from about 1 to 2000 mg, from about 10 to 1000 mg, or from about 25 to 700 mg of active ingredient per unit dosage form. In one embodiment, the unit dose is selected from 12, 18, 25, 27, 40, 50, 60, 90, 100, 135, 200, 250, 300, 400, 450, 500, 600, 675, 700, 800, 900 and 1000 mgs. For example, an oral dosage of from about 25 to 1000 mg is usually convenient, including in one or multiple dosage forms of 10, 12, 18, 25, 27, 40, 50, 60, 90, 100, 135, 200, 250, 300, 400, 450, 500, 600, 675, 700, 800, 900 or 1000 mgs. In certain embodiments, lower dosages may be used, for example, from about 10-100 or 1-50 mgs. Also contemplated are doses of 0.1-50 mg, 0.1-20 mgs., or 0.1-10 mgs. Furthermore, lower doses may be utilized in the case of administration by a non-oral route, as for example, by injection or inhalation.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the compositions provided herein.

In certain embodiments, the compound or composition provided herein can be administered as a single once-a-day dose or preferably as divided doses throughout a day. In particular embodiments, the compound or composition is administered four times per day. In particular embodiments, the compound or composition is administered three times per day. In particular embodiments, the compound or composition is administered two times per day. In particular embodiments, the compound or composition is administered once per day.

In one embodiment, the active ingredient is administered to achieve peak plasma concentrations of the active compound of from about 0.02 to 20 μM, from about 0.2 to about 5 μM or from about 0.5 to 10 μM. For example, this can be achieved by intravenous injection of a 0.1 to 5% solution of active ingredient, optionally in saline, or administered as a bolus of active ingredient. It is to be understood that for any particular subject, specific dosage regimens should be adjusted over time to meet individual needs, and will vary depending upon absorption, inactivation and excretion rates of the drug. The concentrations set forth here are exemplary only and are not intended to limit the scope or practice of the claimed composition. The active ingredient may be administered all at once, or may be divided into a number of smaller doses to be administered at varying intervals of time.

The subject matter has been described in an illustrative manner, and it is to be understood that the terminology used is intended to be in the nature of description rather than of limitation. Thus, it will be appreciated by those of skill in the art that conditions such as choice of solvent, temperature of reaction, volumes, reaction time may vary while still producing the desired compounds. In addition, one of skill in the art will also appreciate that many of the reagents provided in the examples may be substituted with other suitable reagents. See, e.g., Smith & March, Advanced Organic Chemistry, 5^{th} ed. (2001).

### F. COMBINATION THERAPY

Furthermore, it will be understood by those skilled in the art that the compounds, isomers, and pharmaceutically acceptable salts provided herein, including pharmaceutical compositions and formulations containing these compounds, can be used in a wide variety of combination therapies to treat the conditions and diseases described above. Thus, also contemplated herein is the use of compounds, and pharmaceutically acceptable salts provided herein in combination with other active pharmaceutical agents for the treatment of the disease/conditions described herein.

In one embodiment, such additional pharmaceutical agents include without limitation anti-cancer agents (including chemotherapeutic agents and anti-proliferative agents), anti-inflammatory agents, immunomodulatory agents or immunosuppressive agents.

In certain embodiments, the anti-cancer agents include anti-metabolites (e.g., 5-fluoro-uracil, cytarabine, clofarabine, methotrexate, fludarabine and others), antimicrotubule agents (e.g., vinca alkaloids such as vincristine, vinblastine; taxanes such as paclitaxel and docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, satraplatin and CI-973), anthracyclines (e.g., doxrubicin and daunorubicin), antitumor antibiotics (e.g., mitomycin, idarubicin, adriamycin and daunomycin), topoisomerase inhibitors (e.g., etoposide and camptothecins), anti-angiogenesis agents (e.g. Sutent®, sorafenib and Bevacizumab) or any other cytotoxic agents, (e.g. estramustine phosphate, prednimustine), hormones or hormone agonists, antagonists, partial agonists or partial antagonists, kinase inhibitors (such as imatinib), and radiation treatment.

In certain embodiments, the anti-inflammatory agents include matrix metalloproteinase inhibitors, inhibitors of pro-inflammatory cytokines (e.g., anti-TNF molecules, TNF soluble receptors, and IL1) non-steroidal anti-inflammatory drugs (NSAIDs) such as prostaglandin synthase inhibitors (e.g., choline magnesium salicylate and salicylsalicyclic acid), COX-1 or COX-2 inhibitors, glucocorticoid receptor agonists (e.g., corticosteroids, methylprednisone, prednisone, and cortisone) or antifolates such as methotrexate.

The compound or composition provided herein, or pharmaceutically acceptable salt of the compound, may be administered simultaneously with, prior to, or after administration of one or more of the above agents.

Pharmaceutical compositions containing a compound provided herein or pharmaceutically acceptable salt thereof, and one or more of the above agents are also provided.

Also provided, in one embodiment, is a combination therapy that treats or prevents the onset of the symptoms, or associated complications of cancer and related diseases and disorders, said therapy comprising the administration to a subject in need thereof, one of the compounds or compositions disclosed herein, or pharmaceutically acceptable salts thereof, with one or more anti-cancer agents. Also provided, in another embodiment, is a combination therapy that treats or prevents the onset of the symptom of osteoporosis and related diseases and disorders, said therapy comprising the administration to a subject in need thereof, one of the compounds or compositions disclosed herein, or pharmaceutically acceptable salts thereof, with one or more anti-inflammatory or immunomodulatory agents. Also provided, in yet another embodiment, is a combination therapy that treats or prevents the onset of the symptom of rheumatoid arthritis and related diseases and disorders, said therapy comprising the administration to a subject in need thereof, one of the compounds or compositions disclosed herein, or pharmaceutically acceptable salts thereof, with one or more anti-inflammatory or immunomodulatory agents.

### G. PREPARATION OF COMPOUNDS

Starting materials in the synthesis examples provided herein are either available from commercial sources or via literature procedures either as cited or as found, for example in March Advanced Organic Chemistry: Reactions, Mechanisms, and Structure, (1992) 4th Ed.; Wiley Interscience, New York. All commercially available compounds were used without further purification unless otherwise indicated. Proton (¹H) nuclear magnetic resonance (NMR) spectra were recorded on a Bruker Avance 300 MHz NMR spectrometer. Significant peaks are tabulated and typically include: number of protons, and multiplicity (s, singlet; d, double; t, triplet; q, quartet; m, multiplet; br s, broad singlet). Chemical shifts are reported as parts per million (δ) downfield relative to tetramethylsilane. Low resolution mass spectra (MS) were obtained as electrospray ionization (ESI) mass spectra, which were recorded on a Shimadzu HPLC/MS instrument using reverse-phase conditions (acetonitrile/water, 0.05% acetic acid). Preparative HPLC was performed using Varian HPLC systems and Phenomenex columns.

It is understood that in the following description, combinations of substituents and/or variables of the depicted formulae are permissible only if such contributions result in stable compounds under standard conditions.

It will also be appreciated by those skilled in the art that in the process described below, the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R (where R is alkyl, aryl or aralkyl), p-methoxybenzyl, trityl and the like. Suitable protecting groups for carboxylic acid include alkyl, aryl or aralkyl esters.

Protecting groups may be added or removed in accordance with standard techniques, which are well-known to those skilled in the art and as described herein. The use of protecting groups is described in detail in Green, T. W. and P. G. M. Wutz, Protective Groups in Organic Synthesis (1991), 2nd Ed., Wiley-Interscience.

One of ordinary skill in the art could easily ascertain which choices for each substituent are possible for the reaction conditions of each Scheme. Moreover, the substituents are selected from components as indicated in the specification heretofore, and may be attached to starting materials, intermediates, and/or final products according to schemes known to those of ordinary skill in the art.

Also it will be apparent that the compounds provided herein could exist as one or more isomers, that is, E/Z isomers, enantiomers and/or diastereomers. Compounds of formula (I) may be generally prepared as depicted in the following schemes, unless otherwise noted, the various substituents are as defined elsewhere herein.

Standard abbreviations and acronyms as defined in J. Org. Chem. 2007 72(1): 23A-24A are used herein. Other abbreviations and acronyms used herein are as follows:

| | |
|---|---|
| Cy₃P | tricyclohexylphopshine |
| DIEA | diisopropylethylamine |
| DCM | dichloromethane |
| EDCI | N-(3-Dimethylaminopropyl)-N′- |
| | ethylcarbodiimide hydrochloride |
| EtOAc | ethyl acetate |
| EtOH | ethanol |
| HOAc | acetic acid |
| HOBt | N-hydroxybenzotriazole |
| MeOH | methanol |
| TEA | triethylamine |
| Trityl | triphenylmethyl |

In an illustrative method, urea compounds of formula (I) may be routinely prepared according to the synthetic routes outlined in Scheme 1. The commercially available substituted 2-amino-5-bromoazines 1 and the readily available appropriately substituted 4-(tert-butoxycarbonylamino)phenylboronic acids 2 are coupled to give biaryl compounds 3 using a Pd-catalyzed Suzuki coupling protocol, promoted by bases such as, but not limited to, Na₂CO₃ in solvents such as, but not limited to, water and 1,4-dioxane (reaction was done using a mixture of water and dioxane). The reaction can be promoted using heating in a conventional oil bath heating or in a microwave reactor. The tert-butyl carbamoyl groups is cleaved to give the anilines 4 under acidic conditions such as, but not limited to, TFA in DCM or 4N HCl in 1,4-dioxane. The diaryl ureas 6 can be prepared by the reaction of the phenyleneamine derivatives 4 with activated arylcarbamic acid derivatives such as 5, in solvents such as THF or DMF, promoted with bases such as DIEA or DMAP and by heating as necessary at elevated temperatures.

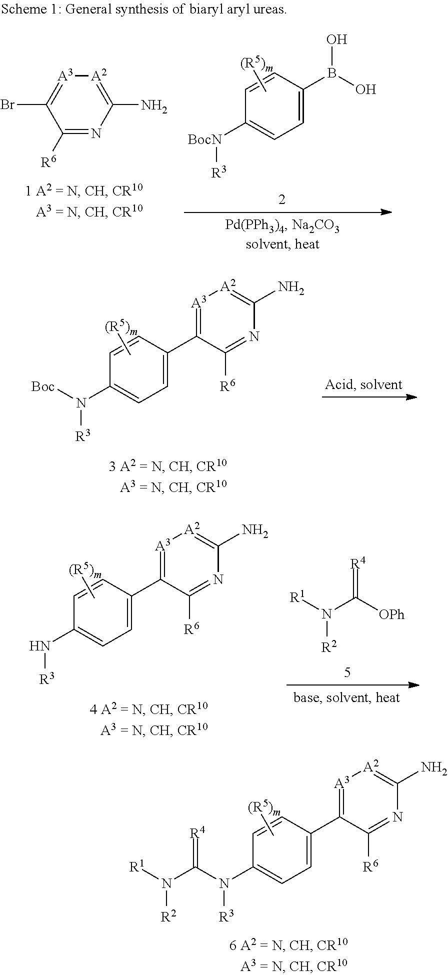

In an illustrative method, urea compounds of formula (I) may also be routinely prepared according to the synthetic route outlined in Scheme 2. The commercially available appropriately substituted bromoaryl amine derivatives 7 are reacted with appropriately substituted 5-(4,4,5,5-tetramethyl-1,3,2-dioxaborolan-2-yl)azine-2-amines 8 using a Pd-catalyzed Suzuki coupling protocol to give the biaryl derivatives 9. The diaryl ureas 10 can be prepared by the reaction of 9 with activated arylcarbamic acid derivatives such as 5, as described in Scheme 1.

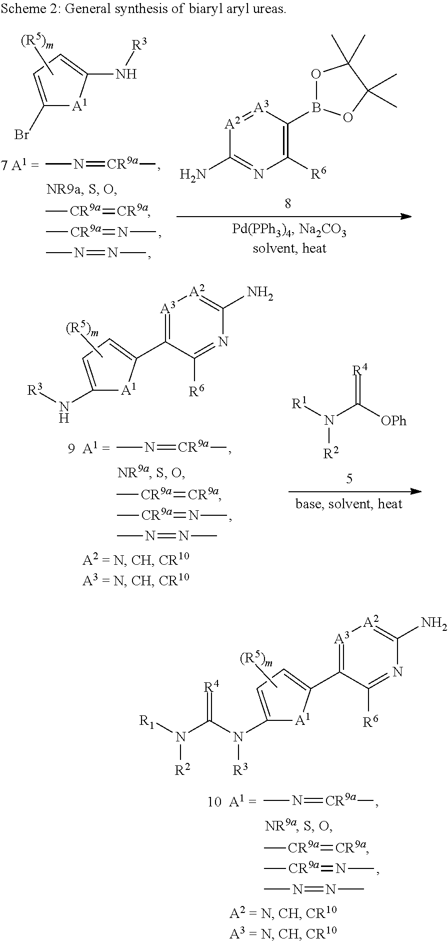

In an illustrative method, the amide compounds of formula (I) may be routinely prepared according to the synthetic routes outlined in Scheme 3. The phenyleneamine derivatives 4 from Scheme 2 can condense with aryl acetyl chlorides 11 in solvents such as DCM or THF, promoted by bases such as DIEA or pyridine, to give the aryl acetamide derivatives 13. The phenyleneamine derivatives 4 can also couple with aryl acetic acids 12 using appropriate coupling reagents, such as, but not limited to, EDCI or HATU, promoted by bases such as DIEA, TEA, or DMAP to give the aryl acetamide derivates 13.

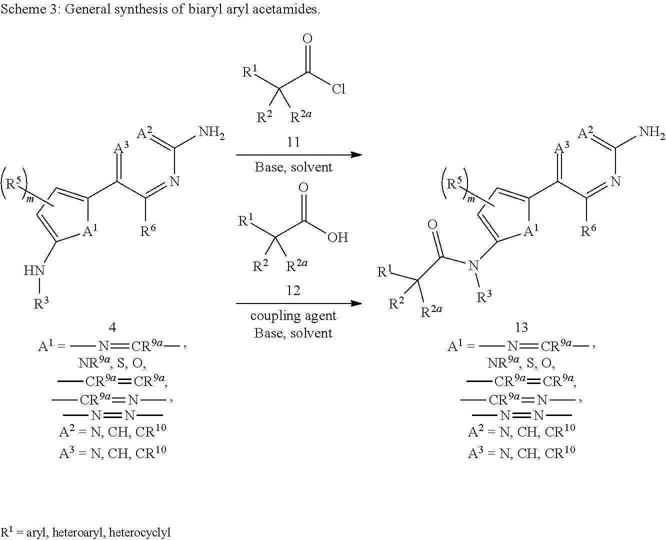

In an illustrative method, urea compounds of formula (I) may also be routinely prepared according to the synthetic routes outlined in Scheme 4. The commercially available appropriately substituted 5-bromo-2-fluoroazine derivatives 14 undergo a nucleophilic substitution at elevated temperature with appropriate amines (15) in solvents such as DMSO or i-PrOH and using bases such as DIEA to give substituted aminoazine derivatives 16. Compounds 16 can subsequently be transformed to the desired compounds 17 as described in Scheme 1 for conversion of compounds 1 to compounds 6.

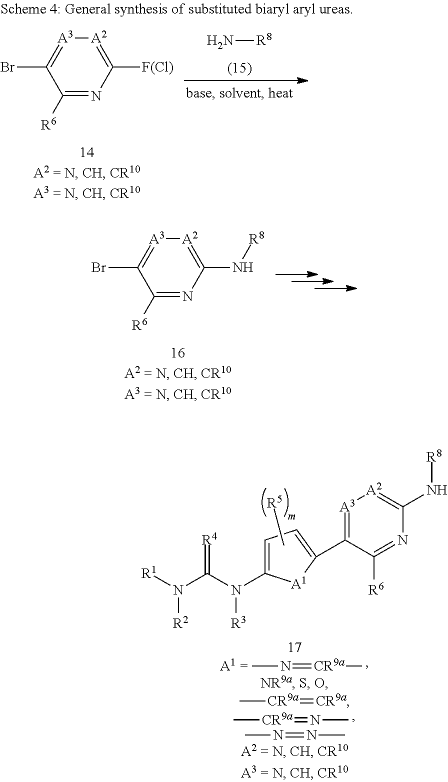

In an illustrative method, urea compounds of formula (I) may also be routinely prepared according to the synthetic routes outlined in Scheme 5. The readily available appropriately substituted 2-fluoroazine derivatives 18 undergo a nucleophilic substitution at elevated temperature with appropriate alcohols (19) in solvents such as DMF and using bases such as sodium hydride or potassium t-butoxide to give substituted alkoxyazine derivatives 19. Compounds 19 can subsequently be transformed to the desired compounds 20 as described in Scheme 1 for conversion of compounds 3 to compounds 6.

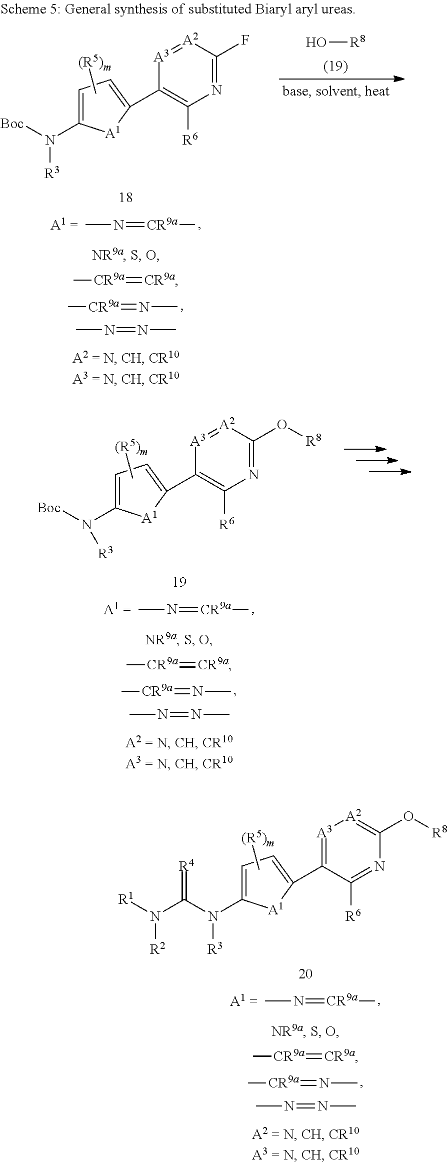

In an illustrative method, aminoalkylpyrimidine derivatives may be routinely prepared according to the synthetic routes outlined in Scheme 6. The readily available appropriately substituted (Z)-2-(4′-nitrophenyl)-3-N,N-dimethylaminopropenals (21) and 2-aminoacetamidine hydrochlorides (22) were condensed in solvents such as, but not limited to, EtOH to form the pyrimidine derivatives 23. Reduction of the nitro groups could be realized optionally at elevated temperatures using reducing systems such as SnCl₂ in an alcohol solvent or metallic iron or tin under acidic conditions, or hydrogenation in the presence of transition metal catalysts. Compounds 24 can subsequently be transformed to compounds of formula (I) as described in Scheme 1 for conversion of compounds 4 to compounds 6.

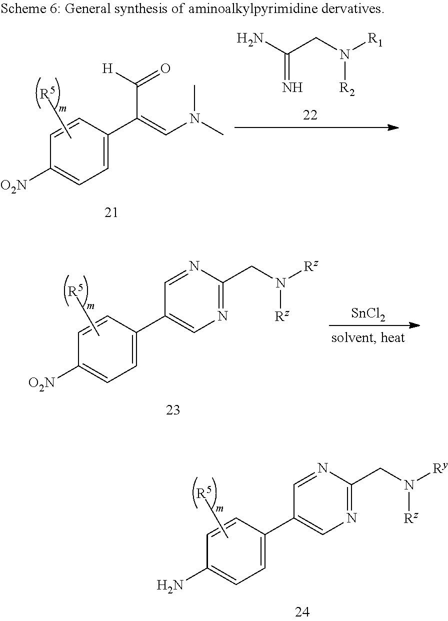

In an illustrative method, aminoalkylazine derivatives may also be routinely prepared according to the synthetic routes outlined in Scheme 7. The readily available 5-bromo-2-iodoazines (25) are coupled with alkynes 26 using a Sonogashira coupling protocol to give the alkynylazine derivatives 26, which are then coupled with appropriately substituted 4-(tert-butoxycarbonylamino)phenylboronic acids 2 using a Pd-catalyzed Suzuki coupling protocol to give the biaryl derivatives 27. The reduction of the alkynes of 27 to alkanes 28 can be realized using palladium on carbon under a hydrogen atmosphere in solvents such as, but not limited to, MeOH or EtOH. The THP group of 28 can be removed with mild acid, such as, but not limited to, pyridinium p-toluenesulfonate, to give the alcohols 29. Activation of the alcohols 29 with methanesulfonic anhydride or methanesulfonyl chloride and bases such as TEA, followed by substitution with amines (30) affords derivatives 31. The tert-butyl carbamate of 31 can then be removed to give the anilines 32 using acids in solvents, such as TFA in DCM or 4N HCl in 1,4-dioxane. Compounds 32 can subsequently be transformed to compounds of formula (I) as described in Scheme 1 for conversion of compounds 4 to compounds 6.

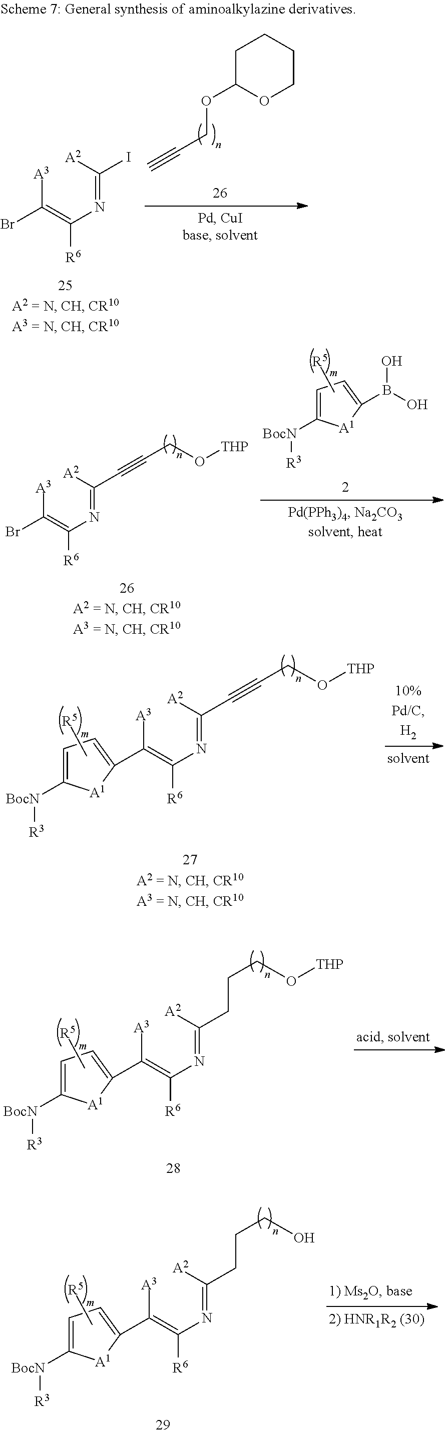

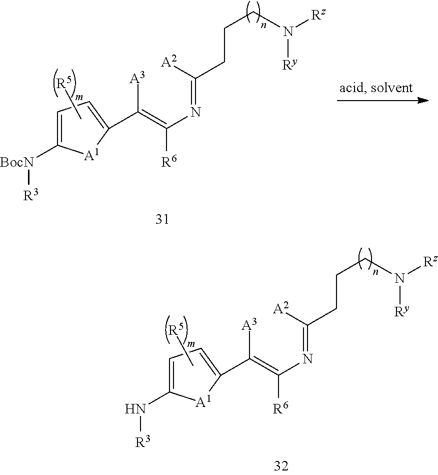

Aryl amine derivatives R¹—NH₂, wherein the aryl group R¹ is a 5-membered isoxazole ring, may be prepared by condensation of appropriate fragments and precursors by methods well known in the art and described in texts such as Gilchrist, T. L., Heterocyclic Chemistry (1992), 2nd Ed., Longman Scientific & Technical and John Wiley & Sons. Scheme 8 shows one example where R⁻NH₂ is 5-substituted-3-aminoisoxazole, whereby an appropriate 3-oxonitrile (35) is treated with hydroxylamine under appropriate conditions of pH and temperature which are described, for example, in Takase et al. Heterocycles 1991 32(6), 1153-1158, to afford the desired aryl amine product (36). This method is particularly applicable for cases in which the atom of R⁹ directly attached to the aromatic ring is highly substituted, for example, is an α,α-dialkyl substituent (See Takase et al. Heterocycles 1991 32(6), 1153-1158). The requisite 3-oxonitriles (35) can be prepared by reaction of an R⁹-containing carboxylic ester (33) with an akali metal salt of acetonitrile (34) (See, for example, U.S. Pat. No. 4,728,743).

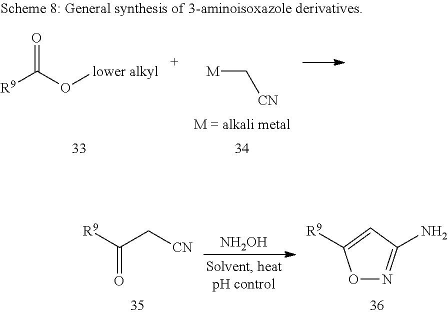

Scheme 9 shows an example for the synthesis of aryl amine derivatives R¹—NH₂, wherein the aryl group R¹ is 3-substituted-5-aminoisoxazole, whereby an appropriate 3-oxonitrile 35, prepared as described in Scheme 8, is treated with hydroxylamine under appropriate conditions of pH and temperature, as described again in Takase et al. Heterocycles 1991 32(6), 1153-1158, to afford the desired aryl amine product (37). This method is particularly applicable for cases in which the atom of R⁹ directly attached to the aromatic ring is not highly substituted, for example, is not an α,α-dialkyl substituent (See Eddington et al. Eur. J. Med. Chem. 2002 37, 635-648), or when R⁹ contains one or more highly electron-withdrawing groups, for example fluorine, or under special conditions of pH and solvent, such as an ethanol and water mixture as described in EP 0220947.

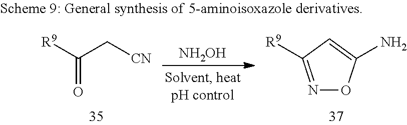

In an illustrative method, the amide compounds of formula (I) may also be routinely prepared according to the synthetic routes outlined in Scheme 10. The amine derivatives 38 can condense with bromoaryl acetic acids 39 using coupling reagents such as, but not limited to, EDCI or HATU, to give the bromoaryl acetamide derivatives 40. The bromides 40 can then be reacted with appropriately substituted 5-(4,4,5,5-tetramethyl-1,3,2-dioxaborolan-2-yl)azine-2-amine 8 using a Pd-catalyzed Suzuki coupling protocol to give the biaryl aryl acetamide derivatives 41.

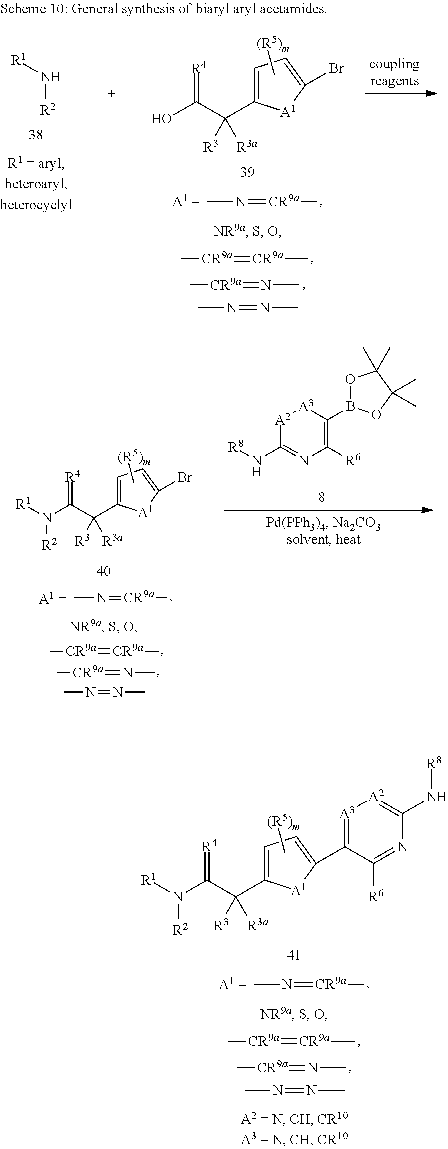

In an illustrative method, the biaryl derivatives may also be routinely prepared according to the synthetic routes outlined in Scheme 11. The bromo-nitro arenes 42 can react with appropriately substituted 5-(4,4,5,5-tetramethyl-1,3,2-dioxaborolan-2-yl)azine-2-amines 8 using a Pd-catalyzed Suzuki coupling protocol to give the nitro-substituted biaryl derivatives 43. The nitro group of 43 can be reduced using palladium on carbon under a hydrogen atmosphere to give biaryl derivatives 44. Compounds 32 can subsequently be transformed to compounds of formula (I) as described in Scheme 1 for conversion of compounds 4 to compounds 6.

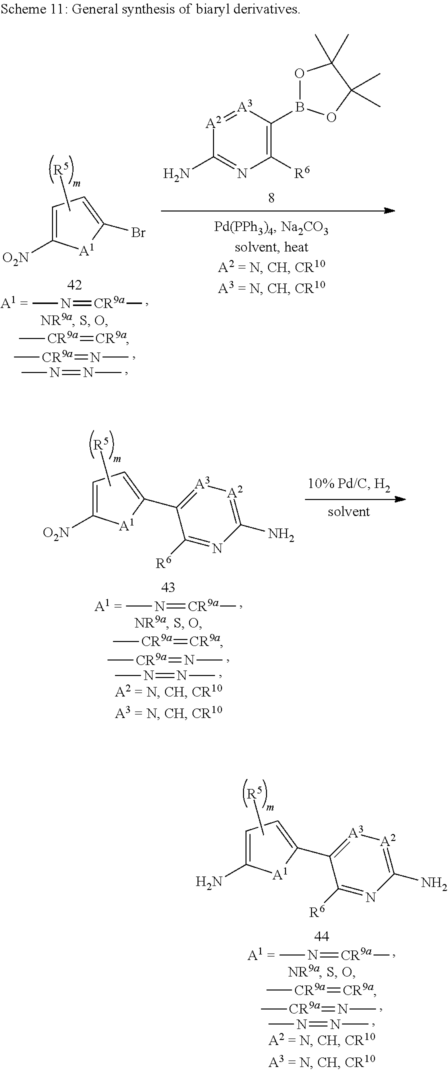

In an illustrative method, urea compounds of formula (I) may also be routinely prepared according to the synthetic routes outlined in Scheme 12. The commercially available appropriately substituted 5-bromo-2-aminoazine derivatives 45 can be protected as N-trityl derivatives 46, which can then be reacted with appropriately substituted 5-(4,4,5,5-tetramethyl-1,3,2-dioxaborolan-2-yl)azin-2-amines 8 using a Pd-catalyzed Suzuki coupling protocol to give the biaryl derivatives 47. The diaryl ureas 48 can be prepared by the reaction of 47 with activated arylcarbamic acid derivatives 5, as described in Scheme 1. The trityl groups of 48 can be removed to give compounds 49 under acidic conditions such as, but not limited to, TFA in DCM or 4N HCl in 1,4-dioxane.

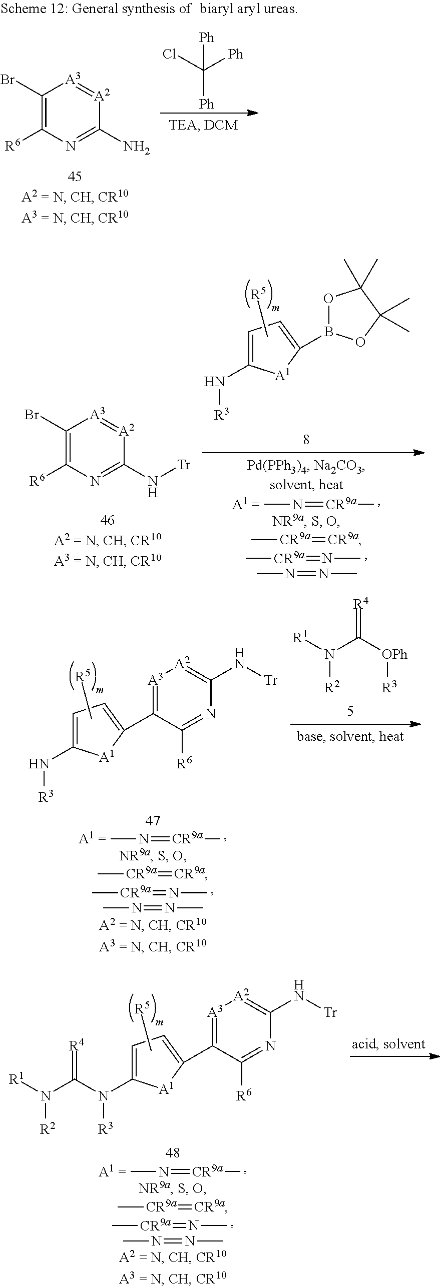

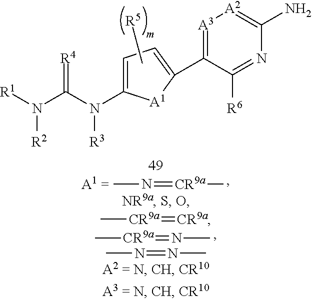

In an illustrative method, 2-amino-5-bromopyridine derivatives may be routinely prepared according to the synthetic route outlined in Scheme 13. The readily available 5-bromo-3-(bromomethyl)pyridin-2-amine hydrobromide 50 (Ref: Seefeld, Mark A.; et al. Journal of Medicinal Chemistry; 46; 9; 2003; 1627-1635) was treated with amines in solvents such as THF to afford the bromopyridine derivatives 51. Compounds 51 may then be further transformed to compounds of formula (I) as described in Scheme 1 for the conversion of compounds 1 to compounds 6.

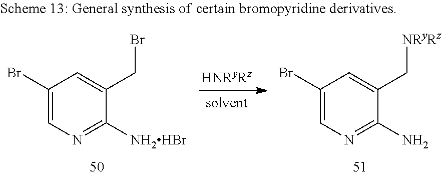

In an illustrative method, certain bicyclic aminoazine derivatives of Formula (I) may be routinely prepared according to the synthetic route outlined in Scheme 14. The readily available azine derivatives 52 can be protected as t-butoxycarbonyl derivatives 53. The alkoxycarbonyl lactams 53 can be reduced to alkoxycarbonylaminal intermediates, which are then trapped with Horner-Wadsworth-Emmons reagent to give the acetate derivatives 54. The ester group of 54 can be reduced with reducing agents, such as, but not limited to, LiBH₄, which also induces the migration of the tert-butyloxycarbonyl group. The tert-butyl carbonates 55 can be hydrolyzed to alcohols 56 with bases such as NaOH in solvents such as MeOH. The azine derivatives 56 can then be coupled with readily available diaryl urea derivatives 57 using a Pd-catalyzed Suzuki coupling protocol to give the biaryl aryl urea derivatives 58.

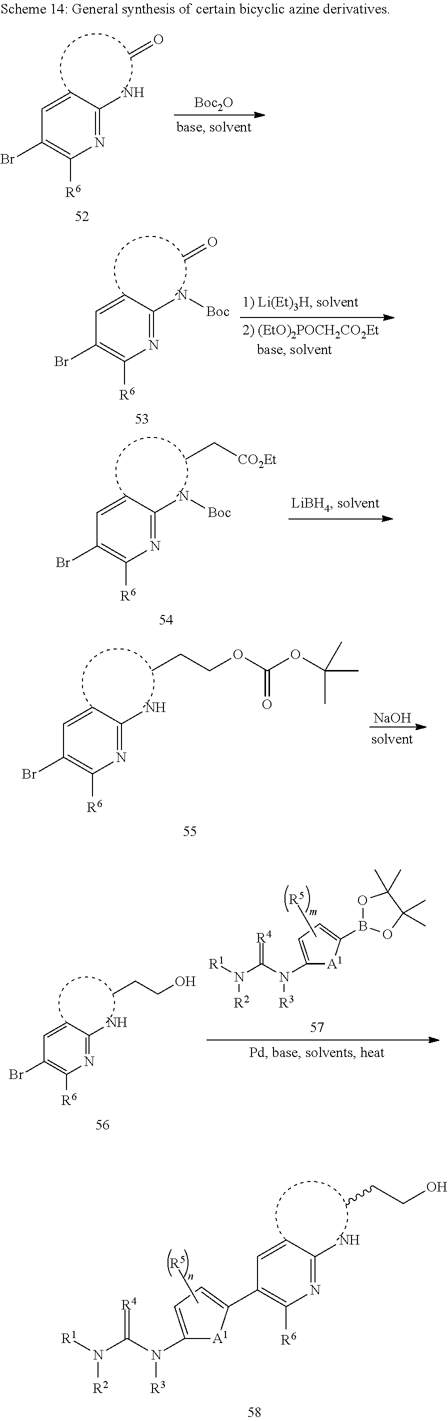

In one embodiment, the azine derivatives 52 can be coupled with readily available diaryl urea derivatives 57 using a Pd-catalyzed Suzuki coupling protocol to give the biaryl aryl urea derivatives 59 as outlined in Scheme 15.

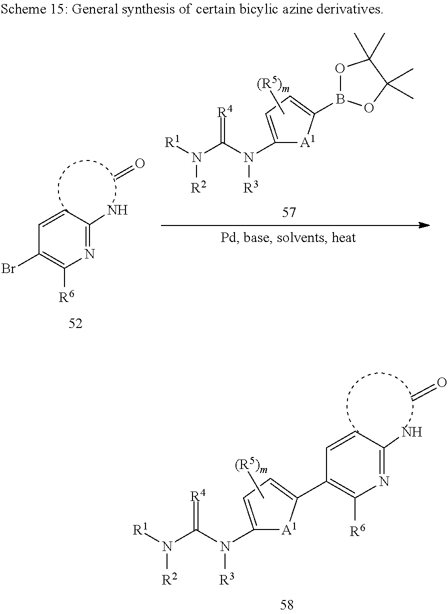

In an illustrative method, the aryl acetamide compounds of formula (I) may also be routinely prepared according to the synthetic route outlined in Scheme 16. The arylamine derivatives 38 can condense with dioxaborolane-substituted aryl acetic acids 60 using coupling reagents, such as, but not limited to, EDCI or HATU, to give the aryl acetamide derivates 61. The condensation can be conducted in solvents such as THF or DMF, promoted with bases such as DIEA or DMAP and by heating as necessary at elevated temperatures. The boronate esters 61 can then be reacted with 5-halogen/sulfonate substituted-azine-2-amines 62 using a Pd-catalyzed Suzuki coupling protocol to give the biaryl aryl acetamide derivatives 41.

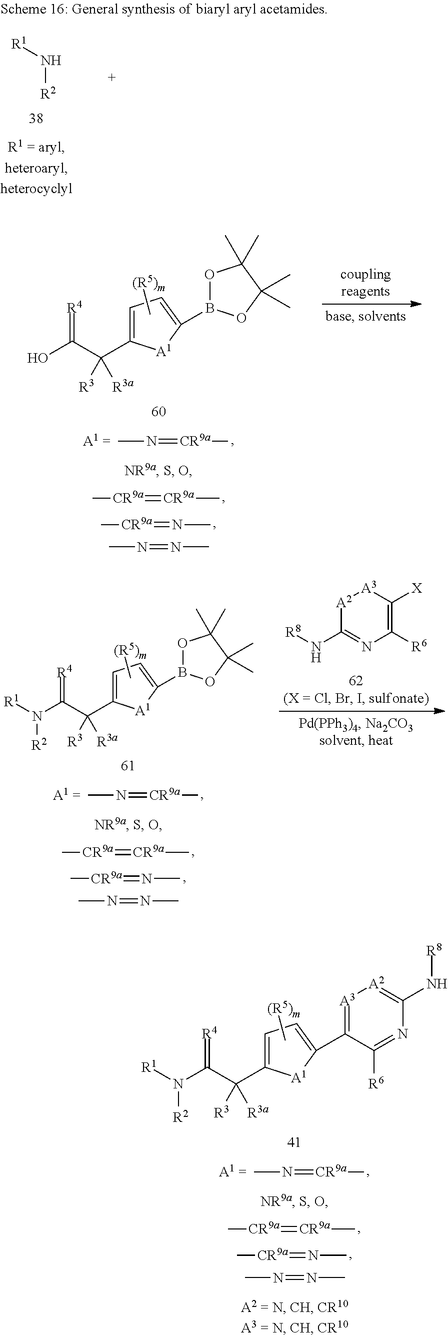

Azole amine derivatives (64) (R¹)ₚ-A-NH₂, wherein the NH₂ group is directly attached to a nitrogen atom of the azole ring, may be prepared by amination of the corresponding azoles using methods well known in the art. Scheme 17 shows one example where (R¹)ₚ-A is 4-substituted-pyrazole 63, whereby the amination can be realized by treating with a base, such as, but not limited to, NaH, and using amination reagents, such as, but not limited to, hydroxylamine-O-sulfonic acid or chloroamine. The reaction can be conducted in solvents such as, but not limited to, DMF and THF. The reaction can be promoted using heating in a conventional oil bath.

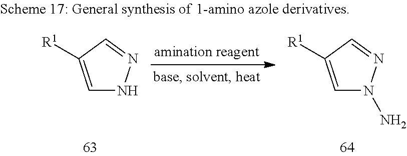

In an illustrative method, aryl acetic acid derivatives may be routinely prepared according to the synthetic route outlined in Scheme 18. The readily available hydroxymethyl aryl derivatives 65 can be activated by reacting with methanesulfonyl chloride in the presence of base such as, but not limited to, triethylamine. The mesylates 66 can be displaced with cyanides, such as, but not limited to, NaCN or KCN, in solvents such as, but not limited to EtOH or DMSO, to give the aryl acetonitrile derivatives 67. The cyano group of 67 can be converted to carboxylic acid group of 68 under acidic conditions, using acids, such as, but not limited to, HCl or sulfuric acid. The reaction can be promoted by heating in a conventional oil bath. The halogen groups of 68 can then undergo Suzuki coupling with 4,4,4′,4′,5,5,5′,5′-octamethyl-2,2′-bi(1,3,2-dioxaborolane). The reaction can be catalyzed with catalysts, such as, but not limited to tetrakis(triphenylphosphine)palladium, dichlorobis(tricyclohexylphosphine)palladium(II), promoted by bases, such as, but not limited to KOAc or NaOAc, in solvents, such as, but not limited to DMSO or 1,4-dioxane to give the aryl acetic acid derivatives 69.

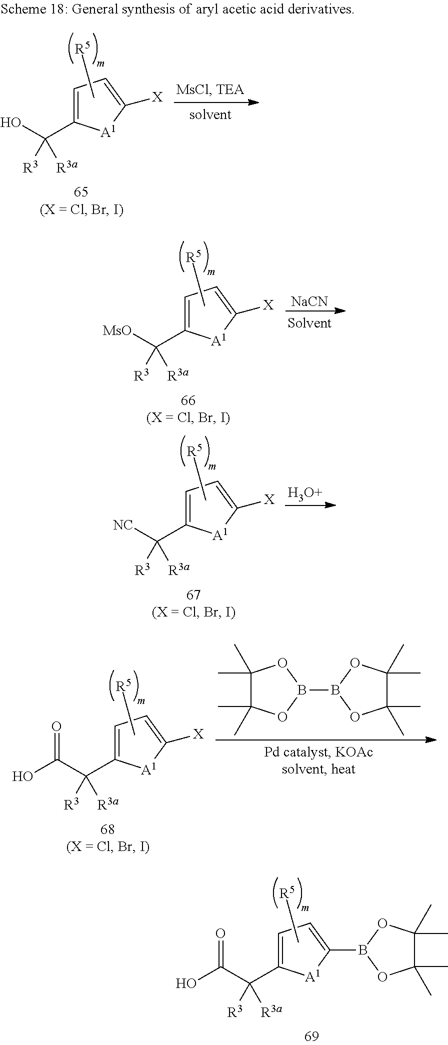

In an illustrative method, aryl acetic acid derivatives may also be routinely prepared according to the synthetic route outlined in Scheme 19. The readily available aryl derivatives 70 with appropriate leaving groups, such as, but not limited to, halogen and sulfonate, can be substituted with a tert-butyl malonate anion generated in the presence of a base such as, but not limited to, sodium hydride. The resulting tert-butyl malonate derivates 71 can be treated with acid, such as, but not limited to, trifluoroacetic acid, to induce the removal of the tert-butyl group and subsequent decarboxylation, to afford the aryl acetate derivates 72. Hydrolysis of the aryl acetate derivates 72 using a base, such as, but not limited to, NaOH, afforded aryl acetic acid derivatives 68.

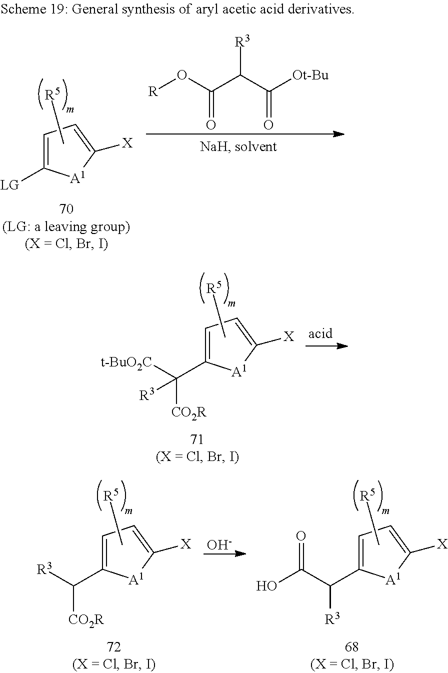

In an illustrative method, 5-halogen/sulfonate substituted-azine-2-amine derivatives may be routinely prepared according to the synthetic route outlined in Scheme 20. The readily available azine-2-amine derivatives 73 can be halogenated using appropriate halogenation reagents, such as, but not limited to, N-chlorosuccinimide, N-bromosuccinimide, or N-iodosuccinimide, to afford 5-halogen substituted-azine-2-amine derivatives 62. Meanwhile, the readily available azine-2-amine derivatives 74 can be sulfonylated using appropriate sulfonlyation reagents, such as, but not limited to, trifluoromethanesulfonic anhydride or trifluoromethanesulfonyl chloride. The reaction can be promoted with bases, such as, but not limited to, pyridine or 2,6-lutidine.

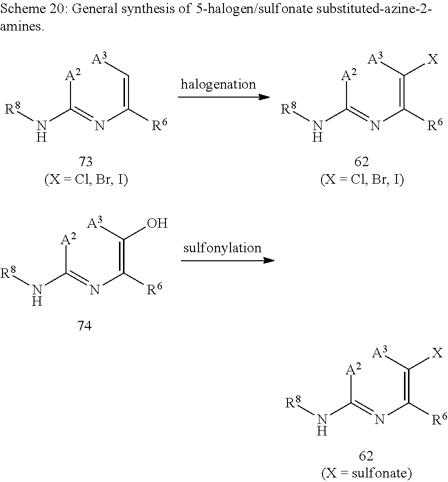

In an illustrative method, the 1,5-naphthyridin-2(1H)-one compounds of formula (I) may be routinely prepared according to the synthetic route outlined in Scheme 21. The appropriately substituted aminopyridine derivatives 75 can undergo Heck coupling with appropriately functionalized acrylates 76 to give pyridyl propenoate derivatives 77. The Heck coupling reaction can be catalyzed with a palladium-based catalyst, such as, but not limited to, Pd(OAc)₂, along with an added ligand, such as, but not limited to, P(o-tolyl)₃. The coupling reaction can be conducted in solvents such as CH₃CN or DMF, promoted with bases such as TEA or Na₂CO₃ and by heating as necessary at elevated temperatures. Pyridyl propenoate derivatives 77 can be cyclized to afford 1,5-naphthyridin-2(1H)-one derivatives 78 using bases, such as, but not limited to, NaOMe or t-BuOK, in solvents, such as, but not limited to, MeOH or DMSO. The reaction can be promoted by heating as necessary at elevated temperatures. The aryl acetamide compounds 79 can be realized by coupling 1,5-naphthyridin-2(1H)-one derivatives 78 with the boronate esters 61 using a Pd-catalyzed Suzuki coupling protocol. Similarly, the biaryl aryl ureas 80 can be synthesized through Suzuki coupling of 78 with diaryl uear boronate esters 57.

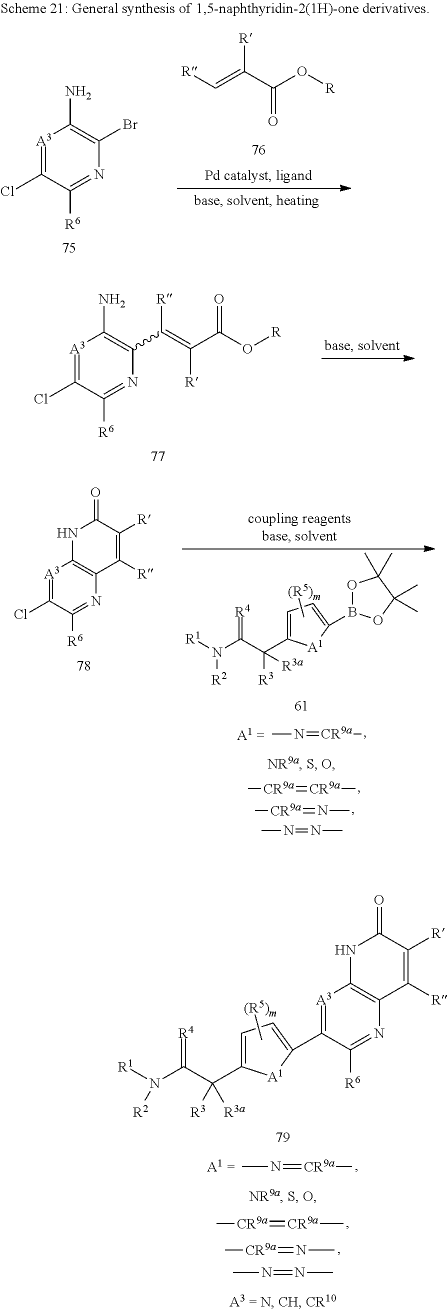

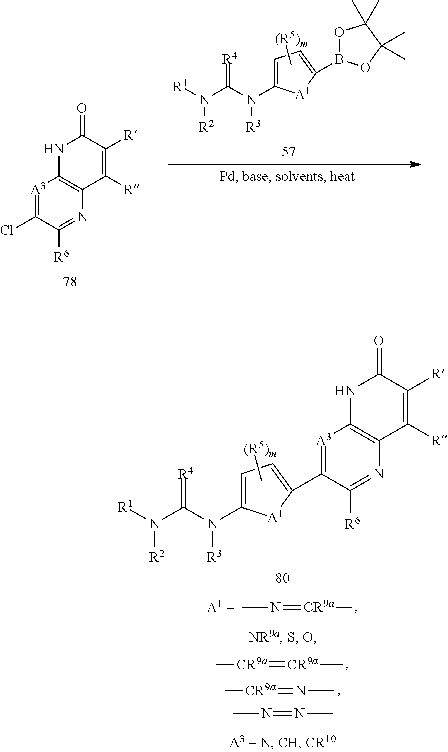

In an illustrative method, the 1,5-naphthyridin-2(1H)-one compounds of formula (I) may also be routinely prepared according to the synthetic route outlined in Scheme 22. The 1,5-naphthyridin-2(1H)-one derivatives 78 can undergo reductive amination with aldehyde derivatives 81 to give aminopyridine derivatives 82. The reaction was accomplished using reducing agents, such as, but not limited to, NaCNBH₃ or Na(OAc)₃BH, and promoted by the addition of acids, such as, but not limited to, AcOH or HCl. Aminopyridines 82 can then be cyclized to afford 1,5-naphthyridin-2(1H)-one derivatives 83 using procedures as described in Scheme 21. The aryl acetamide compounds 84 can be realized by coupling 1,5-naphthyridin-2(1H)-one derivatives 83 with the boronate esters 61 using a Pd-catalyzed Suzuki coupling protocol. Alternatively, 1,5-naphthyridin-2(1H)-one derivatives 78 can be N-alkylated with electrophiles (R¹¹—X) using a base, such as, but not limited to, NaH or K₂CO₃, in a solvent, such as, but not limited to DMF or NMP, to give 83, which can then be transformed to 1,5-naphthyridin-2(1H)-one derivatives 84.

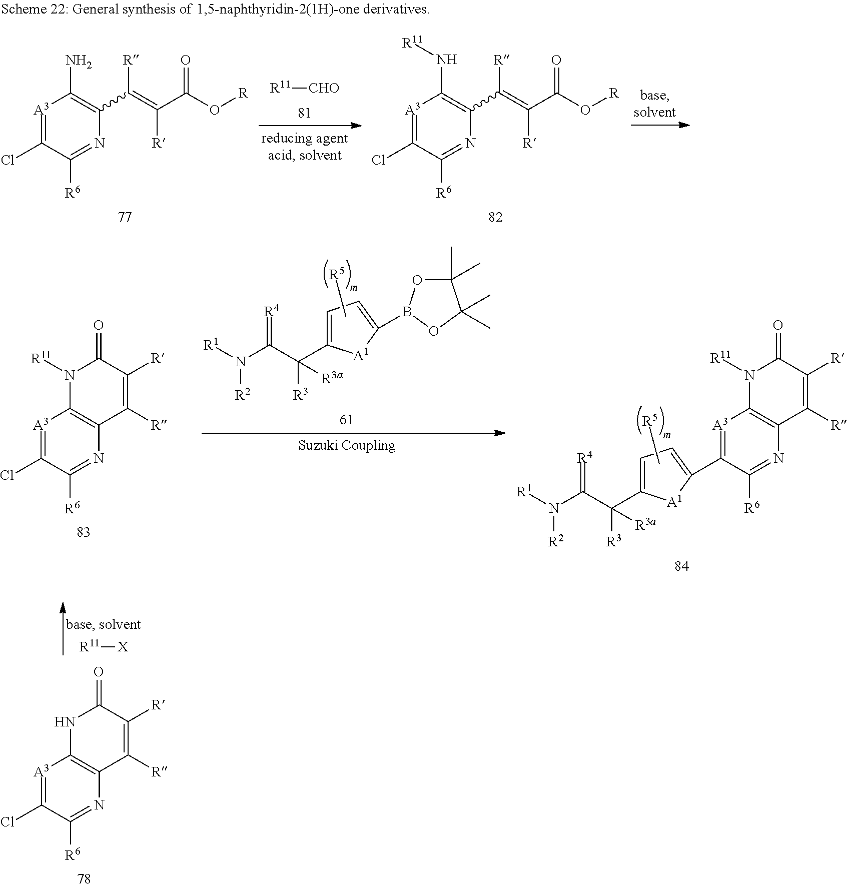

The subject matter has been described in an illustrative manner, and it is to be understood that the terminology used is intended to be in the nature of description rather than of limitation. Thus, it will be appreciated by those of skill in the art that conditions such as choice of solvent, temperature of reaction, volumes, reaction time may vary while still producing the desired compounds. In addition, one of skill in the art will also appreciate that many of the reagents provided in the following examples may be substituted with other suitable reagents. See, e.g., Smith & March, Advanced Organic Chemistry, 5^{th} ed. (2001). Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

### Example 1

### Preparation of 1-(4-(6-aminopyridin-3-yl)phenyl)-3-(5-tert-butylisoxazol-3-yl)urea

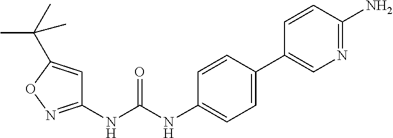 [SMILES: CC(C)(C)c1cc(NC(=O)Nc2ccc(-c3ccc(N)nc3)cc2)no1]

Step 1: 5-(4-aminophenyl)pyridin-2-ylamine (89.3 mg, 63%) was prepared as a solid according to the procedure described in Step 1 of Example 2, substituting 5-bromo-2-aminopyridine for 5-bromo-3-cyano-2-aminopyridine used in Example 2. LC-MS (ESI) m/z 186 (M+H)⁺.

Step 2: 1-[4-(6-aminopyridin-3-yl)-phenyl]-3-(5-tert-butyl-isoxazol-3-yl)urea (80.9 mg, 48%) was prepared as a solid according to the procedure described in Step 2 of Example 2, substituting 5-(4-aminophenyl)pyridin-2-ylamine from Step 1 above for 2-amino-5-(4-aminophenyl)nicotinonitrile used in Example 2. LC-MS (ESI) m/z 352 (M+H)⁺. ¹H NMR (DMSO-d₆) δ: 9.50 (s, 1H), 8.84 (s, 1H), 8.21 (d, J=2.3 Hz, 1H), 7.67 (dd, J=8.7, 2.4 Hz, 1H), 7.49 (s, 4H), 6.45-6.56 (m, 2H), 6.01 (s, 2H), 1.30 (s, 9H).

### Example 2

### Preparation of 1-[4-(6-amino-5-cyanopyridin-3-yl)-phenyl]-3-(5-tert-butylisoxazol-3-yl)urea

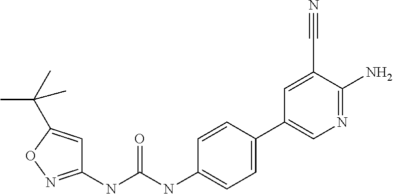 [SMILES: CC(C)(C)c1cc(NC(=O)Nc2ccc(-c3cnc(N)c(C#N)c3)cc2)no1]

Step 1: To a microwave reaction vessel were added 4-(tert-butoxycarbonylamino)phenylboronic acid (180 mg, 0.759 mmol), 5-bromo-3-cyano-2-aminopyridine (170.0 mg, 0.858 mmol), 1,4-dioxane (3.5 mL) and 2M aqueous sodium carbonate (0.94 mL, 1.88 mmol). Argon gas was bubbled through the solution for 5 min, then tetrakis(triphenylphosphine) palladium(0) (40.0 mg, 0.035 mmol) was added and the vial was sealed and heated in a microwave reactor for 20 min at 170° C. The mixture was partitioned between EtOAc (10 mL) and saturated sodium bicarbonate (10 mL) The aqueous layer was separated and extracted with ethyl acetate (3×10 mL). The combined organic layers were washed with brine (10 mL), dried over MgSO₄, filtered and concentrated under reduced pressure to give an oil which was purified by silica gel flash chromatography, eluting with 25-100% EtOAc in hexanes. The purified material was dissolved in DCM (4 mL), excess TFA (2 mL) added and the mixture was stirred at rt for 4 h. The mixture was concentrated to dryness, then EtOAc (8 mL), saturated NaHCO₃ (8 mL) and 1 M aqueous NaOH (1 mL) were added. After confirming basic pH, the layers were shaken and separated and the aqueous layer was extracted with EtOAc (2×5 mL). The combined extracts were dried over MgSO₄, filtered and concentrated under reduced pressure to give 2-amino-5-(4-aminophenyl)nicotinonitrile (113.9 mg, 71%) as a solid, which was sufficiently pure for the next step. LC-MS (ESI) m/z 211 (M+H)⁺.

Step 2: In a 20 mL vial were combined 2-amino-5-(4-aminophenyl)nicotinonitrile from Step 1 (113.9 mg, 0.542 mmol), (5-tert-butylisoxazol-3-yl)carbamic acid phenyl ester (160.0 mg, 0.615 mmol) (WO2006/82404 A1 (2006/08/10)), DMF (3 mL) and DMAP (160.0 mg, 1.310 mmol). The vial was sealed and stirred at 50° C. for 16 h. The mixture was partitioned between water (20 mL) and EtOAc (5 mL), and the separated aqueous layer was extracted with EtOAc (3×5 mL). The combined organic phases were washed with brine (2×5 mL), dried over MgSO₄, filtered and concentrated in the presence of Celite. Purification by flash chromatography, eluting with 0-12% MeOH in DCM, afforded 1-[4-(6-amino-5-cyanopyridin-3-yl)-phenyl]-3-(5-tert-butyl-isoxazol-3-yl)-urea (145.3 mg, 71%) as a solid. LC-MS (ESI) m/z 377 (M+H)⁺. ¹H NMR (DMSO-d₆) δ: 9.54 (s, 1H), 8.89 (s, 1H), 8.55 (d, J=2.4 Hz, 1H), 8.18 (d, J=2.4 Hz, 1H), 7.55-7.66 (m, 2H), 7.47-7.56 (m, 2H), 6.98 (s, 2H), 6.51 (s, 1H), 1.30 (s, 9H).

### Example 3

### Preparation of 1-(5-tert-butylisoxazol-3-yl)-3-(4-(3-oxo-3,4-dihydro-2H-pyrido[3,2-b][1,4]oxazin-7-yl)phenyl)urea

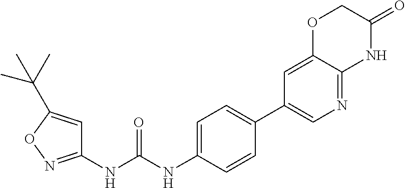 [SMILES: CC(C)(C)c1cc(NC(=O)Nc2ccc(-c3cnc4c(c3)OCC(=O)N4)cc2)no1]

Step 1: 7-(4-aminophenyl)-2H-pyrido[3,2-b][1,4]oxazin-3(4H)-one was synthesized according to the procedure described in Step 1 of Example 2, substituting 7-bromo-2H-pyrido[3,2-b][1,4]oxazin-3(4H)-one (Ref: Savelon, L.; Bizot-Espiard, J. G.; Caignard, D. H.; Pfeiffer, B.; Renard, P.; et al.; Bioorganic & Medicinal Chemistry; English 1998, 6; 133-142) for 5-bromo-3-cyano-2-aminopyridine used in Example 2. LC-MS (ESI) m/z 242 (M+H)⁺.

Step 2: 1-(5-tert-butylisoxazol-3-yl)-3-(4-(3-oxo-3,4-dihydro-2H-pyrido[3,2-b][1,4]oxazin-7-yl)phenyl)urea was prepared as a white solid (18 mg, 25% yield) according to the procedure described in Step 2 of Example 2, substituting 7-(4-aminophenyl)-2H-pyrido[3,2-b][1,4]oxazin-3(4H)-one from Step 1 above for 2-amino-5-(4-aminophenyl)nicotinonitrile used in Example 2. LC-MS (ESI) m/z 408 (M+H)⁺. ¹H NMR (DMSO-d₆) δ: 11.33 (s, 1H), 9.54 (s, 1H), 8.93 (s, 1H), 8.22 (d, 1H), 7.64 (m, 3H), 7.54 (d, 2H), 6.51 (s, 1H), 4.69 (s, 2H), 1.30 (s, 9H).

### Example 4

### Preparation of 1-(5-tert-butylisoxazol-3-yl)-3-(4-(3,4-dihydro-2H-pyrido[3,2-b][1,4]oxazin-7-yl)phenyl)urea

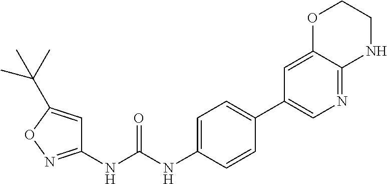 [SMILES: CC(C)(C)c1cc(NC(=O)Nc2ccc(-c3cnc4c(c3)OCCN4)cc2)no1]

Step 1: To 7-(4-aminophenyl)-2H-pyrido[3,2-b][1,4]oxazin-3(4H)-one from Example 3 Step 1 (150 mg, 0.42 mmol) in THF (3 mL) was added 1.0 M BH₃THF (0.85 mL, 0.84 mmol) and the mixture was heated at reflux for 2 h. Analysis by LC-MS indicated that the reaction was nearly complete. The mixture allowed to cool, then quenched by addition of 3N HCl (1.0 mL). After 30 min, the mixture was basified with saturated aq NaHCO₃ and extracted with EtOAc (2×15 mL). The combined organic layers were washed with brine, dried over MgSO₄ and concentrated under reduced pressure. The resulting white solid (150 mg) was used for the next step without further purification. LC-MS (ESI) m/z 228 (M+H)⁺.

Step 2: 1-(5-tert-butylisoxazol-3-yl)-3-(4-(3,4-dihydro-2H-pyrido[3,2-b][1,4]oxazin-7-yl)phenyl)urea was prepared as a white solid (33 mg, 26% yield) according to the procedure described in Step 2 of Example 2, substituting 4-(3,4-dihydro-2H-pyrido[3,2-b][1,4]oxazin-7-yl)aniline from Step 1 above for 2-amino-5-(4-aminophenyl)nicotinonitrile used in Example 2. LC-MS (ESI) m/z 394 (M+H)⁺. ¹H NMR (DMSO-d₆) δ: 9.49 (s, 1H), 8.84 (s, 1H), 7.88 (d, 1H), 7.48 (m, 4H), 7.22 (d, 1H), 6.81 (s, 1H), 6.51 (s, 1H), 4.14 (t, 2H), 3.42 (s, 2H), 1.30 (s, 9H).

### Example 5

### Preparation of 1-(5-tert-butylisoxazol-3-yl)-3-(4-(3-(2-hydroxyethyl)-3,4-dihydro-2H-pyrido[3,2-b][1,4]oxazin-7-yl)phenyl)urea

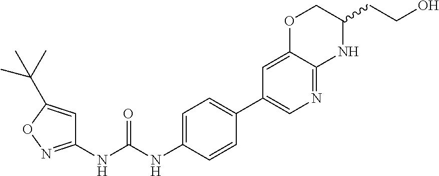 [SMILES: CC(C)(C)c1cc(NC(=O)Nc2ccc(-c3cnc4c(c3)OCC(CCO)N4)cc2)no1]

Step 1: 7-Bromo-2H-pyrido[3,2-b][1,4]oxazin-3(4H)-one (658 mg, 2.86 mmol) was stirred in 10 mL of THF. Di-t-butyl dicarbonate (687 mg, 3.15 mmol) and DMAP (18 mg, 0.15 mmol) were added and the resulting mixture was stirred at rt overnight, whereupon analysis by TLC indicated complete reaction. The mixture was partitioned between EtOAc (20 mL) and brine (15 mL), and the organic layer was dried over MgSO₄ and concentrated under reduced pressure to afford tert-butyl 7-bromo-3-oxo-2H-pyrido[3,2-b][1,4]oxazine-4(3H)-carboxylate (900 mg) as a white solid, which was used directly for the next step.

Step 2: To a stirred mixture of tert-butyl 7-bromo-3-oxo-2H-pyrido[3,2-b][1,4]oxazine-4(3H)-carboxylate from Step 1 (256 mg, 0.78 mmol) in THF (5 mL) at −78° C. was added 1.0 M LiBEt₃H in THF (0.78 mL, 0.78 mmol), and the mixture was stirred at at −78° C. for 30 min. The mixture was allowed to warm to 0° C., then treated with a mixture prepared separately by stirring triethyl phosphonoacetate (308 μL, 1.55 mmol) in THF (5 mL) with 60% NaH in mineral oil (62 mg, 1.55 mmol) at rt for 30 min. The resulting mixture was stirred at rt for 1 h, then partitioned between EtOAc (25 mL) and water (15 mL). The organic layer was washed with brine, dried over MgSO₄, and concentrated under reduced pressure. The residue was purified by silica gel chromatography eluting with 0-30% EtOAc in hexanes to give a mixture of two products (190 mg), one of which corresponds to tert-butyl 7-bromo-3-(2-ethoxy-2-oxoethyl)-2H-pyrido[3,2-b][1,4]oxazine-4(3H)-carboxylate.

Step 3: The mixture from Step 2 (185 mg, 0.46 mmol) in 3 mL of THF was treated with LiBH₄ (20 mg, 0.92 mmol) and LiBEt₃H (46 μL, 0.046 mmol). The resulting mixture was then stirred at rt for 30 min and heated at 60° C. for 3 h. Analysis by LC-MS indicated complete reaction. The mixture was partitioned between EtOAc (20 mL) and saturated aq NH₄Cl (15 mL), and the separated organic layer was washed with brine, dried over MgSO₄, and concentrated under reduced pressure. The residue was purified by silica gel chromatography eluting with 0-35% EtOAc in hexanes to give 2-(7-bromo-3,4-dihydro-2H-pyrido[3,2-b][1,4]oxazin-3-yl)ethyl tert-butyl carbonate as a colorless solid (60 mg, 36% yield).

Step 4: 2-(7-Bromo-3,4-dihydro-2H-pyrido[3,2-b][1,4]oxazin-3-yl)ethyl tert-butyl carbonate from Step 3 (150 mg, 0.42 mmol) was dissolved in MeOH (2 mL) and 3N NaOH (ca. 0.5 mL) was added. The mixture was stirred at rt for 60 h, then extracted with EtOAc (2×15 mL). The combined organic layers were washed with brine, dried over MgSO₄, and concentrated under reduced pressure to give 2-(7-bromo-3,4-dihydro-2H-pyrido[3,2-b][1,4]oxazin-3-yl)ethanol as a colorless oil (99 mg, 92% yield).

Step 5: 1-(5-tert-butylisoxazol-3-yl)-3-(4-(4,4,5,5-tetramethyl-1,3,2-dioxaborolan-2-yl)phenyl)urea (500 mg, 48%) was prepared as a solid according to the procedure described in Step 2 of Example 2, substituting 4-(4,4,5,5-tetramethyl-1,3,2-dioxaborolan-2-yl)aniline for 2-amino-5-(4-aminophenyl)nicotinonitrile used in Example 2. LC-MS (ESI) m/z 386 (M+H)⁺.

Step 6: A stirred mixture of 2-(7-Bromo-3,4-dihydro-2H-pyrido[3,2-b][1,4]oxazin-3-yl)ethyl tert-butyl carbonate from Step 4 (67 mg, 0.26 mmol) and Cs₂CO₃ (254 mg, 0.78 mmol) in dioxane/DMF/H₂O (2:2:1) was treated with 1-(5-tert-butylisoxazol-3-yl)-3-(4-(4,4,5,5-tetramethyl-1,3,2-dioxaborolan-2-yl)phenyl)urea from Step 5 (100 mg, 0.26 mmol) followed by Pd(Ph₃P)₄ (30 mg, 0.026 mmol) under a stream of Argon. The resulting mixture was then heated at 140° C. for 10 min. Analysis by LC-MS indicated complete reaction. The mixture was partitioned between EtOAc (10 mL) and brine (10 mL), and the separated organic layer was dried over MgSO₄ and concentrated under reduced pressure. The residue was purified by reverse phase HPLC to afford 1-(5-tert-butylisoxazol-3-yl)-3-(4-(3-(2-hydroxyethyl)-3,4-dihydro-2H-pyrido[3,2-b][1,4]oxazin-7-yl)phenyl)urea. (26 mg, 23% yield). LC-MS (ESI) m/z 438 (M+H)⁺. ¹H NMR (DMSO-d₆) δ: 11.90 (s, 1H), 9.50 (s, 1H), 8.85 (s, 1H), 7.90 (d, 1H), 7.49 (m, 4H), 7.24 (s, 1H), 6.81 (s, 1H), 6.51 (s, 1H), 4.63 (t, 1H), 4.20 (dd, 1H), 3.84 (m, 1H), 3.58 (m, 3H), 1.64 (m, 2H), 1.30 (s, 9H).

### Example 6

### Preparation of 1-(5-tert-butylisoxazol-3-yl)-3-(4-(5-cyano-6-(2-morpholinoethylamino)pyridin-3-yl)phenyl)urea

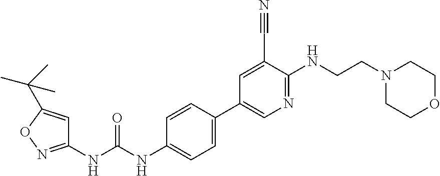 [SMILES: CC(C)(C)c1cc(NC(=O)Nc2ccc(-c3cnc(NCCN4CCOCC4)c(C#N)c3)cc2)no1]

Step 1: 5-bromo-2-(2-morpholinoethylamino)nicotinonitrile (527.1 mg, 77%) was synthesized according to the procedure described in Step 1 of Example 7, substituting 5-bromo-2-chloronicotinitrile for 5-bromo-2-fluoropyridine used in Example 7. LC-MS (ESI) m/z 310, 312 (M+H)⁺.

Step 2: 5-(4-aminophenyl)-2-(2-morpholinoethylamino)nicotinonitrile (505 mg, 96%) was synthesized according to the procedure described in Step 2 of Example 7, substituting 5-bromo-2-(2-morpholinoethylamino)nicotinonitrile from Step 1 above for (5-bromopyridin-2-yl)-(2-morpholin-4-yl-ethyl)amine used in Example 7. LC-MS (ESI) m/z 324 (M+H)⁺.

Step 3: 1-(5-tert-butylisoxazol-3-yl)-3-(4-(5-cyano-6-(2-morpholinoethylamino)pyridin-3-yl)phenyl)urea (414.38 mg, 54%) was prepared as a solid according to the procedure described in Step 2 of Example 2, substituting 5-(4-aminophenyl)-2-(2-morpholinoethylamino)nicotinonitrile from Step 2 above for 2-amino-5-(4-aminophenyl)nicotinonitrile used in Example 2. LC-MS (ESI) m/z 490 (M+H)⁺. ¹H NMR (DMSO-d₆) δ: 9.54 (s, 1H), 8.89 (s, 1H), 8.63 (s, 1H), 8.23 (s, 1H), 7.60 (d, 2H), 7.51 (d, 2H), 7.03 (t, 1H), 6.51 (s, 1H), 3.58-3.51 (m, 6H), 2.43 (br s, 4H), 1.30 (s, 9H).

### Example 7

### Preparation of 1-(5-tert-butyl-isoxazol-3-yl)-3-{4-[6-(2-morpholin-4-yl-ethylamino)-pyridin-3-yl]-phenyl}urea

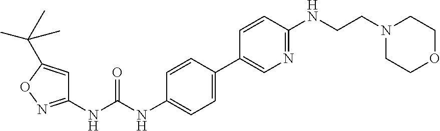 [SMILES: CC(C)(C)c1cc(NC(=O)Nc2ccc(-c3ccc(NCCN4CCOCC4)nc3)cc2)no1]

Step 1: To a microwave reaction vessel were added 2-morpholinoethylamine (0.29 mL, 2.21 mmol), DMSO (5 mL), 5-bromo-2-fluoropyridine (405 mg, 2.301 mmol), and DIEA (0.75 mL, 4.54 mmol). The vial was sealed and heated under microwave irradiation at 180° C. for 20 min. The mixture was partitioned between water (50 mL) and EtOAc (50 mL), then aqueous layer was further extracted with EtOAc (3×50 mL). The combined organic phases were washed with brine (3×50 mL), dried over MgSO₄, filtered, and concentrated in the presence of Celite. Purification by silica gel flash chromatography, eluting with 1-12% MeOH in DCM, afforded (5-bromopyridin-2-yl)-(2-morpholin-4-yl-ethyl)amine (425.3 mg, 67%) as a solid. LC-MS (ESI) m/z 286, 288 (M+H)⁺.

Step 2: [5-(4-aminophenyl)pyridin-2-yl]-(2-morpholin-4-yl-ethyl)amine (214.3 mg, 53%) was prepared as a solid according to the procedure described in Step 1 of Example 2, substituting (5-bromopyridin-2-yl)-(2-morpholin-4-yl-ethyl)amine for 5-bromo-3-cyano-2-aminopyridine used in Example 2. LC-MS (ESI) m/z 299 (M+H)⁺.

Step 3: 1-(5-tert-butyl-isoxazol-3-yl)-3-{4-[6-(2-morpholin-4-yl-ethylamino)-pyridin-3-yl]-phenyl}urea (108.3 mg, 32%) was prepared as a solid according to the procedure described in Step 2 of Example 2, substituting [5-(4-aminophenyl)pyridin-2-yl]-(2-morpholin-4-yl-ethyl)amine from Step 2 above for 2-amino-5-(4-aminophenyl)nicotinonitrile used in Example 2. LC-MS (ESI) m/z 465 (M+H)⁺. ¹H NMR (DMSO-d₆) δ: 9.50 (s, 1H), 8.84 (s, 1H), 8.28 (s, 1H), 7.67 (d, J=8.7 Hz, 1H), 7.49 (s, 4H), 6.38-6.64 (m, 3H), 3.59 (br s, 4H), 3.40 (d, J=6.0 Hz, 2H), 2.41 (br s, 6H), 1.30 (s, 9H).

### Example 8

### Preparation of 1-(4-(6-amino-5-(hydroxymethyl)pyridin-3-yl)phenyl)-3-(5-tert-butylisoxazol-3-yl)urea

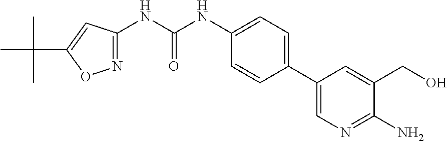 [SMILES: CC(C)(C)c1cc(NC(=O)Nc2ccc(-c3cnc(N)c(CO)c3)cc2)no1]

Step 1: A flask was charged with Pd₂(dba)₃ (91 mg, 0.1 mmol) and tri-cyclohexyl phosphine (Cy₃P) (60 mg, 0.3 mmol) and flushed with nitrogen. DME (2.5 mL), water (1 mL), and EtOH (1 mL) were added, then (2-amino-5-bromopyridin-3-yl)methanol (Ref: Seefeld, Mark A., et al. Journal of Medicinal Chemistry 2003, 46, 1627-1635) (203 mg, 1.00 mmol), K₃PO₄.3H₂O (1.10 g, 4.14 mmol) and 4-(4,4,5,5-tetramethyl-1,3,2-dioxaborolan-2-yl)aniline (219 mg, 1.00 mmol) were added successively. The mixture was heated to 90° C. for 3 h, whereupon analysis by LC-MS indicated presence of the desired product. The mixture was filtered through a Celite plug washing with EtOAc (3×10 mL). To the filtrate was added H₂O (20 mL), and the separated aqueous phase was extracted with ethyl acetate (3×20 mL). The combined organic fractions were dried over Na₂SO₄ and concentrated under reduced pressure. The residue (160 mg) containing (2-amino-5-(4-aminophenyl)pyridin-3-yl)methanol was used directly for the next step.

Step 2: To a solution of (2-amino-5-(4-aminophenyl)pyridin-3-yl)methanol from Step 1 (160 mg, 0.74 mmol) and DIEA (0.1 mL) in THF (3 mL) was added (5-tert-butylisoxazol-3-yl)carbamic acid phenyl ester (193 mg, 0.74 mmol) in THF (3 mL) under a nitrogen atmosphere. The mixture was heated at 50° C. for 13 h, whereupon analysis by TLC indicated that the starting material was consumed. Water (20 mL) was added and the mixture was extracted with ethyl acetate (5×20 mL). The combined organic layers were washed with water and brine, dried over Na₂SO₄ and concentrated under reduced pressure. The residue was purified by preparative TLC eluting with DCM: Methanol=10:1 to afford 1-(4-(6-amino-5-(hydroxymethyl)pyridin-3-yl)phenyl)-3-(5-tert-butylisoxazol-3-yl)urea (22 mg, 8%). LC-MS (ESI) m/z 382 (M+H)⁺. ¹H NMR (DMSO-d₆) δ: 9.51 (s, 1H), 8.87 (s, 1H), 8.17 (s, 1H), 7.70 (s, 1H), 7.52 (m, 4H), 6.52 (s, 1H), 5.81 (s, 2H), 5.25 (m, 1H), 4.42 (m, 2H), 1.31 (s, 9H).

### Example 9

### Preparation of 1-(5-tert-butylisoxazol-3-yl)-3-(4-(7-oxo-5,6,7,8-tetrahydro-1,8-naphthyridin-3-yl)phenyl)urea

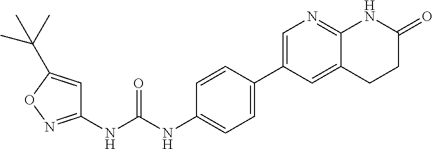 [SMILES: CC(C)(C)c1cc(NC(=O)Nc2ccc(-c3cnc4c(c3)CCC(=O)N4)cc2)no1]

Step 1: Crude 6-(4-aminophenyl)-3,4-dihydro-1,8-naphthyridin-2(1H)-one (170 mg) was synthesized according to the procedure described in Step 1 of Example 8, substituting 6-bromo-3,4-dihydro-1,8-naphthyridin-2(1H)-one (Ref Seefeld, Mark A., et al. Journal of Medicinal Chemistry 2003, 46, 1627-1635) for (2-amino-5-bromopyridin-3-yl)methanol used in Example 8. LC-MS (ESI) m/z 240 (M+H)⁺.

Step 2: 1-(5-tert-butylisoxazol-3-yl)-3-(4-(7-oxo-5,6,7,8-tetrahydro-1,8-naphthyridin-3-yl)phenyl)urea (20 mg, 7%) was synthesized according to the procedure described in Step 2 of Example 8, substituting (6-(4-aminophenyl)-3,4-dihydro-1,8-naphthyridin-2(1H)-one from Step 1 above for (2-amino-5-(4-aminophenyl)pyridin-3-yl)methanol used in Example 8. LC-MS (ESI) m/z 406 (M+H)⁺. ¹H NMR (DMSO-d₆) δ: 10.55 (s, 1H), 9.59 (s, 1H), 9.01 (s, 1H), 8.41 (s, 1H), 7.91 (s, 1H), 7.64 (d, 2H), 7.56 (d, 2H), 6.53 (s, 1H), 2.96 (s, 2H), 2.51 (s, 2H), 1.31 (s, 9H).

### Example 10

### Preparation of 1-(4-(6-amino-2,4-dimethylpyridin-3-yl)phenyl)-3-(5-tert-butylisoxazol-3-yl)urea

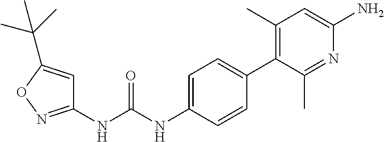 [SMILES: Cc1cc(N)nc(C)c1-c1ccc(NC(=O)Nc2cc(C(C)(C)C)on2)cc1]

Step 1: 5-(4-aminophenyl)-4,6-dimethylpyridin-2-amine was synthesized according to the procedure described in Step 1 of Example 2, substituting 5-bromo-4,6-dimethylpyridin-2-amine for 5-bromo-3-cyano-2-aminopyridine used in Example 2. LC-MS (ESI) m/z 214 (M+H)⁺.

Step 2: 1-(4-(6-Amino-2,4-dimethylpyridin-3-yl)phenyl)-3-(5-tert-butylisoxazol-3-yl)urea (70 mg, 25%) was prepared as a powder according to the procedure described in Step 2 of Example 2, substituting 5-(4-aminophenyl)-4,6-dimethylpyridin-2-amine from Step 1 above for 2-amino-5-(4-aminophenyl)nicotinonitrile used in Example 2. LC-MS (ESI) m/z 380 (M+H)⁺. ¹H NMR (DMSO-d₆) δ: 9.51 (s, 1H), 8.85 (s, 1H), 7.47 (d, 2H), 7.06 (d, 2H), 6.51 (s, 1H), 6.20 (s, 1H), 5.70 (s, 2H), 1.98 (s, 3H), 1.84 (s, 3H), 1.30 (s, 9H).

### Example 11

### Preparation of 1-(4-(6-aminopyridin-3-yl)-3-fluorophenyl)-3-(5-tert-butylisoxazol-3-yl)urea

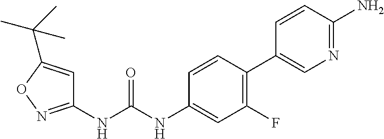 [SMILES: CC(C)(C)c1cc(NC(=O)Nc2ccc(-c3ccc(N)nc3)c(F)c2)no1]

Step 1: 5-(4,4,5,5-tetramethyl-1,3,2-dioxaborolan-2-yl)pyridin-2-amine (1.0 g, 5.45 mmol) in DCM (15 mL) was stirred with di-tert-butyl dicarbonate (1.25 g, 5.73 mmol) and triethylamine (1.80 mL, 12.91 mmol) at rt for 17 h, whereupon analysis by LC-MS indicated the presence of desired product. The mixture was concentrated under reduced pressure and the residue was purified by silica gel chromatography eluting with 5-70% EtOAc in hexanes to afford tert-butyl 5-(4,4,5,5-tetramethyl-1,3,2-dioxaborolan-2-yl)pyridin-2-ylcarbamate (807.9 mg, 46%). LC-MS (ESI) m/z 321 (M+H)⁺.

Step 2: 5-(4-Amino-2-fluorophenyl)pyridin-2-amine (122.4 mg, 77%) was synthesized according to the procedure described in Step 1 of Example 2, substituting 4-bromo-3-fluoroaniline for 5-bromo-3-cyano-2-aminopyridine used in Example 2 and tert-butyl 5-(4,4,5,5-tetramethyl-1,3,2-dioxaborolan-2-yl)pyridin-2-ylcarbamate from Step 1 above for -(tert-butoxycarbonylamino)phenylboronic acid used in Example 2. The t-butyl carbamoyl group cleaved spontaneously during the Suzuki coupling step. LC-MS (ESI) m/z 204 (M+H)⁺.

Step 3: 1-(4-(6-aminopyridin-3-yl)-3-fluorophenyl)-3-(5-tert-butylisoxazol-3-yl)urea (48.3 mg, 22%) was synthesized according to the procedure described in Step 2 of Example 2, substituting 5-(4-amino-2-fluorophenyl)pyridin-2-amine from Step 2 above for 2-amino-5-(4-aminophenyl)nicotinonitrile used in Example 2. LC-MS (ESI) m/z 370 (M+H)⁺. ¹H NMR (DMSO-d₆) δ: 9.60 (s, 1H), 9.03 (s, 1H), 8.08 (s, 1H), 7.54 (d, 2H), 7.39 (t, 1H), 7.18 (d, 1H), 6.52 (t, 2H), 6.09 (s, 2H), 1.30 (s, 9H).

### Example 12

### Preparation of 1-(5-tert-butylisoxazol-3-yl)-3-(4-(5,6,7,8-tetrahydro-1,8-naphthyridin-3-yl)phenyl)urea

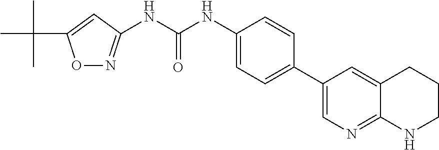 [SMILES: CC(C)(C)c1cc(NC(=O)Nc2ccc(-c3cnc4c(c3)CCCN4)cc2)no1]

Step 1: To a dry three-neck round bottom flask was added NaBH₄ (190 mg, 50.00 mmol), followed by a solution of 6-bromo-3,4-dihydro-1,8-naphthyridin-2(1H)-one (Ref: Seefeld, Mark A., et al. Journal of Medicinal Chemistry 2003, 46, 1627-1635) (227 mg, 10.00 mmol) in anhydrous THF (20 mL). Then a 47% solution of BF₃ etherate (994 mg, 70 mmol) was added dropwise under nitrogen at 0° C., and the mixture was stirred at rt for 2 h, whereupon analysis by LC-MS indicated the presence of the desired product. The reaction was quenched by saturated aq NH₄Cl (5 mL) carefully. The mixture was extracted with EtOAc (3×50 mL). The combined organic layers were dried over MgSO₄, concentrated under reduced pressure to give 6-bromo-1,2,3,4-tetrahydro-1,8-naphthyridine (180 mg. 85% yield). LC-MS (ESI) m/z 212, 214 (M+H)⁺.

Step 2: 4-(5,6,7,8-Tetrahydro-1,8-naphthyridin-3-yl)aniline was prepared as a white solid (42 mg, 38% yield) according to the procedure described in Step 2 of Example 2, substituting 6-bromo-1,2,3,4-tetrahydro-1,8-naphthyridine from the Step 1 above for 5-bromo-3-cyano-2-aminopyridine used in Example 2. LC-MS (ESI) m/z 226 (M+H)⁺.

Step 3: 1-(5-tert-Butylisoxazol-3-yl)-3-(4-(5,6,7,8-tetrahydro-1,8-naphthyridin-3-yl)phenyl)urea (12 mg, 4%) was synthesized according to the procedure described in Step 2 of Example 8, substituting (4-(5,6,7,8-tetrahydro-1,8-naphthyridin-3-yl)aniline from Step 2 above for (2-amino-5-(4-aminophenyl)pyridin-3-yl)methanol used in Example 8. LC-MS (ESI) m/z 392 (M+H)⁺. ¹H NMR (DMSO-d₆) δ: 9.51 (s, 1H), 8.87 (s, 1H), 8.06 (d, 2H), 7.50 (m, 5H), 6.65 (s, 1H), 6.52 (s, 1H), 2.75 (t, 2H), 1.83 (t, 2H), 1.31 (s, 9H).

### Example 13

### Preparation of 1-(4-(6-amino-5-(morpholinomethyl)pyridin-3-yl)phenyl)-3-(5-tert-butylisoxazol-3-yl)urea

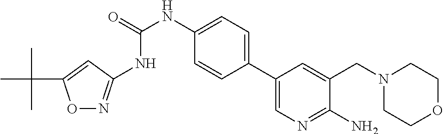 [SMILES: CC(C)(C)c1cc(NC(=O)Nc2ccc(-c3cnc(N)c(CN4CCOCC4)c3)cc2)no1]

Step 1: To a solution of 5-bromo-3-(bromomethyl)pyridin-2-amine hydrobromide (Ref: Seefeld, Mark A., et al. Journal of Medicinal Chemistry 2003, 46, 1627-1635) (500 mg, 1.44 mmol) in THF (5 mL) was added morpoline (313 mg, 3.60 mmol) in dropwise fashion. The mixture was stirred at rt for 3 h, whereupon analysis by LC-MS indicated the presence of the desired product. The reaction mixture was partitioned between EtOAc (20 mL) and water (15 mL), then the separted organic phase was washed with water and brine, dried over Na₂SO₄, and concentrated under reduced pressure to afford 330 mg of crude 5-bromo-3-(morpholinomethyl)pyridin-2-amine, which was dried under vacuum and used directly in the next step. LC-MS (ESI) m/z 272, 274 (M+H)⁺.

Step 2: 5-(4-Aminophenyl)-3-(morpholinomethyl)pyridin-2-amine was synthesized according to the procedure described in Step 1 of Example 2, substituting 5-bromo-3-(morpholinomethyl)pyridin-2-amine from Step 1 above for (5-bromopyridin-2-yl)amine used in Example 2. LC-MS (ESI) m/z 285 (M+H)⁺.

Step 3: 1-(4-(6-amino-5-(morpholinomethyl)pyridin-3-yl)phenyl)-3-(5-tert-butylisoxazol-3-yl)urea (26 mg, 8%) was prepared as a solid according to the procedure described in Step 2 of Example 8, substituting 5-(4-aminophenyl)-3-(morpholinomethyl)pyridin-2-amine from Step 2 above for (2-amino-5-(4-aminophenyl)pyridin-3-yl)methanol used in Example 8. LC-MS (ESI) m/z 451 (M+H)⁺. ¹H NMR (DMSO-d₆) δ: 9.53 (s, 1H), 8.88 (s, 1H), 8.21 (s, 1H), 7.62 (s, 1H), 7.52 (m, 4H), 6.52 (s, 1H), 6.14 (s, 1H), 3.60 (s, 4H), 3.45 (s, 2H), 2.39 (s, 4H), 1.31 (s, 9H).

### Example 14

### 1-[4-(6-aminopyridin-3-yl)-2-fluorophenyl]-3-(5-tert-butylisoxazol-3-yl)-urea

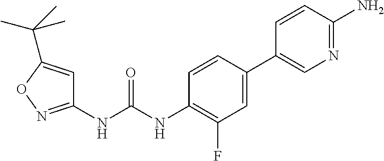 [SMILES: CC(C)(C)c1cc(NC(=O)Nc2ccc(-c3ccc(N)nc3)cc2F)no1]

Step 1: 5-(4-amino-3-fluorophenyl)pyridin-2-ylamine (134.1 mg, 58%) was synthesized as a solid according to the procedure described in Step 1 of Example 2, substituting 4-(tert-butoxycarbonylamino)-3-fluoro-phenylboronic acid for 4-(tert-butoxycarbonylamino)phenylboronic acid used in Example 2. LC-MS (ESI) m/z 204 (M+H)⁺.

Step 2: 1-[4-(6-aminopyridin-3-yl)-2-fluorophenyl]-3-(5-tert-butylisoxazol-3-yl)-urea (21.9 mg, 7.7%) was synthesized as a solid according to the procedure described in Step 2 of Example 2, substituting 5-(4-amino-3-fluorophenyl)pyridin-2-ylamine for 2-amino-5-(4-aminophenyl)nicotinonitrile used in Example 2. LC-MS (ESI) m/z 370 (M+H)⁺. ¹H NMR (MeOH-d₄) δ: 8.09-8.21 (m, 2H), 7.75 (dd, J=8.7, 2.3 Hz, 1H), 7.28-7.44 (m, 2H), 6.66 (d, J=8.7 Hz, 1H), 6.38 (s, 1H), 1.35 (s, 9H).

### Example 15

### 1-(4-(6-aminopyridin-3-yl)-2-chlorophenyl)-3-(5-tert-butylisoxazol-3-yl)urea

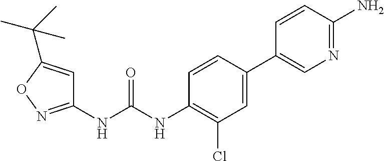 [SMILES: CC(C)(C)c1cc(NC(=O)Nc2ccc(-c3ccc(N)nc3)cc2Cl)no1]

Step 1: 5-(4-amino-3-chlorophenyl)pyridin-2-amine (115.1 mg, 67%) was synthesized according to the procedure described in Step 1 of Example 2, substituting 4-bromo-2-chloroaniline for 5-bromo-3-cyano-2-aminopyridine and tert-butyl 5-(4,4,5,5-tetramethyl-1,3,2-dioxaborolan-2-yl)pyridin-2-ylcarbamate from Step 1 of Example 11 for 4-(tert-butoxycarbonylamino)phenylboronic acid used in Example 2. The Boc group cleaved spontaneously during the Suzuki coupling step. LC-MS (ESI) m/z 220, 222 (M+H)⁺.

Step 2: 1-(4-(6-aminopyridin-3-yl)-2-chlorophenyl)-3-(5-tert-butylisoxazol-3-yl)urea (76.99 mg, 17.6%) was synthesized according to the procedure described in Step 2 of Example 2, substituting 5-(4-amino-3-chlorophenyl)pyridin-2-amine from Step 1 above for 2-amino-5-(4-aminophenyl)nicotinonitrile used in Example 2. LC-MS (ESI) m/z 386, 388 (M+H)⁺. ¹H NMR (DMSO-d₆) δ: 9.63 (s, 1H), 9.05 (s, 1H), 7.94 (d, 1H), 7.80 (d, 2H), 7.45 (dd, 1H), 7.31 (m, 2H), 6.51 (m, 2H), 6.08 (s, 2H), 1.30 (s, 9H).

### Example 16

### Preparation of 1-(4-(6-aminopyridin-3-yl)-3-chlorophenyl)-3-(5-tert-butylisoxazol-3-yl)urea

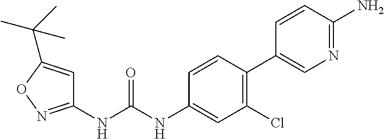 [SMILES: CC(C)(C)c1cc(NC(=O)Nc2ccc(-c3ccc(N)nc3)c(Cl)c2)no1]

Step 1: 5-(4-Amino-2-chlorophenyl)pyridin-2-amine (117 mg, 100%) was synthesized according to the procedure described in Step 1 of Example 2, substituting 4-bromo-3-chloroaniline for 5-bromo-3-cyano-2-aminopyridine and tert-butyl 5-(4,4,5,5-tetramethyl-1,3,2-dioxaborolan-2-yl)pyridin-2-ylcarbamate from Step 1 of Example 11 for 4-(tert-butoxycarbonylamino)phenylboronic acid used in Example 2. The Boc group cleaved spontaneously during the Suzuki coupling step. LC-MS (ESI) m/z 220, 222 (M+H)⁺.

Step 2: 1-(4-(6-aminopyridin-3-yl)-3-chlorophenyl)-3-(5-tert-butylisoxazol-3-yl)urea (27.4 mg, 11.7%) was synthesized according to the procedure described in Step 2 of Example 2, substituting 5-(4-amino-2-chlorophenyl)pyridin-2-amine from Step 1 above for 2-amino-5-(4-aminophenyl)nicotinonitrile used in Example 2. LC-MS (ESI) m/z 386, 388 (M+H)⁺. ¹H NMR (MeOH-d₄) δ: 8.21 (d, 1H), 8.15 (s, 1H), 7.74 (d, 1H), 7.61 (s, 1H), 7.47 (d, 1H), 6.67 (d, 1H), 6.36 (s, 1H), 1.96 (s, 2H), 1.31 (s, 9H).

### Example 17

### Preparation of 1-(6′-amino-[3,3′]bipyridinyl-6-yl)-3-(5-tert-butylisoxazol-3-yl)urea

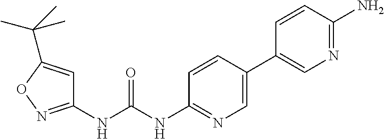 [SMILES: CC(C)(C)c1cc(NC(=O)Nc2ccc(-c3ccc(N)nc3)cn2)no1]

Step 1: To a microwave reaction vessel were added 2-aminopyridine-5-boronic acid pinacol ester (500 mg, 2.27 mmol), 5-bromo-2-aminopyridine (400.0 mg, 2.31 mmol), 1,4-dioxane (10 mL) and 2M aqueous sodium carbonate (2.36 mL, 4.72 mmol). Argon gas was bubbled through the solution for 5 min, then tetrakis(triphenylphosphine) palladium(0) (120 mg, 0.104 mmol) was added. The vial was sealed and heated in a microwave reactor for 18 min at 140° C. Celite was added, and the mixture was concentrated under reduced pressure. Purification by silica gel flash chromatography, eluting with 1-15% MeOH in DCM, gave [3,3′]bipyridinyl-6,6′-diamine (269.7 mg, 63%) as a solid. LC-MS (ESI) m/z 187 (M+H)⁺.

Step 2: 1-(6′-amino-[3,3′]bipyridinyl-6-yl)-3-(5-tert-butylisoxazol-3-yl)urea (239.9 mg, 57%) was synthesized as a solid according to the procedure described in Step 2 of Example 2, substituting [3,3′]bipyridinyl-6,6′-diamine for 2-amino-5-(4-aminophenyl)nicotinonitrile used in Example 2. LC-MS (ESI) m/z 353 (M+H)⁺. ¹H NMR (DMSO-d₆) δ: 10.93 (br s, 1H), 9.67 (s, 1H), 8.52 (d, J=1.7 Hz, 1H), 8.27 (d, J=1.9 Hz, 1H), 8.01 (dd, J=8.7, 2.1 Hz, 1H), 7.73 (dd, J=8.6, 2.2 Hz, 1H), 7.59 (d, J=8.7 Hz, 1H), 6.45-6.64 (m, 2H), 6.12 (s, 2H), 1.31 (s, 9H).

### Example 18

### Preparation of 2-(4-(6-aminopyridin-3-yl)phenyl)-N-(5-tert-butylisoxazol-3-yl)acetamide

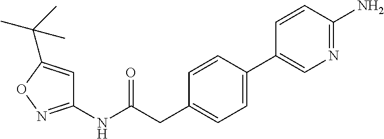 [SMILES: CC(C)(C)c1cc(NC(=O)Cc2ccc(-c3ccc(N)nc3)cc2)no1]

Step 1: To a stirred solution of 4-bromophenylacetic acid (200 mg, 0.93 mmol) in DCM (2 mL) at rt were added sequentially TEA (0.26 mL, 1.87 mmol), HOBt (132 mg, 0.98 mmol), and EDCI (188 mg, 0.98 mmol). After 15 min, 3-amino-5-tert-butylisoxazole (137 mg, 0.93 mmol) was added, and the resulting mixture was stirred at rt for 16 h. Analysia by LC-MS indicated completion of the reaction. The mixture was partitioned between DCM (50 mL) and saturated aq NaHCO₃ (50 mL) The aqueous layer was extracted with DCM (2×50 mL), and the combined organic layers were washed with brine, dried over MgSO₄, and concentrated under reduced pressure. The residue was purified by silica gel column chromatography eluting with 1-10% MeOH in DCM to give 2-(4-bromophenyl)-N-(5-tert-butylisoxazol-3-yl)acetamide (92.4 mg, 29.5%). LC-MS (ESI) m/z 337, 339 (M+H)⁺.

Step 2: 2-(4-(6-aminopyridin-3-yl)phenyl)-N-(5-tert-butylisoxazol-3-yl)acetamide (33.9 mg, 30%) was synthesized according to the procedure described in Step 1 of Example 2, substituting 2-(4-bromophenyl)-N-(5-tert-butylisoxazol-3-yl)acetamide from Step 1 above for 5-bromo-3-cyano-2-aminopyridine, and 5-(4,4,5,5-tetramethyl-1,3,2-dioxaborolan-2-yl)pyridin-2-amine for 4-(tert-butoxycarbonylamino)phenylboronic acid used in Example 2. LC-MS (ESI) m/z 351 (M+H)⁺. ¹H NMR (DMSO-d₆) δ: 11.2 (s, 1H), 8.22 (d, 1H), 7.67 (dd, 1H), 7.51 (d, 2H), 7.33 (d, 2H), 6.57 (s, 1H), 6.51 (d, 1H), 6.05 (s, 2H), 3.66 (s, 2H), 1.27 (s, 9H).

### Example 19

### Preparation of 1-(5-(6-aminopyridin-3-yl)thiophen-2-yl)-3-(5-tert-butylisoxazol-3-yl)urea

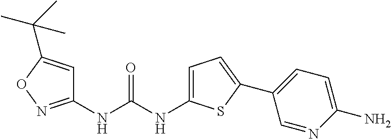 [SMILES: CC(C)(C)c1cc(NC(=O)Nc2ccc(-c3ccc(N)nc3)s2)no1]

Step 1: 5-(5-Nitrothiophen-2-yl)pyridin-2-amine (347.8 mg, 94%) was synthesized according to the procedure described in Step 2 of Example 2, substituting 2-bromo-5-nitrothiophene for 5-bromo-3-cyano-2-aminopyridine, and 5-(4,4,5,5-tetramethyl-1,3,2-dioxaborolan-2-yl)pyridin-2-amine for 4-(tert-butoxycarbonylamino)phenylboronic acid used in Example 2. LC-MS (ESI) m/z 222 (M+H)⁺.

Step 2: To 5-(5-Nitrothiophen-2-yl)pyridin-2-amine from Step 1 above (347.8 mg, 1.57 mmol) i-PrOH was added 10% Pd/C (50 mg). The resulting mixture was stirred at 50° C. under a hydrogen balloon for 17 h, whereupon analysis by LC-MS indicated completion of the reaction. The hot mixture was filtered through Celite washing with MeOH, and the filtrate was concentrated under reduced pressure. The residue was purified by silica gel column chromatography eluting with 1-10% MeOH in DCM to give 5-(5-aminothiophen-2-yl)pyridin-2-amine (119 mg, 40%). LC-MS (ESI) m/z 192 (M+H)⁺.

Step 3: 1-(5-(6-Aminopyridin-3-yl)thiophen-2-yl)-3-(5-tert-butylisoxazol-3-yl)urea (95.9 mg, 50%) was synthesized as a solid according to the procedure described in Step 2 of Example 2, substituting 5-(5-aminothiophen-2-yl)pyridin-2-amine from Step 2 above for 2-amino-5-(4-aminophenyl)nicotinonitrile used in Example 2. LC-MS (ESI) m/z 358 (M+H)⁺. ¹H NMR (DMSO-d₆) δ: 9.71 (d, 2H), 8.12 (s, 2H), 7.56 (d, 1H), 6.97 (d, 1H), 6.57 (d, 2H), 6.51 (s, 1H), 6.46 (d, 1H), 6.05 (s, 2H), 1.29 (s, 9H).

### Example 20

### Preparation of 1-(4-(6-aminopyridin-3-yl)-2,5-difluorophenyl)-3-(5-tert-butylisoxazol-3-yl)urea

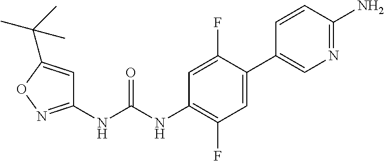 [SMILES: CC(C)(C)c1cc(NC(=O)Nc2cc(F)c(-c3ccc(N)nc3)cc2F)no1]

Step 1: To a stirred solution of triphosgene (450 mg, 1.52 mmol) in EtOAc (5 mL) were added 4-bromo-2,5-difluorophenylamine (300 mg, 1.44 mmol), and a catalytic amount of charcoal. After stirring at rt for 5 min, the reaction mixture was heated at 80° C. for 4 h. The resulting mixture was then cooled to rt and filtered through Celite. The filtrate was concentrated under reduced pressure, and the residue was dissolved in THF (4 mL) and treated with TEA (0.40 mL, 2.87 mmol) and 5-tert-butylisoxazol-3-ylamine (170 mg, 1.21 mmol). The mixture was stirred in a sealed tube at rt for 3 d, whereupon analysis by LC-MS indicated the presence of desired product. Celite was added and the mixture was concentrated under reduced pressure. Purification by silica gel column chromatography eluting with 5-35% EtOAc in hexanes followed by 100% EtOAc yielded 1-(4-bromo-2,5-difluorophenyl)-3-(5-tert-butylisoxazol-3-yl)urea (355 mg, 78%). LC-MS (ESI) m/z 374, 376 (M+H)⁺.

Step 2: 1-(4-(6-aminopyridin-3-yl)-2,5-difluorophenyl)-3-(5-tert-butylisoxazol-3-yl)urea (107.1 mg, 58%) was synthesized as a solid according to the procedure described in Step 1 of Example 2, substituting 1-(4-bromo-2,5-difluorophenyl)-3-(5-tert-butylisoxazol-3-yl)urea for 5-bromo-3-cyano-2-aminopyridine, and 5-(4,4,5,5-tetramethyl-1,3,2-dioxaborolan-2-yl)pyridin-2-amine for 4-(tert-butoxycarbonylamino)phenylboronic acid used in Example 2. LC-MS (ESI) m/z 388 (M+H)⁺. ¹H NMR (DMSO-d₆) δ: 9.90 (s, 1H), 9.01 (s, 1H), 8.12 (s, 1H), 8.05 (dd, 1H), 7.58 (d, 1H), 7.46 (dd, 1H), 6.51 (t, 2H), 6.18 (s, 2H), 1.31 (s, 9H).

### Example 21

### Preparation of 1-(5-tert-butylisoxazol-3-yl)-3-(4-(1,2,3,5-tetrahydropyrido[2,3-e][1,4]oxazepin-7-yl)phenyl)urea

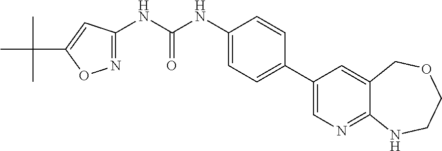 [SMILES: CC(C)(C)c1cc(NC(=O)Nc2ccc(-c3cnc4c(c3)COCCN4)cc2)no1]

Step 1: 4-(1,2,3,5-tetrahydropyrido[2,3-e][1,4]oxazepin-7-yl)aniline (110 mg, 80%) was synthesized according to the procedure described in Step 1 of Example 2, substituting 7-bromo-1,2,3,5-tetrahydropyrido[2,3-e][1,4]oxazepine [ref WO2008/9122 A1 (2008/01/24)] for 5-bromo-3-cyano-2-aminopyridine used in Example 2. LC-MS (ESI) m/z 242 (M+H)⁺.

Step 2: 1-(5-tert-butylisoxazol-3-yl)-3-(4-(1,2,3,5-tetrahydropyrido[2,3-e][1,4]oxazepin-7-yl)phenyl)urea (35 mg, 19%) was synthesized as a solid according to the procedure described in Step 2 of Example 2, substituting 4-(1,2,3,5-tetrahydropyrido[2,3-e][1,4]oxazepin-7-yl)aniline from Step 1 above for 2-amino-5-(4-aminophenyl)nicotinonitrile used in Example 2. LC-MS (ESI) m/z 408 (M+H)⁺. ¹H NMR (DMSO-d₆) δ: 9.52 (s, 1H), 8.88 (s, 1H), 8.30 (d, 1H), 7.79 (d, 1H), 7.58 (d, 2H), 7.52 (d, 2H), 6.52 (d, 1H), 6.38 (s, 1H), 4.55 (s, 2H), 3.75 (t, 2H), 3.16 (d, 2H), 1.31 (s, 9H).

### Example 22

### Preparation of 1-(4-(6-amino-5-((2-hydroxyethoxy)methyl)pyridin-3-yl)phenyl)-3-(5-tert-butylisoxazol-3-yl)urea

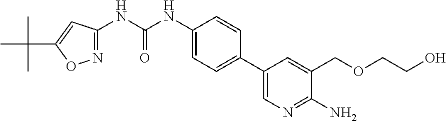 [SMILES: CC(C)(C)c1cc(NC(=O)Nc2ccc(-c3cnc(N)c(COCCO)c3)cc2)no1]

Step 1: To a solution of methyl 2-((2-amino-5-bromopyridin-3-yl)methoxy)acetate [Ref: WO2007/67416 A2 (2007/06/14)] (1.00 g, 3.64 mmol) in 2:1 THF/methanol (15 mL) was added a solution of NaBH₄ (276 mg, 7.27 mmol) in water (1 mL). The reaction mixture was heated to 40° C. for 8 h, then the mixture was extracted with EtOAc (2×30 mL). The combined organic layers were washed with water (20 mL) and brine (20 mL), dried over MgSO₄, and concentrated. The residue was purified by silica gel chromatography to afford 2-((2-amino-5-bromopyridin-3-yl)methoxy)ethanol (640 mg, 71% yield).

Step 2: 4-(1,2,3,5-tetrahydropyrido[2,3-e][1,4]oxazepin-7-yl)aniline (210 mg, 45%) was synthesized according to the procedure described in Step 1 of Example 2, substituting 2-((2-amino-5-bromopyridin-3-yl)methoxy)ethanol from Step 1 above for 5-bromo-3-cyano-2-aminopyridine used in Example 2.

Step 3: 1-(4-(6-amino-5-((2-hydroxyethoxy)methyl)pyridin-3-yl)phenyl)-3-(5-tert-butylisoxazol-3-yl)urea (60 mg, 17%) was synthesized according to the procedure described in Step 2 of Example 2, substituting 4-(1,2,3,5-tetrahydropyrido[2,3-e][1,4]oxazepin-7-yl)aniline from Step 2 above for 2-amino-5-(4-aminophenyl)nicotinonitrile used in Example 2. LC-MS (ESI) m/z 426 (M+H)⁺. ¹H NMR (DMSO-d₆) δ: 9.58 (s, 1H), 9.06 (s, 1H), 8.22 (d, 1H), 7.79 (d, 1H), 7.53 (m, 4H), 6.52 (s, 1H), 6.11 (br s, 2H), 4.78 (s, 1H), 4.46 (s, 2H), 3.58 (s, 2H), 3.52 (d, 2H), 1.31 (s, 9H).

### Example 23

### Preparation of 1-(4-(6-amino-2-methylpyridin-3-yl)phenyl)-3-(5-tert-butylisoxazol-3-yl)urea

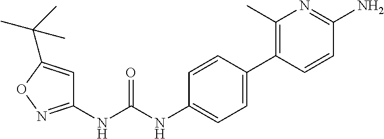 [SMILES: Cc1nc(N)ccc1-c1ccc(NC(=O)Nc2cc(C(C)(C)C)on2)cc1]

Step 1: 5-(4-aminophenyl)-6-methylpyridin-2-amine (60 mg, 79%) was synthesized according to the procedure described in Step 1 of Example 2, substituting 5-bromo-6-methylpyridin-2-amine for 5-bromo-3-cyano-2-aminopyridine used in Example 2. LC-MS (ESI) m/z 200 (M+H)⁺.

Step 2: 1-(4-(6-amino-2-methylpyridin-3-yl)phenyl)-3-(5-tert-butylisoxazol-3-yl)urea (12 mg, 12%) was synthesized as a solid according to the procedure described in Step 2 of Example 2, substituting 5-(4-aminophenyl)-6-methylpyridin-2-amine from Step 1 above for 2-amino-5-(4-aminophenyl)nicotinonitrile used in Example 2. LC-MS (ESI) m/z 366 (M+H)⁺. ¹H NMR (DMSO-d₆) δ: 9.62 (br s, 1H), 8.97 (br s, 1H), 7.47 (d, 2H), 7.21 (d, 3H), 6.51 (s, 1H), 6.34 (d, 1H), 5.86 (br s, 2H), 2.24 (s, 3H), 1.30 (s, 9H).

### Example 24

### Preparation of 1-(4-(6-amino-4-methylpyridin-3-yl)phenyl)-3-(5-tert-butylisoxazol-3-yl)urea

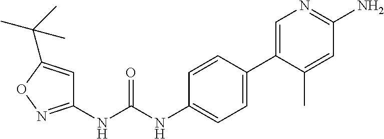 [SMILES: Cc1cc(N)ncc1-c1ccc(NC(=O)Nc2cc(C(C)(C)C)on2)cc1]

Step 1: 5-(4-aminophenyl)-4-methylpyridin-2-amine (71 mg, 93%) was synthesized according to the procedure described in Step 1 of Example 2, substituting 5-bromo-4-methylpyridin-2-amine for 5-bromo-3-cyano-2-aminopyridine used in Example 2. LC-MS (ESI) m/z 200 (M+H)⁺.

Step 2: 1-(4-(6-amino-4-methylpyridin-3-yl)phenyl)-3-(5-tert-butylisoxazol-3-yl)urea (22 mg, 18%) was synthesized as a solid according to the procedure described in Step 2 of Example 2, substituting 5-(4-aminophenyl)-4-methylpyridin-2-amine from Step 1 above for 2-amino-5-(4-aminophenyl)nicotinonitrile used in Example 2. LC-MS (ESI) m/z 366 (M+H)⁺. ¹H NMR (DMSO-d₆) δ: 9.52 (s, 1H), 8.88 (s, 1H), 7.71 (s, 1H), 7.48 (d, 2H), 7.22 (d, 2H), 6.51 (s, 1H), 6.34 (s, 1H), 5.83 (s, 2H), 2.12 (s, 3H), 1.30 (s, 9H).

### Example 25

### Preparation of 1-(6′-amino-2′-methyl-3,3′-bipyridin-6-yl)-3-(5-tert-butylisoxazol-3-yl)urea

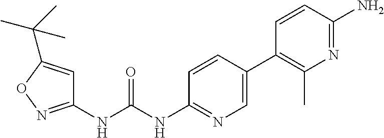 [SMILES: Cc1nc(N)ccc1-c1ccc(NC(=O)Nc2cc(C(C)(C)C)on2)nc1]

Step 1: To a stirred solution of 3-bromo-2-methyl-6-aminopyridine (450 mg, 2.41 mmol) in 6 mL of chloroform were added TEA (0.70 mL, 5.02 mmol) and trityl chloride (675 mg, 2.42 mmol). The resulting mixture was stirred at 50° C. for 14 h, whereupon analysis by LC-MS indicated complete reaction. The mixture was concentrated and the residue was purified by silica gel column chromatography eluting with 5-100% EtOAc in hexanes to give 5-bromo-6-methyl-N-tritylpyridin-2-amine (1.00 g, 97%). LC-MS (ESI) m/z 429, 431 (M+H)⁺.

Step 2: 2-Methyl-N⁶-trityl-3,3′-bipyridine-6,6′-diamine was synthesized according to the procedure described in Step 2 of Example 2, substituting 5-bromo-6-methyl-N-tritylpyridin-2-amine from Step 1 above for 5-bromo-3-cyano-2-aminopyridine, and 5-(4,4,5,5-tetramethyl-1,3,2-dioxaborolan-2-yl)pyridin-2-amine for 4-(tert-butoxycarbonylamino)phenylboronic acid used in Example 2. LC-MS (ESI) m/z 443 (M+H)⁺.

Step 3: In a 20 mL vial were combined 2-methyl-N⁶-trityl-3,3′-bipyridine-6,6′-diamine (250 mg, 0.565 mmol), (5-tert-butylisoxazol-3-yl)carbamic acid phenyl ester (160.0 mg, 0.615 mmol), DMF (2 mL) and DMAP (25 mg, 0.21 mmol). The vial was sealed and stirred at 45° C. for 18 h. 1.1 equiv. of (5-tert-butylisoxazol-3-yl)carbamic acid phenyl ester was added and the reaction mixture was heated for an additional 24 h. The reaction mixture was then concentrated under reduced pressure in the presence of Celite. Purification of the residue by silica gel flash chromatography, eluting with 1-9% MeOH in DCM, gave 1-(5-tert-butylisoxazol-3-yl)-3-(2′-methyl-6′-(tritylamino)-3,3′-bipyridin-6-yl)urea, which was dissolved in small volume of acetone and treated with 4N HCl in 1,4-dioxane (1.0 mL, 1.00 mmol) at rt for 3 d. Additional 4N HCl in 1,4-dioxane (4.0 mL, 4.00 mmol) was added and the mixture was heated at 50° C. for 1 d. The mixture was concentrated under reduced pressure and the residue was partitioned between DCM (50 mL) and saturated aq NaHCO₃ (50 mL) and 1N NaOH (2 mL). The separated aqueous layer was extracted with DCM (3×50 mL), and the combined extracts were dried over MgSO₄, filtered, and evaporated under reduced pressure in the presence of Celite. Purification by silica gel column chromatography eluting with 1-9% MeOH in DCM yielded 1-(6′-amino-2′-methyl-3,3′-bipyridin-6-yl)-3-(5-tert-butylisoxazol-3-yl)urea (25.2 mg, 18%). LC-MS (ESI) m/z 367 (M+H)⁺. ¹H NMR (DMSO-d₆) δ: 10.94 (s, 1H), 9.67 (s, 1H), 8.22 (s, 1H), 7.75 (d, 1H), 7.58 (d, 1H), 7.26 (d, 1H), 6.58 (s, 1H), 6.36 (d, 1H), 5.98 (s, 2H), 2.25 (s, 3H), 1.31 (s, 9H).

### Example 26

### Preparation of 1-(6′-amino-4′-methyl-3,3′-bipyridin-6-yl)-3-(5-tert-butylisoxazol-3-yl)urea

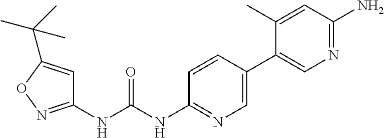 [SMILES: Cc1cc(N)ncc1-c1ccc(NC(=O)Nc2cc(C(C)(C)C)on2)nc1]

Step 1: 5-Bromo-4-methyl-N-tritylpyridin-2-amine was synthesized according to the procedure described in Step 1 of Example 25, substituting 3-bromo-4-methyl-6-aminopyridine for 3-bromo-2-methyl-6-aminopyridine used in Example 25. LC-MS (ESI) m/z 429, 431 (M+H)⁺.

Step 2: 4-Methyl-N-6-trityl-3,3′-bipyridine-6,6′-diamine was synthesized according to the procedure described in Step 2 of Example 2, substituting 5-bromo-4-methyl-N-tritylpyridin-2-amine from Step 1 above for 5-bromo-3-cyano-2-aminopyridine, and 5-(4,4,5,5-tetramethyl-1,3,2-dioxaborolan-2-yl)pyridin-2-amine for 4-(tert-butoxycarbonylamino)phenylboronic acid used in Example 2. LC-MS (ESI) m/z 443 (M+H)⁺.

Step 3: 1-(6′-amino-4′-methyl-3,3′-bipyridin-6-yl)-3-(5-tert-butylisoxazol-3-yl)urea was synthesized according to the procedure described in Step 3 of Example 25, substituting 4-methyl-N⁶-trityl-3,3′-bipyridine-6,6′-diamine from Step 2 above for 2-methyl-N⁶-trityl-3,3′-bipyridine-6,6′-diamine used in Example 25. LC-MS (ESI) m/z 367 (M+H)⁺. ¹H NMR (DMSO-d₆) δ: 10.94 (s, 1H), 9.68 (s, 1H), 8.23 (s, 1H), 7.77 (d, 2H), 7.59 (d, 1H), 6.58 (s, 1H), 6.37 (s, 1H), 5.95 (s, 2H), 2.13 (s, 3H), 1.31 (s, 9H).

### Example 27

### Preparation of 1-(5-tert-butylisoxazol-3-yl)-3-(2-fluoro-4-(6-(2-(piperidin-1-yl)ethylamino)pyridin-3-yl)phenyl)urea

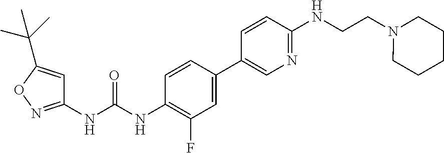 [SMILES: CC(C)(C)c1cc(NC(=O)Nc2ccc(-c3ccc(NCCN4CCCCC4)nc3)cc2F)no1]

Step 1: 5-bromo-N-(2-(piperidin-1-yl)ethyl)pyridin-2-amine (733 mg, 65%) was synthesized as a white solid according to the procedure described in Step 1 of Example 36, substituting 2-(piperidin-1-yl)ethanamine for 2-morpholinoethanamine used in Example 36. LC-MS (ESI) m/z 285,287 (M+H)⁺.

Step 2: tert-Butyl 2-fluoro-4-(6-(2-(piperidin-1-yl)ethylamino)pyridin-3-yl)phenylcarbamate (361 mg, 87%) was synthesized as a brown solid according to the procedure described in Step 2 of Example 40, substituting 5-bromo-N-(2-(piperidin-1-yl)ethyl)pyridin-2-amine for 5-bromo-N-tritylpyridin-2-amine, and 4-(tert-butoxycarbonylamino)-3-fluorophenylboronic acid for 5-(4,4,5,5-tetramethyl-1,3,2-dioxaborolan-2-yl)pyridin-2-amine used in Example 40. LC-MS (ESI) m/z 415 (M+H)⁺.

Step 3: To tert-Butyl 2-fluoro-4-(6-(2-(piperidin-1-yl)ethylamino)pyridin-3-yl)phenylcarbamate from Step 2 (361 mg, 0.87 mmol) in DCM (15 mL) was added excess TFA (8 mL) and the mixture was stirred at rt for 4 h. The mixture was concentrated under reduced pressure to give 5-(4-amino-3-fluorophenyl)-N-(2-(piperidin-1-yl)ethyl)pyridin-2-amine (273 mg, 100%) as a brown oil. LC-MS (ESI) m/z 315 (M+H)⁺.

Step 4: To a solution of 5-(4-amino-3-fluorophenyl)-N-(2-(piperidin-1-yl)ethyl)pyridin-2-amine (132 mg, 0.42 mmol) and triethylamine (106 mg, 1.05 mmol) in DMF (5 mL) were added phenyl 5-tert-butylisoxazol-3-ylcarbamate (120 mg, 0.46 mmol) and a catalytic amount of DMAP. The mixture was stirred at rt overnight, then partitioned between water (20 mL) and EtOAc (10 mL). The aqueous layer was extracted with EtOAc (3×10 mL) and the combined extracts were washed with brine (20 mL), dried over Mg SO₄, filtered and concentrated under reduced pressure. Purification by preparative HPLC gave 1-(5-tert-butylisoxazol-3-yl)-3-(2-fluoro-4-(6-(2-(piperidin-1-yl)ethylamino)pyridin-3-yl)phenyl)urea (37 mg, 18%) as a white solid. LC-MS (ESI) m/z 482 (M+H)⁺. ¹H NMR (DMSO-d₆) δ: 9.87 (br s, 1H), 8.86 (br s, 1H), 8.35 (br s, 1H), 8.13 (t, J=8.3 Hz, 1H), 7.74 (d, J=8.5 Hz, 1H), 7.53 (d, J=12.8 Hz, 1H), 7.41 (d, J=8.5 Hz, 1H), 6.35-6.89 (m, 3H), 5.37-5.62 (m, 1H), 2.91 (s, 2H), 2.64 (d, J=12.2 Hz, 6H), 1.92 (s, 2H), 1.04-1.74 (m, 12H).

### Example 28

### Preparation of 1-(5-tert-butylisoxazol-3-yl)-3-(4-(6-(3-morpholinopropylamino)pyridin-3-yl)phenyl)urea

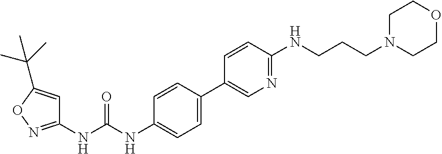 [SMILES: CC(C)(C)c1cc(NC(=O)Nc2ccc(-c3ccc(NCCCN4CCOCC4)nc3)cc2)no1]

Step 1: 5-bromo-N-(3-morpholinopropyl)pyridin-2-amine (566 mg, 94%) was synthesized as an oil according to the procedure described in Step 1 of Example 36, substituting 3-morpholinopropan-1-amine for 2-morpholinoethanamine used in Example 36. LC-MS (ESI) m/z 300, 302 (M+H)⁺.

Step 2: Crude 5-(4-aminophenyl)-N-(3-morpholinopropyl)pyridin-2-amine (104 mg) was synthesized as a brown solid according to the procedure described in Step 1 of Example 31, substituting 5-bromo-N-(3-morpholinopropyl)pyridin-2-amine from Step 1 above for 4-bromo-N-methylaniline, and 4-(4,4,5,5-tetramethyl-1,3,2-dioxaborolan-2-yl)aniline for 2-aminopyridine-5-boronic acid pinacol ester used in Example 31. LC-MS (ESI) m/z 31 (M+H)⁺.

Step 3: 1-(5-tert-butylisoxazol-3-yl)-3-(4-(6-(3-morpholinopropylamino)pyridin-3-yl)phenyl)urea (33 mg, 21%) was synthesized as a white solid according to the procedure described in Step 2 of Example 31, substituting 5-(4-aminophenyl)-N-(3-morpholinopropyl)pyridin-2-amine from Step 2 above for 5-(4-(methylamino)phenyl)pyridin-2-amine used in Example 31. LC-MS (ESI) m/z 479 (M+H)⁺. ¹H NMR (DMSO-d₆) δ: 9.62 (br s, 1H), 8.98 (s, 1H), 8.27 (br s, 1H), 7.66 (d, J=8.5 Hz, 1H), 7.50 (s, 4H), 6.42-6.74 (m, 3H), 5.36-5.59 (m, 1H), 3.29 (br s, 2H), 2.36 (br s, 7H), 1.70 (t, J=6.7 Hz, 2H), 1.30 (s, 9H).

### Example 29

### Preparation of 1-(5-tert-butylisoxazol-3-yl)-3-(4-(6-(2-(1-methylpyrrolidin-2-yl)ethylamino)pyridin-3-yl)phenyl)urea

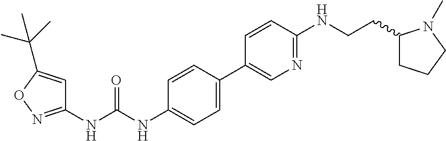 [SMILES: CN1CCCC1CCNc1ccc(-c2ccc(NC(=O)Nc3cc(C(C)(C)C)on3)cc2)cn1]

Step 1: 5-bromo-N-(2-(1-methylpyrrolidin-2-yl)ethyl)pyridin-2-amine (566 mg, 94%) was synthesized as an oil according to the procedure described in Step 1 of Example 36, substituting 2-(1-methylpyrrolidin-2-yl)ethanamine for 2-morpholinoethanamine used in Example 36. LC-MS (ESI) m/z 284, 286 (M+H)⁺.

Step 2: Crude 5-(4-aminophenyl)-N-(2-(1-methylpyrrolidin-2-yl)ethyl)pyridin-2-amine (154 mg) was synthesized as a brown solid according to the procedure described in Step 1 of Example 31, substituting 5-bromo-N-(2-(1-methylpyrrolidin-2-yl)ethyl)pyridin-2-amine from Step 1 above for 4-bromo-N-methylaniline, and 4-(4,4,5,5-tetramethyl-1,3,2-dioxaborolan-2-yl)aniline for 2-aminopyridine-5-boronic acid pinacol ester used in Example 31. LC-MS (ESI) m/z 297 (M+H)⁺.

Step 3: 1-(5-tert-Butylisoxazol-3-yl)-3-(4-(6-((1-ethylpyrrolidin-2-yl)methylamino)pyridin-3-yl)phenyl)urea (39 mg, 16%) was synthesized as a white solid according to the procedure described in Step 2 of Example 31, substituting 5-(4-aminophenyl)-N-(2-(1-methylpyrrolidin-2-yl)ethyl)pyridin-2-amine from Step 2 above for 5-(4-(methylamino)phenyl)pyridin-2-amine used in Example 31. LC-MS (ESI) m/z 463 (M+H)⁺. ¹H NMR (DMSO-d₆) δ: 9.88 (br s, 1H), 9.25 (br s, 1H), 8.28 (br s, 1H), 7.66 (d, J=8.3 Hz, 1H), 7.50 (s, 4H), 6.41-6.73 (m, 3H), 5.31-5.60 (m, 1H), 2.98 (br s, 1H), 2.13 (m, 1H), 1.89 (s, 7H), 1.64 (d, J=6.4 Hz, 2H), 1.38-1.57 (m, 2H), 1.30 (s, 9H).

### Example 30

### Preparation of 1-(5-tert-butylisoxazol-3-yl)-3-(4-(6-((1-ethylpyrrolidin-2-yl)methylamino)pyridin-3-yl)phenyl)urea

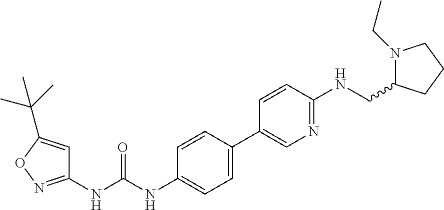 [SMILES: CCN1CCCC1CNc1ccc(-c2ccc(NC(=O)Nc3cc(C(C)(C)C)on3)cc2)cn1]

Step 1: 5-bromo-N-((1-ethylpyrrolidin-2-yl)methyl)pyridin-2-amine (270 mg, 95%) was synthesized as an oil according to the procedure described in Step 1 of Example 36, substituting (1-ethylpyrrolidin-2-yl)methanamine for 2-morpholinoethanamine used in Example 36. LC-MS (ESI) m/z 284, 286 (M+H)⁺.

Step 2: 5-(4-aminophenyl)-N-((1-ethylpyrrolidin-2-yl)methyl)pyridin-2-amine (207 mg, 77%) was synthesized as a solid according to the procedure described in Step 1 of Example 31, substituting 5-bromo-N-((1-ethylpyrrolidin-2-yl)methyl)pyridin-2-amine from Step 1 above for 4-bromo-N-methylaniline, and 4-(4,4,5,5-tetramethyl-1,3,2-dioxaborolan-2-yl)aniline for 2-aminopyridine-5-boronic acid pinacol ester used in Example 31. LC-MS (ESI) m/z 297 (M+H)⁺.

Step 3: 1-(5-tert-butylisoxazol-3-yl)-3-(4-(6-((1-ethylpyrrolidin-2-yl)methylamino)pyridin-3-yl)phenyl)urea (49 mg, 31%) was synthesized as a solid according to the procedure described in Step 2 of Example 31, substituting 5-(4-aminophenyl)-N-((1-ethylpyrrolidin-2-yl)methyl)pyridin-2-amine from Step 2 above for 5-(4-(methylamino)phenyl)pyridin-2-amine used in Example 31. LC-MS (ESI) m/z 463 (M+H)⁺. ¹H NMR (DMSO-d₆) δ: 9.80 (br s, 1H), 9.16 (br s, 1H), 8.27 (br s, 1H), 7.66 (d, J=8.5 Hz, 1H), 7.50 (s, 4H), 6.38-6.70 (m, 3H), 3.08 (d, J=5.7 Hz, 2H), 2.89 (m, 1H), 2.60 (br s, 1H), 2.02-2.39 (m, 3H), 1.49-1.77 (m, 4H), 1.30 (s, 9H), 1.06 (t, J=6.9 Hz, 3H).

### Example 31

### Preparation of 1-(4-(6-aminopyridin-3-yl)phenyl)-3-(5-tert-butylisoxazol-3-yl)-1-methylurea

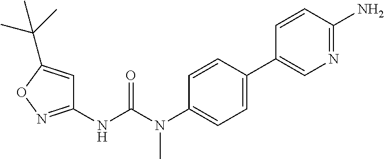 [SMILES: CN(C(=O)Nc1cc(C(C)(C)C)on1)c1ccc(-c2ccc(N)nc2)cc1]

Step 1: To a microwave reaction vessel were added 2-aminopyridine-5-boronic acid pinacol ester (220 mg, 1.0 mmol), 4-bromo-N-methylaniline (184 mg, 1.0 mmol), 1,4-dioxane (4 mL) and 20% aq sodium carbonate (3 mL). Argon gas was bubbled through the solution for 5 min, then tetrakis(triphenylphosphine) palladium(0) (57 mg, 0.05 mmol) was added. The vial was sealed and heated in a microwave reactor for 30 min at 150° C. After cooling to rt, silica gel was added and the mixture was concentrated under reduced pressure. Purification by silica gel flash chromatography eluting with 1:20 MeOH/EtOAc gave 5-(4-(methylamino)phenyl)pyridin-2-amine (79 mg, 39%) as an off white solid. LC-MS (ESI) m/z 200 (M+H)⁺.

Step 2: To a solution of 5-(4-(methylamino)phenyl)pyridin-2-amine (79 mg, 0.40 mmol) from Step 1 in DMF (2 mL) were added phenyl 5-tert-butylisoxazol-3-ylcarbamate (114 mg, 0.44 mmol) and a catalytic amount of DMAP. The solution was stirred at rt overnight, then partitioned between water (20 mL) and EtOAc (10 mL). The aqueous layer was extracted with EtOAc (3×10 mL) and the combined organic extracts were washed with brine (20 mL), dried over Mg SO₄, filtered, and concentrated under reduced pressure. The residue was purified by preparative HPLC to afford 1-(4-(6-aminopyridin-3-yl)phenyl)-3-(5-tert-butylisoxazol-3-yl)-1-methylurea (64 mg, 44%) as a white solid. LC-MS (ESI) m/z 366 (M+H)⁺. ¹H NMR (DMSO-d₆) δ: 9.20 (br s, 1H), 8.27 (br s, 1H), 7.73 (d, J=8.7 Hz, 1H), 7.59 (d, J=7.7 Hz, 2H), 7.32 (d, J=7.5 Hz, 2H), 6.40-6.62 (m, 2H), 6.06 (br s, 2H), 3.27 (s, 3H), 1.28 (s, 9H).

### Example 32

### Preparation of 5-(4-(3-(3-(2-fluoropropan-2-yl)isoxazol-5-yl)ureido)phenyl)pyridin-2-aminium methanesulfonate

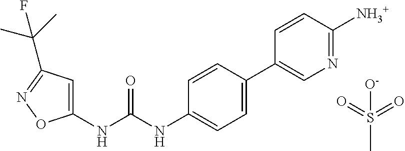

Step 1: To a stirred suspension of 60% NaH/mineral oil (12.48 g, 0.31 mol) in dry THF at 75° C. was added dropwise methyl 2-fluoro-2-methylpropanoate (24 g, 0.2 mol) in dry acetonitrile (16 mL, 0.31 mol) over the course of 45 min. The resulting pale yellow suspension was heated at 70° C. overnight, whereupon analysis by TLC indicated a single new product. After cooling to rt, the mixture was poured into water, acidified to pH˜2 with 2N HCl, and extracted with diethyl ether (1 L). The organic layer was dried over Na₂SO₄ and concentrated under reduced pressure. The residue was purified by silica gel chromatography eluting with 0-30% EtOAc in petroleum ether to afford 4-fluoro-4-methyl-3-oxopentanenitrile as a colorless oil (18 g, 72% yield). LC-MS (ESI) m/z 128 (M−H)⁺.

Step 2: To a stirred solution of 4-fluoro-4-methyl-3-oxopentanenitrile from Step 1 (12.9 g, 0.1 mol) and sodium hydroxide (8.20 g, 0.11 mol) in 1:1 water/EtOH (184 mL) was added hydroxylamine sulfate (17.23 g, 0.11 mol). The mixture was adjusted to pH 7.5 with 1N NaOH, then heated at 80° C. for 15 h. After cooling to rt, the mixture was concentrated to dryness under reduced pressure. The resulting solid was partitioned between water and dichloromethane, and the separated organic layer was washed with brine, dried over MgSO₄, and concentrated under reduced pressure. The residue was purified by silica gel chromatography eluting with 0-10% EtOAc in petroleum ether to afford 3-(2-fluoropropan-2-yl)isoxazol-5-amine as a yellow solid (5 g, 35%). LC-MS (ESI) m/z 145 (M+H)⁺.

Step 3: To a mixture of 3-(2-fluoropropan-2-yl)isoxazol-5-amine (4.32 g, 0.03 mol) and K₂CO₃ (8.28 g, 0.06 mol) in THF (100 mL) at 0° C. was added dropwise a solution of phenyl carbonochloridate (6 mL, 0.045 mol) in THF (50 mL). The mixture was stirred at 0° C. for 1 h, then at 40° C. for 20 h. Analysis by LC-MS and TLC indicated that the starting material was almost completely consumed and a new product had formed. The mixture was poured into water (150 mL) and the resulting mixture was extracted with EtOAc (100 mL). The organic layer was dried over Na₂SO₄ and concentrated under reduced pressure. The residue was purified by silica gel chromatography eluting with 0-4% EtOAc in petroleum ether to afford phenyl 3-(2-fluoropropan-2-yl)isoxazol-5-ylcarbamate as a white solid (6 g, 76%).

Step 4: 5-(4-(3-(3-(2-fluoropropan-2-yl)isoxazol-5-yl)ureido)phenyl)pyridin-2-aminium methanesulfonate (69.2 mg, 52% in 2 steps) was synthesized as a solid according to the procedure described in Step 4 of Example 36, substituting 5-(4-aminophenyl)pyridin-2-amine for 5-(4-aminophenyl)-N-(2-morpholinoethyl)pyridin-2-amine, and phenyl 3-(2-fluoropropan-2-yl)isoxazol-5-ylcarbamate from Step 3 above for phenyl 5-(1-(trifluoromethyl)cyclopropyl)isoxazol-3-ylcarbamate used in Example 36. LC-MS (ESI) m/z 356 (M+H)⁺. ¹H NMR (DMSO-d₆) δ: 13.67 (br s, 1H), 10.56 (s, 1H), 9.28 (s, 1H), 8.18-8.41 (m, 2H), 8.03 (br s, 2H), 7.63 (m, 4H), 7.08 (d, J=9.0 Hz, 1H), 6.18 (s, 1H), 2.43 (s, 3H), 1.72 (s, 3H), 1.65 (s, 3H).

### Example 33

### Preparation of 5-(4-(3-(5-(1-(trifluoromethyl)cyclopropyl)isoxazol-3-yl)ureido)phenyl)pyridin-2-aminium methanesulfonate

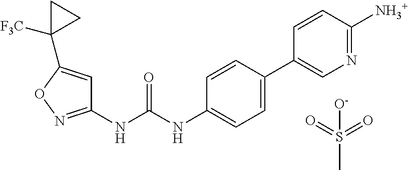

Step 1: Phenyl 5-(1-(trifluoromethyl)cyclopropyl)isoxazol-3-ylcarbamate was synthesized according to the procedure described in Steps 1-3 of Example 32, substituting methyl 1-(trifluoromethyl)cyclopropanecarboxylate for methyl 2-fluoro-2-methylpropanoate used in Example 32.

Step 2: 5-(4-(3-(5-(1-(trifluoromethyl)cyclopropyl)isoxazol-3-yl)ureido)phenyl)pyridin-2-aminium methanesulfonate (54.7 mg, 40%) was synthesized as a solid according to the procedure described in Step 4 of Example 36, substituting 5-(4-aminophenyl)pyridin-2-amine for 5-(4-aminophenyl)-N-(2-morpholinoethyl)pyridin-2-amine used in Example 36. LC-MS (ESI) m/z 404 (M+H)⁺. ¹H NMR (DMSO-d₆) δ: 13.60 (br s, 1H), 9.80 (s, 1H), 9.10 (s, 1H), 8.18-8.38 (m, 2H), 8.02 (br s, 2H), 7.61 (m, 4H), 7.08 (d, J=9.0 Hz, 1H), 6.89 (s, 1H), 2.39 (s, 3H), 1.52 (d, J=12.4 Hz, 4H).

### Example 34

### Preparation of 5-(4-(3-(5-(1,3-difluoro-2-methylpropan-2-yl)isoxazol-3-yl)ureido)phenyl)pyridin-2-aminium methanesulfonate

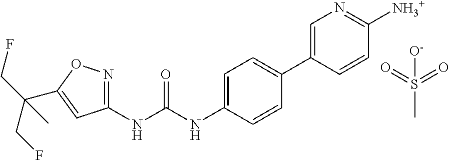

Step 1: Phenyl 5-(1,3-difluoro-2-methylpropan-2-yl)isoxazol-3-ylcarbamate was synthesized according to the procedure described in Steps 1-3 of Example 32, substituting methyl 3-fluoro-2-(fluoromethyl)-2-methylpropanoate for methyl 2-fluoro-2-methylpropanoate used in Example 32.

Step 2: 5-(4-(3-(5-(1,3-difluoro-2-methylpropan-2-yl)isoxazol-3-yl)ureido)phenyl)pyridin-2-aminium methanesulfonate (39.6 mg, 30% in 2 steps) was synthesized as a solid according to the procedure described in Step 4 of Example 36, substituting 5-(4-aminophenyl)pyridin-2-amine for 5-(4-aminophenyl)-N-(2-morpholinoethyl)pyridin-2-amine, and phenyl 5-(1,3-difluoro-2-methylpropan-2-yl)isoxazol-3-ylcarbamate from Step 1 above for phenyl 5-(1-(trifluoromethyl)cyclopropyl)isoxazol-3-ylcarbamate used in Example 36. LC-MS (ESI) m/z 388 (M+H)⁺. ¹H NMR (DMSO-d₆) δ: 13.66 (br s, 1H), 9.73 (s, 1H), 9.04 (s, 1H), 8.28-8.44 (m, 2H), 7.99 (br s, 2H), 7.61 (m, 4H), 7.07 (d, J=9.2 Hz, 1H), 6.89 (s, 1H), 4.74 (s, 2H), 4.58 (s, 2H), 2.35 (s, 3H), 1.34 (s, 3H).

### Example 35

### Preparation of 5-(4-(3-(3-(2-fluoropropan-2-yl)isoxazol-5-yl)ureido)phenyl)pyridin-2-aminium methanesulfonate

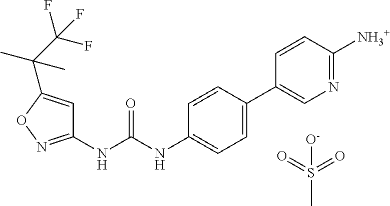

Step 1: Phenyl 5-(1,1,1-trifluoro-2-methylpropan-2-yl)isoxazol-3-ylcarbamate was synthesized according to the procedure described in Steps 1-3 of Example 32, substituting methyl 3,3,3-trifluoro-2,2-dimethylpropanoate for methyl 2-fluoro-2-methylpropanoate used in Example 32.

Step 2: 5-(4-(3-(3-(2-fluoropropan-2-yl)isoxazol-5-yl)ureido)phenyl)pyridin-2-aminium methanesulfonate (96 mg, 70%) was synthesized as a solid according to the procedure described in Step 4 of Example 36, substituting 5-(4-aminophenyl)pyridin-2-amine for 5-(4-aminophenyl)-N-(2-morpholinoethyl)pyridin-2-amine, and phenyl 5-(1,1,1-trifluoro-2-methylpropan-2-yl)isoxazol-3-ylcarbamate from Step 1 above for phenyl 5-(1-(trifluoromethyl)cyclopropyl)isoxazol-3-ylcarbamate used in Example 36. LC-MS (ESI) m/z 406 (M+H)⁺. ¹H NMR (DMSO-d₆) δ: 13.60 (br s, 1H), 9.83 (s, 1H), 9.14 (s, 1H), 8.18-8.38 (m, 2H), 8.02 (br s, 2H), 7.61 (m, 4H), 7.09 (d, J=9.0 Hz, 1H), 6.89 (s, 1H), 2.38 (s, 3H), 1.56 (s, 6H).

### Example 36

### Preparation of 4-(2-(5-(4-(3-(5-(1-(trifluoromethyl)cyclopropyl)isoxazol-3-yl)ureido)phenyl)pyridin-2-ylamino)ethyl)morpholin-4-ium methanesulfonate

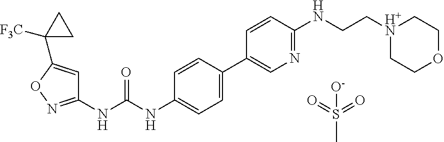

Step 1: To a solution of 5-bromo-2-fluoropyridine (1.2 g, 6.8 mmol) in t-BuOH (10 mL) were added 2-morpholinoethylamine (1.06 g, 8.2 mmol) and a catalytic amount of TsOH, and the solution was stirred at 95° C. overnight. After cooling to rt, the mixture was partitioned between saturated aq NaHCO₃ (80 mL) and EtOAc (60 mL). The aqueous layer was extracted with EtOAc (3×30 mL). The combined organic phases were washed with brine (100 mL), dried over MgSO₄, filtered and concentrated under reduced pressure. The residue was purified by silica gel flash chromatography eluting with 1:5 MeOH in EtOAc to afford 5-bromo-N-(2-morpholinoethyl)pyridin-2-amine (1.36 g, 70%) as a colorless oil. LC-MS (ESI) m/z 286, 288 (M+H)⁺.

Step 2: tert-Butyl 4-(6-(2-morpholinoethylamino)pyridin-3-yl)phenylcarbamate (1.21 g, 67%) was synthesized as a brown solid according to the procedure described in Step 2 of Example 40, substituting 5-bromo-N-(2-morpholinoethyl)pyridin-2-amine from Step 1 above for N6-trityl-3,3′-bipyridine-6,6′-diamine, and 4-(tert-butoxycarbonylamino)phenylboronic acid for 5-(4,4,5,5-tetramethyl-1,3,2-dioxaborolan-2-yl)pyridin-2-amine used in Example 40. LC-MS (ESI) m/z 399 (M+H)⁺.

Step 3: To a solution of 4N HCl/dioxane (40 mL) was added tert-butyl 4-(6-(2-morpholinoethylamino)pyridin-3-yl)phenylcarbamate from Step 2 (1.21 g) in DCM (4 mL) and the mixture was stirred at 45° C. for 2 h. The mixture was concentrated under reduced pressure to give 5-(4-aminophenyl)-N-(2-morpholinoethyl)pyridin-2-amine hydrochloride (1.0 g, 100%) as a brown solid. LC-MS (ESI) m/z 299 (M+H)⁺.

Step 4: To a solution of 5-(4-aminophenyl)-N-(2-morpholinoethyl)pyridin-2-amine hydrochloride (100 mg, 0.33 mmol) and triethylamine (66 mg, 0.66 mmol) in DMF (2 mL) was added phenyl 5-(1-(trifluoromethyl)cyclopropyl)isoxazol-3-ylcarbamate from Step 1 of Example 33 (112 mg, 0.36 mmol) and a catalytic amount of DMAP, and the solution was stirred at rt overnight. The mixture was partitioned between water (20 mL) and EtOAc (10 mL) and the separated aqueous layer was extracted with EtOAc (3×10 mL). The combined organic phases were washed with brine (20 mL), dried over Mg SO₄, filtered, and concentrated under reduced pressure. Purification of the residue by preparative HPLC gave 1-(4-(6-(2-morpholinoethylamino)pyridin-3-yl)phenyl)-3-(5-(1-(trifluoromethyl)cyclopropyl)isoxazol-3-yl)urea (80 mg, 46%) as a solid. LC-MS (ESI) m/z 517 (M+H)⁺. To a mixture of 1-(4-(6-(2-morpholinoethylamino)pyridin-3-yl)phenyl)-3-(5-(1-(trifluoromethyl)cyclopropyl)isoxazol-3-yl)urea (80 mg, 0.15 mmol) in MeCN (10 mL) was added MsOH (15.1 mg, 0.158 mmol.). The mixture was stirred at 55° C. for 2 h, then the mixture was concentrated under reduced pressure. The residue was dissolved in water and lyophilized to give 4-(2-(5-(4-(3-(5-(1-(trifluoromethyl)cyclopropyl)isoxazol-3-yl)ureido)phenyl)pyridin-2-ylamino)ethyl)morpholin-4-ium methanesulfonate (95.1 mg, 100%) as a white solid. LC-MS (ESI) m/z 517 (M+H)⁺. ¹H NMR (DMSO-d₆) δ: 9.79 (s, 1H), 9.10 (s, 1H), 8.35 (s, 1H), 7.87 (d, J=7.7 Hz, 1H), 7.56 (br s, 4H), 7.29 (br s, 1H), 6.90 (s, 1H), 6.75 (d, J=8.3 Hz, 1H), 3.88 (br s, 4H), 3.69 (br s, 2H), 3.33-3.58 (m, 7H), 2.39 (s, 3H), 1.54 (s, 2H), 1.50 (s, 2H).

### Example 37

### Preparation of 4-(2-(5-(4-(3-(5-(1,3-difluoro-2-methylpropan-2-yl)isoxazol-3-yl)ureido)phenyl)pyridin-2-ylamino)ethyl)morpholin-4-ium methanesulfonate

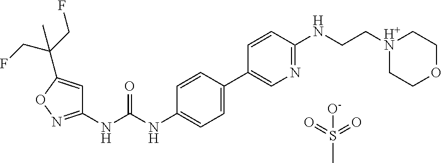

4-(2-(5-(4-(3-(5-(1,3-difluoro-2-methylpropan-2-yl)isoxazol-3-yl)ureido)phenyl)pyridin-2-ylamino)ethyl)morpholin-4-ium methanesulfonate (129.2 mg, 65%) was synthesized as a solid according to the procedure described in Step 4 of Example 36, substituting phenyl 5-(1,3-difluoro-2-methylpropan-2-yl)isoxazol-3-ylcarbamate for phenyl 5-(1-(trifluoromethyl)cyclopropyl)isoxazol-3-ylcarbamate used in Example 36. LC-MS (ESI) m/z 501 (M+H)⁺. ¹H NMR (DMSO-d₆) δ: 9.71 (s, 1H), 9.04 (s, 1H), 8.36 (s, 1H), 7.84 (d, J=7.9 Hz, 1H), 7.55 (br s, 4H), 7.20 (br s, 1H), 6.80 (s, 1H), 6.72 (d, J=8.3 Hz, 1H), 4.74 (s, 2H), 4.58 (s, 2H), 3.87 (br s, 4H), 3.68 (br s. 2H), 3.32-3.61 (m, 7H), 2.37 (s, 3H), 1.34 (s, 3H).

### Example 38

### Preparation of 4,4-(2-(5-(4-(3-(3-(2-fluoropropan-2-yl)isoxazol-5-yl)ureido)phenyl)pyridin-2-ylamino)ethyl)morpholin-4-ium methanesulfonate

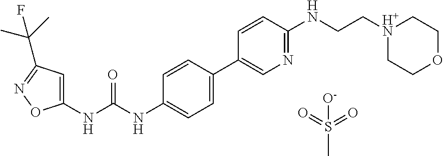

4,4-(2-(5-(4-(3-(3-(2-fluoropropan-2-yl)isoxazol-5-yl)ureido)phenyl)pyridin-2-ylamino)ethyl)morpholin-4-ium methanesulfonate (147.9 mg, 79%) was synthesized as a solid according to the procedure described in Step 4 of Example 36, substituting phenyl 3-(2-fluoropropan-2-yl)isoxazol-5-ylcarbamate for phenyl 5-(1-(trifluoromethyl)cyclopropyl)isoxazol-3-ylcarbamate used in Example 36. LC-MS (ESI) m/z 469 (M+H)⁺. ¹H NMR (DMSO-d₆) δ: 10.52 (s, 1H), 9.23 (s, 1H), 8.36 (s, 1H), 7.83 (d, J=8.1 Hz, 1H), 7.56 (br s, 4H), 7.15 (br s, 1H), 6.71 (d, J=8.3 Hz, 1H), 6.17 (s, 1H), 3.87 (br s, 4H), 3.67 (br s. 2H), 3.38-3.60 (m, 7H), 2.41 (s, 3H), 1.72 (s, 3H), 1.65 (s, 3H).

### Example 39

### Preparation of 4-(2-(5-(4-(3-(5-(1,1,1-trifluoro-2-methylpropan-2-yl)isoxazol-3-yl)ureido)phenyl)pyridin-2-ylamino)ethyl)morpholin-4-ium methanesulfonate

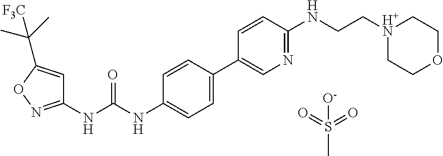

4-(2-(5-(4-(3-(5-(1,1,1-trifluoro-2-methylpropan-2-yl)isoxazol-3-yl)ureido)phenyl)pyridin-2-ylamino)ethyl)morpholin-4-ium methanesulfonate (48.5 mg, 24% in 2 steps) was synthesized as a solid according to the procedure described in Step 4 of Example 36, substituting phenyl 5-(1,1,1-trifluoro-2-methylpropan-2-yl)isoxazol-3-ylcarbamate for phenyl 5-(1-(trifluoromethyl)cyclopropyl)isoxazol-3-ylcarbamate used in Example 36. LC-MS (ESI) m/z 519 (M+H)⁺. ¹H NMR (DMSO-d₆) δ: 9.79 (s, 1H), 9.10 (s, 1H), 8.33 (s, 1H), 8.10 (br s, 1H), 7.45-7.79 (m, 4H), 7.81-7.08 (m, 2H), 6.90 (s, 1H), 3.89 (br s, 4H), 3.66 (br s. 2H), 3.30-3.62 (m, 7H), 2.40 (s, 3H), 1.56 (s, 6H).

### Example 40

### Preparation of 1-(6′-amino-3,3′-bipyridin-6-yl)-3-(5-(1-(trifluoromethyl)cyclopropyl)isoxazol-3-yl)urea

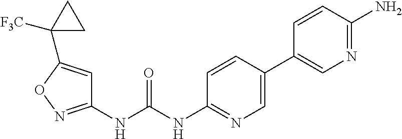 [SMILES: Nc1ccc(-c2ccc(NC(=O)Nc3cc(C4(C(F)(F)F)CC4)on3)nc2)cn1]

Step 1: To a solution of 5-bromopyridin-2-amine (1.73 g, 10 mmol) in DCM (80 mL) were added trityl chloride (3.05 g, 11 mmol) and triethylamine (1.11 g, 11 mmol) and the solution was heated under reflux overnight. After cooling to rt, the mixture was partitioned between DCM (80 mL) and a saturated aq NaHCO₃ (100 mL). The organic layer was washed with brine (100 mL), dried over MgSO₄, filtered and evaporated under reduced pressure. Purification of the residue by silica gel flash chromatography eluting with 1:1 DCM/hexane gave 5-bromo-N-tritylpyridin-2-amine (3.12 g, 75%) as an off white solid. LC-MS (ESI) m/z 416 (M+H)⁺.

Step 2: To a microwave reaction vessel were added 5-bromo-N-tritylpyridin-2-amine (519 mg, 1.25 mmol), 5-(4,4,5,5-tetramethyl-1,3,2-dioxaborolan-2-yl)pyridin-2-amine (275 mg, 1.25 mmol), 1,4-dioxane (6 mL), and 20% aq sodium carbonate (4 mL). Argon gas was bubbled through the solution for 5 min, then tetrakis(triphenylphosphine) palladium(0) (72 mg, 0.0625 mmol) was added, and the vial was sealed and heated in a microwave reactor for 20 min at 150° C. The mixture was partitioned between EtOAc (60 mL) and saturated aq NaHCO₃ (60 mL), and the separated aqueous layer was extracted with EtOAc (2×30 mL). The combined organic phases were washed with brine (100 mL), dried over MgSO₄, filtered and concentrated under reduced pressure. Purification by silica gel flash chromatography eluting with 1:1 EtOAc/DCM afforded N⁶-trityl-3,3′-bipyridine-6,6′-diamine (223 mg, 42%) as an off white solid. LC-MS (ESI) m/z 429 (M+H)⁺.

Step 3: To a 20 mL vial were added N⁶-trityl-3,3′-bipyridine-6,6′-diamine (160 mg, 0.37 mmol), phenyl 5-(1-(trifluoromethyl)cyclopropyl)isoxazol-3-ylcarbamate (174 mg, 0.56 mmol), triethylamine (57 mg, 0.56), DMF (3 mL), and catalytic amount of DMAP. The vial was sealed and stirred at 65° C. for 16 h, then the mixture was cooled and partitioned between water (20 mL) and EtOAc (25 mL). The separated aqueous layer was extracted with EtOAc (3×15 mL), and the combined organic phases were washed with brine (2×5 mL), dried over MgSO₄, filtered and concentrated under reduced pressure. Purification by silica gel flash chromatography eluting with 1:1 EtOAc/DCM gave 1-(5-(1-(trifluoromethyl)cyclopropyl)isoxazol-3-yl)-3-(6′-(tritylamino)-3,3′-bipyridin-6-yl)urea (82 mg, 34%) as a white solid. LC-MS (ESI) m/z 647 (M+H)⁺.

Step 4: To a solution of 5 1-(5-(1-(trifluoromethyl)cyclopropyl)isoxazol-3-yl)-3-(6′-(tritylamino)-3,3′-bipyridin-6-yl)urea (82 mg, 0.13 mmol) in DCM (15 mL) were added TFA (2 mL) and 2 drops of water. The mixture was stirred at rt overnight, then the mixture was concentrated under reduced pressure, and the residue was purified by preparative HPLC to afford 1-(6′-amino-3,3′-bipyridin-6-yl)-3-(5-(1-(trifluoromethyl)cyclopropyl)isoxazol-3-yl)urea (19.2 mg, 38%) as a white solid. LC-MS (ESI) m/z 405 (M+H)⁺. ¹H NMR (DMSO-d₆) δ: 11.05 (br s, 1H), 9.71 (br s, 1H), 8.54 (br s, 1H), 8.28 (s, 1H), 8.03 (t, J=8.0 Hz, 1H), 7.83 (d, J=8.1 Hz, 1H), 7.62 (d, J=8.3 Hz, 1H), 6.95 (s, 1H), 6.54-6.62 (m, 1H), 6.40 (br s, 2H), 1.53 (d, J=9.2 Hz, 4H).

### Example 41

### Preparation of 1-(6′-amino-3,3′-bipyridin-6-yl)-3-(5-(1,3-difluoro-2-methylpropan-2-yl)isoxazol-3-yl)urea

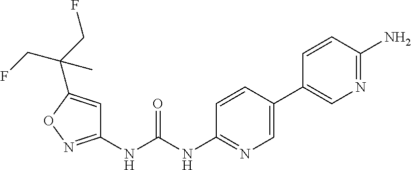 [SMILES: CC(CF)(CF)c1cc(NC(=O)Nc2ccc(-c3ccc(N)nc3)cn2)no1]

1-(6′-amino-3,3′-bipyridin-6-yl)-3-(5-(1,3-difluoro-2-methylpropan-2-yl)isoxazol-3-yl)urea (29.9 mg, 21%) was synthesized as a solid according to the procedure described in Steps 3 and 4 of Example 40, substituting phenyl 5-(1,3-difluoro-2-methylpropan-2-yl)isoxazol-3-ylcarbamate for phenyl 5-(1-(trifluoromethyl)cyclopropyl)isoxazol-3-ylcarbamate used in Example 40. LC-MS (ESI) m/z 389 (M+H)⁺. ¹H NMR (DMSO-d₆) δ: 10.97 (br s, 1H), 9.76 (br s, 1H), 8.60 (br s, 1H), 8.31 (s, 1H), 8.10 (m, J=8.0 Hz, 2H), 7.67 (d, J=8.5 Hz, 1H), 7.44 (d, J=2.1 Hz, 2H), 6.95 (m, 2H), 4.75 (s, 2H), 4.59 (s, 2H) 1.35 (s, 3H).

### Example 42

### Preparation of 1-(6′-amino-3,3′-bipyridin-6-yl)-3-(3-(2-fluoropropan-2-yl)isoxazol-5-yl)urea

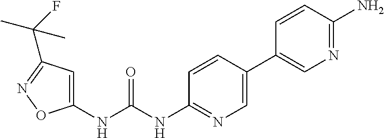 [SMILES: CC(C)(F)c1cc(NC(=O)Nc2ccc(-c3ccc(N)nc3)cn2)on1]

1-(6′-amino-3,3′-bipyridin-6-yl)-3-(3-(2-fluoropropan-2-yl)isoxazol-5-yl)urea (29.9 mg, 21%) was synthesized as a solid according to the procedure described in Steps 3 and 4 of Example 40, substituting phenyl 3-(2-fluoropropan-2-yl)isoxazol-5-ylcarbamate for phenyl 5-(1-(trifluoromethyl)cyclopropyl)isoxazol-3-ylcarbamate used in Example 40. LC-MS (ESI) m/z 357 (M+H)⁺. ¹H NMR (DMSO-d₆) δ: 11.72 (br s, 1H), 9.82 (br s, 1H), 8.56 (s, 1H), 8.28 (s, 1H), 8.04 (d, J=8.5 Hz, 1H), 7.76 (d, J=8.5 Hz, 1H), 7.58 (d, J=8.5 Hz, 1H), 6.55 (d, J=8.3 Hz, 1H), 6.25 (s, 1H), 6.19 (br s, 2H), 1.73 (s, 3H), 1.66 (s, 3H).

### Example 43

### Preparation of 1-(6′-amino-3,3′-bipyridin-6-yl)-3-(5-(1,1,1-trifluoro-2-methylpropan-2-yl)isoxazol-3-yl)urea

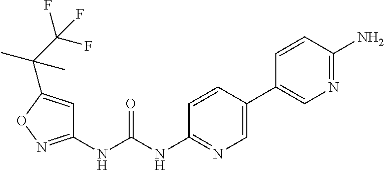 [SMILES: CC(C)(c1cc(NC(=O)Nc2ccc(-c3ccc(N)nc3)cn2)no1)C(F)(F)F]

1-(6′-amino-3,3′-bipyridin-6-yl)-3-(5-(1,1,1-trifluoro-2-methylpropan-2-yl)isoxazol-3-yl)urea (29.9 mg) was synthesized as a solid according to the procedure described in Steps 3 and 4 of Example 40, substituting phenyl 5-(1,1,1-trifluoro-2-methylpropan-2-yl)isoxazol-3-ylcarbamate for phenyl 5-(1-(trifluoromethyl)cyclopropyl)isoxazol-3-ylcarbamate used in Example 40. LC-MS (ESI) m/z 357 (M+H)⁺. ¹H NMR (DMSO-d₆) δ: 11.04 (br s, 1H), 9.74 (s, 1H), 8.58 (s, 1H), 8.29 (s, 1H), 8.05 (d, J=8.5 Hz, 1H), 7.94 (d, J=8.5 Hz, 1H), 7.63 (d, J=9.0 Hz, 1H), 6.96 (s, 1H), 6.74 (d, J=8.1 Hz, 1H), 5.76 (br s, 2H), 1.57 (s, 6H).

### Example 44

### Preparation of 1-(4-(2-aminopyrimidin-5-yl)phenyl)-3-(5-tert-butylisoxazol-3-yl)urea

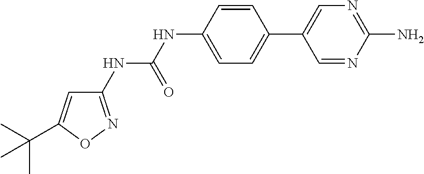 [SMILES: CC(C)(C)c1cc(NC(=O)Nc2ccc(-c3cnc(N)nc3)cc2)no1]

A 10 mL flask was charged with Pd₂(dba)₃ (27 mg, 0.03 mmol) and Cy₃P (18 mg, 0.09 mmol), then DME (2 mL), water (0.5 mL), and EtOH (0.5 mL) were added while flushing with nitrogen. 1-(4-Bromophenyl)-3-(5-tert-butylisoxazol-3-yl)urea (100 mg, 0.30 mmol), K₃PO₄.3H₂O (319 mg) and 2-aminopyridine-4-boronic acid pinacol ester (66 mg, 0.30 mmol) were added successively, and the mixture was heated at 90° C. for 3 h, whereupon analysis by TLC indicated that the starting material was consumed. The mixture was filtered through Celite washing with EtOAc (3×10 mL). Water (20 mL) was added to the filtrate, and the separated aquesous layer was extracted with EtOAc (3×20 mL). The combined organic phases were dried over Na₂SO₄ and concentrated. The residue was triturated with hot methanol and dried to afford 1-(4-(2-aminopyrimidin-5-yl)phenyl)-3-(5-tert-butylisoxazol-3-yl)urea (8 mg, 7% yield). LC-MS (ESI) m/z 353 (M+1)⁺, ¹H NMR (DMSO-d₆) δ: 9.52 (s, 1H), 8.89 (s, 1H), 8.55 (s, 2H), 7.54 (m, 2H), 6.70 (s, 2H), 6.52 (s, 1H), 1.31 (s, 9H).

### Example 45

### Preparation of 1-(5-tert-butylisoxazol-3-yl)-3-{4-[2-(2-morpholin-4-yl-ethylamino)-pyrimidin-5-yl]phenyl}urea

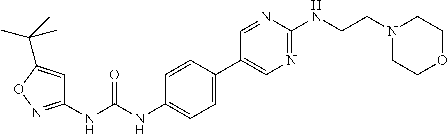 [SMILES: CC(C)(C)c1cc(NC(=O)Nc2ccc(-c3cnc(NCCN4CCOCC4)nc3)cc2)no1]

Step 1: To a 40 mL scintillation vial were added 5-bromo-2-chloropyrimidine (3.12 g, 16.1 mmol), i-PrOH (30 mL), DIEA (5.50 mL, 33.3 mmol), and 2-morpholinoethylamine (2.2 mL, 16.8 mmol). The mixture was heated in the sealed vial for 3 d at 50° C. The mixture was concentrated to and orange oil, which was partitioned between ether (100 mL) and water (100 mL). The separated aqueous layer was extracted with ether (3×50 mL) and EtOAc (100 mL). The combined organic phases were dried over MgSO₄, filtered, and concentrated under reduced pressure to give a thick oil. Tritration with ether then hexanes afforded (5-bromopyrimidin-2-yl)-(2-morpholin-4-yl-ethyl)amine (4.21 g, 91%) as a cream solid, which was sufficiently pure for the next step. LC-MS (ESI) m/z 287, 289 (M+H)⁺.

Step 2: [5-(4-Aminophenyl)pyrimidin-2-yl]-(2-morpholin-4-yl-ethyl)amine (37.5 mg, 50%) was synthesized as a solid according to the procedure described in Step 1 of Example 2, substituting (5-bromopyrimidin-2-yl)-(2-morpholin-4-yl-ethyl)amine for 5-bromo-3-cyano-2-aminopyridine used in Example 2. LC-MS (ESI) m/z 300 (M+H)⁺.

Step 3: 1-(5-tert-Butylisoxazol-3-yl)-3-{4-[2-(2-morpholin-4-yl-ethylamino)-pyrimidin-5-yl]-phenyl}urea (46.4 mg, 80%) was synthesized as a solid according to the procedure described in Step 2 of Example 2, substituting [5-(4-aminophenyl)pyrimidin-2-yl]-(2-morpholin-4-yl-ethyl)amine from Step 2 above for 2-amino-5-(4-aminophenyl)nicotinonitrile used in Example 2. LC-MS (ESI) m/z 466 (M+H)⁺. ¹H NMR (DMSO-d₆) δ: 9.51 (s, 1H), 8.88 (s, 1H), 8.59 (s, 2H), 7.54 (q, J=8.8 Hz, 4H), 7.10 (t, J=5.7 Hz, 1H), 6.51 (s, 1H), 3.53-3.62 (m, 4H), 3.44 (q, J=6.5 Hz, 2H), 2.41 (br s, 6H), 1.30 (s, 9H).

### Example 46

### Preparation of N-(4-(2-aminopyrimidin-5-yl)phenyl)-2-(3-(trifluoromethyl)phenyl)acetamide

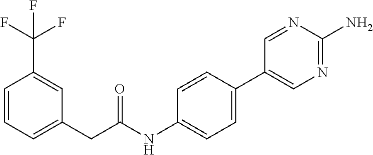 [SMILES: Nc1ncc(-c2ccc(NC(=O)Cc3cccc(C(F)(F)F)c3)cc2)cn1]

Step 1: 5-(4-Aminophenyl)pyrimidin-2-amine (136.8 mg, 59%) was synthesized as a solid according to the procedure described in Step 1 of Example 2, substituting 5-bromo-2-aminopyrimidine for 5-bromo-3-cyano-2-aminopyridine used in Example 2. LC-MS (ESI) m/z 187 (M+H)⁺.

Step 2: To a stirred solution of 3-trifluoromethylphenyl acetic acid (70 mg, 0.34 mmol) in 1.6 mL of DMF were added TEA (0.13 mL, 0.93 mmol), HOBt (50 mg, 0.37 mmol), and EDCI (66 mg, 0.34 mmol). After 15 min, 5-(4-aminophenyl)pyrimidin-2-amine from Step 1 (68.4 mg, 0.37 mmol) was added and the mixture was heated at 50° C. for 16 h. The mixture was concentrated and the residue was purified by silica gel flash chromatography eluting with 1-7% MeOH in DCM to give N-(4-(2-aminopyrimidin-5-yl)phenyl)-2-(3-(trifluoromethyl)phenyl)acetamide (97.6 mg, 75%). LC-MS (ESI) m/z 373 (M+H)⁺; ¹H NMR (DMSO-d₆) δ: 10.31 (s, 1H), 8.54 (s, 2H), 7.74-7.51 (m, 8H), 6.73 (s, 2H), 3.80 (s, 2H).

### Example 47

### Preparation of 1-(5-tert-Butyl-isoxazol-3-yl)-3-{4-[2-(2-morpholin-4-yl-ethoxy)-pyrimidin-5-yl]-phenyl}-urea

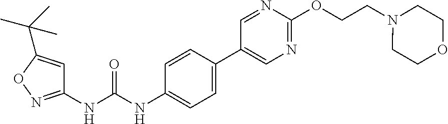 [SMILES: CC(C)(C)c1cc(NC(=O)Nc2ccc(-c3cnc(OCCN4CCOCC4)nc3)cc2)no1]

Step 1: tert-Butyl 4-(2-fluoropyrimidin-5-yl)phenylcarbamate (158.7 mg, 33%) was prepared according to the procedure described in Step 1 of Example 48, substituting 4-(tert-butoxycarbonylamino)phenylboronic acid for 2-methoxy-4-(4,4,5,5-tetramethyl-1,3,2-dioxaborolan-2-yl)aniline, and 5-bromo-2-fluoro-pyrimidine for 5-bromo-2-aminopyrimidine used in Example 48.

Step 2: To a stirred solution of 2-morpholinoethanol (35 mg, 0.27 mmol) in 1 mL of DMF at 0° C. was added 60% NaH/mineral oil (13 mg, 0.33 mmol). After 30 min, tert-butyl 4-(2-fluoropyrimidin-5-yl)phenylcarbamate (73 mg, 0.25 mmol) from Step 1 was added and the mixture was heated at 80° C. for 18 h, whereupon analysis by LC-MS indicated the presence of desired product. The mixture was cooled to rt, and partitioned between EtOAc (10 mL) and water (5 mL) containing a small amount of 1N HCl. The separated aqueous layer was extracted with EtOAc (2×50 mL), and the combined organic phases were washed with brine, dried over MgSO₄, filtered, and concentrated under reduced pressure. The residue was purified by silica gel flash chromatography eluting with 1-10% MeOH in DCM to give tert-butyl 4-(2-(2-morpholinoethoxy)pyrimidin-5-yl)phenylcarbamate (31.3 mg, 29%). LC-MS (ESI) m/z 401 (M+H)⁺.

Step 3: To 4-(2-(2-Morpholinoethoxy)pyrimidin-5-yl)aniline (31.3 mg, 0.078 mmol) in DCM (2 mL) was added excess TFA (1 mL), and the mixture was stirred at rt for 2 h. The mixture was concentrated under reduced pressure, then the residue was partitioned between DCM (15 mL), saturated aq NaHCO₃ (15 mL), and 1N NaOH (2 mL)). The separated aqueous layer was extracted with EtOAc (2×5 mL), and the combined organic phases were dried over MgSO₄, filtered, and concentrated under reduced pressure to give 4-(2-(2-morpholinoethoxy)pyrimidin-5-yl)aniline as a solid, which was sufficiently pure for the next step. LC-MS (ESI) m/z 301 (M+H)⁺.

Step 4: 1-(5-tert-Butyl-isoxazol-3-yl)-3-{4-[2-(2-morpholin-4-yl-ethoxy)-pyrimidin-5-yl]-phenyl}-urea (88.8 mg, 81%) was synthesized as a solid according to the procedure described in Step 2 of Example 2, substituting 4-(2-(2-morpholinoethoxy)pyrimidin-5-yl)aniline from Step 3 above for 2-amino-5-(4-aminophenyl)nicotinonitrile used in Example 2. LC-MS (ESI) m/z 467 (M+H)⁺. ¹H NMR (DMSO-d₆) δ: 9.56 (s, 1H), 8.96 (s, 1H), 8.90 (s, 2H), 7.68 (d, 2H), 7.57 (d, 2H), 6.52 (s, 1H), 4.46 (t, 2H), 3.57 (t, 4H), 2.72 (t, 2H), 2.54-2.42 (m, 2H), 1.29 (s, 9H).

### Example 48

### Preparation of 1-[4-(2-aminopyrimidin-5-yl)-2-methoxy-phenyl]-3-(5-tert-butylisoxazol-3-yl)-urea

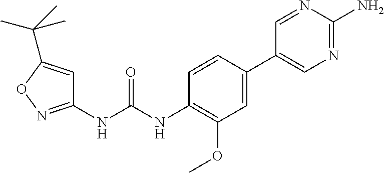 [SMILES: COc1cc(-c2cnc(N)nc2)ccc1NC(=O)Nc1cc(C(C)(C)C)on1]

Step 1: To a microwave reaction vessel were added 2-methoxy-4-(4,4,5,5-tetramethyl-1,3,2-dioxaborolan-2-yl)aniline (300 mg, 1.20 mmol), 5-bromo-2-aminopyrimidine (240 mg, 1.38 mmol), 1,4-dioxane (6 mL) and 2M aq sodium carbonate (1.42 mL, 2.83 mmol). Argon gas was bubbled through the solution for 5 min, then tetrakis(triphenylphosphine) palladium(0) (75.0 mg, 0.065 mmol) was added, and the vial was sealed and heated in a microwave reactor at 170° C. for 20 min. The mixture was partitioned between EtOAc (50 mL) and saturated aq NaHCO₃ (40 mL), and the separated aqueous layer was extracted with EtOAc (3×30 mL). The combined organic phases were washed with brine (40 mL), dried over MgSO₄, filtered and evaporated under reduced pressure to give a solid residue, which was purified by silica gel flash chromatography, eluting with 0-15% MeOH in DCM, to give 5-(4-amino-3-methoxyphenyl)pyrimidin-2-ylamine (182.7 mg, 70%) as a solid. LC-MS (ESI) m/z 217 (M+H)⁺.

Step 2: 1-[4-(2-aminopyrimidin-5-yl)-2-methoxyphenyl]-3-(5-tert-butylisoxazol-3-yl)urea (81.1 mg, 57%) was synthesized as a solid according to the procedure described in Step 2 of Example 2, substituting 5-(4-amino-3-methoxyphenyl)pyrimidin-2-ylamine from Step 1 above for 2-amino-5-(4-aminophenyl)nicotinonitrile used in Example 2. LC-MS (ESI) m/z 383 (M+H)⁺. ¹H NMR (DMSO-d₆) δ: 10.05 (s, 1H), 8.69 (br s, 1H), 8.60 (s, 2H), 8.15 (d, J=8.3 Hz, 1H), 7.27 (d, J=1.7 Hz, 1H), 7.16 (dd, J=8.3, 1.7 Hz, 1H), 6.73 (s, 2H), 6.48 (s, 1H), 3.96 (s, 3H), 1.30 (s, 9H).

### Example 49

### Preparation of 1-(4-(2-amino-4-methylpyrimidin-5-yl)phenyl)-3-(5-tert-butylisoxazol-3-yl)urea

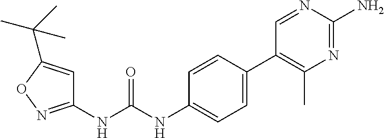 [SMILES: Cc1nc(N)ncc1-c1ccc(NC(=O)Nc2cc(C(C)(C)C)on2)cc1]

Step 1: 5-(4-Aminophenyl)-4-methylpyrimidin-2-amine (114.6 mg, 75%) was synthesized as a solid according to the procedure described in Step 1 of Example 2, substituting 5-bromo-4-methyl-2-aminopyrimidine for 5-bromo-3-cyano-2-aminopyridine used in Example 2. LC-MS (ESI) m/z 201 (M+H)⁺.

Step 2: 1-(4-(2-Amino-4-methylpyrimidin-5-yl)phenyl)-3-(5-tert-butylisoxazol-3-yl)urea (109.7 mg, 52%) was synthesized as a solid according to the procedure described in Step 2 of Example 2, substituting 5-(4-aminophenyl)-4-methylpyrimidin-2-amine from Step 1 above for 2-amino-5-(4-aminophenyl)nicotinonitrile used in Example 2. LC-MS (ESI) m/z 367 (M+H)⁺. ¹H NMR (DMSO-d₆) δ: 9.52 (s, 1H), 8.89 (s, 1H), 8.03 (s, 1H), 7.50 (d, 2H), 7.27 (s, 2H), 6.57 (s, 2H), 6.51 (s, 1H), 3.14 (s, 3H), 1.30 (s, 9H).

### Example 50

### Preparation of 1-[4-(2-amino-4-methoxypyrimidin-5-yl)-phenyl]-3-(5-tert-butylisoxazol-3-yl)-urea

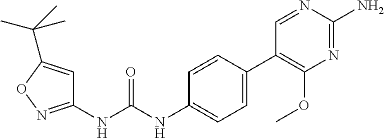 [SMILES: COc1nc(N)ncc1-c1ccc(NC(=O)Nc2cc(C(C)(C)C)on2)cc1]

Step 1: 5-(4-Aminophenyl)-4-methoxypyrimidin-2-ylamine (115.3 mg, 70%) was synthesized as a solid according to the procedure described in Step 1 of Example 2, substituting 5-bromo-4-methoxypyrimidin-2-amine for 5-bromo-3-cyano-2-aminopyridine used in Example 2. LC-MS (ESI) m/z 217 (M+H)⁺.

Step 2: 1-[4-(2-Amino-4-methoxypyrimidin-5-yl)-phenyl]-3-(5-tert-butylisoxazol-3-yl)-urea (115.4 mg, 57%) was synthesized as a solid according to the procedure described in Step 2 of Example 2, substituting 5-(4-aminophenyl)-4-methoxypyrimidin-2-ylamine from Step 1 above for 2-amino-5-(4-aminophenyl)nicotinonitrile used in Example 2. LC-MS (ESI) m/z 383 (M+H)⁺. ¹H NMR (DMSO-d₆) δ: 9.49 (s, 1H), 8.84 (s, 1H), 8.04 (s, 1H), 7.33-7.51 (m, 4H), 6.64 (s, 2H), 6.51 (s, 1H), 3.86 (s, 3H), 1.30 (s, 9H).

### Example 51

### Preparation of 1-(5-tert-butylisoxazol-3-yl)-3-(4-(2-(morpholinomethyl)pyrimidin-5-yl)phenyl)urea

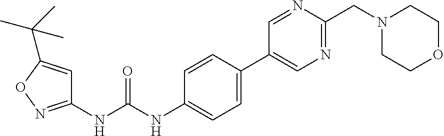 [SMILES: CC(C)(C)c1cc(NC(=O)Nc2ccc(-c3cnc(CN4CCOCC4)nc3)cc2)no1]

Step 1: A stirred mixture of (Z)-2-(4′-nitrophenyl)-3-N,N-dimethylaminopropenal (170 mg, 0.88 mmol) (Ref: Rivault, Freddy; Tranoy-Opalinski, Isabelle; Gesson, Jean-Pierre; Bioorganic & Medicinal Chemistry; 12; 2004; 675-682) and 2-morpholinoacetamidine hydrochloride (170 mg, 1.19 mmol) (Ref: Alker, David; Campbell, Simon F.; Cross, Peter E.; Burges, Roger A.; Carter, Anthony J.; Gardiner, Donald G.; Journal of Medicinal Chemistry; 32; 1989; 2381-2388) in EtOH (2 mL) was sonicated for 3 min and then heated at 80° C. for 16 h, whereupon analysis by LC-MS indicated the presence of the desired product. The mixture was concentrated under reduced pressure and the residue was purified by silica gel flash chromatography eluting with 1-12% MeOH in DCM to give 4-((5-(4-nitrophenyl)pyrimidin-2-yl)methyl)morpholine (88.9 mg, 34%) as a solid. LC-MS (ESI) m/z 301 (M+H)⁺.

Step 2: A stirred mixture of 4-((5-(4-nitrophenyl)pyrimidin-2-yl)methyl)morpholine (88.9 mg, 0.30 mmol) and SnCl₂ (140 mg, 0.62 mmol) in EtOH (5 mL) was heated under reflux for 2 h, whereupon analysis by LC-MS indicated the presence of desired product. After cooling the rt, mixture was concentrated under reduced pressure and the residue was partitioned between DCM (40 mL) and saturated aq NaHCO₃ (40 mL). The aqueous layer was extracted with 3:1 DCM: i-PrOH (3×80 mL) and the combined organic phases were dried over MgSO₄, filtered, and concentrated under reduced pressure to give 4-(2-(morpholinomethyl)pyrimidin-5-yl)aniline as a yellow oil.

Step 3: 1-(5-tert-Butylisoxazol-3-yl)-3-(4-(2-(morpholinomethyl)pyrimidin-5-yl)phenyl)urea (12.9 mg, 8.8%) was synthesized as a solid according to the procedure described in Step 2 of Example 2, substituting 4-(2-(morpholinomethyl)pyrimidin-5-yl)aniline from Step 2 of Example 45 for 2-amino-5-(4-aminophenyl)nicotinonitrile used in Example 2. LC-MS (ESI) m/z 437 (M+H)⁺. ¹H NMR (CDCl₃) δ: 8.93 (m, 2H), 7.67 (d, 2H), 7.53 (d, 2H), 7.41 (q, 2H), 6.02 (br s, 1H), 3.87 (s, 2H), 3.72 (br s, 4H), 2.64 (br s, 4H), 1.30 (s, 9H).

### Example 52

### Preparation of 1-[5-(2-fluoro-1-fluoromethyl-1-methylethyl)isoxazol-3-yl]-3-{4-[2-(2-morpholin-4-yl-ethylamino)pyrimidin-5-yl]phenyl}urea

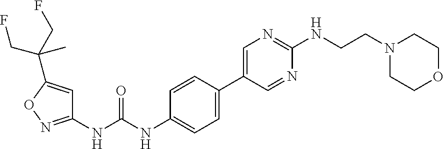 [SMILES: CC(CF)(CF)c1cc(NC(=O)Nc2ccc(-c3cnc(NCCN4CCOCC4)nc3)cc2)no1]

1-[5-(2-Fluoro-1-fluoromethyl-1-methylethyl)isoxazol-3-yl]-3-{4-[2-(2-morpholin-4-yl-ethylamino)pyrimidin-5-yl]phenyl}urea (79.1 mg, 59%) was synthesized as a solid according to the procedure described in Step 2 of Example 2, substituting [5-(4-aminophenyl)pyrimidin-2-yl]-(2-morpholin-4-yl-ethyl)amine from Step 2 of Example 45 for 2-amino-5-(4-aminophenyl)nicotinonitrile, and phenyl 5-(1,3-difluoro-2-methylpropan-2-yl)isoxazol-3-ylcarbamate for (5-tert-butylisoxazol-3-yl)carbamic acid phenyl ester used in Example 2. LC-MS (ESI) m/z 502 (M+H)⁺. ¹H NMR (DMSO-d₆) δ: 9.63 (s, 1H), 8.89 (s, 1H), 8.59 (br s, 2H), 7.45-7.60 (m, 4H), 7.10 (m, 1H), 6.80 (br s, 1H), 4.73 (br s, 2H), 4.58 (br s, 2H), 3.52-3.62 (m, 4H), 3.38-3.50 (m, 2H), 2.30-2.50 (m, 6H), 1.34 (s, 3H).

### Example 53

### Preparation of 1-{4-[2-(2-morpholin-4-yl-ethylamino)-pyrimidin-5-yl]-phenyl}-3-[5-(1-trifluoromethyl-cyclopropyl)-isoxazol-3-yl]urea

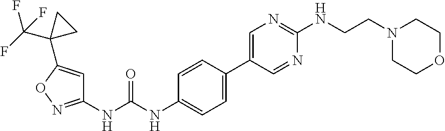 [SMILES: O=C(Nc1ccc(-c2cnc(NCCN3CCOCC3)nc2)cc1)Nc1cc(C2(C(F)(F)F)CC2)on1]

1-{4-[2-(2-Morpholin-4-yl-ethylamino)-pyrimidin-5-yl]-phenyl}-3-[5-(1-trifluoromethyl-cyclopropyl)-isoxazol-3-yl]urea (88.3 mg, 64%) was synthesized as a solid according to the procedure described in Step 2 of Example 2, substituting [5-(4-aminophenyl)pyrimidin-2-yl]-(2-morpholin-4-yl-ethyl)amine from Step 2 of Example 45 for 2-amino-5-(4-aminophenyl)nicotinonitrile, and phenyl 5-(1-(trifluoromethyl)cyclopropyl)isoxazol-3-ylcarbamate for (5-tert-butylisoxazol-3-yl)carbamic acid phenyl ester used in Example 2. LC-MS (ESI) m/z 518 (M+H)⁺. ¹H NMR (DMSO-d₆) δ: 9.68 (s, 1H), 8.90 (s, 1H), 8.59 (br s, 2H), 7.45-7.62 (m, 4H), 7.10 (m, 1H), 6.90 (br s, 1H), 3.52-3.62 (m, 4H), 3.38-3.50 (m, 2H), 3.13-3.20 (m, 2H), 2.30-2.50 (m, 6H), 1.51 (m, 2H).

### Example 54

### Preparation of 1-(4-(2-(2-morpholinoethylamino)pyrimidin-5-yl)phenyl)-3-(3-(trifluoromethyl)phenyl)urea

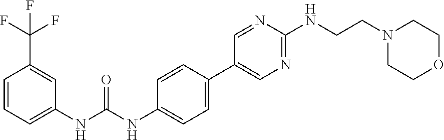 [SMILES: O=C(Nc1ccc(-c2cnc(NCCN3CCOCC3)nc2)cc1)Nc1cccc(C(F)(F)F)c1]

To a stirred solution of [5-(4-aminophenyl)pyrimidin-2-yl]-(2-morpholin-4-yl-ethyl)amine (80 mg, 0.27 mmol) from Step 2 of Example 45 and DMAP (70 mg, 0.57 mmol) in DMF (2 mL) was added 3-(trifluoromethyl)phenyl isocyanate (45 μL, 0.33 mmol). The mixture was heated at 50° C. for 16 h, whereupon analysis by LC-MS indicated the presence of desired product. The mixture was concentrated under reduced pressure and the residue was purified by silica gel flash chromatography eluting with 1-12% MeOH in DCM to give 1-(4-(2-(2-morpholinoethylamino)pyrimidin-5-yl)phenyl)-3-(3-(trifluoromethyl)phenyl)urea (72.4 mg, 56%). LC-MS (ESI) m/z 487 (M+H)⁺. ¹H NMR (DMSO-d₆) δ: 9.07 (s, 1H), 8.88 (s, 1H), 8.59 (s, 2H), 8.02 (s, 1H), 7.54 (m, 6H), 7.32 (d, 1H), 7.09 (t, 1H), 3.57 (br s, 4H), 3.43 (q, 2H), 2.41 (br s, 2H).

### Example 55

### Preparation of 1-(2-fluoro-5-methylphenyl)-3-(4-(2-(2-morpholinoethylamino)pyrimidin-5-yl)phenyl)urea

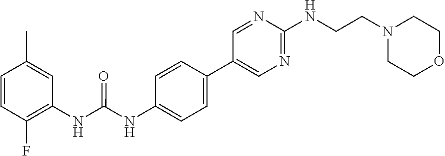 [SMILES: Cc1ccc(F)c(NC(=O)Nc2ccc(-c3cnc(NCCN4CCOCC4)nc3)cc2)c1]

1-(2-Fluoro-5-methylphenyl)-3-(4-(2-(2-morpholinoethylamino)pyrimidin-5-yl)phenyl)urea (74.4 mg, 62%) was synthesized as a solid according to the procedure described in Example 54, substituting 2-fluoro-5-methylphenyl isocyanate for 3-(trifluoromethyl)phenyl isocyanate used in Example 54. LC-MS (ESI) m/z 451 (M+H)⁺. ¹H NMR (DMSO-d₆) δ: 9.14 (s, 1H), 8.59 (d, 2H), 8.49 (d, 1H), 7.99 (dd, 1H), 7.54 (m, 4H), 7.09 (m, 2H), 6.81 (m, 1H), 3.58 (t, 4H), 3.43 (q, 2H), 2.42 (br t., 4H), 2.28 (t, 3H).

### Example 56

### Preparation of 1-(5-tert-butylisoxazol-3-yl)-3-(4-(2-(3-morpholinopropyl)pyrimidin-5-yl)phenyl)urea

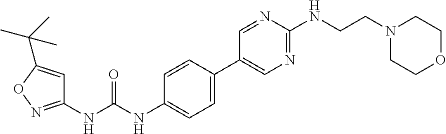 [SMILES: CC(C)(C)c1cc(NC(=O)Nc2ccc(-c3cnc(NCCN4CCOCC4)nc3)cc2)no1]

Step 1: To a stirred solution of 5-bromo-2-iodopyrimidine (548 mg, 1.92 mmol) and 2-prop-2-ynyloxytetrahydropyran (0.28 mL, 1.99 mmol) in DMF (2 mL) were added TEA (0.60 mL, 4.31 mmol) and CuI (20 mg, 0.11 mmol). Argon was bubbled through the solution for 5 min before PdCl₂(PPh₃)₂ (65 mg, 0.093 mmol) was added. The mixture was stirred at rt for 3 h, whereupon analysis by LC-MS indicated the presence of desired product. The solution was partitioned between EtOAc (50 mL) and brine (50 mL), and the aqueous layer was extracted with EtOAc (3×40 mL). The combined organic phases were dried over MgSO₄, filtered, and concentrated under reduced pressure, and the residue was purified by silica gel flash chromatography eluting with 5-100% EtOAc in hexanes to give 5-(4-bromophenyl)-2-(3-(tetrahydro-2H-pyran-2-yloxy)prop-1-ynyl)pyrimidine (474 mg, 83%). LC-MS (ESI) m/z 298 (M+H)⁺.

Step 2: To a microwave reaction vessel were added 4-(tert-butoxycarbonylamino)phenylboronic acid (540 mg, 2.28 mmol), 5-(4-bromophenyl)-2-(3-(tetrahydro-2H-pyran-2-yloxy)prop-1-ynyl)pyrimidine (694 mg, 2.33 mmol), 1,4-dioxane (10 mL), and 2M aq sodium carbonate (2.5 mL, 4.91 mmol). Argon gas was bubbled through the solution for 5 min, then tetrakis(triphenylphosphine) palladium(0) (120 mg, 0.10 mmol) was added, and the vial was sealed and heated in a microwave reactor at 170° C. for 18 min, whereupon analysis by LC-MS indicated the presence of desired product. The mixture was concentrated under reduced pressure and the residue was purified by silica gel flash chromatography, eluting with 1-12% MeOH in DCM, to give impure tert-butyl 4-(2-(3-(tetrahydro-2H-pyran-2-yloxy)prop-1-ynyl)pyrimidin-5-yl)phenylcarbamate. This material was dissolved in EtOH (30 mL), 10% Pd/C (250 mg) was added, and the resulting mixture was stirred under a hydrogen balloon at 60° C. for 2 h, whereupon analysis by LC-MS showed the presence of desired product. The mixture was filtered through Celite, the filtrate was concentrated under reduced pressure, and the residue was purified by silica gel flash chromatography eluting with 5-60% EtOAc in hexanes to give tert-butyl 4-(2-(3-(tetrahydro-2H-pyran-2-yloxy)propyl)pyrimidin-5-yl)phenylcarbamate (208.2 mg, 22%). LC-MS (ESI) m/z 414 (M+H)⁺.

Step 3: A solution of tert-butyl 4-(2-(3-(tetrahydro-2H-pyran-2-yloxy)propyl)pyrimidin-5-yl)phenylcarbamate (208.2 mg, 0.88 mmol) in MeOH (2.0 mL) was stirred with pyridiniump-toluenesulfonate (50 mg, 0.20 mmol) at rt for 16 h. More pyridiniump-toluenesulfonate (150 mg, 0.60 mmol) was added, and the mixture was stirred at 50° C. for 4 h, whereupon analysis by LC-MS indicated that all starting material was consumed. The mixture was concentrated under reduced pressure, and the residue was purified by silica gel flash chromatography eluting with 1-12% MeOH in DCM to give tert-butyl 4-(2-(3-hydroxypropyl)pyrimidin-5-yl)phenylcarbamate (125.1 mg, 43%). LC-MS (ESI) m/z 330 (M+H)⁺.

Step 4: To a stirred solution of tert-butyl 4-(2-(3-hydroxypropyl)pyrimidin-5-yl)phenylcarbamate (125.1 mg, 0.38 mmol) in THF (2 mL) were added TEA (0.11 mL, 0.79 mmol) and methanesulfonic anhydride (73 mg, 0.42 mmol), and the mixture was stirred at rt for 1 h. Morpholine (0.17 mL, 1.95 mmol) was added and the mixture was stirred at rt for 1 h. Additional morpholine (0.17 mL, 1.95 mmol) and a catalytic quantity of NaI were addedm and the resulting mixture was stirred at rt for 19 h. The mixture was concentrated under reduced pressure, and the residue was purified by silica gel flash chromatography eluting with 1-15% MeOH in DCM to give tert-butyl 4-(2-(3-(piperidin-1-yl)propyl)pyrimidin-5-yl)phenylcarbamate (67.9 mg, 44%). LC-MS (ESI) m/z 397 (M+H)⁺.

Step 5: To a stirred solution of tert-butyl 4-(2-(3-(piperidin-1-yl)propyl)pyrimidin-5-yl)phenylcarbamate (67.9 mg, 0.17 mmol) in DCM (4 mL) was added TFA (2.00 mL, 30.0 mmol). The mixture was stirred at rt for 2 h, then concentrated reduced pressure. The residue was partitioned between DCM (8 mL) and saturated aq NaHCO₃ (8 mL), and the separated aqueous layer was extracted with DCM (3×10 mL). The combined organic phases were concentrated under reduced pressure to give the title compound as a solid (21 mg, 41%). LC-MS (ESI) m/z 297 (M+H)⁺.

Step 6: 1-(5-tert-Butylisoxazol-3-yl)-3-(4-(2-(3-morpholinopropyl)pyrimidin-5-yl)phenyl)urea (5.13 mg, 18%) was synthesized as an acetate salt according to the procedure described in step 2 of Example 2, substituting 4-(2-(3-(piperidin-1-yl)propyl)pyrimidin-5-yl)aniline from Step 5 above for 2-amino-5-(4-aminophenyl)nicotinonitrile used in Example 2. LC-MS (ESI) m/z 465 (M+H)⁺. ¹H NMR (CDCl₃) δ: 9.52 (br s, 1H), 8.85 (s, 2H), 8.78 (br s, 1H), 7.68 (d, 2H), 7.53 (d, 2H), 5.95 (s, 1H), 3.72 (t, 4H), 3.20 (br s, 3H), 3.04 (t, 2H), 2.52 (m, 5H), 2.10 (m, 3H), 1.37 (s, 9H), 1.26 (br s, 2H).

### Example 57

### Preparation of 1-(5-tert-butylisoxazol-3-yl)-3-(4-(2-(2-(dimethylamino)ethylamino)pyrimidin-5-yl)phenyl)urea

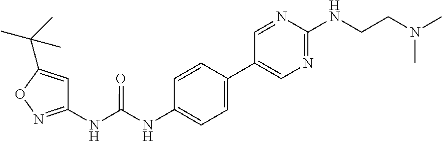 [SMILES: CN(C)CCNc1ncc(-c2ccc(NC(=O)Nc3cc(C(C)(C)C)on3)cc2)cn1]

Step 1: [5-(4-Aminophenyl)pyrimidin-2-yl]-(2-methoxyethyl)amine (277 mg, 66%) was synthesized according to the procedure described in Step 1 of Example 2, substituting N¹-(5-bromopyrimidin-2-yl)-N²,N²-dimethylethane-1,2-diamine for 5-bromo-3-cyano-2-aminopyridine used in Example 2. LC-MS (ESI) m/z 258 (M+H)⁺.

Step 2: 1-(5-tert-Butylisoxazol-3-yl)-3-(4-(2-(2-(dimethylamino)ethylamino)pyrimidin-5-yl)phenyl)urea (84 mg, 16%) was synthesized as an acetate salt according to the procedure described in Step 2 of Example 2, substituting [5-(4-aminophenyl)pyrimidin-2-yl]-(2-methoxyethyl)amine from Step 1 above for 2-amino-5-(4-aminophenyl)nicotinonitrile used in Example 2. LC-MS (ESI) m/z 424 (M+H)⁺. ¹H NMR (DMSO-d₆) δ: 9.55 (br s, 1H), 8.92 (br s, 1H), 8.59 (s, 2H), 7.54 (q, 4H), 7.10 (t, 1H0, 6.51 (s, 1H), 3.42 (q, 2H), 2.24 (s, 6H), 1.91 (s, 2H), 1.30 (s, 9H).

### Example 58

### Preparation of 1-(5-tert-butylisoxazol-3-yl)-3-{4-[2-(2-methoxyethylamino)pyrimidin-5-yl]-phenyl}urea

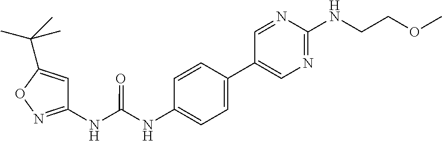 [SMILES: COCCNc1ncc(-c2ccc(NC(=O)Nc3cc(C(C)(C)C)on3)cc2)cn1]

Step 1: [5-(4-Aminophenyl)pyrimidin-2-yl]-(2-methoxyethyl)amine (380 mg, 96%) was synthesized as a solid according to the procedure described in Step 1 of Example 2, substituting 5-bromo-N-(2-methoxyethyl)pyrimidin-2-amine for 5-bromo-3-cyano-2-aminopyridine used in Example 2. LC-MS (ESI) m/z 245 (M+H)⁺.

Step 2: 1-(5-tert-butylisoxazol-3-yl)-3-{4-[2-(2-methoxyethylamino)pyrimidin-5-yl]-phenyl}urea (292.9 mg, 46%) was synthesized as a solid according to the procedure described in Step 2 of Example 2, substituting [5-(4-aminophenyl)pyrimidin-2-yl]-(2-methoxyethyl)amine from Step 1 above for 2-amino-5-(4-aminophenyl)nicotinonitrile used in Example 2. LC-MS (ESI) m/z 411 (M+H)⁺. ¹H NMR (DMSO-d₆) δ: 9.52 (s, 1H), 8.89 (s, 1H), 8.59 (s, 2H), 7.46-7.65 (m, 4H), 7.24 (br s, 1H), 6.51 (s, 1H), 3.47 (br s, 4H), 3.27 (s, 3H), 1.30 (s, 9H).

### Example 59

### Preparation of 1-[4-(6-aminopyridin-3-yl)-2-fluorophenyl]-3-(5-tert-butylisoxazol-3-yl)-urea

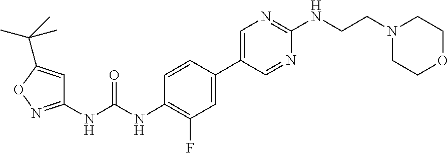 [SMILES: CC(C)(C)c1cc(NC(=O)Nc2ccc(-c3cnc(NCCN4CCOCC4)nc3)cc2F)no1]

Step 1: [5-(4-Amino-3-fluorophenyl)pyrimidin-2-yl]-(2-morpholin-4-yl-ethyl)amine (205.6 mg, 57%) was synthesized as a solid according to the procedure described in Step 1 of Example 2, substituting 4-(tert-butoxycarbonylamino)-3-fluorophenylboronic acid for 4-(tert-butoxycarbonylamino)phenylboronic acid, and (5-bromopyrimidin-2-yl)-(2-morpholin-4-yl-ethyl)amine for 5-bromo-3-cyano-2-aminopyridine used in Example 2. LC-MS (ESI) m/z 218 (M+H)⁺.

Step 2: 1-[4-(6-Aminopyridin-3-yl)-2-fluorophenyl]-3-(5-tert-butylisoxazol-3-yl)-urea (130.9 mg, 42%) was synthesized as a solid according to the procedure described in Step 2 of Example 2, substituting [5-(4-amino-3-fluorophenyl)pyrimidin-2-yl]-(2-morpholin-4-yl-ethyl)amine from Step 1 above for 2-amino-5-(4-aminophenyl)nicotinonitrile used in Example 2. LC-MS (ESI) m/z 484 (M+H)⁺. ¹H NMR (DMSO-d₆) δ: 9.85 (s, 1H), 8.86 (br s, 1H), 8.64 (s, 2H), 8.15 (t, J=8.6 Hz, 1H), 7.61 (dd, J=12.8, 1.7 Hz, 1H), 7.45 (d, J=8.5 Hz, 1H), 7.20 (t, J=5.7 Hz, 1H), 6.50 (s, 1H), 3.57 (t, J=4.4 Hz, 4H), 3.44 (q, J=6.4 Hz, 2H), 2.42 (br s, 6H), 1.30 (s, 9H).

### Example 60

### Preparation of 1-(5-tert-butylisoxazol-3-yl)-3-(4-(2-(2-(piperidin-1-yl)ethylamino)pyrimidin-5-yl)phenyl)urea

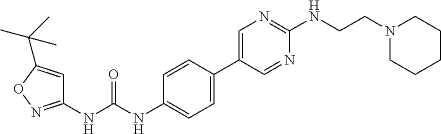 [SMILES: CC(C)(C)c1cc(NC(=O)Nc2ccc(-c3cnc(NCCN4CCCCC4)nc3)cc2)no1]

Step 1: 5-(4-Aminophenyl)-N-(2-(piperidin-1-yl)ethyl)pyrimidin-2-amine was synthesized according to the procedure described in Step 1 of Example 2, substituting 5-bromo-N-(2-(piperidin-1-yl)ethyl)pyrimidin-2-amine for 5-bromo-3-cyano-2-aminopyridine used in Example 2. LC-MS (ESI) m/z 298 (M+H)⁺.

Step 2: 1-(5-tert-Butylisoxazol-3-yl)-3-(4-(2-(2-(piperidin-1-yl)ethylamino)pyrimidin-5-yl)phenyl)urea (26.7 mg, 18%) was synthesized according to the procedure described in Step 2 of Example 2, substituting 5-(4-aminophenyl)-N-(2-(piperidin-1-yl)ethyl)pyrimidin-2-amine from Step 1 above for 2-amino-5-(4-aminophenyl)nicotinonitrile used in Example 2. LC-MS (ESI) m/z 464 (M+H)⁺. ¹H NMR (DMSO-d₆) δ: 9.53 (s, 1H), 8.92 (s, 1H), 8.59 (s, 2H), 7.53 (q, 4H), 7.05 (br s, 1H), 6.51 (s, 1H), 3.42 (br s, 2H), 2.40 (br s, 6H), 1.51 (br s, 6H), 1.30 (s, 9H).

### Example 61

### Preparation of 1-(5-tert-butylisoxazol-3-yl)-3-{5-[2-(2-morpholin-4-yl-ethylamino)pyrimidin-5-yl]pyridin-2-yl}urea

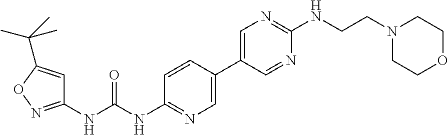 [SMILES: CC(C)(C)c1cc(NC(=O)Nc2ccc(-c3cnc(NCCN4CCOCC4)nc3)cn2)no1]

Step 1: [5-(6-Amino-pyridin-3-yl)-pyrimidin-2-yl]-(2-morpholin-4-yl-ethyl)-amine (215.3 mg, 62%) was synthesized as a solid according to the procedure described in Step 1 of Example 17, substituting (5-bromopyrimidin-2-yl)-(2-morpholin-4-yl-ethyl)amine from Step 1 of Example 45 for 5-bromo-2-aminopyridine used in Example 17. LC-MS (ESI) m/z 301 (M+H)⁺.

Step 2: 1-(5-tert-Butylisoxazol-3-yl)-3-{5-[2-(2-morpholin-4-yl-ethylamino)pyrimidin-5-yl]-pyridin-2-yl}urea (49.7 mg, 32%) was synthesized as a solid according to the procedure described in Step 2 of Example 2, substituting [5-(6-Amino-pyridin-3-yl)-pyrimidin-2-yl]-(2-morpholin-4-yl-ethyl)-amine from Step 1 above for 2-amino-5-(4-aminophenyl)nicotinonitrile used in Example 2. LC-MS (ESI) m/z 467 (M+H)⁺. ¹H NMR (DMSO-d₆) δ: 10.90 (br s, 1H), 9.70 (s, 1H), 8.65 (s, 2H), 8.58 (s, 1H), 8.07 (d, J=8.7 Hz, 1H), 7.61 (d, J=8.5 Hz, 1H), 7.16-7.28 (m, 1H), 6.58 (s, 1H), 3.57 (br s, 4H), 3.44 (q, J=6.1 Hz, 2H), 2.41 (br s, 6H), 1.31 (s, 9H).

### Example 62

### Preparation of 1-(5-(2-(tert-butylamino)pyrimidin-5-yl)pyridin-2-yl)-3-(5-tert-butylisoxazol-3-yl)urea

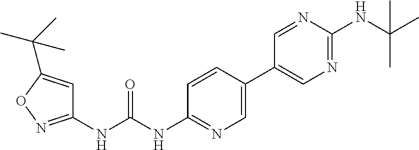 [SMILES: CC(C)(C)Nc1ncc(-c2ccc(NC(=O)Nc3cc(C(C)(C)C)on3)nc2)cn1]

Step 1: 5-Bromo-N-tert-butylpyrimidin-2-amine (95.5 mg, 18%) was synthesized according to the procedure described in Step 1 of Example 45, substituting tert-butylamine for 2-morpholinoethylamine used in Example 45. LC-MS (ESI) m/z 230, 232 (M+H)⁺.

Step 2: 5-(6-Aminopyridin-3-yl)-N-tert-butylpyrimidin-2-amine (64.2 mg, 65%) was synthesized according to the procedure described in Step 1 of Example 17, substituting 5-bromo-N-tert-butylpyrimidin-2-amine from Step 1 above for 5-bromo-2-aminopyridine used in Example 17. LC-MS (ESI) m/z 244 (M+H)⁺.

Step 3: 1-(5-(2-(tert-Butylamino)pyrimidin-5-yl)pyridin-2-yl)-3-(5-tert-butylisoxazol-3-yl)urea (30.7 mg, 28%) was synthesized according to the procedure described in Step 2 of Example 2, substituting 5-(6-aminopyridin-3-yl)-N-tert-butylpyrimidin-2-amine from Step 2 above for 2-amino-5-(4-aminophenyl)nicotinonitrile used in Example 2. LC-MS (ESI) m/z 410 (M+H)⁺. ¹H NMR (DMSO-d₆) δ: 10.91 (br s, 1H), 9.69 (s, 1H), 8.65 (s, 2H), 8.58 (s, 1H), 8.08 (d, 1H), 7.61 (d, 1H), 6.98 (s, 1H), 6.58 (s, 1H), 1.41 (s, 9H), 1.31 (s, 9H).

### Example 63

### Preparation of 1-(5-tert-butylisoxazol-3-yl)-3-(5-(2-(tetrahydro-2H-pyran-4-ylamino)pyrimidin-5-yl)pyridin-2-yl)urea

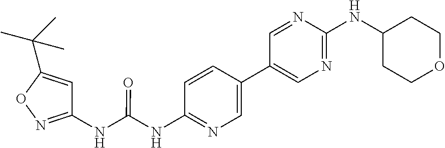 [SMILES: CC(C)(C)c1cc(NC(=O)Nc2ccc(-c3cnc(NC4CCOCC4)nc3)cn2)no1]

Step 1: 5-Bromo-N-(tetrahydro-2H-pyran-4-yl)pyrimidin-2-amine (504.4 mg, 76%) was synthesized according to the procedure described in Step 1 of Example 45, substituting tetrahydro-2H-pyran-4-amine for 2-morpholinoethylamine used in Example 45. LC-MS (ESI) m/z 258, 260 (M+H)⁺.

Step 2: 5-(6-Aminopyridin-3-yl)-N-(tetrahydro-2H-pyran-4-yl)pyrimidin-2-amine (373.7 mg, 74%) was synthesized according to the procedure described in Step 1 of Example 17, substituting 5-bromo-N-(tetrahydro-2H-pyran-4-yl)pyrimidin-2-amine from Step 1 above for 5-bromo-2-aminopyridine used in Example 17. LC-MS (ESI) m/z 272 (M+H)⁺.

Step 3: 1-(5-tert-Butylisoxazol-3-yl)-3-(5-(2-(tetrahydro-2H-pyran-4-ylamino)pyrimidin-5-yl)pyridin-2-yl)urea (160 mg, 27%) was synthesized according to the procedure described in Step 2 of Example 2, substituting 5-(6-aminopyridin-3-yl)-N-(tetrahydro-2H-pyran-4-yl)pyrimidin-2-amine from Step 2 above for 2-amino-5-(4-aminophenyl)nicotinonitrile used in Example 2. LC-MS (ESI) m/z 438 (M+H)⁺. ¹H NMR (DMSO-d₆) δ: 10.90 (br s, 1H), 9.70 (s, 1H), 8.65 (s, 2H), 8.58 (s, 1H), 8.06 (d, 1H), 7.62 (d, 1H), 7.40 (d, 1H), 6.58 (s, 1H), 4.10 (q, 1H), 3.93 (m, 3H), 3.41 (d, 2H), 3.17 (d, 3H), 1.84 (d, 2H), 1.54 (m, 2H), 1.31 (s, 9H).

### Example 64

### Preparation of 1-(5-tert-butylisoxazol-3-yl)-3-[5-(2-cyclopropylaminopyrimidin-5-yl)-pyridin-2-yl]-urea

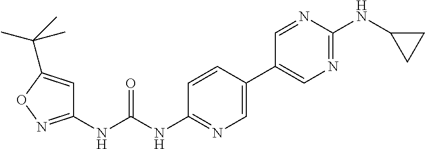 [SMILES: CC(C)(C)c1cc(NC(=O)Nc2ccc(-c3cnc(NC4CC4)nc3)cn2)no1]

Step 1: To a 20 mL microwave reaction vial were added 5-bromo-2-chloropyrimidine (500 mg, 2.59 mmol), i-PrOH (7 mL), DIEA (0.90 mL, 5.45 mmol), and cyclopropylamine (0.20 mL, 2.85 mmol). The vial was sealed and heated to 80° C. for 19 h, then the mixture was cooled, Celite was added and the mixture was concentrated under reduced pressure. The residue was purified by silica gel flash chromatography, eluting with 0-100% EtOAc in hexanes, to afford (5-bromopyrimidin-2-yl)cyclopropylamine (522.2 mg, 95%) as a solid. LC-MS (ESI) m/z 214, 216 (M+H)⁺.

Step 2: [5-(6-Aminopyridin-3-yl)pyrimidin-2-yl]cyclopropylamine (275.3 mg, 55%) was synthesized as a solid according to the procedure described in Step 1 of Example 17, substituting (5-bromopyrimidin-2-yl)cyclopropylamine from Step 1 above for 5-bromo-2-aminopyridine used in Example 17. LC-MS (ESI) m/z 228 (M+H)⁺.

Step 3: 1-(5-tert-butylisoxazol-3-yl)-3-[5-(2-cyclopropylamino-pyrimidin-5-yl)-pyridin-2-yl]urea (62.0 mg, 26%) was synthesized as a solid according to the procedure described in Step 2 of Example 2, substituting [5-(6-aminopyridin-3-yl)pyrimidin-2-yl]cyclopropylamine from Step 2 above for 2-amino-5-(4-aminophenyl)nicotinonitrile used in Example 2. LC-MS (ESI) m/z 394 (M+H)⁺. ¹H NMR (DMSO-d₆) δ: 10.90 (s, 1H), 9.70 (s, 1H), 8.65 (br s, 2H), 8.59 (s, 1H), 8.06 (m, 1H), 7.61 (d, J=8.6 Hz, 1H), 7.40 (d, J=8.6 Hz, 1H), 6.58 (s, 1H), 3.41 (m, 1H), 1.77-1.88 (m, 2H), 1.45-1.60 (m, 2H), 1.31 (s, 9H).

### Example 65

### Preparation of 1-(5-tert-butylisoxazol-3-yl)-3-(5-(2-(isopropylamino)pyrimidin-5-yl)pyridin-2-yl)urea

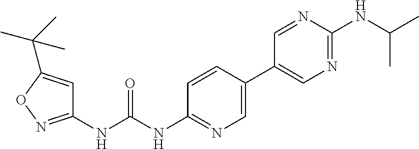 [SMILES: CC(C)Nc1ncc(-c2ccc(NC(=O)Nc3cc(C(C)(C)C)on3)nc2)cn1]

Step 1: 5-Bromo-N-isopropylpyrimidin-2-amine (505.5 mg, 90%) was synthesized according to the procedure described in Step 1 of Example 45, substituting isopropylamine for 2-morpholinoethylamine used in Example 45. LC-MS (ESI) m/z 216, 218 (M+H)⁺.

Step 2: 5-(6-Aminopyridin-3-yl)-N-isopropylpyrimidin-2-amine (424.9 mg, 85%) was synthesized according to the procedure described in Step 1 of Example 17, substituting 5-(6-aminopyridin-3-yl)-N-isopropylpyrimidin-2-amine from Step 1 above for 5-bromo-2-aminopyridine used in Example 17. LC-MS (ESI) m/z 230 (M+H)⁺.

Step 3: 1-(5-tert-butylisoxazol-3-yl)-3-(5-(2-(isopropylamino)pyrimidin-5-yl)pyridin-2-yl)urea (70.4 mg, 27%) was synthesized according to the procedure described in Step 2 of Example 2, substituting 5-(6-aminopyridin-3-yl)-N-isopropylpyrimidin-2-amine from Step 2 above for 2-amino-5-(4-aminophenyl)nicotinonitrile used in Example 2. LC-MS (ESI) m/z 396 (M+H)⁺. ¹H NMR (DMSO-d₆) δ: 10.90 (br s, 1H), 9.69 (br s, 1H), 8.64 (s, 2H), 8.57 (br s, 1H), 8.06 (d, 1H), 7.61 (d, 1H), 7.24 (d, 1H), 6.58 (s, 1H), 3.99-4.17 (m, 1H), 1.31 (s, 9H), 1.17 (d, 6H).

### Example 66

### Preparation of N-(5-(2-(cyclopropylamino)pyrimidin-5-yl)pyridine-2-yl)-2-(3-(trifluoromethyl)phenyl)acetamide

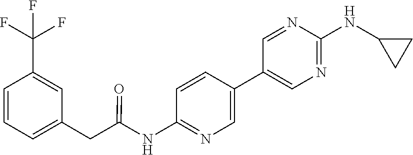 [SMILES: O=C(Cc1cccc(C(F)(F)F)c1)Nc1ccc(-c2cnc(NC3CC3)nc2)cn1]

To a stirred solution of [5-(6-aminopyridin-3-yl)pyrimidin-2-yl]cyclopropylamine (80 mg, 0.35 mmol) from Step 2 of Example 64 in DCM (1.0 mL) was added TEA (0.10 mL, 0.72 mmol), followed by 3-trifluoromethylphenyl acetyl chloride (76 mg, 0.34 mmol). The mixture was stirred at rt for 3 d, then partitioned between DCM (50 mL) and saturated aq NaHCO₃ (50 mL). The separated aqueous layer was extracted with DCM (3×30 mL) and the combined organic layers were dried over MgSO₄, filtered, and concentrated under reduced pressure. The residue was purified by silica gel flash chromatography eluting with 1-9% MeOH in DCM to give N-(5-(2-(cyclopropylamino)pyrimidin-5-yl)pyridine-2-yl)-2-(3-(trifluoromethyl)phenyl)acetamide (25.2 mg, 18%). LC-MS (ESI) m/z 414 (M+H)⁺. ¹H NMR (DMSO-d₆) δ: 10.88 (br s, 1H), 8.67 (s, 2H), 8.63 (s, 1H), 8.07 (q, 2H), 7.73 (s, 1H), 7.77-7.54 (m, 4H), 3.88 (s, 2H), 2.74 (m, 1H), 0.68 (d, 2H), 0.49 (br s, 2H).

### Example 67

### Preparation of N-(5-(2-(isopropylamino)pyrimidin-5-yl)pyridin-2-yl)-2-(3-(trifluoromethyl)phenyl)acetamide

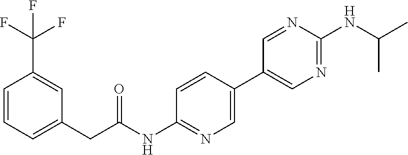 [SMILES: CC(C)Nc1ncc(-c2ccc(NC(=O)Cc3cccc(C(F)(F)F)c3)nc2)cn1]

N-(5-(2-(isopropylamino)pyrimidin-5-yl)pyridin-2-yl)-2-(3-(trifluoromethyl)phenyl)acetamide (30.8 mg, 22%) was synthesized according to the procedure described in Example 66, substituting 5-(6-aminopyridin-3-yl)-N-isopropylpyrimidin-2-amine from Step 2 of Example 65 for [5-(6-aminopyridin-3-yl)pyrimidin-2-yl]cyclopropylamine used in Example 66. LC-MS (ESI) m/z 416 (M+H)⁺. ¹H NMR (DMSO-d₆) δ: 10.87 (br s, 1H), 8.64 (br s, 2H), 8.61 (s, 1H), 8.06 (q, 2H), 7.73 (s, 1H), 7.69-7.52 (m, 3H), 7.24 (d, 1H), 4.08 (m, 1H), 3.88 (s, 2H), 1.16 (d, 6H).

### Example 68

### Preparation of 5-(4-(3-(5-(1,3-difluoro-2-methylpropan-2-yl)isoxazol-3-yl)ureido)-3-methoxyphenyl)pyridin-2-aminium methanesulfonate

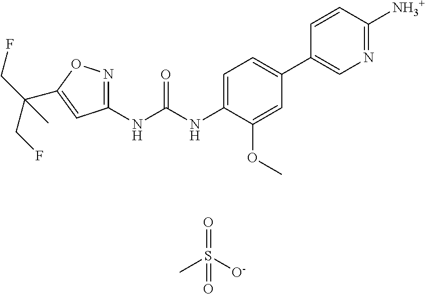

Step 1: 5-(4-Amino-3-methoxyphenyl)pyridin-2-amine (211 mg, 65%) was synthesized as a purple solid using a procedure analogous to that described in Step 2 of Example 40, substituting 2-methoxy-4-(4,4,5,5-tetramethyl-1,3,2-dioxaborolan-2-yl)aniline for 5-(4,4,5,5-tetramethyl-1,3,2-dioxaborolan-2-yl)pyridin-2-amine, and 5-bromopyridin-2-amine for 5-bromo-N-tritylprydin-2-amine used in Step 2 of Example 40. LC-MS (ESI) m/z 216 (M+H)⁺.

Step 2: 1-(4-(6-Aminopyridin-3-yl)-2-methoxyphenyl)-3-(5-(1,3-difluoro-2-methylpropan-2-yl)isoxazol-3-yl)urea (62.8 mg, 38%) was synthesized as a solid using a procedure analogous to that described in Step 4 of Example 36, substituting 5-(4-amino-3-methoxyphenyl)pyridin-2-amine from Step 1 of this example for 5-(4-aminophenyl)-N-(2-morpholinoethyl)pyridin-2-amine, and phenyl 5-(1,3-difluoro-2-methylpropan-2-yl)isoxazol-3-ylcarbamate from Step 1 of Example 34 for phenyl 5-(1-(trifluoromethyl)cyclopropyl)isoxazol-3-ylcarbamate used in Step 4 of Example 36. LC-MS (ESI) m/z 419 (M+H)⁺.

Step 3: 5-(4-(3-(5-(1,3-Difluoro-2-methylpropan-2-yl)isoxazol-3-yl)ureido)-3-methoxyphenyl)pyridin-2-aminium methanesulfonate (78 mg, 100%) was synthesized as a solid using the procedure analogous to that described in Step 3 of Example 89, substituting 1-(4-(6-aminopyridin-3-yl)-2-methoxyphenyl)-3-(5-(1,3-difluoro-2-methylpropan-2-yl)isoxazol-3-yl)urea from Step 2 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d6) δ 13.66 (br s, 1H), 10.22 (br s, 1H), 8.75 (br s, 1H), 8.12-8.47 (m, 3H), 8.00 (br s, 2H), 7.33 (br s, 1H), 7.24 (d, J=8.1 Hz, 1H), 7.08 (d, J=9.0 Hz, 1H), 6.78 (br s, 1H), 4.41-4.88 (m, 4H), 3.98 (br s, 3H), 2.34 (br s, 3H), 1.34 (br s, 3H). LC-MS (ESI) m/z 419 (M+H)⁺.

### Example 69

### Preparation of 5-(4-(3-(5-tert-butylisoxazol-3-yl)ureido)-3-methoxyphenyl)pyridin-2-aminium methanesulfonate

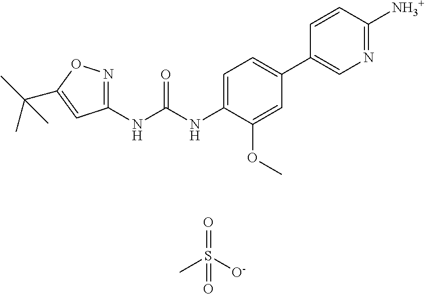

Step 1: 1-(4-(6-Aminopyridin-3-yl)-2-methoxyphenyl)-3-(5-tert-butylisoxazol-3-yl)urea (71 mg, 47%) was synthesized as a solid using a procedure analogous to that described in Step 4 of Example 36, substituting phenyl 5-tert-butylisoxazol-3-ylcarbamate for phenyl 5-(1-(trifluoromethyl)cyclopropyl)isoxazol-3-ylcarbamate, and 5-(4-amino-3-methoxyphenyl)pyridin-2-amine from Step 1 of Example 68 for 5-(4-aminophenyl)-N-(2-morpholinoethyl)pyridin-2-amine hydrochloride used in Example 36. LC-MS (ESI) m/z 383 (M+H)⁺.

Step 2: 5-(4-(3-(5-tert-Butylisoxazol-3-yl)ureido)-3-methoxyphenyl)pyridin-2-aminium methanesulfonate (89.8 mg, 100%) was synthesized as a solid using the procedure analogous to that described in Step 3 of Example 89, substituting 1-(4-(6-aminopyridin-3-yl)-2-methoxyphenyl)-3-(5-tert-butylisoxazol-3-yl)urea from Step 1 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 13.64 (br s, 1H), 10.10 (s, 1H), 8.77 (br s, 1H), 8.26-8.38 (m, 2H), 8.22 (d, J=8.5 Hz, 1H), 7.93 (br s, 2H), 7.32 (s, 1H), 7.23 (d, J=9.6 Hz, 1H), 7.06 (d, J=9.0 Hz, 1H), 6.48 (s, 1H), 3.97 (s, 3H), 2.34 (s, 3H), 1.30 (s, 9H). LC-MS (ESI) m/z 383 (M+H)⁺.

### Example 70

### Preparation of 5-(4-(3-(3-tert-butylisoxazol-5-yl)ureido)phenyl)pyridin-2-aminium methanesulfonate

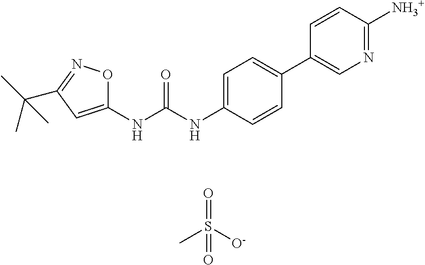

Step 1: To a stirred solution of 3-tert-butylisoxazol-5-amine (1.04 g, 4 mmol) and phenyl carbonochloridate (624 mg, 4 mmol) in DCM (30 mL) was added triethylamine (484 mg, 4.8 mmol) at room temperature. The reaction mixture was stirred at rt for overnight and then diluted with DCM (80 mL). The resulting mixture was washed sequentially with sat aq NaHCO₃ and brine. The organic layer was separated, dried over MgSO₄, filtered, and concentrated under reduced pressure. The residue was purified by silica gel chromatography eluting with 1:10 EtOAc/DCM to afford phenyl 3-tert-butylisoxazol-5-ylcarbamate (871 mg, 84%) as a white solid. LC-MS (ESI) m/z 261 (M+H)⁺.

Step 2: 1-(4-(6-Aminopyridin-3-yl)phenyl)-3-(3-tert-butylisoxazol-5-yl)urea (98 mg, 56%) was synthesized as a white solid according to the procedure described in Step 4 of Example 36, substituting 5-(4-aminophenyl)pyridin-2-amine from Step 1 of Example 1 for 5-(4-aminophenyl)-N-(2-morpholinoethyl)pyridin-2-amine, and phenyl 3-tert-butylisoxazol-5-ylcarbamate from Step 1 of this example for phenyl 5-(1-(trifluoromethyl)cyclopropyl)isoxazol-3-ylcarbamate used in Step 4 Example 36. LC-MS (ESI) m/z 352 (M+H)⁺.

Step 3: 5-(4-(3-(3-tert-Butylisoxazol-5-yl)ureido)phenyl)pyridin-2-aminium methanesulfonate was synthesized (126 mg, 100%) as a white solid using the procedure analogous to that described in Step 3 of Example 89, substituting 1-(4-(6-aminopyridin-3-yl)phenyl)-3-(3-tert-butylisoxazol-5-yl)urea from Step 2 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 13.67 (br s, 1H), 10.28 (br s, 1H), 9.16 (br s, 1H), 8.17-8.38 (m, 2H), 8.02 (br s, 2H), 7.62 (br s, 4H), 7.08 (d, J=9.2 Hz, 1H), 6.07 (s, 1H), 2.40 (s, 3H), 1.26 (br s, 9H). LC-MS (ESI) m/z 352 (M+H)⁺.

### Example 71

### Preparation of 2-(4-(6-aminopyridin-3-yl)phenyl)-N-(5-tert-butylisoxazol-3-yl) propanamide

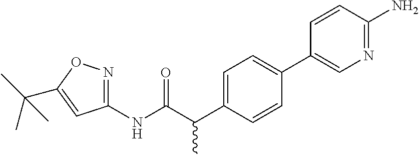 [SMILES: CC(C(=O)Nc1cc(C(C)(C)C)on1)c1ccc(-c2ccc(N)nc2)cc1]

Step 1: N-(5-tert-Butylisoxazol-3-yl)-2-(4-chlorophenyl)propanamide (300 mg, 99%) was synthesized as a white solid using the procedure analogous to that described in Step 1 of Example 18, substituting 2-(4-chlorophenyl)propanoic acid for 2-(4-bromophenyl)acetic acid used in Example 18. LC-MS (ESI) m/z 307 (M+H)⁺.

Step 2: To a mixture of N-(5-tert-butylisoxazol-3-yl)-2-(4-chlorophenyl)propanamide from Step 1 of this example (450 mg, 1.47 mmol), 5-(4,4,5,5-tetramethyl-1,3,2-dioxaborolan-2-yl)pyridin-2-amine (356 mg, 1.61 mmol), and cesium fluoride (1.11 g, 7.35 mmol) in CH₃CN (18 mL)/water (3 mL), was added dichlorobis(tricyclohexylphosphine)palladium(II) (87 mg, 0.11 mmol). The mixture was flushed thoroughly with argon, and then heated in a microwave reactor for 30 min at 150° C. After cooled to rt, the reaction mixture was diluted with EtOAc (80 mL) and washed sequentially with sat aq ammonium chloride and brine. The organic layer was separated, dried over MgSO₄, filtered, and concentrated under reduced pressure. The residue was purified by silica gel chromatography eluting with 5:1 EtOAc/hexanes to afford 2-(4-(6-aminopyridin-3-yl)phenyl)-N-(5-tert-butylisoxazol-3-yl)propanamide (27 mg, 5%) as a white solid. ¹H NMR (300 MHz, DMSO-d₆) δ 10.97-11.28 (m, 1H), 8.21 (d, J=2.1 Hz, 1H), 7.67 (dd, J=2.4, 8.7 Hz, 1H), 7.52 (d, J=8.3 Hz, 1H), 7.28-7.44 (m, 3H), 6.56-6.65 (m, 1H), 6.51 (d, J=8.7 Hz, 1H), 6.06 (s, 2H), 3.90 (q, J=6.8 Hz, 1H), 1.35-1.50 (m, 3H), 1.26 (s, 9H). LC-MS (ESI) m/z 365 (M+H)⁺.

### Example 72

### Preparation of 5-(4-(2-oxo-2-(5-(1,1,1-trifluoro-2-methylpropan-2-yl)isoxazol-3-ylamino)ethyl)phenyl)pyridin-2-aminium acetate

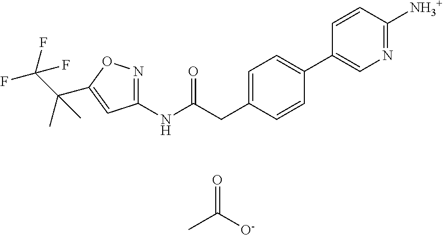

Step 1: 2-(4-Bromophenyl)-N-(5-(1,1,1-trifluoro-2-methylpropan-2-yl)isoxazol-3-yl)acetamide (320 mg, 79%) was synthesized as a white solid using the procedure analogous to that described in Step 1 of Example 18, substituting 5-(1,1,1-trifluoro-2-methylpropan-2-yl)isoxazol-3-amine from Step 1 of Example 35 for 5-tert-butyl-isoxazol-3-amine used in Example 18. LC-MS (ESI) m/z 393 (M+H)⁺.

Step 2: 5-(4-(2-oxo-2-(5-(1,1,1-trifluoro-2-methylpropan-2-yl)isoxazol-3-ylamino)ethyl)phenyl)pyridin-2-aminium acetate (320 mg, 79%) was synthesized as a white solid using the procedure analogous to that described in Step 2 of Example 89, substituting 2-(4-bromophenyl)-N-(5-(1,1,1-trifluoro-2-methylpropan-2-yl)isoxazol-3-yl)acetamide from Step 1 of this example for 5-bromo-N-(2-methoxyethyl)pyridin-2-amine, and 5-(4,4,5,5-tetramethyl-1,3,2-dioxaborolan-2-yl)pyridin-2-amine for N-(5-tert-butylisoxazol-3-yl)-2-(4-(4,4,5,5-tetramethyl-1,3,2-dioxaborolan-2-yl)phenyl)acetamide used in Step 2 of Example 89. ¹H NMR (300 MHz, DMSO-d₆) δ 11.36 (s, 1H), 8.22 (br s, 1H), 7.69 (dd, J=2.4, 8.7 Hz, 1H), 7.52 (d, J=8.1 Hz, 2H), 7.34 (d, J=8.1 Hz, 2H), 6.94 (s, 1H), 6.54 (d, J=8.7 Hz, 1H), 6.09 (br s, 2H), 3.70 (s, 2H), 1.91 (s, 3H), 1.53 (s, 6H). LC-MS (ESI) m/z 405 (M+H)⁺.

### Example 73

### Preparation of 2-(4-(6-aminopyridin-3-yl)phenyl)-N-(5-(1-(trifluoromethyl)cyclopropyl)isoxazol-3-yl)acetamide

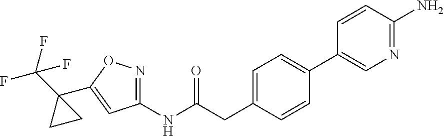 [SMILES: Nc1ccc(-c2ccc(CC(=O)Nc3cc(C4(C(F)(F)F)CC4)on3)cc2)cn1]

Step 1: 2-(4-Bromophenyl)-N-(5-(1-(trifluoromethyl)cyclopropyl)isoxazol-3-yl)acetamide (178 mg, 44%) was synthesized as a white solid using the procedure analogous to that described in Step 1 of Example 18, substituting 5-(1-(trifluoromethyl)cyclopropyl)isoxazol-3-amine from Step 1 of Example 33 for 5-(2,3,3-trimethylbutan-2-yl)isoxazol-3-amine used in Example 18. LC-MS (ESI) m/z 390 (M+H)⁺.

Step 2: 2-(4-(6-Aminopyridin-3-yl)phenyl)-N-(5-(1-(trifluoromethyl)cyclopropyl)isoxazol-3-yl)acetamide (3 mg, 1.7%) was synthesized as a solid according to the procedure described in Step 2 of Example 89, substituting 2-(4-bromophenyl)-N-(5-(1-(trifluoromethyl)cyclopropyl)isoxazol-3-yl)acetamide from Step 1 of this example for 5-bromo-N-(2-methoxyethyl)pyridin-2-amine, and 5-(4,4,5,5-tetramethyl-1,3,2-dioxaborolan-2-yl)pyridin-2-amine for N-(5-tert-butylisoxazol-3-yl)-2-(4-(4,4,5,5-tetramethyl-1,3,2-dioxaborolan-2-yl)phenyl)acetamide used in Step 2 of Example 89. ¹H NMR (300 MHz, DMSO-d₆) δ 11.35 (s, 1H), 8.22 (s, 1H), 7.67 (dd, J=2.4, 8.7 Hz, 1H), 7.51 (d, J=8.1 Hz, 2H), 7.33 (d, J=8.1 Hz, 2H), 6.92 (s, 1H), 6.52 (d, J=8.5 Hz, 1H), 6.04 (s, 2H), 3.68 (s, 2H), 1.06-1.38 (m, 4H). LC-MS (ESI) m/z 403 (M+H)⁺.

### Example 74

### Preparation of 2-(4-(2-aminopyrimidin-5-yl)phenyl)-N-(5-tert-butylisoxazol-3-yl)acetamide

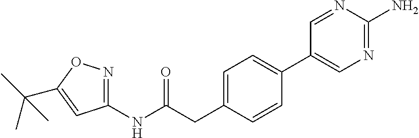 [SMILES: CC(C)(C)c1cc(NC(=O)Cc2ccc(-c3cnc(N)nc3)cc2)no1]

2-(4-(2-Aminopyrimidin-5-yl)phenyl)-N-(5-tert-butylisoxazol-3-yl)acetamide (21 mg, 20%) was synthesized as a white solid using the procedure analogous to that described in Step 2 of Example 89, substituting 5-bromopyrimidin-2-amine for 5-bromo-N-(2-methoxyethyl)pyridin-2-amine used in Step 2 of Example 89. ¹H NMR (300 MHz, DMSO-d₆) δ 11.19 (s, 1H), 8.55 (s, 2H), 7.57 (d, J=8.1 Hz, 2H), 7.37 (d, J=8.1 Hz, 2H), 6.75 (s, 2H), 6.56 (s, 1H), 3.68 (s, 2H), 1.27 (s, 9H). LC-MS (ESI) m/z 352 (M+H)⁺.

### Example 75

### Preparation of N-(5-tert-butylisoxazol-3-yl)-2-(4-(2-(2-morpholinoethylamino)pyrimidin-5-yl)phenyl)acetamide

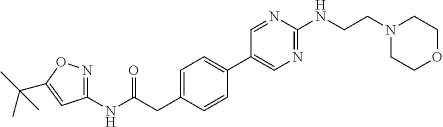 [SMILES: CC(C)(C)c1cc(NC(=O)Cc2ccc(-c3cnc(NCCN4CCOCC4)nc3)cc2)no1]

Step 1: To a solution of 5-bromo-2-chloropyrimidine (193 mg, 1.0 mmol) in THF (6 mL) was added 2-morpholinoethanamine (130 mg, 1.1 mmol) and DIEA (142 mg, 1.1 mmol). The reaction solution was heated in a sealed tube at 80° C. for 20 h. After cooled to rt, the reaction mixture was concentrated under reduced pressure. The residue was purified by silica gel chromatography eluting with 1:10 MeOH/EtOAc to afford 5-bromo-N-(2-morpholinoethyl)pyrimidin-2-amine (216 mg, 75%) as a light yellow oil. LC-MS (ESI) m/z 288 (M+H)⁺.

Step 2: N-(5-tert-butylisoxazol-3-yl)-2-(4-(2-(2-morpholinoethylamino)pyrimidin-5-yl)phenyl)acetamide (67 mg, 47%) was synthesized as a white solid using the procedure analogous to that described in Step 2 of Example 89, substituting 5-bromo-N-(2-morpholinoethyl)pyrimidin-2-amine from Step 1 of this example for 5-bromo-N-(2-methoxyethyl)pyridin-2-amine used in Step 2 of Example 89. ¹H NMR (300 MHz, DMSO-d₆) δ 11.18 (s, 1H), 8.60 (s, 2H), 7.57 (d, J=8.3 Hz, 2H), 7.37 (d, J=8.1 Hz, 2H), 7.13 (t, J=5.7 Hz, 1H), 6.56 (s, 1H), 3.68 (s, 2H), 3.51-3.62 (m, 4H), 3.44 (q, J=6.4 Hz, 2H), 3.29 (br s, 2H), 2.41 (br s, 4H), 1.27 (s, 9H). LC-MS (ESI) m/z 465 (M+H)⁺.

### Example 76

### Preparation of 2-(6′-amino-3,3′-bipyridin-6-yl)-N-(5-tert-butylisoxazol-3-yl)acetamide

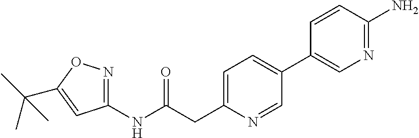 [SMILES: CC(C)(C)c1cc(NC(=O)Cc2ccc(-c3ccc(N)nc3)cn2)no1]

Step 1: 2-(5-Bromopyridin-2-yl)acetic acid acetamide was synthesized as a white solid using the procedure analogous to that described in Jones, Gurnos, et al. Tetrahedron. 1997, vol. 53. p. 8257-8268. ¹H NMR (300 MHz, DMSO-d₆) δ 11.12 (br s, 1H), 8.72 (d, J=2.3 Hz, 1H), 8.15 (dd, J=2.4, 8.4 Hz, 1H), 7.47 (d, J=8.3 Hz, 1H), 3.83 (s, 2H).

Step 2: 2-(5-Bromopyridin-2-yl)-N-(5-tert-butylisoxazol-3-yl)acetamide (320 mg, 51%) was synthesized as a yellow solid using the procedure analogous to that described in Step 1 of Example 18, substituting 2-(5-bromopyridin-2-yl)acetic acid form Step 1 of this example for 2-(4-bromophenyl)acetic acid used in Example 18. LC-MS (ESI) m/z 339 (M+H)⁺.

Step 3: 2-(6′-Amino-3,3′-bipyridin-6-yl)-N-(5-tert-butylisoxazol-3-yl)acetamide (54 mg, 43%) was synthesized as a white solid using the procedure analogous to that described in Step 2 of Example 89, substituting 2-(5-bromopyridin-2-yl)-N-(5-tert-butylisoxazol-3-yl)acetamide from Step 2 of this example for 5-bromo-N-(2-methoxyethyl)pyridin-2-amine, and 5-(4,4,5,5-tetramethyl-1,3,2-dioxaborolan-2-yl)pyridin-2-amine for N-(5-tert-butylisoxazol-3-yl)-2-(4-(4,4,5,5-tetramethyl-1,3,2-dioxaborolan-2-yl)phenyl)acetamide used in Example 89. ¹H NMR (300 MHz, DMSO-d₆) δ 11.20 (s, 1H), 8.70 (d, J=2.1 Hz, 1H), 8.28 (d, J=2.3 Hz, 1H), 7.93 (dd, J=2.4, 8.2 Hz, 1H), 7.74 (dd, J=2.4, 8.7 Hz, 1H), 7.39 (d, J=8.1 Hz, 1H), 6.46-6.67 (m, 2H), 6.15 (s, 2H), 3.88 (s, 2H), 1.28 (s, 9H). LC-MS (ESI) m/z 352 (M+H)⁺.

### Example 77

### Preparation of 2-(5-(2-aminopyrimidin-5-yl)pyridin-2-yl)-N-(5-tert-butylisoxazol-3-yl)acetamide

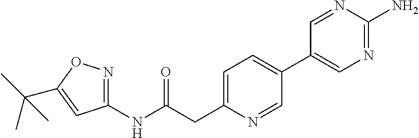 [SMILES: CC(C)(C)c1cc(NC(=O)Cc2ccc(-c3cnc(N)nc3)cn2)no1]

Step 1: To a mixture of 5-bromopyrimidin-2-amine (500 mg, 2.87 mmol), 4,4,4′,4′,5,5,5′,5′-octamethyl-2,2′-bi(1,3,2-dioxaborolane) (802 mg, 3.16 mmol), and potassium acetate (844 mg, 8.61 mmol) in dioxane (20 mL) was added[1,1′-bis(diphenylphosphino)ferrocene]dichloropalladium(II) (117 mg, 0.14 mmol). The reaction mixture was flushed thoroughly with argon and heated in a sealed tube at 110° C. for overnight. After cooled to rt, the reaction mixture was diluted with EtOAc (60 mL) and filtered through a Celite plug. The filtrate was washed sequentially with water and brine. The organic layer was separated, dried over MgSO₄, filtered through a short silica gel plug. The filtrate was concentrated under reduced pressure. The residue was sonicated with DCM/hexane (1:3, 6 mL), the precipitate was collected through filtration, and dried to afford 5-(4,4,5,5-tetramethyl-1,3,2-dioxaborolan-2-yl)pyrimidin-2-amine (441 mg, 69%) as a white solid. LC-MS (ESI) m/z 222 (M+H)⁺.

Step 2: 2-(5-(2-Aminopyrimidin-5-yl)pyridin-2-yl)-N-(5-tert-butylisoxazol-3-yl)acetamide (24 mg, 19%) was synthesized as a white solid using the procedure analogous to that described in Step 2 of Example 89, substituting 5-(4,4,5,5-tetramethyl-1,3,2-dioxaborolan-2-yl)pyrimidin-2-amine from Step 1 of this example for 5-(4,4,5,5-tetramethyl-1,3,2-dioxaborolan-2-yl)pyridin-2-amine, and 2-(5-bromopyridin-2-yl)-N-(5-tert-butylisoxazol-3-yl)acetamide for 5-bromo-N-(2-methoxyethyl)pyridin-2-amine used in Step 2 of Example 89. ¹H NMR (300 MHz, CHLOROFORM-d) δ 10.39 (br s, 1H), 8.76 (d, J=1.7 Hz, 1H), 8.54 (s, 2H), 7.80 (dd, J=2.0, 8.0 Hz, 1H), 7.37 (d, J=7.9 Hz, 1H), 6.66 (s, 1H), 5.30 (br s, 2H), 3.94 (s, 2H), 1.33 (s, 9H). LC-MS (ESI) m/z 353 (M+H)⁺.

### Example 78

### Preparation of 2-(4-(6-aminopyridin-3-yl)-2-fluorophenyl)-N-(5-tert-butylisoxazol-3-yl)acetamide

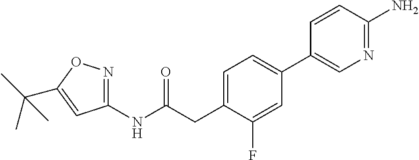 [SMILES: CC(C)(C)c1cc(NC(=O)Cc2ccc(-c3ccc(N)nc3)cc2F)no1]

Step 1: 2-(4-Bromo-2-fluorophenyl)-N-(5-tert-butylisoxazol-3-yl)acetamide (240 mg, 52%) was synthesized as a white solid using the procedure analogous to that described in Step 1 of Example 18, substituting 2-(4-bromo-2-fluorophenyl)acetic acid for 2-(4-bromophenyl)acetic acid used in Example 18. LC-MS (ESI) m/z 356 (M+H)⁺.

Step 2: 2-(4-(6-Aminopyridin-3-yl)-2-fluorophenyl)-N-(5-tert-butylisoxazol-3-yl)acetamide (51 mg, 49%) was synthesized as a white solid using the procedure analogous to that described in Step 2 of Example 89, substituting 2-(4-bromo-2-fluorophenyl)-N-(5-tert-butylisoxazol-3-yl)acetamide from Step 1 of this example for 5-bromo-N-(2-methoxyethyl)pyridin-2-amine, and 5-(4,4,5,5-tetramethyl-1,3,2-dioxaborolan-2-yl)pyridin-2-amine for 5-(4,4,5,5-tetramethyl-1,3,2-dioxaborolan-2-yl)pyridin-2-amine used in Step 2 of Example 89. ¹H NMR (300 MHz, DMSO-d₆) δ 11.21 (s, 1H), 8.28 (d, J=2.3 Hz, 1H), 7.75 (dd, J=2.4, 8.7 Hz, 1H), 7.28-7.47 (m, 3H), 6.43-6.63 (m, 2H), 6.20 (br s, 2H), 3.75 (s, 2H), 1.28 (s, 9H). LC-MS (ESI) m/z 369 (M+H)⁺.

### Example 79

### Preparation of 2-(4-(2-aminopyrimidin-5-yl)-2-fluorophenyl)-N-(5-tert-butylisoxazol-3-yl)acetamide

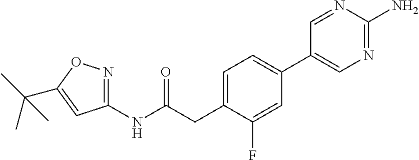 [SMILES: CC(C)(C)c1cc(NC(=O)Cc2ccc(-c3cnc(N)nc3)cc2F)no1]

2-(4-(2-Aminopyrimidin-5-yl)-2-fluorophenyl)-N-(5-tert-butylisoxazol-3-yl)acetamide (37 mg, 36%) was synthesized as a white solid using the procedure analogous to that described in Step 2 of Example 40, substituting 2-(4-bromo-2-fluorophenyl)-N-(5-tert-butylisoxazol-3-yl)acetamide from Step 1 of Example 78 for 5-bromo-N-tritylprydin-2-amine, and 5-(4,4,5,5-tetramethyl-1,3,2-dioxaborolan-2-yl)pyrimidin-2-amine from Step 1 of Example 77 for 5-(4,4,5,5-tetramethyl-1,3,2-dioxaborolan-2-yl)pyridin-2-amine used in Example 40. ¹H NMR (300 MHz, DMSO-d₆) δ 11.22 (s, 1H), 8.61 (s, 2H), 7.30-7.60 (m, 3H), 6.84 (s, 2H), 6.56 (s, 1H), 3.77 (s, 2H), 1.28 (s, 9H). LC-MS (ESI) m/z 370 (M+H)⁺.

### Example 80

### Preparation of 1-(4-(6-aminopyridin-3-yl)phenyl)-3-(5-(1-hydroxy-2-methylpropan-2-yl)isoxazol-3-yl)urea

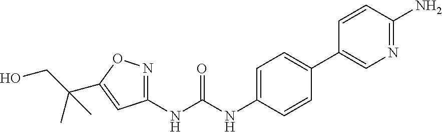 [SMILES: CC(C)(CO)c1cc(NC(=O)Nc2ccc(-c3ccc(N)nc3)cc2)no1]

Step 1: A solution of methyl 3-hydroxy-2,2-dimethylpropanoate (5.00 g, 38 mmol), N,N-diisopropylethylamine (7.30 g, 57 mmol) and tert-butyldimethylchlorosilane (6.80 g, 45 mmol) in dry DMF (70 mL) was stirred at room temperature for 12 h. The reaction solution was quenched with water (225 mL) and extracted with diethyl ether (3×50 mL). The combined organic extracts were washed with water (100 mL), brine (100 mL), then dried over MgSO₄. Concentration under reduced pressure afforded methyl 3-(tert-butyldimethylsilyloxy)-2,2-dimethylpropanoate as colorless oil (9.36 g, 100%). It was used in the next step without further purification. ¹H NMR (300 MHz, CDCl₃) δ 3.64 (s, 3H), 3.55 (s, 2H), 1.13 (s, 6H), 0.85 (s, 9H), 0.0 (s, 6H).

Step 2: 5-Hydroxy-4,4-dimethyl-3-oxopentanenitrile was prepared using a procedure analogous to that described in Steps 1 of Example 32, substituting methyl 3-(tert-butyldimethylsilyloxy)-2,2-dimethylpropanoate from Step 1 of this example for 2-fluoro-2-methylpropanoate used in Example 32.

Step 3: 2-(3-Aminoisoxazol-5-yl)-2-methylpropan-1-ol was prepared using a procedure analogous to that described in Steps 2 of Example 32, substituting 5-hydroxy-4,4-dimethyl-3-oxopentanenitrile from Step 2 of this example for 4-fluoro-4-methyl-3-oxopentanenitrile used in Example 32.

Step 4: Phenyl 5-(1-hydroxy-2-methylpropan-2-yl)isoxazol-3-ylcarbamate as a colorless solid (120 mg, 72%) was prepared using procedures analogous to those described in Steps 3 of Example 32, substituting 2-(3-aminoisoxazol-5-yl)-2-methylpropan-1-ol from Step 3 of this example for 3-(2-fluoropropan-2-yl)isoxazol-5-amine used in Example 32. ¹H NMR (300 MHz, CDCl₃) δ8.30 (brs, 1H), 7.42-7.43 (m, 2H), 7.26 (m, 1H), 7.18-7.21 (m, 2H), 6.65 (s, 1H), 3.67 (s, 2H), 1.98 (brs, 1H), 1.32 (s, 6H).

Step 5: 1-(4-(6-Aminopyridin-3-yl)phenyl)-3-(5-(1-hydroxy-2-methylpropan-2-yl)isoxazol-3-yl)urea (151 mg, 76%) was synthesized as a solid using the procedure analogous to that described in Step 4 of Example 36, substituting phenyl 5-(1-hydroxy-2-methylpropan-2-yl)isoxazol-3-ylcarbamate from Step 4 of this example for phenyl 5-(1-(trifluoromethyl)cyclopropyl)isoxazol-3-ylcarbamate, and 5-(4-aminophenyl)pyridin-2-amine for 5-(4-aminophenyl)-N-(2-morpholinoethyl)pyridin-2-amine used in Step 4 of Example 36. ¹H NMR (300 MHz, DMSO-d₆) δ 9.49 (s, 1H), 8.84 (s, 1H), 8.21 (d, J=1.9 Hz, 1H), 7.67 (dd, J=2.4, 8.6 Hz, 1H), 7.49 (s, 4H), 6.44-6.63 (m, 2H), 6.01 (s, 2H), 4.97 (t, J=5.5 Hz, 1H), 3.45 (d, J=5.5 Hz, 2H), 1.23 (s, 6H). LC-MS (ESI) m/z 383 (M+H)⁺.

### Example 81

### Preparation of 5-(4-(3-(3-(1-hydroxy-2-methylpropan-2-yl)isoxazol-5-yl)ureido)phenyl)pyridin-2-aminium methanesulfonate

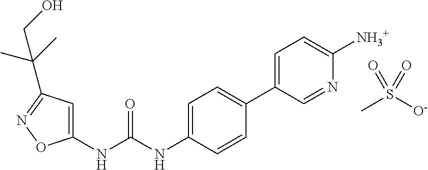

Step 1: To a stirred solution of NaOH (3.4 g, 85.0 mmol) in water (20 mL) at rt was added hydroxylamine hydrochloride (2.2 g, 31.1 mmol) and 5-hydroxy-4,4-dimethyl-3-oxopentanenitrile (4.0 g, 28.3 mmol) from Step 2 of Example 80. The resulting mixture was heated at 55° C. for 3 h. LC-MS indicated the completion of the reaction. After cooled to rt, the reaction mixture was extracted with DCM (3×50 mL). The combined organic layers were dried over MgSO₄, evaporated under reduced pressure. The residue was purified by silica gel chromatography, eluting with 0-75% EtOAc in hexanes, to give 2-(5-aminoisoxazol-3-yl)-2-methylpropan-1-ol (2.15 g, 49%) as colorless oil. LC-MS (ESI) m/z 157 (M+H)⁺.

Step 2: Phenyl (3-(1-hydroxy-2-methylpropan-2-yl)isoxazol-5-yl)carbamate (566 mg, 42% yield) was prepared using a procedure analogous to that described in Step 3 of Example 32, substituting 2-(5-aminoisoxazol-3-yl)-2-methylpropan-1-ol from Step 1 of this example for 3-(2-fluoropropan-2-yl)isoxazol-5-amine used in Example 32. LC-MS (ESI) m/z 277 (M+H)⁺.

Step 3: 5-(4-(3-(3-(1-Hydroxy-2-methylpropan-2-yl)isoxazol-5-yl)ureido)phenyl)pyridin-2-aminium methanesulfonate was synthesized as a white powder (76 mg, 29%) was prepared using a procedure analogous to that described in Step 4 of Example 36, substituting phenyl (3-(1-hydroxy-2-methylpropan-2-yl)isoxazol-5-yl)carbamate from Step 2 of this example for phenyl 5-(1-(trifluoromethyl)cyclopropyl)isoxazol-3-ylcarbamate, and 5-(4-aminophenyl)pyridin-2-amine for 5-(4-aminophenyl)-N-(2-morpholinoethyl)pyridin-2-amine hydrochloride used in Example 36. ¹H NMR (300 MHz, DMSO-d₆) δ 10.25 (s, 1H), 9.13 (s, 1H), 8.31 (dd, J=2.1, 9.2 Hz, 1H), 8.24 (s, 1H), 8.00 (br s, 2H), 7.51-7.70 (m, 4H), 7.07 (d, J=9.2 Hz, 1H), 6.06 (s, 1H), 2.39 (s, 4H), 1.19 (s, 6H). LC-MS (ESI) m/z 368 (M+H)⁺.

### Example 82

### Preparation of 5-(4-(3-(3-(1-hydroxy-2-methylpropan-2-yl)-1-methyl-1H-pyrazol-5-yl)ureido)phenyl)pyridin-2-aminium methanesulfonate

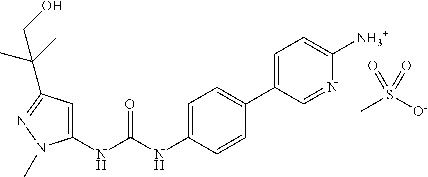

Step 1: A stirred mixture of 5-hydroxy-4,4-dimethyl-3-oxopentanenitrile from Step 1 of Example 81 (750 mg, 5.3 mmol) and N-methylhydrazine (0.57 mL, 10.6 mmol) in EtOH (10 mL) was heated at 90° C. for 3 h. LC-MS indicated the completion of the reaction. After cooled to rt, most of the volatile organics were removed under reduced pressure. The residue was purified by silica gel flash chromatography, eluting with 0-7% 2N NH₃ in MeOH/EtOAc to give 2-(5-amino-1-methyl-1H-pyrazol-3-yl)-2-methylpropan-1-ol (200 mg, 22%) as a brown oil. LC-MS (ESI) m/z 170 (M+H)⁺.

Step 2: Phenyl (3-(1-hydroxy-2-methylpropan-2-yl)-1-methyl-1H-pyrazol-5-yl)carbamate (165 mg, 48% yield) was prepared using a procedure analogous to that described in Step 3 of Example 32, substituting 2(5-amino-1-methyl-1H-pyrazol-3-yl)-2-methylpropan-1-ol from Step 1 of this example for 3-(2-fluoropropan-2-yl)isoxazol-5-amine used in Example 32. LC-MS (ESI) m/z 290 (M+H)⁺.

Step 3: 5-(4-(3-(3-(1-Hydroxy-2-methylpropan-2-yl)-1-methyl-1H-pyrazol-5-yl)ureido)phenyl)pyridin-2-aminium methanesulfonate (80 mg, 31%) was prepared as a white powder using a procedure analogous to that described in Step 4 of Example 36, substituting Phenyl (3-(1-hydroxy-2-methylpropan-2-yl)-1-methyl-1H-pyrazol-5-yl)carbamate from Step 2 of this example for phenyl 5-(1-(trifluoromethyl)cyclopropyl)isoxazol-3-ylcarbamate, and 5-(4-aminophenyl)pyridin-2-amine for 5-(4-aminophenyl)-N-(2-morpholinoethyl)pyridin-2-amine hydrochloride used in Example 36. ¹H NMR (300 MHz, DMSO-d₆) δ 9.18 (s, 1H), 8.77 (s, 1H), 8.30 (dd, J=2.1, 9.2 Hz, 1H), 8.23 (s, 1H), 8.00 (br s, 2H), 7.60 (s, 4H), 7.07 (d, J=9.2 Hz, 1H), 6.10 (s, 1H), 3.63 (s, 3H), 3.37 (s, 3H), 2.40 (s, 4H), 1.16 (s, 6H). LC-MS (ESI) m/z 381 (M+H)⁺.

### Example 83

### Preparation of 5-(4-(2-((3-(1-hydroxy-2-methylpropan-2-yl)isoxazol-5-yl)amino)-2-oxoethyl)phenyl)pyridin-2-aminium methanesulfonate

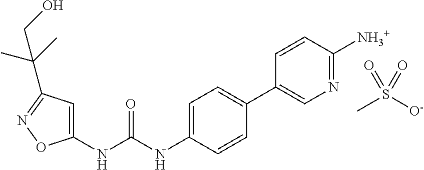

Step 1: To a stirred solution of 2-(4-bromophenyl)acetyl chloride (543 mg, 2.3 mmol) in DCM (5 mL) at rt was added sat. aq NaHCO₃ solution (3 mL), followed by 2-(5-aminoisoxazol-3-yl)-2-methylpropan-1-ol from Step 1 of Example 81 (363 mg, 2.3 mmol) in DCM (5 mL). The resulting mixture was stirred at rt over the weekend. The reaction mixture was then partitioned between DCM and sat NaHCO₃. The organic layer was washed with brine, dried over Na₂SO₄, and evaporated under reduced pressure. The residue was purified by silica gel flash chromatography, eluting with 0-50% EtOAc in hexanes, to give 2-(4-bromophenyl)-N-(3-(1-hydroxy-2-methylpropan-2-yl)isoxazol-5-yl)acetamide as a colorless oil (90 mg, 11%). LC-MS (ESI) m/z 353, 355 (M+H)⁺.

Step 2: To a stirred solution of 2-(4-bromophenyl)-N-(3-(1-hydroxy-2-methylpropan-2-yl)isoxazol-5-yl)acetamide (90 mg, 0.25 mmol) in MeCN (5 mL) was added 5-(4,4,5,5-tetramethyl-1,3,2-dioxaborolan-2-yl)pyridin-2-amine (67 mg, 0.30 mmol), dichloro[1,1′-bis(diphenylphosphino)ferrocene]palladium(II) dichloromethane adduct (9 mg, 0.013 mmol), and 2M aq Na₂CO₃ (0.76 mL, 1.52 mmol). The resulting mixture was flushed with argon for 2 min before it was capped and heated at 140° C. in a microwave reactor for 10 min. LC-MS indicated the completion of the reaction. The reaction mixture was then diluted with brine, extracted with EtOAc (3×). The combined organic layer, dried over Na₂SO₄, filtered, and concentrated under reduced pressure to give an oil, which was purified by silica gel flash chromatography, eluting with 0-90% EtOAc in hexanes, to give 2-(4-(6-aminopyridin-3-yl)phenyl)-N-(3-(1-hydroxy-2-methylpropan-2-yl)isoxazol-5-yl)acetamide as a white solid (31 mg, 34%). LC-MS (ESI) m/z 367 (M+H)⁺.

Step 3: To a stirred mixture of 12-(4-(6-aminopyridin-3-yl)phenyl)-N-(3-(1-hydroxy-2-methylpropan-2-yl)isoxazol-5-yl)acetamide (31 mg, 0.085 mmol) in EtOH (2 mL) was added methanesulfonic acid (5.5 μL, 0.085 mmol.). The mixture was stirred at 60° C. for 30 min before it was cooled to rt and concentrated under reduced pressure. The residue was dissolved in water and lyophilized to give 5-(4-(2-((3-(1-hydroxy-2-methylpropan-2-yl)isoxazol-5-yl)amino)-2-oxoethyl)phenyl)pyridin-2-aminium methanesulfonate (35 mg, 95%) as a white powder. ¹H NMR (300 MHz, DMSO-d₆) δ 11.80 (s, 1H), 8.30 (dd, J=2.1, 9.2 Hz, 1H), 8.25 (s, 1H), 8.05 (br s, 2H), 7.63 (d, J=8.1 Hz, 2H), 7.42 (d, J=8.1 Hz, 2H), 7.09 (d, J=9.2 Hz, 1H), 6.17 (s, 1H), 3.74 (s, 2H), 3.39 (br s, 3H), 2.35 (s, 3H), 1.17 (s, 6H). LC-MS (ESI) m/z 367 (M+H)⁺.

### Example 84

### Preparation of 5-(4-(2-(3-tert-butylisoxazol-5-ylamino)-2-oxoethyl)phenyl)pyridin-2-aminium methanesulfonate

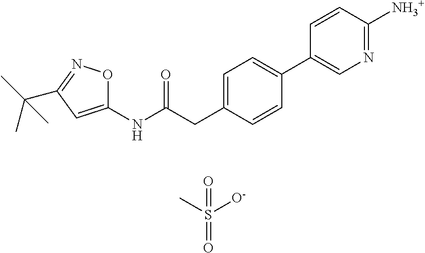

Step 1: 2-(4-Bromophenyl)-N-(3-tert-butylisoxazol-5-yl)acetamide (241 mg, 36%) was synthesized as a white solid using the procedure analogous to that described in Step 1 of Example 18, substituting 3-tert-butylisoxazol-5-amine for 5-tert-butylisoxazol-3-amine used in Example 18. LC-MS (ESI) m/z 339 (M+H)⁺.

Step 2: 2-(4-(6-Aminopyridin-3-yl)phenyl)-N-(3-tert-butylisoxazol-5-yl)acetamide (61 mg, 37%) was synthesized as a white solid using the procedure analogous to that described in Step 2 of Example 40, substituting 2-(4-bromophenyl)-N-(3-tert-butylisoxazol-5-yl)acetamide from Step 1 of this example for 5-bromo-N-tritylprydin-2-amine used in Step 2 of Example 40. LC-MS (ESI) m/z 352 (M+H)⁺.

Step 3: 5-(4-(2-(3-tert-Butylisoxazol-5-ylamino)-2-oxoethyl)phenyl)pyridin-2-aminium methanesulfonate (78.5 mg, 100%) was synthesized as a white solid using the procedure analogous to that described in Step 3 of Example 89, substituting 2-(4-(6-aminopyridin-3-yl)phenyl)-N-(3-tert-butylisoxazol-5-yl)acetamide from Step 2 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 13.75 (br s, 1H), 11.82 (s, 1H), 8.20-8.38 (m, 2H), 8.06 (br s, 2H), 7.63 (d, J=8.1 Hz, 2H), 7.43 (d, J=8.1 Hz, 2H), 7.09 (d, J=9.2 Hz, 1H), 6.19 (s, 1H), 3.75 (s, 2H), 2.37 (s, 3H), 1.23 (s, 9H). LC-MS (ESI) m/z 352 (M+H)⁺.

### Example 85

### Preparation of 5-(4-(2-(5-tert-butylisoxazol-3-ylamino)-2-oxoethyl)phenyl)-3-methylpyridin-2-aminium methanesulfonate

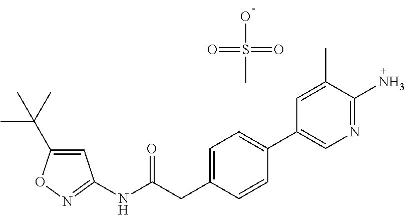

Step 1: A mixture of 2-(4-(4,4,5,5-tetramethyl-1,3,2-dioxaborolan-2-yl)phenyl)acetic acid (4.0 g, 14.8 mmol) and O-(7-azabenzotriazol-1-yl)-N,N,N′,N′-tetramethyluronium hexafluorophosphate (5.63 g, 14.8 mmol) in DMF (30 mL) was stirred at rt for 15 min. 5-tert-Butylisoxazol-3-amine (1.78 g, 12.3 mmol), DIEA (4.3 mL, 24.7 mmol), and additional DMF (20 mL) were added. The reaction mixture was stirred at rt for 2 h. The mixture was partitioned between EtOAc (100 mL) and water (100 mL), and the aqueous layer was separated and extracted with EtOAc (2×100 mL). The combined organic layers were washed with brine (2×100 mL), dried over MgSO₄, filtered, and concentrated under reduced pressure to give an orange-brown residue. The residue was triturated with methanol (10 mL) followed by diethyl ether (15 mL), and the solid was collected via vacuum filtration to give N-(5-tert-butylisoxazol-3-yl)-2-(4-(4,4,5,5-tetramethyl-1,3,2-dioxaborolan-2-yl)phenyl)acetamide (3.7 g, 75%) as a white solid. ¹H NMR (300 MHz, DMSO-d₆) δ 11.20 (s, 1H), 7.63 (d, J=7.9 Hz, 2H), 7.33 (d, J=7.7 Hz, 2H), 6.55 (s, 1H), 3.69 (s, 2H), 1.29 (s, 12H), 1.27 (s, 9H). LC-MS (ESI) m/z 385 (M+H)⁺.

Step 2: To a microwave reaction vial was added N-(5-tert-butylisoxazol-3-yl)-2-(4-(4,4,5,5-tetramethyl-1,3,2-dioxaborolan-2-yl)phenyl)acetamide from Step 1 of this example (150 mg, 0.39 mmol), 5-bromo-3-methylpyridin-2-amine (80.4 mg, 0.43 mmol), 2M aq sodium carbonate (0.48 mL, 0.97 mmol), 1,4-dioxane (3 mL), and tetrakis(triphenylphosphine) palladium(0) (22.5 mg, 0.02 mmol). The vial was purged with argon, sealed, and heated in a microwave reactor at 170° C. for 20 min. The reaction mixture was then filtered through filter paper to remove solid impurities, then most of the volatile solvent of the filtrate was evaporated under reduced pressure. The crude product was purified by preparative HPLC eluting with 12-75% acetonitrile in water to give 2-(4-(6-amino-5-methylpyridin-3-yl)phenyl)-N-(5-tert-butylisoxazol-3-yl)acetamide (30 mg, 21%) as a white solid. LC-MS (ESI) m/z 365 (M+H)⁺.

Step 3: A mixture of 4-(2-(4-(6-amino-5-methylpyridin-3-yl)phenyl)-N-(5-tert-butylisoxazol-3-yl)acetamide from Step 2 of this example (30 mg, 0.08 mmol) and methanesulfonic acid (7.9 mg, 0.08 mmol) in ethanol (10 mL) was stirred at 60° C. for 2 h. The solvent was evaporated under reduced pressure. Water was added and the mixture was frozen and lyophilized to give 5-(4-(2-(5-tert-butylisoxazol-3-ylamino)-2-oxoethyl)phenyl)-3-methylpyridin-2-aminium methanesulfonate (25 mg, 66%) as an off-white solid. ¹H NMR (300 MHz, DMSO-d₆) δ 11.23 (s, 1H), 8.25 (s, 1H), 8.15 (s, 1H), 7.81 (br s, 2H), 7.64 (d, J=8.3 Hz, 2H), 7.42 (d, J=8.1 Hz, 2H), 6.56 (s, 1H), 3.72 (s, 2H), 2.31 (s, 3H), 2.27 (s, 3H), 1.27 (s, 9H). LC-MS (ESI) m/z 365 (M+H)⁺.

### Example 86

### Preparation of 5-(4-(2-(5-tert-butylisoxazol-3-ylamino)-2-oxoethyl)phenyl)-4-methylpyridin-2-aminium methanesulfonate

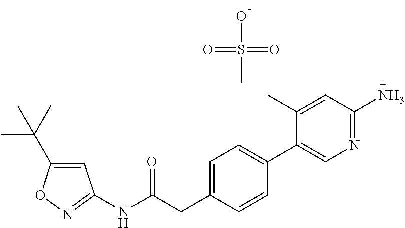

5-(4-(2-(5-tert-Butylisoxazol-3-ylamino)-2-oxoethyl)phenyl)-4-methylpyridin-2-aminium methanesulfonate (90 mg, 30%) was obtained as an off-white solid using procedures analogous to those described in Steps 2-3 of Example 85, substituting 5-bromo-4-methylpyridin-2-amine for 5-bromo-3-methylpyridin-2-amine used in Example 85. ¹H NMR (300 MHz, DMSO-d₆) δ 11.25 (s, 1H), 7.85 (br s, 2H), 7.79 (s, 1H), 7.39-7.46 (m, 2H), 7.31-7.37 (m, 2H), 6.89 (s, 1H), 6.58 (s, 1H), 3.73 (s, 2H), 2.31 (s, 3H), 2.25 (s, 3H), 1.28 (s, 9H). LC-MS (ESI) m/z 365 (M+H)⁺.

### Example 87

### Preparation of 5-(4-(2-(5-tert-butylisoxazol-3-ylamino)-2-oxoethyl)phenyl)-6-methylpyridin-2-aminium methanesulfonate

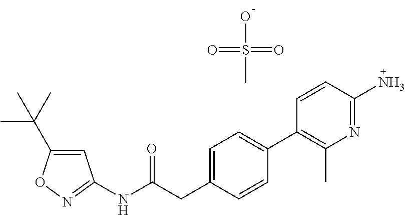

5-(4-(2-(5-tert-Butylisoxazol-3-ylamino)-2-oxoethyl)phenyl)-6-methylpyridin-2-aminium methanesulfonate (20 mg, 33%) was obtained as an off-white solid using procedures analogous to those described in Steps 2-3 of Example 85, substituting 5-bromo-6-methylpyridin-2-amine for 5-bromo-3-methylpyridin-2-amine used in Example 85. ¹H NMR (300 MHz, DMSO-d₆) δ 11.25 (s, 1H), 7.85 (d, J=9.0 Hz, 1H), 7.77 (br s, 2H), 7.39-7.49 (m, 2H), 7.30-7.37 (m, 2H), 6.90 (d, J=9.0 Hz, 1H), 6.57 (s, 1H), 3.73 (br s, 2H), 2.39 (s, 3H), 2.32 (s, 3H), 1.27 (s, 9H). LC-MS (ESI) m/z 365 (M+H)⁺.

### Example 88

### Preparation of 5-(4-(2-(5-(1-hydroxy-2-methylpropan-2-yl)isoxazol-3-ylamino)-2-oxoethyl)phenyl)pyridin-2-aminium methanesulfonate

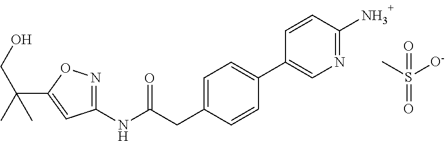

Step 1: 2-(4-Bromophenyl)-N-(5-(1-hydroxy-2-methylpropan-2-yl)isoxazol-3-yl)acetamide (172 mg, 31%) was synthesized as a white solid using the procedure analogous to that described in Step 1 of Example 18, substituting 2-(3-aminoisoxazol-5-yl)-2-methylpropan-1-ol from Step 3 of Example 80 for 5-tert-butylisoxazol-3-amine used in Example 18. LC-MS (ESI) m/z 354 (M+H)⁺.

Step 2: 2-(4-(6-Aminopyridin-3-yl)phenyl)-N-(5-(1-hydroxy-2-methylpropan-2-yl)isoxazol-3-yl)acetamide (50 mg, 28%) was synthesized as a white solid using the procedure analogous to that described in Step 2 of Example 40, substituting 2-(4-bromophenyl)-N-(5-(1-hydroxy-2-methylpropan-2-yl)isoxazol-3-yl)acetamide from Step 1 of this example for 5-bromo-N-tritylprydin-2-amine used in Example 40. LC-MS (ESI) m/z 367 (M+H)⁺.

Step 3: 5-(4-(2-(5-(1-Hydroxy-2-methylpropan-2-yl)isoxazol-3-ylamino)-2-oxoethyl)phenyl)pyridin-2-aminium methanesulfonate (63.7 mg, 100%) was synthesized as a white solid using the procedure analogous to that described in Step 3 of Example 89, substituting 2-(4-(6-aminopyridin-3-yl)phenyl)-N-(5-(1-hydroxy-2-methylpropan-2-yl)isoxazol-3-yl)acetamide from Step 2 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 13.67 (br s, 1H), 11.20 (s, 1H), 8.18-8.32 (m, 2H), 7.84 (br s, 2H), 7.61 (d, J=8.1 Hz, 2H), 7.42 (d, J=8.1 Hz, 2H), 6.98-7.09 (m, 1H), 6.58 (s, 1H), 4.94 (br s, 1H), 3.71 (s, 2H), 3.42 (s, 2H), 2.33 (s, 3H), 1.20 (s, 6H). LC-MS (ESI) m/z 367 (M+H)⁺.

### Example 89

### Preparation of 5-(4-(2-(5-tert-butylisoxazol-3-ylamino)-2-oxoethyl)phenyl)-N-(2-methoxyethyl)pyridin-2-aminium methanesulfonate

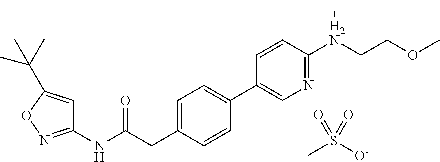

Step 1: To a solution of 5-bromo-2-fluoropyridine (1 g, 5.68 mmol) and 2-methoxyethanamine (0.978 mL, 11.36 mmol) in DMSO (8 mL) was added N,N-diisopropylethylamine (2.97 mL, 11.36 mmol). The mixture was heated at 180° C. for 2 h. LC-MS showed the formation of product. The reaction mixture was cooled to rt and partitioned between EtOAc and water. The organic layer was separated, dried over MgSO₄, and concentrated under reduced pressure. The residue obtained was purified by silica gel chromatography eluting with EtOAc and hexanes to afford 5-bromo-N-(2-methoxyethyl)pyridin-2-amine (1.4 g, 99%). LC-MS (ESI) m/z 231, 233 (M+H)⁺.

Step 2: To a solution of N-(5-tert-butylisoxazol-3-yl)-2-(4-(4,4,5,5-tetramethyl-1,3,2-dioxaborolan-2-yl)phenyl)acetamide (150 mg, 0.39 mmol) from step 1 of Example 85 and 5-bromo-N-(2-methoxyethyl)pyridin-2-amine from Step 1 of this example (90.1 mg, 0.39 mmol) in dioxane (3 mL) was added 2M aq Na₂CO₃ (0.487 mL, 0.975 mmol) and tetrakis(triphenylphosphine)palladium(0) (22.5 mg, 0.019 mmol). The mixture was flushed thoroughly with argon, and then heated in a microwave reactor for 20 min at 160° C. LC-MS showed the formation of product. The mixture was filtered through a celite plug using methanol as eluent and the filtrate was concentrated under reduced pressure. The residue was dissolved in DMF (10 mL) and purified by HPLC to afford N-(5-tert-butylisoxazol-3-yl)-2-(4-(6-(2-methoxyethylamino)pyridin-3-yl)phenyl)acetamide (47 mg, 29%) as a white solid. ¹H NMR (300 MHz, DMSO-d₆) δ 11.20 (s, 1H), 8.28 (d, J=2.3 Hz, 1H), 7.67 (dd, J=2.4, 8.8 Hz, 1H), 7.51 (d, J=8.1 Hz, 2H), 7.34 (d, J=8.1 Hz, 2H), 6.65-6.78 (m, 1H), 6.53-6.64 (m, 2H), 3.67 (s, 2H), 3.33 (s, 4H), 3.28 (s, 3H), 1.27 (s, 9H). LC-MS (ESI) m/z 409 (M+H)⁺.

Step 3: To a solution of N-(5-tert-butylisoxazol-3-yl)-2-(4-(6-(2-methoxyethylamino)pyridin-3-yl)phenyl)acetamide (47 mg, 0.115 mmol) from step 2 of Example 89 in ethanol (5 mL) was added methanesulfonic acid (11 mg, 0.115 mmol). The reaction mixture was heated at 60° C. for 2 h after which the solvent was evaporated under reduced pressure and water was added to the residue. It was then lyophilized to afford 5-(4-(2-(5-tert-butylisoxazol-3-ylamino)-2-oxoethyl)phenyl)-N-(2-methoxyethyl)pyridin-2-aminium methanesulfonate (50.84 mg, 87%) as a white solid. ¹H NMR (300 MHz, DMSO-d₆) δ 11.23 (s, 1H), 8.71 (br s, 1H), 8.08-8.36 (m, 2H), 7.63 (d, J=8.1 Hz, 2H), 7.43 (d, J=8.3 Hz, 2H), 7.17 (d, J=9.2 Hz, 1H), 6.56 (s, 1H), 3.72 (s, 2H), 3.57 (s, 7H), 2.31 (s, 3H), 1.27 (s, 9H). LC-MS (ESI) m/z 409 (M+H)⁺.

### Example 90

### Preparation of 5-(4-(3-(3-(1-fluoro-2-methylpropan-2-yl)-1-methyl-1H-pyrazol-5-yl)ureido)phenyl)pyridin-2-aminium acetate

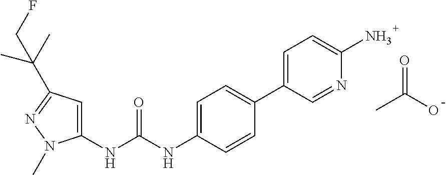

To a stirred solution of 1-(4-(6-aminopyridin-3-yl)phenyl)-3-(3-(1-hydroxy-2-methylpropan-2-yl)-1-methyl-1H-pyrazol-5-yl)urea (100 mg, 0.26 mmol) from Example 82 in DCM (10 mL) was added Deoxo-Fluor (116 mL, 0.63 mmol) dropwise. The resulting mixture was stirred at rt for 2 h before additional Deoxo-Fluor (116 mL, 0.63 mmol) was added, and the reaction mixture was stirred for additional 1 h. The dark brown reaction mixture was quenched with sat. NaHCO₃, and extracted with DCM. The organic layer was washed with brine, dried over Na₂SO₄, filtered, and concentrated under reduced pressure to give an oil which was purified twice by preparative HPLC, eluting with 10-80% CH₃CN over 40 min, to give 5-(4-(3-(3-(1-fluoro-2-methylpropan-2-yl)-1-methyl-1H-pyrazol-5-yl)ureido)phenyl)pyridin-2-aminium acetate (8 mg, 7%) as a white powder. ¹H NMR (300 MHz, DMSO-d₆) δ 9.07 (s, 1H), 8.74 (s, 1H), 8.20 (d, J=2.3 Hz, 1H), 7.66 (dd, J=2.4, 8.6 Hz, 1H), 7.49 (s, 4H), 6.51 (d, J=8.5 Hz, 1H), 6.06 (s, 1H), 5.98 (s, 2H), 3.63 (s, 3H), 2.77 (d, J=19.0 Hz, 2H), 1.90 (s, 3H), 1.36 (s, 3H), 1.29 (s, 3H). LC-MS (ESI) m/z 383 (M+H)⁺.

### Example 91

### Preparation of 2-(4-(6-aminopyridin-3-yl)phenyl)-N-(3-tert-butyl-1H-pyrazol-1-yl)acetamide and 2-(4-(6-aminopyridin-3-yl)phenyl)-N-(5-tert-butyl-1H-pyrazol-1-yl)acetamide

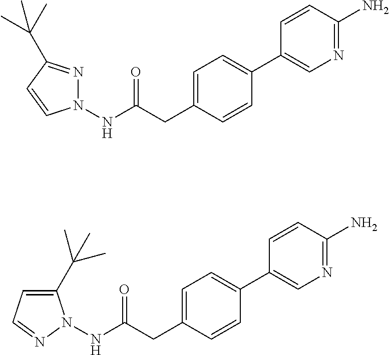 [SMILES: CC(C)(C)c1ccn(NC(=O)Cc2ccc(-c3ccc(N)nc3)cc2)n1 ; CC(C)(C)c1ccnn1NC(=O)Cc1ccc(-c2ccc(N)nc2)cc1]

Step 1: To a solution of 3-tert-butyl-1H-pyrazole (500 mg, 4.028 mmol) in DMF (7 mL) at 0° C. was added NaH (60%, 193 mg, 4.83 mmol). The solution was stirred at rt for 10 min. Chloramine (0.3M in ether, 20.13 mL, 6.042 mmol) was added at 0° C. and the reaction mixture was stirred at rt for overnight. LC-MS showed formation of the product. The reaction mixture was quenched with aq NH₄Cl and extracted with EtOAc. The organic layer was washed with water and brine, dried over MgSO₄, and concentrated uder reduced pressure. Proton NMR of the crude product showed a mixture of regioisomers (3-tert-butyl-1H-pyrazol-1-amine and 5-tert-butyl-1H-pyrazol-1-amine). Such mixture was used for the next step without purification. LC-MS (ESI) m/z 140 (M+H)⁺.

NH₂Cl Preparation:

To a solution of NH₄Cl (2.696 g) in ether (99 mL) at −5° C. was added 28% of NH₄OH (4.23 mL) followed by slow addition of Clorox (64.8 mL) and the reaction mixture was stirred for 1 h at −5° C. The reaction mixture was then transferred to a separatory funnel, and the ether layer was separated, washed with brine, dried with anhydrous K₂CO₃ for 1 h at −40° C. and was used for the next reaction (expected concentration is 0.3M).

Step 2: To a solution of 2-(4-bromophenyl)acetic acid (423 mg, 1.79 mmol) in DMF (6 mL) was added HATU (752 mg, 1.96 mmol). The reaction mixture was stirred for 10 min at rt after which a mixture of 3-tert-butyl-1H-pyrazol-1-amine and 5-tert-butyl-1H-pyrazol-1-amine (250 mg, 1.79 mmol) and DIEA (0.626 mL, 3.59 mmol) was added. Reaction mixture was further stirred at rt for 2 h. LC-MS showed formation of the product. The reaction mixture was partitioned between EtOAc and water, dried over MgSO₄, and concentrated under reduced pressure. The residue obtained was purified by silica gel chromatography eluting with EtOAcand hexanes to afford a mixture of 2-(4-bromophenyl)-N-(3-tert-butyl-1H-pyrazol-1-yl)acetamide and 2-(4-bromophenyl)-N-(5-tert-butyl-1H-pyrazol-1-yl)acetamide (150 mg, 25%). LC-MS (ESI) m/z 336,338 (M+H)⁺.

Step 3: To a solution of 2-(4-bromophenyl)-N-(3-tert-butyl-1H-pyrazol-1-yl)acetamide and 2-(4-bromophenyl)-N-(5-tert-butyl-1H-pyrazol-1-yl)acetamide (150 mg, 0.44 mmol) from Step 2 of this example and 5-(4,4,5,5-tetramethyl-1,3,2-dioxaborolan-2-yl)pyridin-2-amine (97.5 mg, 0.444 mmol) in dioxane (6 mL) was added 2M aq Na₂CO₃ (0.555 mL, 1.11 mmol) and 1,1′-Bis(diphenylphosphino)ferrocene]dichloropalladium(II) (36.24 mg, 0.044 mmol). The mixture was flushed thoroughly with argon, and then heated in a microwave reactor for 20 min at 160° C. LC-MS showed formation of the product. The mixture was filtered through a Celite plug using methanol as eluent and the filtrates were concentrated under reduced pressure. The residue was dissolved in DMF (10 mL) and purified by HPLC using a mixture of water and acetonitrile 10-75% as eluents and diphenyl column as the stationary phase to afford a mixture of 2-(4-(6-aminopyridin-3-yl)phenyl)-N-(3-tert-butyl-1H-pyrazol-1-yl)acetamide and 2-(4-(6-aminopyridin-3-yl)phenyl)-N-(5-tert-butyl-1H-pyrazol-1-yl)acetamide. LC-MS (ESI) m/z 350 (M+H)⁺.

### Example 92

### Preparation of 5-(4-(2-(5-tert-butylisoxazol-3-ylamino)-2-oxoethyl)phenyl)-N-(2-(methylsulfonyl)ethyl)pyridin-2-aminium methanesulfonate

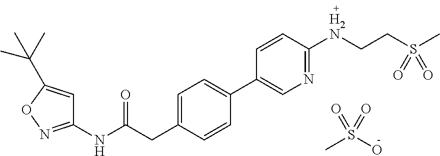

Step 1: 5-Bromo-N-(2-(methylsulfonyl)ethyl)pyridin-2-amine was obtained (610 mg, 38%) using a procedure analogous to that described in Step 1 of Example 89, substituting 2-(methylsulfonyl)ethanamine for 2-methoxyethanamine used in Example 89. LC-MS (ESI) m/z 279, 281 (M+H)⁺.

Step 2: To a solution of N-(5-tert-butylisoxazol-3-yl)-2-(4-(4,4,5,5-tetramethyl-1,3,2-dioxaborolan-2-yl)phenyl)acetamide (225 mg, 0.585 mmol) from Step 1 of Example 85 and 5-bromo-N-(2-(methylsulfonyl)ethyl)pyridin-2-amine (97.5 mg, 0.444 mmol) from Step 1 of this example in acetonitrile was added 2M aq Na₂CO₃ (0.731 mL, 1.46 mmol) and 1,1′-bis(diphenylphosphino)ferrocene]dichloropalladium(II) (47.7 mg, 0.0585 mmol). The mixture was flushed thoroughly with argon, heated in a microwave reactor for 20 min at 160° C. twice. LC-MS showed formation of the product. The mixture was filtered through a Celite plug using methanol as eluent and the filtrates were concentrated under reduced pressure. The residue was dissolved in DMF (10 mL) and purified by HPLC using a mixture of water and acetonitrile 10-75% as eluents and diphenyl column as the stationary phase to afford N-(5-tert-butylisoxazol-3-yl)-2-(4-(6-(2-(methylsulfonyl)ethylamino)pyridin-3-yl)phenyl)acetamide (52 mg, 20%). ¹H NMR (300 MHz, DMSO-d₆) δ 11.20 (s, 1H), 8.34 (d, J=2.3 Hz, 1H), 7.73 (dd, J=2.4, 8.7 Hz, 1H), 7.53 (d, J=8.1 Hz, 2H), 7.35 (d, J=8.3 Hz, 2H), 6.94 (t, J=5.7 Hz, 1H), 6.51-6.73 (m, 2H), 3.59-3.82 (m, 4H), 3.38 (t, J=6.8 Hz, 2H), 3.02 (s, 3H), 1.27 (s, 9H).

LC-MS (ESI) m/z 457 (M+H)⁺.

Step 3: 5-(4-(2-(5-tert-Butylisoxazol-3-ylamino)-2-oxoethyl)phenyl)-N-(2-(methylsulfonyl)ethyl)pyridin-2-aminium methanesulfonate (57.65 mg, 92%) was obtained using a procedure analogous to that described in Step 3 of Example 89, substituting N-(5-tert-butylisoxazol-3-yl)-2-(4-(6-(2-(methylsulfonyl)ethylamino)pyridin-3-yl)phenyl)acetamide from Step 2 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 11.23 (s, 1H), 8.03-8.37 (m, 3H), 7.63 (d, J=7.5 Hz, 2H), 7.42 (d, J=7.7 Hz, 2H), 7.09 (br s, 1H), 6.56 (s, 1H), 3.64-4.00 (m, 5H), 3.08 (s, 4H), 2.31 (d, J=3.6 Hz, 3H), 1.27 (s, 9H). LC-MS (ESI) m/z 457 (M+H)⁺.

### Example 93

### Preparation of 5-(4-(2-(5-tert-butylisoxazol-3-ylamino)-2-oxoethyl)phenyl)-3-cyanopyridin-2-aminium methanesulfonate

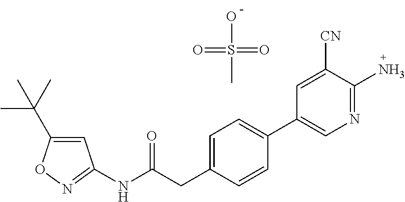

5-(4-(2-(5-tert-Butylisoxazol-3-ylamino)-2-oxoethyl)phenyl)-3-cyanopyridin-2-aminium methanesulfonate (43 mg, 18%) was obtained as an off-white solid using procedures analogous to those described in Steps 2-3 of Example 85, substituting 2-amino-5-bromonicotinonitrile for 5-bromo-3-methylpyridin-2-amine used in Example 85. ¹H NMR (300 MHz, DMSO-d₆) δ 11.21 (s, 1H), 8.55 (d, J=2.3 Hz, 1H), 8.24 (d, J=2.4 Hz, 1H), 7.61 (d, J=8.1 Hz, 2H), 7.37 (d, J=8.1 Hz, 2H), 6.56 (s, 1H), 4.64-4.82 (m, 2H), 3.69 (s, 2H), 2.32 (s, 3H), 1.27 (s, 9H). LC-MS (ESI) m/z 376 (M+H)⁺.

### Example 94

### Preparation of 5-(4-(2-(5-tert-butylisoxazol-3-ylamino)-2-oxoethyl)phenyl)-3-fluoropyridin-2-aminium methanesulfonate

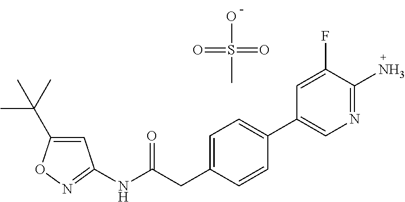

Step 1: To a mixture of 3-fluoropyridin-2-amine (300 mg, 2.68 mmol) in acetonitrile (15 mL) was added N-bromosuccinimide (238 mg, 1.34 mmol). Shielded from light using an aluminum foil, the reaction mixture was stirred at rt for 30 min. Additional N-bromosuccinimide (238 mg, 1.34 mmol) and acetonitrile (5 mL) were added, and the reaction mixture was stirred at rt for another 30 min. The mixture was then partitioned between EtOAc (50 mL) and water (50 mL), and the aqueous layer was separated and extracted with EtOAc (2×50 mL). The combined organic layers were washed with brine (2×50 mL), dried over MgSO₄, filtered and concentrated under reduced pressure. The crude product was purified by silica gel chromatography, eluting with 0-40% EtOAc in hexanes to give 5-bromo-3-fluoropyridin-2-amine as a white solid (350 mg, 68%). ¹H NMR (300 MHz, CHLOROFORM-d) δ 7.95 (s, 1H), 7.39 (dd, J=1.7, 9.8 Hz, 1H), 4.66 (br s, 2H). LC-MS (ESI) m/z 191 and 193 (M+H)⁺.

Step 2: To a microwave reaction vial was added N-(5-tert-butylisoxazol-3-yl)-2-(4-(4,4,5,5-tetramethyl-1,3,2-dioxaborolan-2-yl)phenyl)acetamide from Step 1 of Example 85 (200 mg, 0.52 mmol), 5-bromo-3-fluoropyridin-2-amine from Step 1 of this example (149 mg, 0.78 mmol), 2M aq sodium carbonate (0.78 mL, 1.56 mmol), 1,4-dioxane (10 mL), and tetrakis(triphenylphosphine) palladium(0) (60.1 mg, 0.052 mmol). The vial was purged with argon, sealed, and heated in a microwave reactor at 170° C. for 30 min. The mixture was partitioned between EtOAc (50 mL) and water (50 mL), and the aqueous layer was separated and extracted with EtOAc (2×40 mL). The combined organic layers were washed with brine (2×30 mL), dried over MgSO₄, filtered and concentrated under reduced pressure. The crude product was purified by silica gel chromatography, eluting with 0-70% EtOAc in hexanes to give 2-(4-(6-amino-5-fluoropyridin-3-yl)phenyl)-N-(5-tert-butylisoxazol-3-yl)acetamide (45 mg, 23%) as an off-white solid. LC-MS (ESI) m/z 369 (M+H)⁺.

Step 3: A mixture of 2-(4-(6-amino-5-fluoropyridin-3-yl)phenyl)-N-(5-tert-butylisoxazol-3-yl)acetamide from Step 2 of this example (45 mg, 0.12 mmol) and methanesulfonic acid (11.7 mg, 0.12 mmol) in ethanol (10 mL) was stirred at 60° C. for 2 h. The solvent was evaporated under reduced pressure. Water was added and the mixture was frozen and lyophilized to give 5-(4-(2-(5-tert-butylisoxazol-3-ylamino)-2-oxoethyl)phenyl)-3-fluoropyridin-2-aminium methanesulfonate (49 mg, 86%) as an off-white solid. ¹H NMR (300 MHz, DMSO-d₆) δ 11.22 (s, 1H), 8.13 (s, 1H), 7.52-7.71 (m, 5H), 7.39 (d, J=8.1 Hz, 2H), 6.56 (s, 1H), 3.70 (s, 2H), 2.32 (s, 3H), 1.27 (s, 9H). LC-MS (ESI) m/z 369 (M+H)⁺.

### Example 95

### Preparation of 5-(4-(2-(5-tert-butylisoxazol-3-ylamino)-2-oxoethyl)phenyl)-6-fluoropyridin-2-aminium methanesulfonate

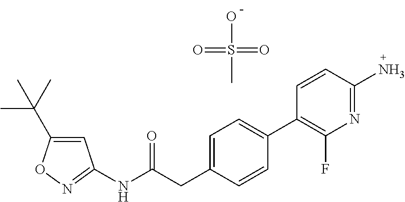

Step 1: 5-Bromo-6-fluoropyridin-2-amine (600 mg, 35%) was obtained as a white solid using a procedure analogous to that described in Step 1 of Example 94, substituting 6-fluoropyridin-2-amine for 3-fluoropyridin-2-amine used in Example 94. ¹H NMR (300 MHz, Chloroform-d) δ 7.62 (t, J=8.6 Hz, 1H), 6.28 (dd, J=1.2, 8.4 Hz, 1H), 4.58 (br s, 2H). LC-MS (ESI) m/z 191 and 193 (M+H)⁺.

Step 2: 5-(4-(2-(5-tert-Butylisoxazol-3-ylamino)-2-oxoethyl)phenyl)-6-fluoropyridin-2-aminium methanesulfonate (34 mg, 14%) was obtained as an off-white solid using procedures analogous to those described in Steps 2-3 of Example 94, substituting 5-bromo-6-fluoropyridin-2-amine from Step 1 of this example for 5-bromo-3-fluoropyridin-2-amine used in Example 94. ¹H NMR (300 MHz, DMSO-d₆) δ 11.21 (s, 1H), 7.66 (dd, J=8.2, 10.8 Hz, 1H), 7.38-7.47 (m, 2H), 7.30-7.37 (m, 2H), 6.57 (s, 1H), 6.42 (d, J=8.3 Hz, 1H), 3.67 (s, 2H), 2.30 (s, 3H), 1.27 (s, 9H). LC-MS (ESI) m/z 369 (M+H)⁺.

### Example 96

### Preparation of 5-(4-(2-(3-tert-butylisoxazol-5-ylamino)-2-oxoethyl)phenyl)-N-(2-morpholinoethyl)pyridin-2-aminium methanesulfonate

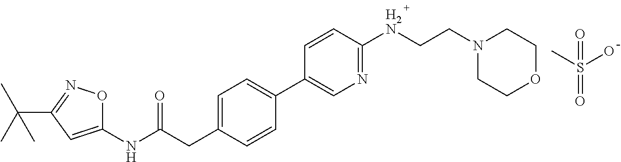

Step 1: To a mixture of 5-bromo-2-fluoropyridine (5.2 g, 29.5 mmol) and 2-morpholinoethanamine (3.83 g, 29.5 mmol) in t-BuOH (40 mL) was added p-toluenesulfonic acid (112 mg, 0.59 mmol). The reaction mixture was heated in a sealed tube at 90° C. for overnight. After cooled to rt, the reaction mixture was concentrated under reduced pressure. The residue was purified by silica gel chromatography eluting with EtOAc to afford 5-bromo-N-(2-morpholinoethyl)pyridin-2-amine (4.6 g, 55%) as a colorless oil. LC-MS (ESI) m/z 287 (M+H)⁺.

Step 2: N-(3-tert-Butylisoxazol-5-yl)-2-(4-(4,4,5,5-tetramethyl-1,3,2-dioxaborolan-2-yl)phenyl)acetamide (821 mg, 51%) was synthesized as a white solid using the procedure analogous to that described in Step 1 of Example 85, substituting 3-tert-butylisoxazol-5-amine for 5-tert-butylisoxazol-3-amine used in Example 85. LC-MS (ESI) m/z 385 (M+H)⁺.

Step 3: N-(3-tert-Butylisoxazol-5-yl)-2-(4-(6-(2-morpholinoethylamino)pyridin-3-yl)phenyl)acetamide (83.4 mg, 9%) was synthesized as a white solid using the procedure analogous to that described in Step 2 of Example 85, substituting 5-bromo-N-(2-morpholinoethyl)pyridin-2-amine from Step 1 of this example for 5-bromo-3-methylpyridin-2-amine, and N-(3-tert-butylisoxazol-5-yl)-2-(4-(4,4,5,5-tetramethyl-1,3,2-dioxaborolan-2-yl)phenyl)acetamide from Step 2 of this example for N-(5-tert-butylisoxazol-3-yl)-2-(4-(4,4,5,5-tetramethyl-1,3,2-dioxaborolan-2-yl)phenyl)acetamide used in Step 2 of Example 85. LC-MS (ESI) m/z 464 (M+H)⁺.

Step 4: 5-(4-(2-(3-tert-Butylisoxazol-5-ylamino)-2-oxoethyl)phenyl)-N-(2-morpholinoethyl)pyridin-2-aminium methanesulfonate (101.5 mg, 100%) was synthesized as a white solid using the procedure analogous to that described in Step 3 of Example 89, substituting N-(3-tert-butylisoxazol-5-yl)-2-(4-(6-(2-morpholinoethylamino)pyridin-3-yl)phenyl)acetamide from Step 3 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 11.80 (s, 1H), 8.36 (d, J=2.3 Hz, 1H), 7.81 (d, J=7.5 Hz, 1H), 7.56 (d, J=8.1 Hz, 2H), 7.37 (d, J=8.1 Hz, 2H), 7.11 (br s, 1H), 6.69 (d, J=8.7 Hz, 1H), 6.19 (s, 1H), 3.86 (br s, 4H), 3.58-3.76 (m, 6H), 3.33 (br, 4H), 2.32 (s, 3H), 1.23 (s, 9H). LC-MS (ESI) m/z 464 (M+H)⁺.

### Example 97

### Preparation of 1-(4-(6-aminopyridin-3-yl)phenyl)-3-(3-(1-fluoro-2-methylpropan-2-yl)isoxazol-5-yl)urea

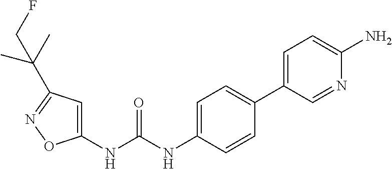 [SMILES: CC(C)(CF)c1cc(NC(=O)Nc2ccc(-c3ccc(N)nc3)cc2)on1]

Step 1: Phenyl (3-(1-fluoro-2-methylpropan-2-yl)isoxazol-5-yl)carbamate (60 mg, 24%) was prepared using a procedure analogous to that described in Example 90, substituting phenyl (3-(1-hydroxy-2-methylpropan-2-yl)isoxazol-5-yl)carbamate from Example 81 for 1-(4-(6-aminopyridin-3-yl)phenyl)-3-(3-(1-hydroxy-2-methylpropan-2-yl)-1-methyl-1H-pyrazol-5-yl)urea used in Example 90. LC-MS (ESI) m/z 279 (M+H)⁺.

Step 2: 1-(4-(6-aminopyridin-3-yl)phenyl)-3-(3-(1-fluoro-2-methylpropan-2-yl)isoxazol-5-yl)urea (20 mg, 25%) was prepared using a procedure analogous to that described in described in Step 4 of Example 36, substituting phenyl (3-(1-fluoro-2-methylpropan-2-yl)isoxazol-5-yl)carbamate from Step 1 of this example for phenyl 5-(1-(trifluoromethyl)cyclopropyl)isoxazol-3-ylcarbamate, and 5-(4-aminophenyl)pyridin-2-amine for 5-(4-aminophenyl)-N-(2-morpholinoethyl)pyridin-2-amine hydrochloride used in Example 36. ¹H NMR (300 MHz, DMSO-d₆) δ 9.16 (s, 1H), 8.22 (d, J=2.3 Hz, 1H), 7.67 (dd, J=2.4, 8.6 Hz, 1H), 7.51 (s, 4H), 6.51 (d, J=8.7 Hz, 1H), 6.21-6.36 (m, 1H), 6.12 (s, 1H), 6.00 (s, 2H), 1.91-2.05 (m, 2H), 1.90 (s, 2H), 1.80 (d, J=6.8 Hz, 1H), 1.52-1.73 (m, 2H), 0.88 (t, J=7.4 Hz, 2H). LC-MS (ESI) m/z 370 (M+H)⁺.

### Example 98

### Preparation of 5-(4-(3-(5-(3-methyloxetan-3-yl)isoxazol-3-yl)ureido)phenyl)pyridin-2-aminium methanesulfonate

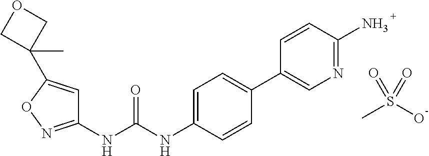

Step 1: To a stirred solution of 3-methyloxetane-3-carboxylic acid (5.3 g, 46 mmol) in MeCN (50 mL) was added K₂CO₃ (8.2 g, 60 mmol) and benzyl bromide (5.4 mL, 46 mmol). The resulting mixture was refluxed for 7 h before it was cooled to rt and partitioned between EtOAc (100 mL) and water (50 mL). The organic layer was washed with brine, dried over Na₂SO₄, filtered, and concentrated under reduced pressure to give an oil which was purified by silica gel flash chromatography, eluting with 0-30% EtOAc in hexanes, to give benzyl 3-methyloxetane-3-carboxylate (3.3 g, 35%) as a colorless oil. ¹H NMR (300 MHz, CHLOROFORM-d) δ 7.38 (s, 5H), 5.22 (s, 2H), 4.98 (d, J=6.0 Hz, 2H), 4.43 (d, J=5.8 Hz, 2H), 1.65 (s, 3H).

Step 2: To a stirred solution of t-BuOK (2.9 g, 25.8 mmol) in THF (10 mL) under argon was added a mixture of benzyl 3-methyloxetane-3-carboxylate (3.3 g, 15.9 mmol)/MeCN (1.3 mL, 15.9 mmol)/THF (10 mL) slowly over 10 min. The resulting mixture was stirred at rt for over night before it was quenched with 3N HCl to pH˜2. The mixture was extracted with DCM (2×50 mL). The combined organic layers was washed with brine, dried over MgSO₄, filtered, and concentrated under reduced pressure to give an oil which was purified by silica gel flash chromatography, eluting with 0-75% EtOAc in hexanes, to give 3-(3-methyloxetan-3-yl)-3-oxopropanenitrile (1.38 g, 56%) as a light brown solid. ¹H NMR (300 MHz, CHLOROFORM-d) δ 4.92 (d, J=6.6 Hz, 2H), 4.54 (d, J=6.4 Hz, 2H), 3.68 (s, 2H), 1.68 (s, 3H).

Step 3: 5-(3-Methyloxetan-3-yl)isoxazol-3-amine was prepared using a procedure analogous to that described in Step 2 of Example 32, substituting 3-(3-methyloxetan-3-yl)-3-oxopropanenitrile from Step 2 of this example for 4-fluoro-4-methyl-3-oxopentanenitrile used in Example 32. LC-MS (ESI) m/z 155 (M+H)⁺. Step 4: 5-(4-(3-(5-(3-methyloxetan-3-yl)isoxazol-3-yl)ureido)phenyl)pyridin-2-aminium methanesulfonate (50 mg, 22%) was prepared using procedures analogous to those described in Step 3-4 of Example 32, substituting 5-(3-methyloxetan-3-yl)isoxazol-3-amine from Step 3 of this example for 3-(2-fluoropropan-2-yl)isoxazol-5-amine used in Example 32. ¹H NMR (300 MHz, DMSO-d₆) δ 10.39 (s, 1H), 9.14 (s, 1H), 8.15-8.31 (m, 2H), 7.73 (br s, 2H), 7.54-7.66 (m, 4H), 7.00 (d, J=10.0 Hz, 1H), 6.20 (s, 1H), 4.77 (d, J=5.7 Hz, 2H), 4.51 (d, J=5.7 Hz, 2H), 2.37 (s, 3H), 1.63 (s, 3H). LC-MS (ESI) m/z 366 (M+H)⁺.

### Example 99

### Preparation of 5-(4-(2-((5-(3-methyloxetan-3-yl)isoxazol-3-yl)amino)-2-oxoethyl)phenyl)pyridin-2-aminium methanesulfonate

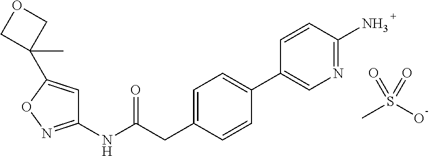

5-(4-(2-((5-(3-methyloxetan-3-yl)isoxazol-3-yl)amino)-2-oxoethyl)phenyl)pyridin-2-aminium methanesulfonate (350 mg, 56%) was prepared using procedures analogous to those described in Steps 1-3 of Example 83, substituting 5-(3-methyloxetan-3-yl)isoxazol-3-amine from Example 98 for 2-(5-aminoisoxazol-3-yl)-2-methylpropan-1-ol used in Example 83. ¹H NMR (300 MHz, DMSO-d₆) δ 11.95 (s, 1H), 8.20-8.36 (m, 2H), 8.04 (br s, 2H), 7.64 (d, J=8.1 Hz, 2H), 7.43 (d, J=8.3 Hz, 2H), 7.08 (d, J=9.2 Hz, 1H), 6.32 (s, 1H), 4.74 (d, J=5.7 Hz, 2H), 4.49 (d, J=5.8 Hz, 2H), 3.17-3.53 (m, 2H), 2.39 (s, 3H), 1.60 (s, 3H). LC-MS (ESI) m/z 365 (M+H)⁺.

### Example 100

### Preparation of 5-(4-(2-(5-(1-methylcyclopropyl)isoxazol-3-ylamino)-2-oxoethyl)phenyl)pyridin-2-aminium methanesulfonate

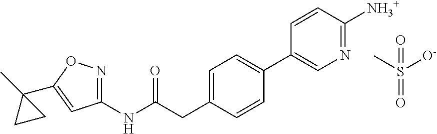

Step 1: A stirred suspension of sodium hydride (9.3 g, 60% dispersion in mineral oil, 233 mmol) in dry THF (100 mL) was heated to 75° C. To this suspension was added a mixture of methyl 1-methylcyclopropanecarboxylate (17 g, 149 mmol) and dry acetonitrile (9.1 g, 233 mmol) dropwise over the course of 45 min. The resulting suspension was heated at 70° C. for overnight. After cooled to rt, the reaction mixture was poured into water (400 mL) and resulting mixture was extracted with diethyl ether (2×200 mL). The aqueous layer was seperated, acidified to pH˜2 with aq 2N hydrochloric acid and extracted with diethyl ether (2×300 mL). The combined organic layers were dried over MgSO₄, filtered, and concentrated under reduced pressure to afford 3-(1-methylcyclopropyl)-3-oxopropanenitrile (14 g, 54%) as a yellow oil. ¹H NMR (300 MHz, CHLOROFORM-d) δ 3.59 (s, 2H), 1.41 (s, 2H), 1.33-1.38 (m, 2H), 1.29 (s, 1H), 0.85-0.94 (m, 1H), 0.70-0.80 (m, 1H). LC-MS (ESI) m/z 124 (M+H)⁺.

Step 2: To a stirred solution of NaOH (3.58 g, 89.4 mmol) and 3-(1-methylcyclopropyl)-3-oxopropanenitrile (10 g, 81.3 mmol) from Step 1 of this example in water (50 mL) and ethanol (50 mL) was added hydroxylamine sulfate (14.7 g, 89.4 mmol). The reaction mixture was adjusted to pH˜7.5 with aqueous 2 N sodium hydroxide solution, then heated at 80° C. for overnight. After cooled to rt, the solvents were removed under reduced pressure. The resulting residue was partitioned between water (300 mL) and dichloromethane (400 mL). The organic layer was seperated, washed with brine, dried over MgSO₄, filtered, and concentrated under reduced pressure to afford 5-(1-methylcyclopropyl)isoxazol-3-amine (8.1 g, 72%) as a white solid. ¹H NMR (300 MHz, CHLOROFORM-d) δ 4.83 (s, 1H), 4.43 (br s, 2H), 1.40 (s, 3H), 0.89-1.00 (m, 2H), 0.70-0.82 (m, 2H). LC-MS (ESI) m/z 139 (M+H)⁺.

Step 3: 2-(4-Bromophenyl)-N-(5-(1-methylcyclopropyl)isoxazol-3-yl)acetamide (637 mg, 41%) was synthesized as a white solid using the procedure analogous to that described in Step 1 of Example 18, substituting 5-(1-methylcyclopropyl)isoxazol-3-amine from Step 2 of this example for 5-(tert-butyl)isoxazol-3-amine used in Example 18. LC-MS (ESI) m/z 338 (M+H)⁺.

Step 4: 2-(4-(6-Aminopyridin-3-yl)phenyl)-N-(5-(1-methylcyclopropyl)isoxazol-3-yl)acetamide (60 mg, 16%) was synthesized as a white solid using the procedure analogous to that described in Step 2 of Example 40, substituting 2-(4-bromophenyl)-N-(5-(1-methylcyclopropyl)isoxazol-3-yl)acetamide from Step 3 of this example for 5-bromo-N-tritylprydin-2-amine used in Step 2 of Example 40. LC-MS (ESI) m/z 349 (M+H)⁺.

Step 5: 5-(4-(2-(5-(1-Methylcyclopropyl)isoxazol-3-ylamino)-2-oxoethyl)phenyl)pyridin-2-aminium methanesulfonate was synthesized as a white solid (77.4 mg, 100%) using the procedure analogous to that described in Step 3 of Example 89, substituting 2-(4-(6-aminopyridin-3-yl)phenyl)-N-(5-(1-methylcyclopropyl)isoxazol-3-yl)acetamide from Step 4 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ11.80 (s, 1H), 8.23-8.43 (m, 2H), 8.11 (br s, 2H), 7.63 (d, J=8.1 Hz, 3H), 7.41 (d, J=8.1 Hz, 2H), 7.09 (d, J=9.2 Hz, 1H), 5.93 (s, 1H), 3.74 (br s, 2H), 2.40 (s, 3H), 1.35 (s, 3H), 0.90 (m, 2H), 0.77-0.86 (m, 2H). LC-MS (ESI) m/z 349 (M+H)⁺.

### Example 101

### Preparation of 5-(4-(2-(3-(2-fluoropropan-2-yl)isoxazol-5-ylamino)-2-oxoethyl)phenyl)pyridin-2-aminium methanesulfonate

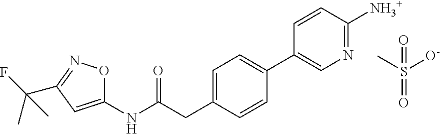

Step 1: 2-(4-Bromophenyl)-N-(3-(2-fluoropropan-2-yl)isoxazol-5-yl)acetamide (481 mg, 15%) was synthesized as a white solid using the procedure analogous to that described in Step 1 of Example 18, substituting 3-(2-fluoropropan-2-yl)isoxazol-5-amine from Step 2 of Example 32 for 5-(tert-butyl)isoxazol-3-amine used in Example 18. LC-MS (ESI) m/z 342 (M+H)⁺.

Step 2: 2-(4-(6-Aminopyridin-3-yl)phenyl)-N-(3-(2-fluoropropan-2-yl)isoxazol-5-yl)acetamide (40 mg, 12%) was synthesized as a white solid using the procedure analogous to that described in Step 2 of Example 40, substituting 2-(4-bromophenyl)-N-(3-(2-fluoropropan-2-yl)isoxazol-5-yl)acetamide from Step 1 of this example for 5-bromo-N-tritylprydin-2-amine used in Step 2 Example 40. LC-MS (ESI) m/z 355 (M+H)⁺.

Step 3: 5-(4-(2-(3-(2-Fluoropropan-2-yl)isoxazol-5-ylamino)-2-oxoethyl)phenyl)pyridin-2-aminium methanesulfonate (51.4 mg, 100%) as a white solid using the procedure analogous to that described in Step 3 of Example 89, substituting 2-(4-(6-aminopyridin-3-yl)phenyl)-N-(3-(2-fluoropropan-2-yl)isoxazol-5-yl)acetamide from Step 2 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 12.02 (br s, 1H), 8.23-8.54 (m, 2H), 8.08 (br s, 2H), 7.64 (d, J=8.1 Hz, 2H), 7.43 (d, J=7.9 Hz, 2H), 7.09 (d, J=9.0 Hz, 1H), 6.30 (s, 1H), 3.78 (br s, 2H), 2.37 (s, 3H), 1.57-1.87 (m, 6H). LC-MS (ESI) m/z 355 (M+H)⁺.

### Example 102

### Preparation of 5-(4-(2-((5-(2,2-difluoro-1-methylcyclopropyl)isoxazol-3-yl)amino)-2-oxoethyl)phenyl)pyridin-2-aminium acetate

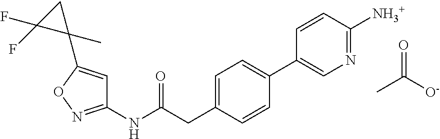

Step 1: 5-(2,2-Difluoro-1-methylcyclopropyl)isoxazol-3-amine (3.9 g, 61% over three steps) was prepared using procedures analogous to those described in Steps 1-3 of Example 98, substituting 2,2-difluoro-1-methylcyclopropanecarboxylic acid for 3-methyloxetane-3-carboxylic acid used in Example 98. LC-MS (ESI) m/z 175 (M+H)⁺.

Step 2: 5-(4-(2-((5-(2,2-Difluoro-1-methylcyclopropyl)isoxazol-3-yl)amino)-2-oxoethyl)phenyl)pyridin-2-aminium acetate (73 mg, 28%) was prepared using procedures analogous to those described in Steps 1-3 of Example 83, substituting 5-(2,2-difluoro-1-methylcyclopropyl)isoxazol-3-amine from Step 1 of this example for 2-(5-aminoisoxazol-3-yl)-2-methylpropan-1-ol used in Example 83. ¹H NMR (300 MHz, DMSO-d₆) δ 8.22 (d, J=2.3 Hz, 1H), 7.68 (dd, J=2.4, 8.7 Hz, 1H), 7.52 (d, J=8.3 Hz, 2H), 7.33 (d, J=8.3 Hz, 2H), 6.52 (d, J=8.5 Hz, 1H), 6.24 (s, 1H), 6.05 (s, 2H), 3.71 (s, 2H), 2.14 (ddd, J=5.4, 8.3, 13.8 Hz, 1H), 1.89 (s, 2H), 1.79 (ddd, J=6.2, 8.1, 12.6 Hz, 1H), 1.50 (s, 3H). LC-MS (ESI) m/z 385 (M+H)⁺.

### Example 103

### Preparation of 5-(4-(2-((5-(1-(fluoromethyl)cyclopropyl)isoxazol-3-yl)amino)-2-oxoethyl)phenyl)pyridin-2-aminium acetate

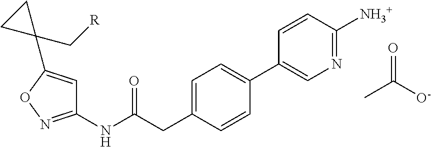

Step 1: (1-(3-aminoisoxazol-5-yl)cyclopropyl)methanol (860 mg, 16%) was prepared using procedures analogous to those described in Steps 1-3 of Example 80, substituting ethyl 1-(hydroxymethyl)cyclopropanecarboxylate for methyl 3-hydroxy-2,2-dimethylpropanoate used in Example 80.

Step 2: To a stirred solution of (1-(3-aminoisoxazol-5-yl)cyclopropyl)methanol (860 mg, 5.58 mmol) in pyridine (10 mL) at rt was added 2-(4-bromophenyl)acetyl chloride (1.56 g, 6.70 mmol). The resulting mixture was stirred at rt for 1 h before DMAP (50 mg, 0.41 mmol) was added and the stirred continued for 16 h. The reaction mixture was then heated at 40° C. for another 16 h and then cooled to rt and triturated with water twice. The oil residue was partitioned between EtOAc and sat. NH₄Cl. The organic layer was washed with brine, dried over Na₂SO₄, filtered, and concentrated under reduced pressure to give an oil which was purified by silica gel flash chromatography, eluting with 0-90% EtOAc in hexanes, to give 2-(4-bromophenyl)-N-(5-(1-(hydroxymethyl)cyclopropyl)isoxazol-3-yl)acetamide.

(1-(3-(2-(4-bromophenyl)acetamido)isoxazol-5-yl)cyclopropyl)methyl 2-(4-bromophenyl)acetate (600 mg, 1.10 mmol) was also isolated from this reaction.

(1-(3-(2-(4-bromophenyl)acetamido)isoxazol-5-yl)cyclopropyl)methyl 2-(4-bromophenyl)acetate (600 mg, 1.10 mmol) from above was taken up in 10 mL of MeOH/THF (1:1, v/v) and stirred with 3N NaOH (0.75 mL, 2.20 mmol) at rt for 1 h. The reaction mixture was then diluted with brine and extracted with EtOAC. The organic layer was washed with brine, dried over Na₂SO₄, filtered, and concentrated under reduced pressure. The residue was purified by silica gel flash chromatography, eluting with 0-90% EtOAc in hexanes, to give additional 2-(4-bromophenyl)-N-(5-(1-(hydroxymethyl)cyclopropyl)isoxazol-3-yl)acetamide ((400 mg combined weight, 20%). LC-MS (ESI) m/z 351, 353 (M+H)⁺.

Step 3: 2-(4-Bromophenyl)-N-(5-(1-(fluoromethyl)cyclopropyl)isoxazol-3-yl)acetamide (130 mg, 32%) was prepared using a procedure analogous to that described in Example 90, substituting 2-(4-bromophenyl)-N-(5-(1-(hydroxymethyl)cyclopropyl)isoxazol-3-yl)acetamide from Step 2 of this example for 1-(4-(6-aminopyridin-3-yl)phenyl)-3-(3-(1-hydroxy-2-methylpropan-2-yl)-1-methyl-1H-pyrazol-5-yl)urea used in Example 90. LC-MS (ESI) m/z 353, 355 (M+H)⁺.

Step 4: 5-(4-(2-((5-(1-(Fluoromethyl)cyclopropyl)isoxazol-3-yl)amino)-2-oxoethyl)phenyl)pyridin-2-aminium acetate (65 mg, 44%) was prepared using a procedure analogous to that described in Step 2 of Example 83, substituting 2-(4-bromophenyl)-N-(5-(1-(fluoromethyl)cyclopropyl)isoxazol-3-yl)acetamide from Step 3 of this example for 2-(4-bromophenyl)-N-(3-(1-hydroxy-2-methylpropan-2-yl)isoxazol-5-yl)acetamide used in Example 83. ¹H NMR (300 MHz, DMSO-d₆) δ 8.23 (d, J=2.3 Hz, 1H), 7.68 (dd, J=2.4, 8.5 Hz, 1H), 7.52 (d, J=8.1 Hz, 2H), 7.34 (d, J=8.3 Hz, 2H), 6.52 (d, J=8.5 Hz, 1H), 6.28 (s, 1H), 6.05 (s, 2H), 3.73 (s, 2H), 3.34 (br s, 2H), 2.54-2.63 (m, 3H), 1.91 (s, 3H), 1.50-1.75 (m, 1H). LC-MS (ESI) m/z 367 (M+H)⁺.

### Example 104

### Preparation of 6′-(3-(5-(1-methylcyclopropyl)isoxazol-3-yl)ureido)-3,3′-bipyridin-6-aminium methanesulfonate

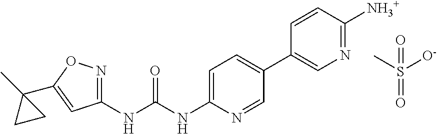

Step 1: Phenyl 5-(1-methylcyclopropyl)isoxazol-3-ylcarbamate (761 mg, 43%) was synthesized as a white solid using the procedure analogous to that described in Step 1 of Example 70, substituting 3-(1-methylcyclopropyl)isoxazol-5-amine from step 2 of Example 100 for 3-tert-butylisoxazol-5-amine used in Step 1 of Example 70. LC-MS (ESI) m/z 259 (M+H)⁺.

Step 2: To a stirred solution of phenyl 5-(1-methylcyclopropyl)isoxazol-3-ylcarbamate (258 mg, 1.0 mmol) from Step 1 of this example and N^{β}-trityl-3,3′-bipyridine-6,6′-diamine from Step 2 of Example 40 (428 mg, 1.0 mmol) in DMF (5 mL) was added triethyl amine (150 mg, 1.5 mmol) and a catalytic amount of DMAP. The reaction solution was stirred at 50° C. for 3 d. After cooled to rt, the reaction mixture was treated with water (15 mL) and filtered. The precipitates were collected and purified by silica gel chromatography eluting with 1:3 EtOAc/hexanes to afford 1-(5-(1-methylcyclopropyl)isoxazol-3-yl)-3-(6′-(tritylamino)-3,3′-bipyridin-6-yl)urea (316 mg, 53%) as a white solid. LC-MS (ESI) m/z 594 (M+H)⁺.

Step 3: To a stirred solution of 1-(5-(1-methylcyclopropyl)isoxazol-3-yl)-3-(6′-(tritylamino)-3,3′-bipyridin-6-yl)urea (316 mg, 0.53 mmol) from Step 2 in DCM (10 mL) was added TFA (3 mL) and water (0.1 mL). The reaction mixture was stirred at rt for 3 h, and then concentrated under reduced pressure. The residue was purified by HPLC using a mixture of 10-80% water and acetonitrile as eluents (diphenyl column) to afford 1-(6′-amino-3,3′-bipyridin-6-yl)-3-(5-(1-methylcyclopropyl)isoxazol-3-yl)urea (117 mg, 63%) as a white solid. LC-MS (ESI) m/z 351 (M+H)⁺.

Step 4: 6′-(3-(5-(1-Methylcyclopropyl)isoxazol-3-yl)ureido)-3,3′-bipyridin-6-aminium methanesulfonate (150.6 mg, 100%) was prepared as a white solid using the procedure analogous to that described in Step 3 of Example 89, substituting 1-(6′-amino-3,3′-bipyridin-6-yl)-3-(5-(1-methylcyclopropyl)isoxazol-3-yl)urea from Step 3 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 11.37 (br s, 1H), 9.85 (s, 1H), 8.66 (d, J=2.1 Hz, 1H), 8.27-8.42 (m, 2H), 7.94-8.18 (m, 3H), 7.68 (d, J=8.7 Hz, 1H), 7.08 (d, J=10.0 Hz, 1H), 5.89 (s, 1H), 2.33 (s, 2H), 1.38 (s, 3H), 0.92-1.01 (m, 2H), 0.80-0.89 (m, 2H). LC-MS (ESI) m/z 351 (M+H)⁺.

### Example 105

### Preparation of 5-(4-(2-(5-tert-butylisoxazol-3-ylamino)-2-oxoethyl)phenyl)-N-(2-morpholinoethyl)pyridin-2-aminium methanesulfonate

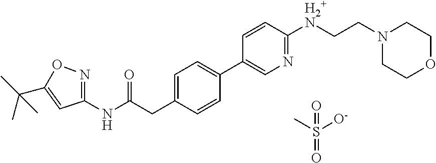

Step 1: N-(5-tert-Butylisoxazol-3-yl)-2-(4-(6-(2-morpholinoethylamino)pyridin-3-yl)phenyl)acetamide (80 mg, 17%) was synthesized as a white solid using the procedure analogous to that described in Step 2 of Example 85, substituting 5-bromo-N-(2-morpholinoethyl)pyridin-2-amine from Stepl of Example 36 for 5-bromo-3-methylpyridin-2-amine used in Example 85. LC-MS (ESI) m/z 464 (M+H)⁺.

Step 2: 5-(4-(2-(5-tert-Butylisoxazol-3-ylamino)-2-oxoethyl)phenyl)-N-(2-morpholinoethyl)pyridin-2-aminium methanesulfonate (97.2 mg, 100%) was synthesized as a white solid using the procedure analogous to that described in Step 3 of Example 89, substituting N-(5-tert-butylisoxazol-3-yl)-2-(4-(6-(2-morpholinoethylamino)pyridin-3-yl)phenyl)acetamide from Step 1 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 11.22 (s, 1H), 8.36 (d, J=1.9 Hz, 1H), 7.84 (d, J=8.3 Hz, 1H), 7.56 (d, J=8.1 Hz, 2H), 7.37 (d, J=8.1 Hz, 2H), 7.21 (br s, 1H), 6.73 (d, J=8.7 Hz, 1H), 6.56 (s, 1H), 3.87 (br s, 4H), 3.69 (br s, 4H), 3.33 (d, J=4.7 Hz, 6H), 2.35 (s, 3H), 1.27 (s, 9H). LC-MS (ESI) m/z 464 (M+H)⁺.

### Example 106

### Preparation of 5-(4-(3-(5-(1-methylcyclopropyl)isoxazol-3-yl)ureido)phenyl)pyridin-2-aminium methanesulfonate

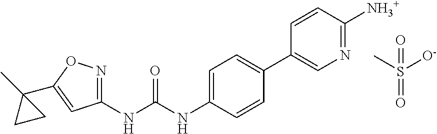

Step 1: 1-(4-(6-Aminopyridin-3-yl)phenyl)-3-(5-(1-methylcyclopropyl)isoxazol-3-yl)urea (60 mg, 34%) was synthesized as a solid according to the procedure described in Step 4 of Example 36, substituting 5-(4-aminophenyl)pyridin-2-amine for 5-(4-aminophenyl)-N-(2-morpholinoethyl)pyridin-2-amine, and phenyl 5-(1-methylcyclopropyl)isoxazol-3-ylcarbamate Step 1 of Example 104 for phenyl 5-(1-(trifluoromethyl)cyclopropyl)isoxazol-3-ylcarbamate used in Example 36. LC-MS (ESI) m/z 350 (M+H)⁺.

Step 2: 5-(4-(3-(5-(1-Methylcyclopropyl)isoxazol-3-yl)ureido)phenyl)pyridin-2-aminium methanesulfonate (77.3 mg, 100%) was synthesized as a white solid using the procedure analogous to that described in Step 3 of Example 89, substituting 1-(4-(6-aminopyridin-3-yl)phenyl)-3-(5-(1-methylcyclopropyl)isoxazol-3-yl)urea from Step 1 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 13.68 (br s, 1H), 10.25 (s, 1H), 9.13 (s, 1H), 8.16-8.45 (m, 2H), 8.00 (br s, 2H), 7.50-7.78 (m, 4H), 7.07 (d, J=9.2 Hz, 1H), 5.82 (s, 1H), 2.37 (s, 3H), 1.37 (s, 3H), 0.94 (br s, 2H), 0.77-0.87 (m, 2H). LC-MS (ESI) m/z 350 (M+H)⁺.

### Example 107

### Preparation of 1-(2-((5-(4-(3-(5-(tert-butyl)isoxazol-3-yl)ureido)phenyl)pyridin-2-yl)amino)ethyl)-4,4-difluoropiperidin-1-ium methanesulfonate

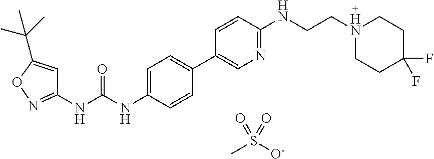

Step 1: 5-Bromo-N-(2-(4,4-difluoropiperidin-1-yl)ethyl)pyridin-2-amine (1.47 g, 76%) was obtained using a procedure analogous to that described in Step 1 of Example 89, substituting 2-(4,4-difluoropiperidin-1-yl)ethanamine for 2-methoxyethanamine used in Example 89. LC-MS (ESI) m/z 320, 322 (M+H)⁺.

Step 2: To a solution of 5-bromo-N-(2-(4,4-difluoropiperidin-1-yl)ethyl)pyridin-2-amine (600 mg, 1.875 mmol) from Step 1 of this example and tert-butyl 4-(4,4,5,5-tetramethyl-1,3,2-dioxaborolan-2-yl)phenylcarbamate (444 mg, 1.875 mmol) in dioxane (12 mL) was added 2M aq Na₂CO₃ (2.34 mL, 4.68 mmol) and tetrakis(triphenylphosphine)palladium (0) (108 mg, 0.093 mmol). The mixture was flushed thoroughly with argon and heated in an oil bath at 110° C. over night. LC-MS showed formation of the product. After cooled to rt, the reaction mixture was filtered through a Celite plug using methanol as eluent. The filtrate was then concentrated under reduced pressure and the residue obtained was purified by silica gel chromatography eluting with DCM and methanol to afford tert-butyl 4-(6-(2-(4,4-difluoropiperidin-1-yl)ethylamino)pyridin-3-yl)phenylcarbamate (307 mg, 38%). LC-MS (ESI) m/z 433 (M+H)⁺.

Step 3: tert-Butyl 4-(6-(2-(4,4-difluoropiperidin-1-yl)ethylamino)pyridin-3-yl)phenylcarbamate (310 mg, 0.717 mmol) from Step 2 of this example was stirred with a solution of 4N HCl in dioxane (8 mL) for 1 h. The reaction mixture was then concentrated under reduced pressure to afford 5-(4-aminophenyl)-N-(2-(4,4-difluoropiperidin-1-yl)ethyl)pyridin-2-amine hydrochloride which was used for the next step without purification.

Step 4: To a stirred solution of 5-(4-aminophenyl)-N-(2-(4,4-difluoropiperidin-1-yl)ethyl)pyridin-2-amine hydrochloride (200 mg, 0.543 mmol) from Step 3 of this example in DMF (5 mL) was added TEA (0.378 mL, 2.715 mmol) followed by phenyl 5-tert-butylisoxazol-3-ylcarbamate (154.8 mg, 0.597 mmol) (WO2006/82404 A1 (2006/08/10) and DMAP (13.2 mg, 0.108 mmol). The mixture was stirred at rt for over night. LC-MS showed formation of the product. The mixture was partitioned between EtOAc and water. The organic layer was separated, dried over MgSO₄, concentrated under reduced pressure. The residue obtained was purified by silica gel chromatography eluting with dichloromethane and methanol to afford 1-(5-(tert-butyl)isoxazol-3-yl)-3-(4-(6-((2-(4,4-difluoropiperidin-1-yl)ethyl)amino)pyridin-3-yl)phenyl)urea (121 mg, 45%). ¹H NMR (300 MHz, DMSO-d₆) δ 9.50 (s, 1H), 8.85 (s, 1H), 8.28 (d, J=2.3 Hz, 1H), 7.67 (dd, J=2.4, 8.8 Hz, 1H), 7.39-7.58 (m, 4H), 6.43-6.63 (m, 3H), 3.40 (d, J=6.2 Hz, 2H), 2.56 (t, J=6.2 Hz, 6H), 1.85-2.11 (m, 4H), 1.30 (s, 9H). LC-MS (ESI) m/z 499 (M+H)⁺.

Step 5: 1-(2-((5-(4-(3-(5-(tert-butyl)isoxazol-3-yl)ureido)phenyl)pyridin-2-yl)amino)ethyl)-4,4-difluoropiperidin-1-ium methanesulfonate (124.51 mg, 90%) was obtained using a procedure analogous to that described in Step 3 of Example 89, substituting 1-(5-(tert-butyl)isoxazol-3-yl)-3-(4-(6-((2-(4,4-difluoropiperidin-1-yl)ethyl)amino)pyridin-3-yl)phenyl)urea from Step 4 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 9.56 (s, 1H), 8.96 (s, 1H), 8.35 (d, J=2.1 Hz, 1H), 7.82 (dd, J=2.0, 8.8 Hz, 1H), 7.47-7.63 (m, 4H), 7.12 (br s, 1H), 6.71 (d, J=8.7 Hz, 1H), 6.51 (s, 1H), 3.65 (br s, 3H), 3.40 (br s, 5H), 2.37 (s, 4H), 2.31 (br s, 3H), 1.30 (s, 9H). LC-MS (ESI) m/z 499 (M+H)⁺.

### Example 108

### Preparation of 1-(2-(5-(4-(2-(5-tert-butylisoxazol-3-ylamino)-2-oxoethyl)phenyl)pyridin-2-ylamino)ethyl)-4,4-difluoropiperidinium methanesulfonate

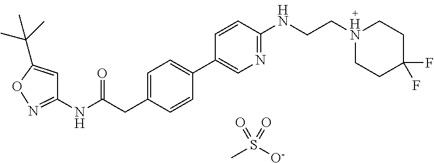

Step 1: 1-(2-(5-(4-(Carboxymethyl)phenyl)pyridin-2-ylamino)ethyl)-4,4-difluoropiperidinium acetate was obtained using a procedure analogous to that described in Step 2 of Example 107, substituting 2-(4-(4,4,5,5-tetramethyl-1,3,2-dioxaborolan-2-yl)phenyl)acetic acid for tert-butyl 4-(4,4,5,5-tetramethyl-1,3,2-dioxaborolan-2-yl)phenylcarbamate used in Example 107. LC-MS showed formation of the product. The mixture was filtered through a celite plug using methanol as eluent. The filtrates were concentrated under reduced pressure. The residue was dissolved in DMF (10 mL) and purified by HPLC using a mixture of water and acetonitrile 10-75% as eluents and diphenyl column as the stationary phase to afford 1-(2-(5-(4-(carboxymethyl)phenyl)pyridin-2-ylamino)ethyl)-4,4-difluoropiperidinium acetate (50 mg, 18%). LC-MS (ESI) m/z 376 (M+H)⁺.

Step 2: N-(5-tert-butylisoxazol-3-yl)-2-(4-(6-(2-(4,4-difluoropiperidin-1-yl)ethylamino)pyridin-3-yl)phenyl)acetamide (20 mg, 15%) was obtained using a procedure analogous to that described in Step 2 of Example 91, substituting 1-(2-(5-(4-(carboxymethyl)phenyl)pyridin-2-ylamino)ethyl)-4,4-difluoropiperidinium acetate from Step 1 of this example for 2-(4-bromophenyl)acetic acid used in Example 91. ¹H NMR (300 MHz, DMSO-d₆) δ 11.20 (s, 1H), 8.29 (d, J=2.3 Hz, 1H), 7.68 (dd, J=2.4, 8.8 Hz, 1H), 7.52 (d, J=8.1 Hz, 2H), 7.34 (d, J=8.3 Hz, 2H), 6.49-6.63 (m, 3H), 3.66 (s, 2H), 3.35 (s, 3H), 2.55 (d, J=6.4 Hz, 5H), 1.85-2.09 (m, 4H), 1.27 (s, 9H). LC-MS (ESI) m/z 498 (M+H)⁺.

Step 3: 1-(2-(5-(4-(2-(5-tert-Butylisoxazol-3-ylamino)-2-oxoethyl)phenyl)pyridin-2-ylamino)ethyl)-4,4-difluoropiperidinium methanesulfonate (20.48 mg, 87%) was obtained using a procedure analogous to that described in Step 3 of Example 89, substituting N-(5-tert-butylisoxazol-3-yl)-2-(4-(6-(2-(4,4-difluoropiperidin-1-yl)ethylamino)pyridin-3-yl)phenyl)acetamide from Step 2 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 11.21 (s, 1H), 8.36 (d, J=2.1 Hz, 1H), 7.82 (d, J=7.5 Hz, 1H), 7.56 (d, J=8.3 Hz, 2H), 7.37 (d, J=8.3 Hz, 2H), 7.13 (br s, 1H), 6.70 (d, J=8.5 Hz, 1H), 6.56 (s, 1H), 3.56-3.81 (m, 4H), 3.20-3.53 (m, 8H), 2.30 (s, 6H), 1.27 (s, 9H). LC-MS (ESI) m/z 498 (M+H)⁺.

### Example 109

### Preparation of 4-(2-(5-(4-(2-oxo-2-(5-(1,1,1-trifluoro-2-methylpropan-2-yl)isoxazol-3-ylamino)ethyl)phenyl)pyridin-2-ylamino)ethyl)morpholin-4-ium methanesulfonate

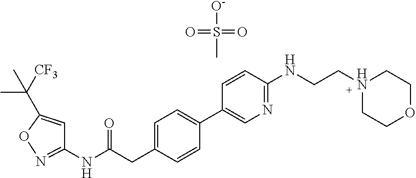

Step 1: 2-(4-(4,4,5,5-Tetramethyl-1,3,2-dioxaborolan-2-yl)phenyl)-N-(5-(1,1,1-trifluoro-2-methylpropan-2-yl)isoxazol-3-yl)acetamide (0.67 g, 45%) was obtained as a white solid using a procedure analogous to that described in Step 1 of Example 85, substituting 5-(1,1,1-trifluoro-2-methylpropan-2-yl)isoxazol-3-amine from Step 1 of Example 35 for 5-tert-butylisoxazol-3-amine used in Example 85. ¹H NMR (300 MHz, DMSO-d₆) δ 11.38 (s, 1H), 7.63 (d, J=7.9 Hz, 2H), 7.33 (d, J=7.7 Hz, 2H), 6.93 (s, 1H), 3.71 (s, 2H), 1.53 (s, 6H), 1.29 (s, 12H). LC-MS (ESI) m/z 439 (M+H)⁺.

Step 2: To a microwave reaction vial was added 2-(4,4,5,5-tetramethyl-1,3,2-dioxaborolan-2-yl)phenyl)-N-(5-(1,1,1-trifluoro-2-methylpropan-2-yl)isoxazol-3-yl)acetamide from Step 1 of this example (350 mg, 0.80 mmol), 5-bromo-N-(2-morpholinoethyl)pyridin-2-amine from Example 96 (343 mg, 1.20 mmol), 2M aq sodium carbonate (1.20 mL, 2.40 mmol), 1,4-dioxane (10 mL), and tetrakis(triphenylphosphine) palladium(0) (92.4 mg, 0.08 mmol). The vial was purged with argon, sealed, and heated at 110° C. for 4 h. The reaction mixture was filtered through filter paper to remove solid impurities, and then the filtrate was concentrated under reduced pressure. The residue was partitioned between EtOAc (50 mL) and water (50 mL), and the aqueous layer was separated and extracted with EtOAc (2×30 mL). The combined organic layers were washed with brine (2×30 mL), dried over MgSO₄, filtered and concentrated under reduced pressure. The crude product was purified by silica gel chromatography, eluting with 0-10% methanol in dichloromethane to give 2-(4-(6-(2-morpholinoethylamino)pyridin-3-yl)phenyl)-N-(5-(1,1,1-trifluoro-2-methylpropan-2-yl)isoxazol-3-yl)acetamide (180 mg, 44%) as a light brown solid. ¹H NMR (300 MHz, DMSO-d₆) δ 11.38 (s, 1H), 8.29 (d, J=2.3 Hz, 1H), 7.68 (dd, J=2.4, 8.7 Hz, 1H), 7.52 (d, J=8.3 Hz, 2H), 7.34 (d, J=8.1 Hz, 2H), 6.94 (s, 1H), 6.47-6.63 (m, 2H), 3.69 (s, 2H), 3.54-3.64 (m, 4H), 3.34-3.48 (m, 3H), 2.37-2.47 (m, 4H), 1.53 (s, 6H). LC-MS (ESI) m/z 518 (M+H)⁺.

Step 3: A mixture of 2-(4-(6-(2-morpholinoethylamino)pyridin-3-yl)phenyl)-N-(5-(1,1,1-trifluoro-2-methylpropan-2-yl)isoxazol-3-yl)acetamide from this example (180 mg, 0.35 mmol) and methanesulfonic acid (33.5 mg, 0.35 mmol) in ethanol (20 mL) was stirred at 60° C. for 2 h. The solvent was evaporated under reduced pressure. Water was added and the mixture was frozen and lyophilized to give 4-(2-(5-(4-(2-oxo-2-(5-(1,1,1-trifluoro-2-methylpropan-2-yl)isoxazol-3-ylamino)ethyl)phenyl)pyridin-2-ylamino)ethyl)morpholin-4-ium methanesulfonate (200 mg, 94%) as a light brown solid. ¹H NMR (300 MHz, DMSO-d₆) δ 11.39 (s, 1H), 8.37 (s, 1H), 7.80 (d, J=8.7 Hz, 1H), 7.56 (d, J=7.9 Hz, 2H), 7.37 (d, J=8.1 Hz, 2H), 7.07 (br s, 1H), 6.93 (s, 1H), 6.68 (d, J=8.5 Hz, 1H), 3.85 (br s, 4H), 3.71 (s, 2H), 3.59-3.68 (m, 2H), 3.12-3.47 (m, 6H), 2.31 (s, 3H), 1.53 (s, 6H). LC-MS (ESI) m/z 518 (M+H)⁺.

### Example 110

### Preparation of 5-(4-(2-(5-tert-butylisoxazol-3-ylamino)-2-oxoethyl)phenyl)-N-methylpyridin-2-aminium methanesulfonate

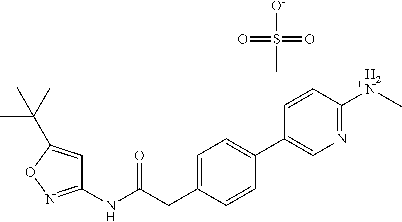

Step 1: To a microwave reaction vial was added N-(5-tert-butylisoxazol-3-yl)-2-(4-(4,4,5,5-tetramethyl-1,3,2-dioxaborolan-2-yl)phenyl)acetamide from Step 1 of Example 85 (350 mg, 0.91 mmol), 5-bromo-N-methylpyridin-2-amine (256 mg, 1.37 mmol), 2M aq sodium carbonate (1.37 mL, 2.73 mmol), acetonitrile (10 mL), and [1,1′-bis(diphenylphosphino)ferrocene]palladium(II) dichloride dichloromethane adduct (74.3 mg, 0.091 mmol). The vial was purged with argon, sealed, and heated in a microwave reactor at 150° C. for 15 min. The mixture was partitioned between EtOAc (50 mL) and water (50 mL), and the aqueous layer was separated and extracted with EtOAc (2×40 mL). The combined organic layers were washed with brine (2×30 mL), dried over MgSO₄, filtered and concentrated under reduced pressure. The crude product was purified by silica gel chromatography, eluting with 0-70% EtOAc in hexanes to give N-(5-tert-butylisoxazol-3-yl)-2-(4-(6-(methylamino)pyridin-3-yl)phenyl)acetamide (165 mg, 50%) as an off-white solid. ¹H NMR (300 MHz, DMSO-d₆) δ 11.20 (s, 1H), 8.30 (d, J=2.3 Hz, 1H), 7.69 (dd, J=2.4, 8.7 Hz, 1H), 7.52 (d, J=8.1 Hz, 2H), 7.34 (d, J=8.3 Hz, 2H), 6.61 (d, J=4.7 Hz, 1H), 6.57 (s, 1H), 6.52 (d, J=8.7 Hz, 1H), 3.66 (s, 2H), 2.80 (d, J=4.9 Hz, 3H), 1.27 (s, 9H). LC-MS (ESI) m/z 365 (M+H)⁺.

Step 2: A mixture of N-(5-tert-butylisoxazol-3-yl)-2-(4-(6-(methylamino)pyridin-3-yl)phenyl)acetamide from Step 1 of this example (165 mg, 0.45 mmol) and methanesulfonic acid (43.6 mg, 0.45 mmol) in ethanol (20 mL) was stirred at 60° C. for 2 h. The solvent was evaporated under reduced pressure. Water was added and the mixture was frozen and lyophilized to give 5-(4-(2-(5-tert-butylisoxazol-3-ylamino)-2-oxoethyl)phenyl)-N-methylpyridin-2-aminium methanesulfonate (185 mg, 89%) as an off-white solid. ¹H NMR (300 MHz, DMSO-d₆) δ 11.23 (s, 1H), 8.63 (br s, 1H), 8.21 (d, J=9.2 Hz, 1H), 8.17 (s, 1H), 7.63 (d, J=8.3 Hz, 2H), 7.43 (d, J=8.1 Hz, 2H), 7.08 (d, J=9.2 Hz, 1H), 6.56 (s, 1H), 3.72 (s, 2H), 2.97 (d, J=3.2 Hz, 3H), 2.32 (s, 3H), 1.27 (s, 9H). LC-MS (ESI) m/z 365 (M+H)⁺.

### Example 111

### Preparation of 5-(4-(2-(5-tert-butylisoxazol-3-ylamino)-2-oxoethyl)phenyl)-N-ethylpyridin-2-aminium methanesulfonate

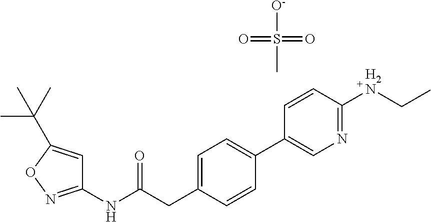

5-(4-(2-(5-tert-Butylisoxazol-3-ylamino)-2-oxoethyl)phenyl)-N-ethylpyridin-2-aminium methanesulfonate (140 mg, 33%) was obtained as an off-white solid using procedures analogous to those described in Steps 1-2 of Example 110, substituting 5-bromo-N-ethylpyridin-2-amine for 5-bromo-N-methylpyridin-2-amine used in Example 110. ¹H NMR (300 MHz, DMSO-d₆) δ 11.23 (s, 1H), 8.64 (br s, 1H), 8.21 (dd, J=1.8, 9.3 Hz, 1H), 8.15 (s, 1H), 7.63 (d, J=8.1 Hz, 2H), 7.43 (d, J=8.1 Hz, 2H), 7.09 (d, J=9.2 Hz, 1H), 6.56 (s, 1H), 3.72 (s, 2H), 3.36-3.42 (m, 2H), 2.33 (s, 3H), 1.27 (s, 9H), 1.19-1.26 (m, 3H). LC-MS (ESI) m/z 379 (M+H)⁺.

### Example 112

### Preparation of 5-(4-(3-(5-(2,2-difluoro-1-methylcyclopropyl)isoxazol-3-yl)ureido)phenyl)pyridin-2-aminium methanesulfonate

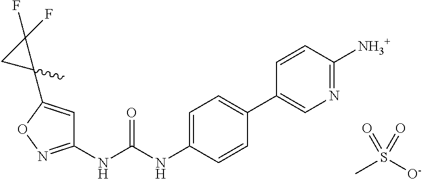

Step 1: 4-Chlorophenyl (5-(2,2-difluoro-1-methylcyclopropyl)isoxazol-3-yl)carbamate (115 mg, 41%) was prepared using a procedure analogous to that described in Step 3 of Example 32, substituting 5-(2,2-difluoro-1-methylcyclopropyl)isoxazol-3-amine from Step 1 of Example 102 for 3-(2-fluoropropan-2-yl)isoxazol-5-amine, and 4-chlorophenyl carbonochloridate for phenyl carbonochloridate used in Example 32.

Step 2: 5-(4-(3-(5-(2,2-Difluoro-1-methylcyclopropyl)isoxazol-3-yl)ureido)phenyl)pyridin-2-aminium methanesulfonate (68 mg, 40%) was prepared using a procedure analogous to that described in described in Step 4 of Example 36, substituting 4-chlorophenyl (5-(2,2-difluoro-1-methylcyclopropyl)isoxazol-3-yl)carbamate from Step 1 of this example for phenyl 5-(1-(trifluoromethyl)cyclopropyl)isoxazol-3-ylcarbamate, and 5-(4-aminophenyl)pyridin-2-amine for 5-(4-aminophenyl)-N-(2-morpholinoethyl)pyridin-2-amine hydrochloride used in Example 36. ¹H NMR (300 MHz, DMSO-d₆) δ 10.44 (s, 1H), 9.20 (s, 1H), 8.31 (dd, J=1.9, 9.2 Hz, 1H), 8.24 (s, 1H), 8.00 (br s, 2H), 7.45-7.69 (m, 4H), 7.07 (d, J=9.2 Hz, 1H), 6.11 (s, 1H), 2.38 (s, 3H), 2.14 (ddd, J=5.4, 8.2, 13.7 Hz, 1H), 1.70-1.93 (m, 1H), 1.52 (s, 3H). LC-MS (ESI) m/z 386 (M+H)⁺.

### Example 113

### Preparation of 5-(4-(2-(5-tert-butylisoxazol-3-ylamino)-2-oxoethyl)phenyl)-3-methylpyrazin-2-aminium methanesulfonate

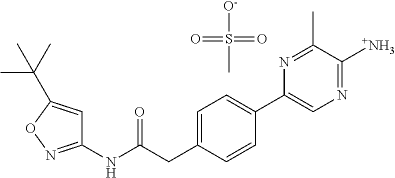

5-(4-(2-(5-tert-Butylisoxazol-3-ylamino)-2-oxoethyl)phenyl)-3-methylpyrazin-2-aminium methanesulfonate (148 mg, 49%) was obtained as light yellow solid using procedures analogous to those described in Steps 1-2 of Example 110, substituting 5-bromo-3-methylpyrazin-2-amine for 5-bromo-N-methylpyridin-2-amine used in Example 110. ¹H NMR (300 MHz, DMSO-d₆) δ 11.22 (br s, 1H), 8.33 (s, 1H), 7.89 (d, J=8.1 Hz, 2H), 7.38 (d, J=8.1 Hz, 2H), 6.56 (s, 1H), 3.70 (br s, 2H), 2.45 (s, 3H), 2.35 (s, 3H), 1.27 (s, 9H). LC-MS (ESI) m/z 366 (M+H)⁺.

### Example 114

### Preparation of 5-(4-(2-(5-tert-butylisoxazol-3-ylamino)-2-oxoethyl)phenyl)-3-carbamoylpyrazin-2-aminium methanesulfonate

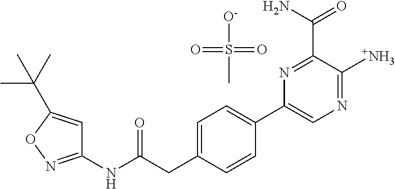

5-(4-(2-(5-tert-Butylisoxazol-3-ylamino)-2-oxoethyl)phenyl)-3-carbamoylpyrazin-2-aminium methanesulfonate (73 mg, 17%) was obtained as a brown solid using procedures analogous to those described in Steps 1-2 of Example 110, substituting 5-bromo-3-methylpyrazin-2-amine for 5-bromo-N-methylpyridin-2-amine used in Example 110. ¹H NMR (300 MHz, DMSO-d₆) δ 11.21 (br s, 1H), 8.82 (s, 1H), 8.28 (br s, 2H), 8.10 (d, J=8.1 Hz, 2H), 7.70 (br s, 2H), 7.38 (d, J=7.9 Hz, 2H), 6.57 (s, 1H), 3.70 (br s, 2H), 2.34 (s, 3H), 1.27 (s, 9H). LC-MS (ESI) m/z 395 (M+H)⁺.

### Example 115

### Preparation of 5-(4-(2-(5-tert-butylisoxazol-3-ylamino)-2-oxoethyl)phenyl)-3-chloropyridin-2-aminium methanesulfonate

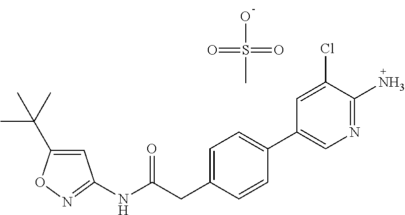

Step 1: 5-Bromo-3-chloropyridin-2-amine (824 mg, 51%) was obtained as an off-white solid using a procedure analogous to that described in Step 1 of Example 94, substituting 3-chloropyridin-2-amine for 3-fluoropyridin-2-amine used in Example 94. ¹H NMR (300 MHz, DMSO-d₆) δ 7.99 (d, J=1.9 Hz, 1H), 7.85 (d, J=2.1 Hz, 1H), 6.55 (br s, 2H). LC-MS (ESI) m/z 207, 209 and 211 (M+H)⁺.

Step 2: 5-(4-(2-(5-tert-Butylisoxazol-3-ylamino)-2-oxoethyl)phenyl)-3-chloropyrazin-2-aminium methanesulfonate (100 mg, 27%) was obtained as a pink solid using procedures analogous to those described in Steps 1-2 of Example 110, substituting 5-bromo-3-chloropyridin-2-amine for 5-bromo-N-methylpyridin-2-amine used in Example 110. ¹H NMR (300 MHz, DMSO-d₆) δ 11.22 (br s, 1H), 8.29 (d, J=7.9 Hz, 2H), 7.64 (d, J=8.1 Hz, 2H), 7.39 (d, J=8.1 Hz, 2H), 6.56 (s, 1H), 3.70 (s, 2H), 2.34 (s, 3H), 1.27 (s, 9H). LC-MS (ESI) m/z 385 (M+H)⁺.

### Example 116

### Preparation of 5-(4-(2-(5-tert-butylisoxazol-3-ylamino)-2-oxoethyl)phenyl)-3-(trifluoromethyl)pyridin-2-aminium methanesulfonate

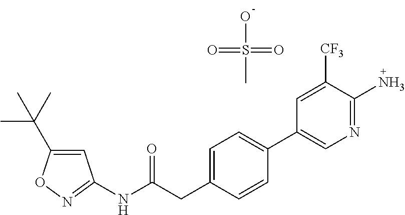

Step 1: 5-Bromo-3-(trifluoromethyl)pyridin-2-amine (766 mg, 52%) was obtained as a white solid using a procedure analogous to that described in Step 1 of Example 94, substuting 3-(trifluoromethyl)pyridin-2-amine for 3-fluoropyridin-2-amine used in Example 94. ¹H NMR (300 MHz, DMSO-d₆) δ 8.28 (s, 1H), 7.92 (s, 1H), 6.72 (br s, 2H). LC-MS (ESI) m/z 241 and 243 (M+H)⁺.

Step 2: 5-(4-(2-(5-tert-Butylisoxazol-3-ylamino)-2-oxoethyl)phenyl)-3-(trifluoromethyl)pyridin-2-aminium methanesulfonate (150 mg, 27%) was obtained as an orange-pink solid using procedures analogous to those described in Steps 1-2 of Example 110, substituting 5-bromo-3-(trifluoromethyl)pyridin-2-amine for 5-bromo-N-methylpyridin-2-amine used in Example 110. ¹H NMR (300 MHz, DMSO-d₆) δ 11.21 (br s, 1H), 8.54 (br s, 1H), 8.11 (br s, 1H), 7.63 (d, J=8.1 Hz, 2H), 7.33-7.47 (m, 2H), 6.56 (s, 1H), 3.69 (s, 2H), 2.34 (s, 3H), 1.27 (s, 9H). LC-MS (ESI) m/z 419 (M+H)⁺.

### Example 117

### Preparation of N-(5-tert-butylisoxazol-3-yl)-2-(4-(6-(2-(1,2,2,6,6-pentamethylpiperidin-4-ylidene)ethylamino)pyridin-3-yl)phenyl)acetamide

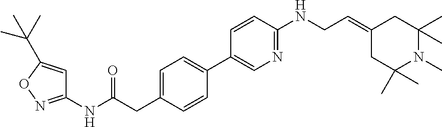 [SMILES: CN1C(C)(C)CC(=CCNc2ccc(-c3ccc(CC(=O)Nc4cc(C(C)(C)C)on4)cc3)cn2)CC1(C)C]

Step 1: 5-Bromo-N-(2-(1,2,2,6,6-pentamethylpiperidin-4-ylidene)ethyl)pyridin-2-amine (270 mg, 75%) was obtained using a procedure analogous to that described in Step 1 of Example 89, substituting 2-(1,2,2,6,6-pentamethylpiperidin-4-ylidene)ethanamine (K. R. Dahnke, et. al. US 2008/0306082, 2008) for 2-methoxyethanamine used in Example 89. LC-MS (ESI) m/z 320, 322 (M+H)⁺.

Step 2: 4-(2-(5-(4-(Carboxymethyl)phenyl)pyridin-2-ylamino)ethylidene)-1,2,2,6,6-pentamethylpiperidinium acetate (70 mg, 24%) was obtained using a procedure analogous to that described in Step 2 of Example 107, substituting 2-(4-(4,4,5,5-tetramethyl-1,3,2-dioxaborolan-2-yl)phenyl)acetic acid for tert-butyl 4-(4,4,5,5-tetramethyl-1,3,2-dioxaborolan-2-yl)phenylcarbamate, and 5-bromo-N-(2-(1,2,2,6,6-pentamethylpiperidin-4-ylidene)ethyl)pyridin-2-amine from Step 1 of this example for 5-bromo-N-(2-(4,4-difluoropiperidin-1-yl)ethyl)pyridin-2-amine used in Example 107. LC-MS (ESI) m/z 408 (M+H)⁺.

Step 3: N-(5-tert-Butylisoxazol-3-yl)-2-(4-(6-(2-(1,2,2,6,6-pentamethylpiperidin-4-ylidene)ethylamino)pyridin-3-yl)phenyl)acetamide (14 mg, 16%) was obtained using a procedure analogous to that described in Step 2 of Example 91, substituting 4-(2-(5-(4-(carboxymethyl)phenyl)pyridin-2-ylamino)ethylidene)-1,2,2,6,6-pentamethylpiperidinium acetate from Step 2 of this example for 2-(4-bromophenyl)acetic acid used in Example 91. ¹H NMR (300 MHz, DMSO-d₆) δ 11.20 (s, 1H), 8.29 (d, J=2.3 Hz, 1H), 7.67 (dd, J=2.4, 8.8 Hz, 1H), 7.51 (d, J=8.1 Hz, 2H), 7.34 (d, J=8.3 Hz, 2H), 6.68 (t, J=5.2 Hz, 1H), 6.48-6.61 (m, 2H), 5.36 (t, J=6.4 Hz, 1H), 3.91 (t, J=5.8 Hz, 2H), 3.67 (s, 2H), 2.10-2.22 (m, 5H), 2.00 (s, 2H), 1.18-1.37 (m, 9H), 1.00 (d, J=8.3 Hz, 12H). LC-MS (ESI) m/z 530 (M+H)⁺.

### Example 118

### Preparation of N-(3-(2-fluoropropan-2-yl)isoxazol-5-yl)-2-(4-(6-(2-morpholinoethylamino)pyridin-3-yl)phenyl)acetamide

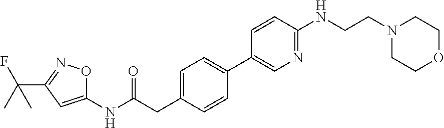 [SMILES: CC(C)(F)c1cc(NC(=O)Cc2ccc(-c3ccc(NCCN4CCOCC4)nc3)cc2)on1]

Step 1: N-(2-Morpholinoethyl)-5-(4,4,5,5-tetramethyl-1,3,2-dioxaborolan-2-yl)pyridin-2-amine (1.81 g, 71%) was synthesized as a brown solid using the procedure analogous to that described in Step 1 of Example 77, substituting 5-bromo-N-(2-morpholinoethyl)pyridin-2-amine from Step 1 of Example 7 for 5-bromopyrimidin-2-amine used in Example 77. LC-MS (ESI) m/z 334 (M+H)⁺.

Step 2: N-(3-(2-fluoropropan-2-yl)isoxazol-5-yl)-2-(4-(6-(2-morpholinoethylamino)pyridin-3-yl)phenyl)acetamide (9.8 mg, 1.8%) was synthesized as a solid according to the procedure described in Step 2 of Example 40, substituting N-(2-morpholinoethyl)-5-(4,4,5,5-tetramethyl-1,3,2-dioxaborolan-2-yl)pyridin-2-amine from Step 1 of this example for 5-(4,4,5,5-tetramethyl-1,3,2-dioxaborolan-2-yl)pyridin-2-amine, and 2-(4-bromophenyl)-N-(3-(2-fluoropropan-2-yl)isoxazol-5-yl)acetamide from Step 1 of Example 101 for 5-bromo-N-tritylprydin-2-amine used in Example 40. ¹H NMR (300 MHz, DMSO-d₆) δ 8.29 (d, J=2.3 Hz, 1H), 7.68 (dd, J=2.4, 8.7 Hz, 1H), 7.52 (d, J=8.3 Hz, 2H), 7.33 (d, J=8.1 Hz, 2H), 6.49-6.64 (m, 2H), 6.28 (s, 1H), 3.70 (s, 2H), 3.54-3.65 (m, 4H), 3.40 (br, 4H), 2.42 (m, 4H), 1.57-1.73 (m, 6H). LC-MS (ESI) m/z 468 (M+H)⁺.

### Example 119

### Preparation of 5-(4-(2-(3-tert-butylisoxazol-5-ylamino)-2-oxoethyl)phenyl)-6-fluoropyridin-2-aminium methanesulfonate

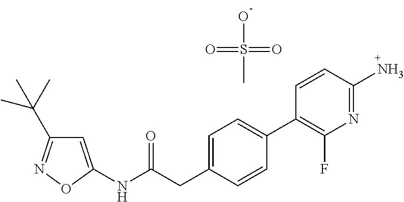

Step 1: N-(3-tert-Butylisoxazol-5-yl)-2-(4-(4,4,5,5-tetramethyl-1,3,2-dioxaborolan-2-yl)phenyl)acetamide (126 mg, 12%) was obtained as a faint yellow solid using a procedure analogous to that described in Step 1 of Example 85, substituting 3-tert-butylisoxazol-5-amine for 5-tert-butylisoxazol-3-amine used in Example 85. LC-MS (ESI) m/z 385 (M+H)⁺.

Step 2: 5-(4-(2-(3-tert-Butylisoxazol-5-ylamino)-2-oxoethyl)phenyl)-6-fluoropyridin-2-aminium methanesulfonate (19 mg, 36%) was obtained as a sticky red solid using procedures analogous to those described in Steps 1-2 of Example 110, substituting 5-bromo-6-fluoropyridin-2-amine from Example 95 for 5-bromo-N-methylpyridin-2-amine used in Example 110. ¹H NMR (300 MHz, DMSO-d₆) δ 11.79 (s, 1H), 7.66 (dd, J=8.3, 10.7 Hz, 1H), 7.39-7.46 (m, 2H), 7.30-7.38 (m, 2H), 6.41 (dd, J=1.8, 8.2 Hz, 1H), 6.20 (s, 1H), 3.70 (s, 2H), 2.33 (s, 3H), 1.23 (s, 9H). LC-MS (ESI) m/z 369 (M+H)⁺.

### Example 120

### Preparation of 6-(4-(2-((5-(tert-butyl)isoxazol-3-yl)amino)-2-oxoethyl)phenyl)-1,2,3,4-tetrahydro-1,8-naphthyridin-1-ium methanesulfonate

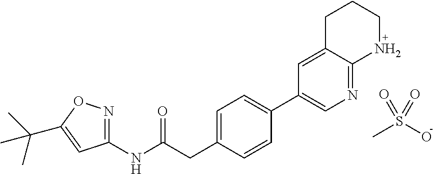

6-(4-(2-((5-(tert-Butyl)isoxazol-3-yl)amino)-2-oxoethyl)phenyl)-1,2,3,4-tetrahydro-1,8-naphthyridin-1-ium methanesulfonate (222 mg, 58%) was prepared using procedures analogous to those described in Steps 2-3 of Example 83, substituting N-(5-(tert-butyl)isoxazol-3-yl)-2-(4-(4,4,5,5-tetramethyl-1,3,2-dioxaborolan-2-yl)phenyl)acetamide for 5-(4,4,5,5-tetramethyl-1,3,2-dioxaborolan-2-yl)pyridin-2-amine, and 6-iodo-1,2,3,4-tetrahydro-1,8-naphthyridine for 2-(4-bromophenyl)-N-(3-(1-hydroxy-2-methylpropan-2-yl)isoxazol-5-yl)acetamide used in Example 83. ¹H NMR (300 MHz, DMSO-d₆) δ 11.23 (s, 1H), 8.31 (br s, 1H), 8.11 (d, J=5.5 Hz, 2H), 7.63 (d, J=8.3 Hz, 2H), 7.42 (d, J=8.1 Hz, 2H), 6.56 (s, 1H), 3.71 (s, 2H), 3.45 (br s, 2H), 2.87 (t, J=5.7 Hz, 2H), 2.35 (s, 3H), 1.74-1.98 (m, 2H), 1.27 (s, 9H). LC-MS (ESI) m/z 386 (M+H)⁺.

### Example 121

### Preparation of 5-(4-(3-(5-(1-(trifluoromethyl)cyclobutyl)isoxazol-3-yl)ureido)phenyl)pyridin-2-aminium methanesulfonate

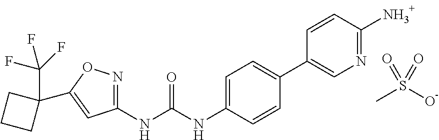

Step 1: 5-(1-(Trifluoromethyl)cyclobutyl)isoxazol-3-amine (1.9 g, 90%) was prepared using procedures analogous to those described in Steps 2-3 of Example 98, substituting ethyl 1-(trifluoromethyl)cyclobutanecarboxylate for benzyl 3-methyloxetane-3-carboxylate used in Example 98. LC-MS (ESI) m/z 207 (M+H)⁺.

Step 2: 4-Chlorophenyl (5-(1-(trifluoromethyl)cyclobutyl)isoxazol-3-yl)carbamate (252 mg, 72%) was prepared using a procedure analogous to that described in Step 3 of Example 32, substituting 5-(1-(trifluoromethyl)cyclobutyl)isoxazol-3-amine from Step 1 of this example for 3-(2-fluoropropan-2-yl)isoxazol-5-amine, and 4-chlorophenyl carbonochloridate for phenyl carbonochloridate used in Example 32.

Step 3: 5-(4-(3-(5-(1-(Trifluoromethyl)cyclobutyl)isoxazol-3-yl)ureido)phenyl)pyridin-2-aminium methanesulfonate (135 mg, 40%) was prepared using a procedure analogous to that described in described in Step 4 of Example 36, substituting 4-chlorophenyl (5-(1-(trifluoromethyl)cyclobutyl)isoxazol-3-yl)carbamate from Step 2 of this example for phenyl 5-(1-(trifluoromethyl)cyclopropyl)isoxazol-3-ylcarbamate, and 5-(4-aminophenyl)pyridin-2-amine for 5-(4-aminophenyl)-N-(2-morpholinoethyl)pyridin-2-amine hydrochloride used in Example 36. ¹H NMR (300 MHz, DMSO-d₆) δ 10.56 (s, 1H), 9.21 (s, 1H), 8.30 (dd, J=2.1, 9.4 Hz, 1H), 8.24 (s, 1H), 7.96 (br s, 1H), 7.51-7.71 (m, 4H), 7.06 (d, J=9.2 Hz, 1H), 6.12 (s, 1H), 3.45 (br s, 1H), 2.53-2.68 (m, 4H), 2.37 (s, 3H), 1.93-2.18 (m, 2H). LC-MS (ESI) m/z 418 (M+H)⁺.

### Example 122

### Preparation of N-(5-tert-butylisoxazol-3-yl)-2-(4-(6-(2-(3-methyloxetan-3-yl)ethylamino)pyridin-3-yl)phenyl)acetamide

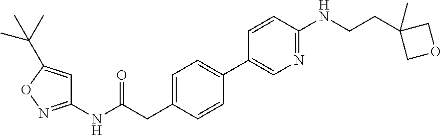 [SMILES: CC1(CCNc2ccc(-c3ccc(CC(=O)Nc4cc(C(C)(C)C)on4)cc3)cn2)COC1]

Step 1: To a stirred solution of 3-(bromomethyl)-3-methyloxetane (500 mg, 3.029 mmol) in ethanol (8 mL) was added sodium cyanide (170 mg, 3.48 mmol). The resulting mixture was heated at 80° C. overnight. The reaction mixture was filtered through a Celite plug using dichloromethane as eluent. The filtrates were evaporated under reduced pressure to afford 2-(3-methyloxetan-3-yl)acetonitrile (335 mg, 99%). ¹H NMR (300 MHz, CHLOROFORM-d) δ 4.39-4.60 (m, 4H), 2.73 (s, 2H), 1.49 (s, 3H).

Step 2: To a stirred solution of 2-(3-methyloxetan-3-yl)acetonitrile (320 mg, 2.88 mmol) in 7 N ammonia in methanol (10 mL) was added 10% Pd/C (64 mg) and PtO₂ (64 mg). The reaction mixture was stirred under a H₂ atmosphere at 50 Psi overnight. The reaction mixture was filtered through a Celite plug and the filtrates were evaporated under reduced pressure. The crude product was used for the next step.

Step 3: 5-Bromo-N-(2-(3-methyloxetan-3-yl)ethyl)pyridin-2-amine (83 mg, 18%) was obtained using a procedure analogous to that described in Step 1 of Example 89, substituting 2-(3-methyloxetan-3-yl)ethanamine from Step 2 of this example for 2-methoxyethanamine used in Example 89. LC-MS (ESI) m/z 271, 273 (M+H)⁺.

Step 4: N-(5-tert-butylisoxazol-3-yl)-2-(4-(6-(2-(3-methyloxetan-3-yl)ethylamino)pyridin-3-yl)phenyl)acetamide (3.48 mg, 8%) was obtained using a procedure analogous to that described in Step 2 of Example 107, substituting 5-bromo-N-(2-(3-methyloxetan-3-yl)ethyl)pyridin-2-amine from Step 2 of this example for 5-bromo-N-(2-(4,4-difluoropiperidin-1-yl)ethyl)pyridin-2-amine, and N-(5-tert-butylisoxazol-3-yl)-2-(4-(4,4,5,5-tetramethyl-1,3,2-dioxaborolan-2-yl)phenyl)acetamide from Step 1 of Example 85 for tert-butyl 4-(4,4,5,5-tetramethyl-1,3,2-dioxaborolan-2-yl)phenylcarbamate used in Example 107. ¹H NMR (300 MHz, DMSO-d₆) δ 11.22 (br s, 1H), 8.38 (d, J=2.3 Hz, 1H), 7.78 (dd, J=2.4, 8.9 Hz, 1H), 7.54 (d, J=8.1 Hz, 2H), 7.34 (d, J=8.1 Hz, 2H), 6.57 (s, 1H), 6.48 (d, J=8.7 Hz, 1H), 4.82 (br s, 1H), 3.67 (s, 2H), 3.48 (t, J=7.1 Hz, 2H), 3.24-3.44 (m, 2H), 3.09 (d, J=10.4 Hz, 1H), 1.85-2.03 (m, 1H), 1.51-1.75 (m, 2H), 1.27 (s, 9H), 1.07 (s, 3H). LC-MS (ESI) m/z 449 (M+H)⁺.

### Example 123

### Preparation of 5-(4-(2-(5-tert-butylisoxazol-3-ylamino)-2-oxoethyl)-3,5-difluorophenyl)pyridin-2-aminium methanesulfonate

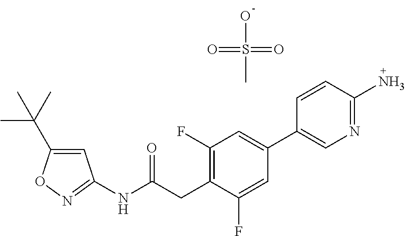

Step 1: 2-(4-Bromo-2,6-difluorophenyl)-N-(5-tert-butylisoxazol-3-yl)acetamide (2.9 g, 52%) was obtained as an off-white solid using a procedure analogous to that described in Step 1 of Example 85, substituting 2-(4-bromo-2,6-difluorophenyl)acetic acid (Crowley, Patrick, Jelf, et al. WO2005/123698; 2005) for 2-(4-(4,4,5,5-tetramethyl-1,3,2-dioxaborolan-2-yl)phenyl)acetic acid used in Example 85. ¹H NMR (300 MHz, DMSO-d₆) δ 11.34 (s, 1H), 7.49 (d, J=7.0 Hz, 2H), 6.54 (s, 1H), 3.77 (s, 2H), 1.27 (s, 9H). LC-MS (ESI) m/z 373 and 375 (M+H)⁺.

Step 2: 5-(4-(2-(5-tert-Butylisoxazol-3-ylamino)-2-oxoethyl)-3,5-difluorophenyl)pyridin-2-aminium methanesulfonate (100 mg, 36%) was obtained as a pink solid using procedures analogous to those described in Steps 1-2 of Example 110, substituting 2-(4-bromo-2,6-difluorophenyl)-N-(5-tert-butylisoxazol-3-yl)acetamide from Step 1 of this example for N-(5-tert-butylisoxazol-3-yl)-2-(4-(4,4,5,5-tetramethyl-1,3,2-dioxaborolan-2-yl)phenyl)acetamide, and 5-(4,4,5,5-tetramethyl-1,3,2-dioxaborolan-2-yl)pyridin-2-amine for 5-bromo-N-methylpyridin-2-amine used in Example 110. ¹H NMR (300 MHz, DMSO-d₆) δ 11.35 (s, 1H), 8.39 (d, J=1.7 Hz, 1H), 8.26-8.34 (m, 1H), 7.91 (br s, 2H), 7.52 (d, J=8.7 Hz, 2H), 7.01 (d, J=9.2 Hz, 1H), 6.54 (s, 1H), 3.83 (s, 2H), 2.33 (s, 3H), 1.28 (s, 9H). LC-MS (ESI) m/z 387 (M+H)⁺.

### Example 124

### Preparation of 2-(4-(6-aminopyridin-3-yl)phenyl)-N-(5-(1-(trifluoromethyl)cyclobutyl)isoxazol-3-yl)acetamide

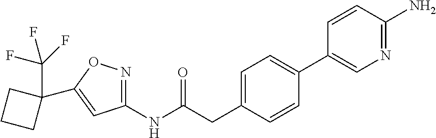 [SMILES: Nc1ccc(-c2ccc(CC(=O)Nc3cc(C4(C(F)(F)F)CCC4)on3)cc2)cn1]

Step 1: To a stirred solution of 5-(1-(trifluoromethyl)cyclobutyl)isoxazol-3-amine (885 mg, 4.3 mmol) from Step 1 of Example 121 in 10 mL of DMF was added 2-(4-bromophenyl)acetic acid (1.11 g, 5.2 mmol), HATU (2.12 g, 5.6 mmol), and DIEA (1.49 mL, 8.6 mmol). The resulting mixture was then heated at 60° C. for 1 h. LC-MS indicated the presence of product. After cooled to rt, the reaction mixture was diluted with sat. NaHCO₃ and extracted with EtOAc. The organic layer was then washed with sat. NaHCO₃ and brine, dried over Na₂SO₄ MgSO₄, filtered, and concentrated under reduced pressure. The residue was purified by silica gel flash chromatography, eluting with 0-35% EtOAc in hexanes, to give 2-(4-bromophenyl)-N-(5-(1-(trifluoromethyl)cyclobutyl)isoxazol-3-yl)acetamide (175 mg, 10%) as an oil. LC-MS (ESI) m/z 403, 405 (M+H)⁺.

Step 2: 2-(4-(6-Aminopyridin-3-yl)phenyl)-N-(5-(1-(trifluoromethyl)cyclobutyl)isoxazol-3-yl)acetamide (4.3 mg, 2.4%) was prepared using a procedure analogous to that described in Step 2 of Example 83, substituting 2-(4-bromophenyl)-N-(5-(1-(trifluoromethyl)cyclobutyl)isoxazol-3-yl)acetamide from Step 1 of this example for 2-(4-bromophenyl)-N-(3-(1-hydroxy-2-methylpropan-2-yl)isoxazol-5-yl)acetamide used in Example 83. ¹H NMR (300 MHz, METHANOL-d₄) δ 8.11 (br s, 1H), 7.77 (d, J=8.3 Hz, 1H), 7.52 (d, J=7.2 Hz, 2H), 7.41 (d, J=7.3 Hz, 2H), 6.69 (d, J=7.7 Hz, 1H), 3.77 (br s, 2H), 2.18-2.81 (m, 4H), 1.78-2.16 (m, 2H). LC-MS (ESI) m/z 417 (M+H)⁺.

### Example 125

### Preparation of 5-(4-(2-(5-tert-butylisoxazol-3-ylamino)-2-oxoethyl)-2-fluorophenyl)pyridin-2-aminium methanesulfonate

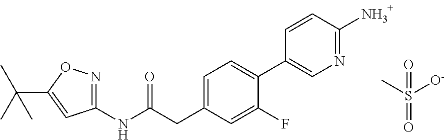

Step 1: N-(5-tert-butylisoxazol-3-yl)-2-(4-chloro-3-fluorophenyl)acetamide (1.3 g, 50%) was synthesized as a white solid using the procedure analogous to that described in Step 1 of Example 18, substituting 2-(4-chloro-3-fluorophenyl)acetic acid for 2-(4-bromophenyl)acetic acid used in Example 18. LC-MS (ESI) m/z 311 (M+H)⁺.

Step 2: 2-(4-(6-Aminopyridin-3-yl)-3-fluorophenyl)-N-(5-tert-butylisoxazol-3-yl)acetamide (161 mg, 10%) was synthesized as a solid according to the procedure described in Step 2 of Example 71, substituting N-(5-tert-butylisoxazol-3-yl)-2-(4-chloro-3-fluorophenyl)acetamide from Step 1 of this example for N-(5-tert-butylisoxazol-3-yl)-2-(4-chlorophenyl)propanamide used in Example 71. LC-MS (ESI) m/z 369 (M+H)⁺.

Step 3: 5-(4-(2-(5-tert-Butylisoxazol-3-ylamino)-2-oxoethyl)-2-fluorophenyl)pyridin-2-aminium methanesulfonate (203.8 mg, 100%) was synthesized as a white solid using the procedure analogous to that described in Step 3 of Example 89, substituting 2-(4-(6-aminopyridin-3-yl)-3-fluorophenyl)-N-(5-tert-butylisoxazol-3-yl)acetamide from Step 2 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 11.25 (s, 1H), 8.06-8.21 (m, 2H), 7.97 (br s, 2H), 7.53 (t, J=8.2 Hz, 1H), 7.21-7.38 (m, 2H), 7.04 (d, J=9.2 Hz, 2H), 6.56 (s, 1H), 3.75 (s, 2H), 2.30 (s, 3H), 1.27 (s, 9H). LC-MS (ESI) m/z 369 (M+H)⁺.

### Example 126

### Preparation of 5-(4-(2-oxo-2-(4-(trifluoromethyl)-1H-pyrazol-1-ylamino)ethyl)phenyl)pyridin-2-aminium methanesulfonate

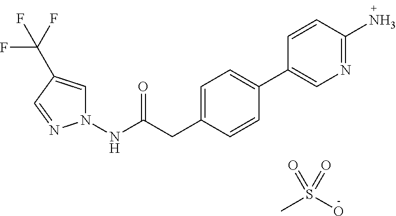

Step 1: 4-(Trifluoromethyl)-1H-pyrazol-1-amine along with 4-(trifluoromethyl)-1H-pyrazole (1:1 mixture) was obtained using a procedure analogous to that described in Step 1 of Example 91, substituting 4-(trifluoromethyl)-1H-pyrazole for 3-tert-butyl-1H-pyrazole used in Example 91.

Step 2: 2-(4-Bromophenyl)-N-(4-(trifluoromethyl)-1H-pyrazol-1-yl)acetamide (282 mg, 24%) was obtained using a procedure analogous to that described in Step 2 of Example 91, substituting a mixture of 4-(trifluoromethyl)-1H-pyrazol-1-amine and 4-(trifluoromethyl)-1H-pyrazole from Step 1 of this example for the mixture of 5-tert-butyl-1H-pyrazol-1-amine and 3-tert-butyl-1H-pyrazol-1-amine used in Example 91. ¹H NMR (300 MHz, DMSO-d₆) δ 12.22 (s, 1H), 8.56 (s, 1H), 7.96 (s, 1H), 7.49-7.62 (m, 2H), 7.30 (d, J=8.3 Hz, 2H), 3.66 (s, 2H).

Step 3: 2-(4-(6-Aminopyridin-3-yl)phenyl)-N-(4-(trifluoromethyl)-1H-pyrazol-1-yl)acetamide (145 mg, 49%) was obtained using a procedure analogous to that described in Step 2 of Example 107, substituting 2-(4-bromophenyl)-N-(4-(trifluoromethyl)-1H-pyrazol-1-yl)acetamide from Step 2 of this example for 5-bromo-N-(2-(4,4-difluoropiperidin-1-yl)ethyl)pyridin-2-amine, and 5-(4,4,5,5-tetramethyl-1,3,2-dioxaborolan-2-yl)pyridin-2-amine for tert-butyl 4-(4,4,5,5-tetramethyl-1,3,2-dioxaborolan-2-yl)phenylcarbamate used in Example 107. ¹H NMR (300 MHz, DMSO-d₆) δ 12.20 (br s, 1H), 8.57 (s, 1H), 8.24 (d, J=2.1 Hz, 1H), 7.96 (s, 1H), 7.69 (dd, J=2.4, 8.7 Hz, 1H), 7.54 (d, J=8.1 Hz, 2H), 7.25-7.46 (m, 2H), 6.52 (d, J=8.5 Hz, 1H), 6.06 (s, 2H), 3.66 (s, 2H). LC-MS (ESI) m/z 362 (M+H)⁺.

Step 4: 5-(4-(2-Oxo-2-(4-(trifluoromethyl)-1H-pyrazol-1-ylamino)ethyl)phenyl)pyridin-2-aminium methanesulfonate (172 mg, 97%) was obtained using a procedure analogous to that described in Step 3 of Example 89, substituting 2-(4-(6-aminopyridin-3-yl)phenyl)-N-(4-(trifluoromethyl)-1H-pyrazol-1-yl)acetamide from Step 3 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 13.38-14.02 (m, 1H), 12.24 (s, 1H), 8.56 (s, 1H), 8.21-8.34 (m, 2H), 7.81-8.03 (m, 3H), 7.65 (d, J=8.1 Hz, 2H), 7.45 (d, J=8.3 Hz, 2H), 7.05 (d, J=7.9 Hz, 1H), 3.72 (s, 2H), 2.31 (br s, 3H). LC-MS (ESI) m/z 362 (M+H)⁺.

### Example 127

### Preparation of 3-fluoro-5-(4-(2-oxo-2-(5-(1-(trifluoromethybcyclopropybisoxazol-3-ylamino)ethyl)phenyl)pyridin-2-aminium methanesulfonate

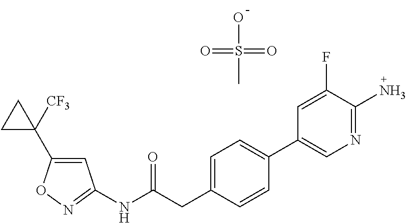

Step 1: 2-(4-(4,4,5,5-Tetramethyl-1,3,2-dioxaborolan-2-yl)phenyl)-N-(5-(1-(trifluoromethyl)cyclopropyl)isoxazol-3-yl)acetamide (3.7 g, 45%) was obtained as a white solid using a procedure analogous to that described in Step 1 of Example 85, substituting 5-(1-(trifluoromethyl)cyclopropyl)isoxazol-3-amine for 5-tert-butylisoxazol-3-amine, and TEA for DIEA used in Example 85, respectively. ¹H NMR (300 MHz, DMSO-d₆) δ 11.37 (s, 1H), 7.63 (d, J=7.9 Hz, 2H), 7.32 (d, J=7.7 Hz, 2H), 6.91 (s, 1H), 3.71 (s, 2H), 1.51 (br s, 2H), 1.48 (br s, 2H), 1.29 (s, 12H). LC-MS (ESI) m/z 437 (M+H)⁺.

Step 2: 3-Fluoro-5-(4-(2-oxo-2-(5-(1-(trifluoromethyl)cyclopropyl)isoxazol-3-ylamino)ethyl)phenyl)pyridin-2-aminium methanesulfonate (90 mg, 38%) was obtained as a white solid using procedures analogous to those described in Steps 2-3 of Example 109, substituting 2-(4-(4,4,5,5-tetramethyl-1,3,2-dioxaborolan-2-yl)phenyl)-N-(5-(1-(trifluoromethyl)cyclopropyl)isoxazol-3-yl)acetamide from Step 1 of this example for 2-(4-(4,4,5,5-tetramethyl-1,3,2-dioxaborolan-2-yl)phenyl)-N-(5-(1,1,1-trifluoro-2-methylpropan-2-yl)isoxazol-3-yl)acetamide, and 5-bromo-3-fluoropyridin-2-amine from Example 94 for 5-bromo-N-(2-morpholinoethyl)pyridin-2-amine used in Example 109. ¹H NMR (300 MHz, DMSO-d₆) δ 11.39 (s, 1H), 8.15-8.22 (m, 1H), 8.14 (br s, 1H), 7.64 (d, J=8.1 Hz, 2H), 7.40 (d, J=8.1 Hz, 2H), 6.92 (s, 1H), 3.73 (s, 2H), 2.34 (s, 3H), 1.52 (d, J=2.6 Hz, 2H), 1.48 (br s, 2H). LC-MS (ESI) m/z 421 (M+H)⁺.

### Example 128

### Preparation of 3-chloro-5-(4-(2-((5-(3-methyloxetan-3-yl)isoxazol-3-yl)amino)-2-oxoethyl)phenyl)pyridin-2-aminium methanesulfonate

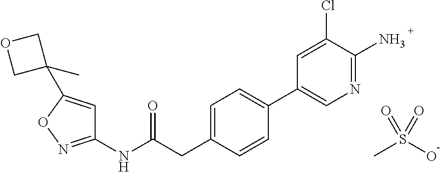

Step 1: N-(5-(3-Methyloxetan-3-yl)isoxazol-3-yl)-2-(4-(4,4,5,5-tetramethyl-1,3,2-dioxaborolan-2-yl)phenyl)acetamide (750 mg, 43%) was prepared using a procedure analogous to that described in Step 1 of Example 124, substituting 5-(3-Methyloxetan-3-yl)isoxazol-3-amine from Step 3 of Example 98 for 5-(1-(trifluoromethyl)cyclobutyl)isoxazol-3-amine, and 2-(4-(4,4,5,5-tetramethyl-1,3,2-dioxaborolan-2-yl)phenyl)acetic acid for 2-(4-bromophenyl)acetic acid used in Example 124. LC-MS (ESI) m/z 399 (M+H)⁺.

Step 2: 3-Chloro-5-(4-(2-((5-(3-methyloxetan-3-yl)isoxazol-3-yl)amino)-2-oxoethyl)phenyl)pyridin-2-aminium methanesulfonate (80 mg, 32%) was prepared using procedures analogous to those described in Steps 2-3 of Example 83, substituting N-(5-(3-methyloxetan-3-yl)isoxazol-3-yl)-2-(4-(4,4,5,5-tetramethyl-1,3,2-dioxaborolan-2-yl)phenyl)acetamide from Step 1 of this example for 5-(4,4,5,5-tetramethyl-1,3,2-dioxaborolan-2-yl)pyridin-2-amine, and 5-bromo-3-chloropyridin-2-amine for 2-(4-bromophenyl)-N-(3-(1-hydroxy-2-methylpropan-2-yl)isoxazol-5-yl)acetamide used in Example 83. ¹H NMR (300 MHz, DMSO-d₆) δ 11.93 (s, 1H), 8.27 (s, 1H), 8.07 (d, J=12.2 Hz, 1H), 7.61 (d, J=7.7 Hz, 2H), 7.38 (d, J=7.7 Hz, 2H), 6.88 (br s, 2H), 6.32 (s, 1H), 4.74 (d, J=5.8 Hz, 2H), 4.49 (d, J=5.7 Hz, 2H), 3.74 (s, 2H), 2.31 (s, 3H), 1.60 (s, 3H). LC-MS (ESI) m/z 399 (M+H)⁺.

### Example 129

### Preparation of 5-(4-(2-(5-tert-butylisoxazol-3-ylamino)-2-oxoethyl)phenyl)-6-(trifluoromethybpyridin-2-aminium methanesulfonate

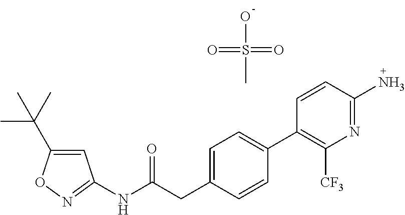

Step 1: 5-Bromo-6-(trifluoromethyl)pyridin-2-amine (350 mg, 47%) was obtained as a brown solid using a procedure analogous to that described in Step 1 of Example 94, substituting 6-(trifluoromethyl)pyridin-2-amine for 3-fluoropyridin-2-amine used in Example 94. ¹H NMR (300 MHz, CHLOROFORM-d) δ 7.70 (d, J=8.7 Hz, 1H), 6.55 (d, J=8.7 Hz, 1H), 4.74 (br s, 2H). LC-MS (ESI) m/z 241 and 243 (M+H)⁺.

Step 2: 5-(4-(2-(5-tert-Butylisoxazol-3-ylamino)-2-oxoethyl)phenyl)-6-(trifluoromethyl)pyridin-2-aminium methanesulfonate (180 mg, 45%) was obtained as a pink solid using procedures analogous to those described in Steps 1-2 of Example 110, substituting 5-bromo-6-(trifluoromethyl)pyridin-2-amine for 5-bromo-N-methylpyridin-2-amine used in Example 110. ¹H NMR (300 MHz, DMSO-d₆) δ 11.24 (s, 1H), 7.40 (d, J=8.5 Hz, 1H), 7.34 (d, J=8.1 Hz, 2H), 7.18-7.25 (m, 2H), 6.71 (d, J=8.7 Hz, 1H), 6.59 (s, 1H), 3.70 (s, 2H), 2.31 (s, 3H), 1.28 (s, 9H). LC-MS (ESI) m/z 419 (M+H)⁺.

### Example 130

### Preparation of 3-methyl-5-(4-(2-oxo-2-(5-(1-(trifluoromethybcyclopropybisoxazol-3-ylamino)ethyl)phenyl)pyridin-2-aminium methanesulfonate

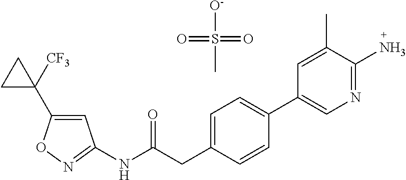

3-Methyl-5-(4-(2-oxo-2-(5-(1-(trifluoromethyl)cyclopropyl)isoxazol-3-ylamino)ethyl)phenyl)pyridin-2-aminium methanesulfonate (75 mg, 26%) was obtained as an off-white solid using procedures analogous to those described in Steps 2-3 of Example 109, substituting 2-(4-(4,4,5,5-tetramethyl-1,3,2-dioxaborolan-2-yl)phenyl)-N-(5-(1-(trifluoromethyl)cyclopropyl)isoxazol-3-yl)acetamide from Example 127 for 2-(4-(4,4,5,5-tetramethyl-1,3,2-dioxaborolan-2-yl)phenyl)-N-(5-(1,1,1-trifluoro-2-methylpropan-2-yl)isoxazol-3-yl)acetamide, and 5-bromo-3-methylpyridin-2-amine for 5-bromo-N-(2-morpholinoethyl)pyridin-2-amine used in Example 109, respectively. ¹H NMR (300 MHz, DMSO-d₆) δ 11.40 (s, 1H), 8.24 (s, 1H), 8.16 (s, 1H), 7.82 (br s, 2H), 7.65 (d, J=8.3 Hz, 2H), 7.42 (d, J=8.1 Hz, 2H), 6.92 (s, 1H), 3.74 (s, 2H), 2.33 (s, 3H), 2.27 (s, 3H), 1.52 (d, J=3.0 Hz, 2H), 1.48 (br s, 2H). LC-MS (ESI) m/z 417 (M+H)⁺.

### Example 131

### Preparation of 5-(4-(2-(5-tert-butylisoxazol-3-ylamino)-2-oxoethyl)phenyl)-6-methoxypyridin-2-aminium methanesulfonate

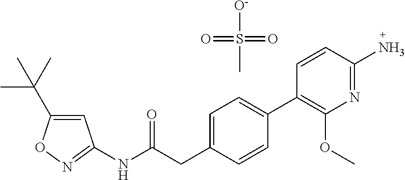

Step 1: 5-Bromo-6-methoxypyridin-2-amine (300 mg, 23%) was obtained as an off-white solid using a procedure analogous to that described in Step 1 of Example 94, substituting 6-methoxypyridin-2-amine for 3-fluoropyridin-2-amine used in Example 94. ¹H NMR (300 MHz, CHLOROFORM-d) δ 7.48 (d, J=8.1 Hz, 1H), 5.99 (d, J=8.1 Hz, 1H), 4.32 (br s, 2H), 3.92 (s, 3H). LC-MS (ESI) m/z 203 and 205 (M+H)⁺.

Step 2: 5-(4-(2-(5-tert-Butylisoxazol-3-ylamino)-2-oxoethyl)phenyl)-6-methoxypyridin-2-aminium methanesulfonate (75 mg, 20%) was obtained as a white solid using procedures analogous to those described in Steps 1-2 of Example 110, substituting 5-bromo-6-methoxypyridin-2-amine for 5-bromo-N-methylpyridin-2-amine used in Example 110. ¹H NMR (300 MHz, DMSO-d₆) δ 11.19 (br s, 1H), 7.36-7.46 (m, 3H), 7.23-7.33 (m, 2H), 6.59-6.69 (m, 2H), 6.57 (br s, 1H), 6.17 (d, J=8.1 Hz, 1H), 3.79 (s, 3H), 3.65 (br s, 2H), 2.36 (s, 3H), 1.27 (s, 9H). LC-MS (ESI) m/z 381 (M+H)⁺.

### Example 132

### Preparation of 5-(4-(2-(5-tert-butylisoxazol-3-ylamino)-2-oxoethyl)phenyl)-6-chloropyridin-2-aminium methanesulfonate

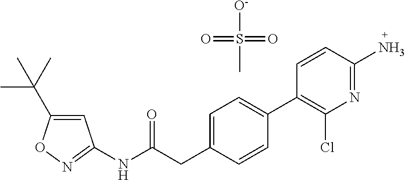

Step 1: 5-Bromo-6-chloropyridin-2-amine (500 mg, 31%) was obtained as a white solid using a procedure analogous to that described in Step 1 of Example 94, substituting 6-chloropyridin-2-amine for 3-fluoropyridin-2-amine used in Example 94. LC-MS (ESI) m/z 207, 209, and 211 (M+H)⁺.

Step 2: 5-(4-(2-(5-tert-Butylisoxazol-3-ylamino)-2-oxoethyl)phenyl)-6-chloropyridin-2-aminium methanesulfonate (65 mg, 52%) was obtained as an off-white solid using procedures analogous to those described in Steps 1-2 of Example 110, substituting 5-bromo-6-chloropyridin-2-amine for 5-bromo-N-methylpyridin-2-amine used in Example 110. ¹H NMR (300 MHz, DMSO-d₆) δ 11.23 (s, 1H), 7.42 (d, J=8.3 Hz, 1H), 7.34 (s, 4H), 6.58 (s, 1H), 6.48 (d, J=8.3 Hz, 1H), 3.69 (s, 2H), 2.33 (s, 3H), 1.28 (s, 9H). LC-MS (ESI) m/z 385 (M+H)⁺.

### Example 133

### Preparation of 3-chloro-5-(4-(2-oxo-2-(5-(1-(trifluoromethyl)cyclopropyl)isoxazol-3-ylamino)ethyl)phenyl)pyridin-2-aminium methanesulfonate

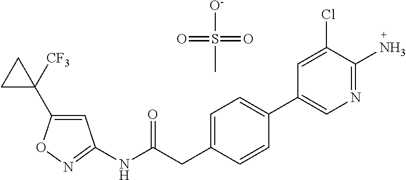

3-Chloro-5-(4-(2-oxo-2-(5-(1-(trifluoromethyl)cyclopropyl)isoxazol-3-ylamino)ethyl)phenyl)pyridin-2-aminium methanesulfonate (95 mg, 37%) was obtained as an off-white solid using procedures analogous to those described in Steps 2-3 of Example 109, substituting 2-(4-(4,4,5,5-tetramethyl-1,3,2-dioxaborolan-2-yl)phenyl)-N-(5-(1-(trifluoromethyl)cyclopropyl)isoxazol-3-yl)acetamide from Example 127 for 2-(4-(4,4,5,5-tetramethyl-1,3,2-dioxaborolan-2-yl)phenyl)-N-(5-(1,1,1-trifluoro-2-methylpropan-2-yl)isoxazol-3-yl)acetamide, and 5-bromo-3-chloropyridin-2-amine from Example 115 for 5-bromo-N-(2-morpholinoethyl)pyridin-2-amine used in Example 109. ¹H NMR (300 MHz, DMSO-d₆) δ 11.39 (s, 1H), 8.37 (d, J=1.7 Hz, 1H), 8.29 (d, J=1.9 Hz, 1H), 7.65 (d, J=8.3 Hz, 2H), 7.40 (d, J=8.3 Hz, 2H), 6.92 (s, 1H), 3.73 (s, 2H), 2.36 (s, 3H), 1.52 (br s, 2H), 1.48 (br s, 2H). LC-MS (ESI) m/z 437 (M+H)⁺.

### Example 134

### Preparation of 2-(4-(6-amino-2-fluoropyridin-3-yl)phenyl)-N-(5-(1-methylcyclopropyl)isoxazol-3-yl)acetamide

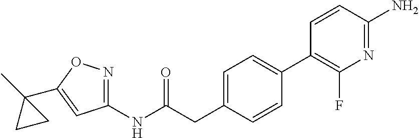 [SMILES: CC1(c2cc(NC(=O)Cc3ccc(-c4ccc(N)nc4F)cc3)no2)CC1]

Step 1: N-(5-(1-Methylcyclopropyl)isoxazol-3-yl)-2-(4-(4,4,5,5-tetramethyl-1,3,2-dioxaborolan-2-yl)phenyl)acetamide was synthesized as a light yellow solid using the procedure analogous to that described in Step 1 of Example 85, substituting 5-(1-methylcyclopropyl)isoxazol-3-amine from Step 2 of Example 100 for 5-tert-butylisoxazol-3-amine used in Example 85. LC-MS (ESI) m/z 419 (M+H)⁺.

Step 2: 2-(4-(6-Amino-2-fluoropyridin-3-yl)phenyl)-N-(5-(1-methylcyclopropyl)isoxazol-3-yl)acetamide (12 mg, 6%) was synthesized as a solid according to the procedure described in Step 1 of Example 85, substituting N-(5-(1-methylcyclopropyl)isoxazol-3-yl)-2-(4-(4,4,5,5-tetramethyl-1,3,2-dioxaborolan-2-yl)phenyl)acetamide from Step 1 of this example for N-(5-tert-butylisoxazol-3-yl)-2-(4-(4,4,5,5-tetramethyl-1,3,2-dioxaborolan-2-yl)phenyl)acetamide, and 5-bromo-6-fluoropyridin-2-amine for 5-bromo-3-methylpyridin-2-amine used in Example 85. ¹H NMR (300 MHz, DMSO-d₆) δ 11.81 (br s, 1H), 7.66 (dd, J=8.3, 10.7 Hz, 1H), 7.38-7.50 (m, 2H), 7.28-7.37 (m, 2H), 6.36-6.54 (m, 3H), 5.94 (s, 1H), 3.70 (s, 2H), 1.35 (s, 3H), 0.86-0.95 (m, 2H), 0.76-0.85 (m, 2H). LC-MS (ESI) m/z 367 (M+H)⁺.

### Example 135

### Preparation of 5-(4-(2-((5-(3-methyloxetan-3-yl)isoxazol-3-yl)amino)-2-oxoethyl)phenyl)-3-(trifluoromethyl)pyridin-2-aminium methanesulfonate

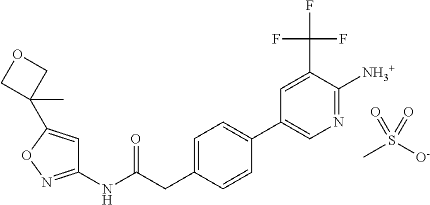

5-(4-(2-((5-(3-Methyloxetan-3-yl)isoxazol-3-yl)amino)-2-oxoethyl)phenyl)-3-(trifluoromethyl)pyridin-2-aminium methanesulfonate (90 mg, 34%) was prepared using procedures analogous to those described in Steps 2-3 of Example 83, substituting N-(5-(3-methyloxetan-3-yl)isoxazol-3-yl)-2-(4-(4,4,5,5-tetramethyl-1,3,2-dioxaborolan-2-yl)phenyl)acetamide from Step 1 of Example 128 for 5-(4,4,5,5-tetramethyl-1,3,2-dioxaborolan-2-yl)pyridin-2-amine, and 5-bromo-3-(trifluoromethyl)pyridin-2-amine for 2-(4-bromophenyl)-N-(3-(1-hydroxy-2-methylpropan-2-yl)isoxazol-5-yl)acetamide used in Example 83. ¹H NMR (300 MHz, DMSO-d₆) δ 11.94 (s, 1H), 8.54 (br s, 1H), 8.10 (d, J=13.8 Hz, 1H), 7.64 (d, J=6.2 Hz, 2H), 7.39 (d, J=7.2 Hz, 2H), 6.32 (s, 1H), 4.74 (d, J=5.7 Hz, 2H), 4.49 (d, J=5.8 Hz, 2H), 3.75 (br s, 2H), 2.29-2.43 (m, 2H), 1.60 (s, 2H), 1.09-1.31 (m, 1H). LC-MS (ESI) m/z 433 (M+H)⁺.

### Example 136

### Preparation of 5-(4-(2-(5-tert-butylisoxazol-3-ylamino)-2-oxoethyl)phenyl)-3-methoxypyridin-2-aminium methanesulfonate

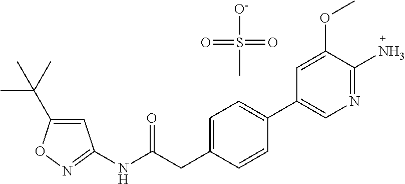

Step 1: 5-Bromo-3-methoxypyridin-2-amine (500 mg, 31%) was obtained as a tan solid using a procedure analogous to that described in Step 1 of Example 94, substituting 3-methoxypyridin-2-amine for 3-fluoropyridin-2-amine used in Example 94. ¹H NMR (300 MHz, CHLOROFORM-d) δ 7.73 (d, J=1.7 Hz, 1H), 7.02 (s, 1H), 4.69 (br s, 2H), 3.86 (s, 3H). LC-MS (ESI) m/z 203 and 205 (M+H)⁺.

Step 2: 5-(4-(2-(5-tert-Butylisoxazol-3-ylamino)-2-oxoethyl)phenyl)-3-methoxypyridin-2-aminium methanesulfonate (80 mg, 32%) was obtained as a white solid using procedures analogous to those described in Steps 1-2 of Example 110, substituting 5-bromo-3-methoxypyridin-2-amine for 5-bromo-N-methylpyridin-2-amine used in Example 110. ¹H NMR (300 MHz, DMSO-d₆) δ 11.22 (s, 1H), 7.94 (br s, 2H), 7.82 (s, 1H), 7.70 (d, J=3.8 Hz, 2H), 7.67 (s, 1H), 7.43 (d, J=8.1 Hz, 2H), 6.56 (s, 1H), 4.03 (s, 3H), 3.72 (s, 2H), 2.33 (s, 3H), 1.27 (s, 9H). LC-MS (ESI) m/z 381 (M+H)⁺.

### Example 137

### Preparation of 6-fluoro-5-(4-(2-oxo-2-(5-(1-(trifluoromethyl)cyclopropyl)isoxazol-3-ylamino)ethyl)phenyl)pyridin-2-aminium methanesulfonate

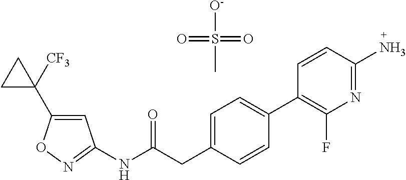

6-Fluoro-5-(4-(2-oxo-2-(5-(1-(trifluoromethyl)cyclopropyl)isoxazol-3-ylamino)ethyl)phenyl)pyridin-2-aminium methanesulfonate (28 mg, 11%) was obtained as an sticky brown solid using procedures analogous to those described in Steps 2-3 of Example 109, substituting 2-(4-(4,4,5,5-tetramethyl-1,3,2-dioxaborolan-2-yl)phenyl)-N-(5-(1-(trifluoromethyl)cyclopropyl)isoxazol-3-yl)acetamide from Example 127 for 2-(4-(4,4,5,5-tetramethyl-1,3,2-dioxaborolan-2-yl)phenyl)-N-(5-(1,1,1-trifluoro-2-methylpropan-2-yl)isoxazol-3-yl)acetamide, and 5-bromo-6-fluoropyridin-2-amine from Example 95 for 5-bromo-N-(2-morpholinoethyl)pyridin-2-amine used in Example 109. ¹H NMR (300 MHz, DMSO-d₆) δ 11.38 (s, 1H), 7.66 (dd, J=8.3, 10.7 Hz, 1H), 7.40-7.44 (m, 2H), 7.31-7.37 (m, 2H), 6.93 (s, 1H), 6.41 (dd, J=1.7, 8.3 Hz, 1H), 3.70 (s, 2H), 2.33 (s, 3H), 1.51 (br s, 2H), 1.48 (br s, 2H). LC-MS (ESI) m/z 421 (M+H)⁺.

### Example 138

### Preparation of 3-fluoro-5-(4-(2-(5-(1-methylcyclopropyl)isoxazol-3-ylamino)-2-oxoethyl)phenyl)pyridin-2-aminium methanesulfonate

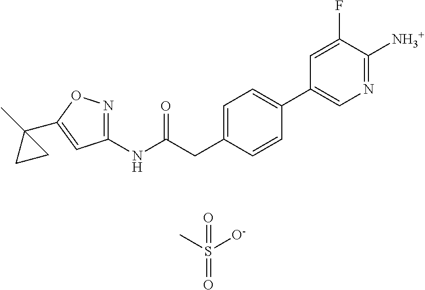

Step 1: 2-(4-(6-Amino-5-fluoropyridin-3-yl)phenyl)-N-(5-(1-methylcyclopropyl)isoxazol-3-yl)acetamide (156 mg, 45%) was synthesized as a solid according to the procedure described in Step 1 of Example 85, substituting N-(5-(1-methylcyclopropyl)isoxazol-3-yl)-2-(4-(4,4,5,5-tetramethyl-1,3,2-dioxaborolan-2-yl)phenyl)acetamide from Step 1 of Example 134 for N-(5-tert-butylisoxazol-3-yl)-2-(4-(4,4,5,5-tetramethyl-1,3,2-dioxaborolan-2-yl)phenyl)acetamide, and 5-bromo-3-fluoropyridin-2-amine for 5-bromo-3-methylpyridin-2-amine used in Example 85. LC-MS (ESI) m/z 367 (M+H)⁺.

Step 2: 3-Fluoro-5-(4-(2-(5-(1-methylcyclopropyl)isoxazol-3-ylamino)-2-oxoethyl)phenyl)pyridin-2-aminium methanesulfonate (198 mg, 100%) was synthesized as a solid according to the procedure described in Step 3 of Example 89, substituting 2-(4-(6-amino-5-fluoropyridin-3-yl)phenyl)-N-(5-(1-methylcyclopropyl)isoxazol-3-yl)acetamide from Step 1 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 11.79 (s, 1H), 8.17-8.27 (m, 1H), 8.14 (d, J=1.7 Hz, 1H), 7.64 (d, J=8.3 Hz, 3H), 7.39 (d, J=8.3 Hz, 2H), 6.36-6.54 (br, 2H), 5.94 (s, 1H), 3.73 (s, 2H), 2.35 (s, 3H), 1.35 (s, 3H), 0.86-0.95 (m, 2H), 0.77-0.86 (m, 2H). LC-MS (ESI) m/z 367 (M+H)⁺.

### Example 139

### Preparation of 5-(3-fluoro-4-(2-oxo-2-(5-(1-(trifluoromethyl)cyclopropyl)isoxazol-3-ylamino)ethyl)phenyl)pyridin-2-aminium methanesulfonate

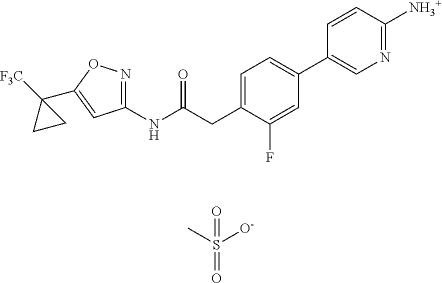

Step 1: 2-(4-Bromo-2-fluorophenyl)-N-(5-(1-(trifluoromethyl)cyclopropyl)isoxazol-3-yl)acetamide was synthesized as a white solid (410 mg, 71%) using the procedure analogous to that described in Step 1 of Example 18, substituting 5-(1-(trifluoromethyl)cyclopropyl)isoxazol-3-amine from Step 1 of Example 33 for 5-tert-butylisoxazol-3-amine, and 2-(4-bromo-2-fluorophenyl)acetic acid for 2-(4-bromophenyl)acetic acid used in Example 18. LC-MS (ESI) m/z 408 (M+H)⁺.

Step 2: 2-(4-(6-Aminopyridin-3-yl)-2-fluorophenyl)-N-(5-(1-(trifluoromethyl)cyclopropyl)isoxazol-3-yl)acetamide (112 mg, 27%) was synthesized as a solid using the procedure analogous to that described in Step 2 of Example 40, substituting 2-(4-bromo-2-fluorophenyl)-N-(5-(1-(trifluoromethyl)cyclopropyl)isoxazol-3-yl)acetamide from Step 1 of this example for 5-bromo-N-tritylprydin-2-amine used in Example 40. LC-MS (ESI) m/z 421 (M+H)⁺.

Step 3: 5-(3-Fluoro-4-(2-oxo-2-(5-(1-(trifluoromethyl)cyclopropyl)isoxazol-3-ylamino)ethyl)phenyl)pyridin-2-aminium methanesulfonate (139 mg, 100%) was synthesized as a solid using the procedure analogous that described in Step 3 of Example 89, substituting 2-(4-(6-aminopyridin-3-yl)-2-fluorophenyl)-N-(5-(1-(trifluoromethyl)cyclopropyl)isoxazol-3-yl)acetamide from Step 2 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 13.84 (br s, 1H), 11.44 (br s, 1H), 8.26-8.51 (m, 2H), 8.04 (br s, 2H), 7.37-7.71 (m, 3H), 7.06 (d, J=9.0 Hz, 1H), 6.91 (s, 1H), 3.83 (s, 2H), 2.38 (s, 3H), 1.37-1.70 (m, 4H). LC-MS (ESI) m/z 421 (M+H)⁺.

### Example 140

### Preparation of 2-(4-(6-amino-2-fluoropyridin-3-yl)phenyl)-N-(5-(3-methyloxetan-3-yl)isoxazol-3-yl)acetamide

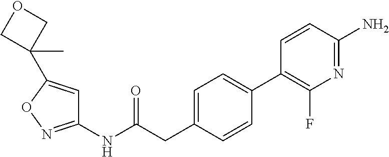 [SMILES: CC1(c2cc(NC(=O)Cc3ccc(-c4ccc(N)nc4F)cc3)no2)COC1]

2-(4-(6-Amino-2-fluoropyridin-3-yl)phenyl)-N-(5-(3-methyloxetan-3-yl)isoxazol-3-yl)acetamide (65 mg, 35%) was prepared using procedures analogous to those described in Steps 2-3 of Example 83, substituting N-(5-(3-methyloxetan-3-yl)isoxazol-3-yl)-2-(4-(4,4,5,5-tetramethyl-1,3,2-dioxaborolan-2-yl)phenyl)acetamide from Step 1 of Example 128 for 5-(4,4,5,5-tetramethyl-1,3,2-dioxaborolan-2-yl)pyridin-2-amine, and 5-bromo-6-fluoropyridin-2-amine for 2-(4-bromophenyl)-N-(3-(1-hydroxy-2-methylpropan-2-yl)isoxazol-5-yl)acetamide used in Example 83. ¹H NMR (300 MHz, DMSO-d₆) δ 11.92 (br s, 1H), 7.58-7.78 (m, 1H), 7.24-7.52 (m, 4H), 6.37-6.54 (m, 3H), 6.32 (br s, 1H), 4.74 (d, J=4.3 Hz, 2H), 4.42-4.57 (m, 2H), 3.72 (br s, 2H), 1.60 (br s, 3H). LC-MS (ESI) m/z 383 (M+H)⁺.

### Example 141

### Preparation of N-(5-tert-butylisoxazol-3-yl)-2-(4-(2-oxo-2,3-dihydrooxazolo[4,5-b]pyridin-6-yl)phenyl)acetamide

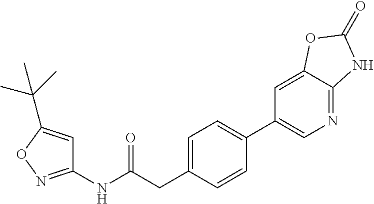 [SMILES: CC(C)(C)c1cc(NC(=O)Cc2ccc(-c3cnc4[nH]c(=O)oc4c3)cc2)no1]

N-(5-tert-butylisoxazol-3-yl)-2-(4-(2-oxo-2,3-dihydrooxazolo[4,5-b]pyridin-6-yl)phenyl)acetamide (2.29 mg, 2%) was obtained using a procedure analogous to that described in Step 2 of Example 92, substituting 6-bromooxazolo[4,5-b]pyridin-2(3H)-one for 5-bromo-N-(2-(methylsulfonyl)ethyl)pyridin-2-amine used in Example 92. ¹H NMR (300 MHz, DMSO-d₆) δ 12.51 (br s, 1H), 11.23 (s, 1H), 8.36 (s, 1H), 7.98 (s, 1H), 7.66 (d, J=8.1 Hz, 2H), 7.41 (d, J=8.1 Hz, 2H), 6.57 (s, 1H), 3.71 (s, 2H), 1.14-1.39 (m, 9H). LC-MS (ESI) m/z 393 (M+H)⁺.

### Example 142

### Preparation of 6-fluoro-5-(4-(2-oxo-2-(5-(1,1,1-trifluoro-2-methylpropan-2-yl)isoxazol-3-ylamino)ethyl)phenyl)pyridin-2-aminium methanesulfonate

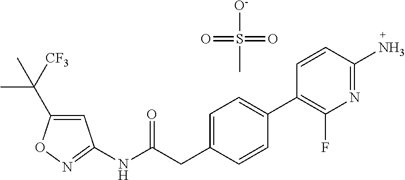

6-Fluoro-5-(4-(2-oxo-2-(5-(1,1,1-trifluoro-2-methylpropan-2-yl)isoxazol-3-ylamino)ethyl)phenyl)pyridin-2-aminium methanesulfonate (60 mg, 11%) was obtained as a white solid using procedures analogous to those described in Steps 2-3 of Example 109, substituting 5-bromo-6-fluoropyridin-2-amine from Example 95 for 5-bromo-N-(2-morpholinoethyl)pyridin-2-amine used in Example 109. ¹H NMR (300 MHz, DMSO-d₆) δ 11.38 (s, 1H), 7.66 (dd, J=8.4, 10.8 Hz, 1H), 7.39-7.47 (m, 2H), 7.30-7.39 (m, 2H), 6.94 (s, 1H), 6.66 (br s, 2H), 6.42 (d, J=8.3 Hz, 1H), 3.70 (s, 2H), 2.35 (s, 3H), 1.53 (s, 6H). LC-MS (ESI) m/z 423 (M+H)⁺.

### Example 143

### Preparation of 5-(4-(2-((3-cyclobutylisoxazol-5-yl)amino)-2-oxoethyl)phenyl)pyridin-2-aminium methanesulfonate

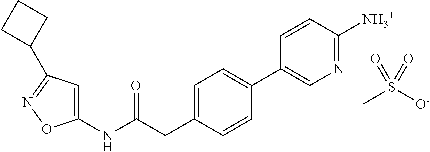

Step 1: 3-Cyclobutylisoxazol-5-amine (267 mg, 55%) was prepared using procedures analogous to those described in Steps 1-3 of Example 98, substituting cyclobutanecarboxylic acid for 3-methyloxetane-3-carboxylic acid used in Example 98. LC-MS (ESI) m/z 139 (M+H)⁺.

Step 2: 2-(4-Bromophenyl)-N-(3-cyclobutylisoxazol-5-yl)acetamide (175 mg, 27%) was prepared using a procedure analogous to that described in Step 1 of Example 124, substituting 3-cyclobutylisoxazol-5-amine from Step 1 of this example for 5-(1-(trifluoromethyl)cyclobutyl)isoxazol-3-amine used in Example 124. LC-MS (ESI) m/z 335, 337 (M+H)⁺.

Step 3: 5-(4-(2-((3-Cyclobutylisoxazol-5-yl)amino)-2-oxoethyl)phenyl)pyridin-2-aminium methanesulfonate (100 mg, 43%) was prepared using procedures analogous to those described in Steps 2-3 of Example 83, substituting 2-(4-bromophenyl)-N-(3-cyclobutylisoxazol-5-yl)acetamide from Step 2 of this example for 2-(4-bromophenyl)-N-(3-(1-hydroxy-2-methylpropan-2-yl)isoxazol-5-yl)acetamide used in Example 83. ¹H NMR (300 MHz, DMSO-d₆) δ 11.83 (s, 1H), 8.15-8.31 (m, 2H), 7.78 (br s, 2H), 7.62 (d, J=8.1 Hz, 2H), 7.41 (d, J=8.1 Hz, 2H), 7.01 (d, J=9.0 Hz, 1H), 6.18 (s, 1H), 3.75 (s, 2H), 3.43-3.58 (m, 1H), 2.35 (s, 3H), 1.79-2.32 (m, 6H). LC-MS (ESI) m/z 349 (M+H)⁺.

### Example 144

### Preparation of 5-(4-(2-((5-(1-methylcyclobutyl)isoxazol-3-yl)amino)-2-oxoethyl)phenyl)pyridin-2-aminium methanesulfonate

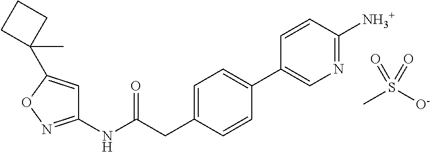

Step 1: 5-(1-Methylcyclobutyl)isoxazol-3-amine (2.1 g, 55%) was prepared using procedures analogous to those described in Steps 1-3 of Example 98, substituting 1-methylcyclobutanecarboxylic acid for 3-methyloxetane-3-carboxylic acid used in Example 98. LC-MS (ESI) m/z 153 (M+H)⁺.

Step 2: 2-(4-Bromophenyl)-N-(5-(1-methylcyclobutyl)isoxazol-3-yl)acetamide (312 mg, 68%) was prepared using a procedure analogous to that described in Step 1 of Example 124, substituting 5-(1-methylcyclobutyl)isoxazol-3-amine from Step 1 of this example for 5-(1-(trifluoromethyl)cyclobutyl)isoxazol-3-amine used in Example 124. LC-MS (ESI) m/z 349, 351 (M+H)⁺.

Step 3: 5-(4-(2-((5-(1-Methylcyclobutyl)isoxazol-3-yl)amino)-2-oxoethyl)phenyl)pyridin-2-aminium methanesulfonate (235 mg,) was prepared using procedures analogous to those described in Steps 2-3 of Example 83, substituting 2-(4-bromophenyl)-N-(5-(1-methylcyclobutyl)isoxazol-3-yl)acetamide from Step 2 of this example for 2-(4-bromophenyl)-N-(3-(1-hydroxy-2-methylpropan-2-yl)isoxazol-5-yl)acetamide used in Example 83. ¹H NMR (300 MHz, DMSO-d₆) δ 11.85 (s, 1H), 8.19-8.38 (m, 2H), 8.00 (br s, 2H), 7.63 (d, J=8.1 Hz, 2H), 7.43 (d, J=8.1 Hz, 2H), 7.07 (d, J=9.2 Hz, 1H), 6.15 (s, 1H), 3.75 (s, 2H), 2.24-2.43 (m, 5H), 1.73-2.11 (m, 4H), 1.42 (s, 3H). LC-MS (ESI) m/z 363 (M+H)⁺.

### Example 145

### Preparation of 3-methyl-5-(4-(2-((5-(3-methyloxetan-3-yl)isoxazol-3-yl)amino)-2-oxoethyl)phenyl)pyridin-2-aminium methanesulfonate

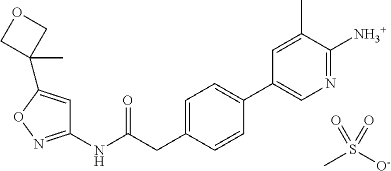

3-Methyl-5-(4-(2-((5-(3-methyloxetan-3-yl)isoxazol-3-yl)amino)-2-oxoethyl)phenyl)pyridin-2-aminium methanesulfonate (85 mg, 34%) was prepared using procedures analogous to those described in Steps 2-3 of Example 83, substituting N-(5-(3-methyloxetan-3-yl)isoxazol-3-yl)-2-(4-(4,4,5,5-tetramethyl-1,3,2-dioxaborolan-2-yl)phenyl)acetamide from Step 1 of Example 128 for 5-(4,4,5,5-tetramethyl-1,3,2-dioxaborolan-2-yl)pyridin-2-amine, and 5-bromo-3-methylpyridin-2-amine for 2-(4-bromophenyl)-N-(3-(1-hydroxy-2-methylpropan-2-yl)isoxazol-5-yl)acetamide used in Example 83. ¹H NMR (300 MHz, DMSO-d₆) δ 11.96 (s, 1H), 8.09-8.35 (m, 2H), 7.94 (br s, 2H), 7.66 (d, J=7.9 Hz, 2H), 7.43 (d, J=7.9 Hz, 2H), 6.32 (s, 1H), 4.74 (d, J=5.7 Hz, 2H), 4.49 (d, J=5.7 Hz, 2H), 3.18-3.66 (m, 5H), 2.41 (s, 2H), 2.27 (s, 2H), 1.60 (s, 2H). LC-MS (ESI) m/z 379 (M+H)⁺.

### Example 146

### Preparation of 3-fluoro-5-(4-(2-((5-(3-methyloxetan-3-yl)isoxazol-3-yl)amino)-2-oxoethyl)phenyl)pyridin-2-aminium methanesulfonate

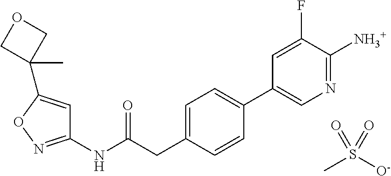

3-Fluoro-5-(4-(2-((5-(3-methyloxetan-3-yl)isoxazol-3-yl)amino)-2-oxoethyl)phenyl)pyridin-2-aminium methanesulfonate (130 mg, 51%) was prepared using procedures analogous to those described in Steps 2-3 of Example 83, substituting N-(5-(3-methyloxetan-3-yl)isoxazol-3-yl)-2-(4-(4,4,5,5-tetramethyl-1,3,2-dioxaborolan-2-yl)phenyl)acetamide from Step 1 of Example 128 for 5-(4,4,5,5-tetramethyl-1,3,2-dioxaborolan-2-yl)pyridin-2-amine, and 5-bromo-3-fluoropyridin-2-amine for 2-(4-bromophenyl)-N-(3-(1-hydroxy-2-methylpropan-2-yl)isoxazol-5-yl)acetamide used in Example 83. ¹H NMR (300 MHz, DMSO-d₆) δ 11.95 (s, 1H), 8.30 (br s, 1H), 8.16 (s, 1H), 7.66 (d, J=7.7 Hz, 2H), 7.42 (d, J=8.1 Hz, 2H), 6.32 (s, 1H), 4.74 (d, J=5.8 Hz, 2H), 4.49 (d, J=5.7 Hz, 2H), 3.77 (s, 2H), 2.41 (br s, 2H), 2.39 (br s, 3H), 1.60 (s, 3H). LC-MS (ESI) m/z 383 (M+H)⁺.

### Example 147

### N-(5-tert-butylisoxazol-3-yl)-2-(4-(2-oxo-2,3-dihydro-1H-imidazo[4,5-b]pyridin-6-yl)phenyl)acetamide

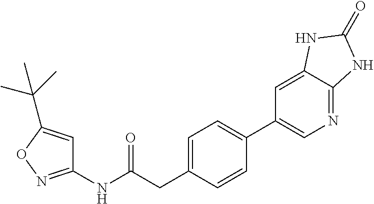 [SMILES: CC(C)(C)c1cc(NC(=O)Cc2ccc(-c3cnc4[nH]c(=O)[nH]c4c3)cc2)no1]

N-(5-tert-Butylisoxazol-3-yl)-2-(4-(2-oxo-2,3-dihydro-1H-imidazo[4,5-b]pyridin-6-yl)phenyl)acetamide (4.62 mg, 5%) was obtained using a procedure analogous to that described in Step 2 of Example 92, substituting 6-bromo-1H-imidazo[4,5-b]pyridin-2(3H)-one for 5-bromo-N-(2-(methylsulfonyl)ethyl)pyridin-2-amine used in Example 92. ¹H NMR (300 MHz, DMSO-d₆) δ 11.23 (br s, 1H), 8.15 (d, J=1.9 Hz, 1H), 7.60 (d, J=8.1 Hz, 2H), 7.31-7.49 (m, 3H), 6.57 (s, 1H), 3.70 (s, 2H), 1.27 (s, 9H). LC-MS (ESI) m/z 392 (M+H)⁺.

### Example 148

### Preparation of 6′-(2-(5-tert-butylisoxazol-3-ylamino)-2-oxoethyl)-5′-fluoro-3,3′-bipyridin-6-aminium methanesulfonate

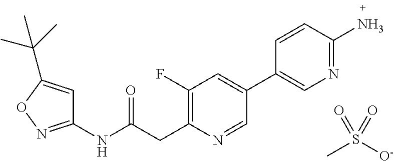

Step 1: To a stirred solution of tert-butyl methyl malonate (898 mg, 5.15 mmol) in DMF (10 mL) at 0° C. was added NaH (60% in mineral oil, 247.2 mg, 6.18 mmol). The reaction mixture was stirred for 20 min at rt. The reaction mixture was then cooled to 0° C. and 5-bromo-2,3-difluoropyridine (1.0 g, 5.15 mmol) was added. LC-MS showed formation of the product. The mixture was partitioned between EtOAc and water and the organic layer was separated, dried over MgSO₄, concentrated under reduced pressure to afford 1-tert-butyl 3-methyl 2-(5-bromo-3-fluoropyridin-2-yl)malonate which was used for the next step without purification.

Step 2: A solution of 1-tert-butyl 3-methyl 2-(5-bromo-3-fluoropyridin-2-yl)malonate (2.48 g, 7.12 mmol) from Step 1 of this example in a mixture of TFA (10 mL) and DCM (10 mL) was stirred at rt for 1 h. LC-MS showed formation of the product. The solvents were evaporated under reduced pressure to afford 5-bromo-3-fluoro-2-(2-methoxy-2-oxoethyl)pyridinium 2,2,2-trifluoroacetate. LC-MS (ESI) m/z 248, 250 (M+H)⁺.

Step 3: To a solution of 5-bromo-3-fluoro-2-(2-methoxy-2-oxoethyl)pyridinium 2,2,2-trifluoroacetate (300 mg, 1.209 mmol) in MeOH (5 mL) at 0° C. was added aq 1M NaOH (2.41 mL, 2.41 mmol). The reaction mixture was stirred at rt overnight. The solvents were evaporated under reduced pressure and the residue was dissolved in water and acidified with 1N HCl. The resulting mixture was extracted with a mixture of DCM and methanol to afford 2-(5-bromo-3-fluoropyridin-2-yl)acetic acid (235 mg, 82%). ¹H NMR (300 MHz, DMSO-d₆) δ 12.73 (s, 1H), 8.53 (s, 1H), 8.19 (dd, J=1.5, 9.2 Hz, 1H), 3.81 (d, J=2.3 Hz, 2H). LC-MS (ESI) m/z 234, 236 (M+H)⁺.

Step 4: 2-(5-Bromo-3-fluoropyridin-2-yl)-N-(5-tert-butylisoxazol-3-yl)acetamide (150 mg, 44%) was obtained using a procedure analogous to that described in Step 2 of Example 91, substituting 5-tert-butylisoxazol-3-amine for 3-tert-butyl-1H-pyrazole and 5-tert-butyl-1H-pyrazol-1-amine used in Example 91. ¹H NMR (300 MHz, DMSO-d₆) δ 11.30 (s, 1H), 8.54 (s, 1H), 8.19 (dd, J=1.7, 9.2 Hz, 1H), 6.55 (s, 1H), 3.95 (d, J=1.7 Hz, 2H), 1.28 (s, 9H). LC-MS (ESI) m/z 356, 358 (M+H)⁺.

Step 5: 2-(6′-Amino-5-fluoro-3,3′-bipyridin-6-yl)-N-(5-tert-butylisoxazol-3-yl)acetamide (29 mg, 19%) was obtained using a procedure analogous to that described in Step 2 of Example 89, substituting 2-(5-bromo-3-fluoropyridin-2-yl)-N-(5-tert-butylisoxazol-3-yl)acetamide from Step 4 of this example for 5-bromo-N-(2-methoxyethyl)pyridin-2-amine used in Example 89. ¹H NMR (300 MHz, DMSO-d₆) δ 11.28 (s, 1H), 8.62 (s, 1H), 8.36 (d, J=2.3 Hz, 1H), 7.94 (dd, J=1.7, 11.3 Hz, 1H), 7.81 (dd, J=2.4, 8.7 Hz, 1H), 6.48-6.61 (m, 2H), 6.27 (s, 2H), 3.95 (s, 2H), 1.28 (s, 9H). LC-MS (ESI) m/z 370 (M+H)⁺.

Step 6: 6′-(2-(5-tert-Butylisoxazol-3-ylamino)-2-oxoethyl)-5′-fluoro-3,3′-bipyridin-6-aminium methanesulfonate (30.86 mg, 86%) was obtained using a procedure analogous to that described in Step 3 of Example 89, substituting 2-(6′-amino-5-fluoro-3,3′-bipyridin-6-yl)-N-(5-tert-butylisoxazol-3-yl)acetamide from Step 5 of this example for N-(5-tert-butylisoxazol-3-yl)-2-(4-(6-(2-methoxyethylamino)pyridin-3-yl)phenyl)acetamide used in Example 89. ¹H NMR (300 MHz, DMSO-d₆) δ 11.32 (s, 1H), 8.71 (s, 1H), 8.29-8.51 (m, 2H), 8.10 (dd, J=1.5, 10.7 Hz, 3H), 7.08 (d, J=9.2 Hz, 1H), 6.56 (s, 1H), 4.01 (s, 2H), 2.34 (d, J=3.0 Hz, 3H), 1.28 (s, 9H). LC-MS (ESI) m/z 370 (M+H)⁺.

### Example 149

### Preparation of 2-(4-(6-aminopyridin-3-yl)phenyl)-N-(5-(1-(difluoromethyl)cyclopropyl)isoxazol-3-yl)acetamide

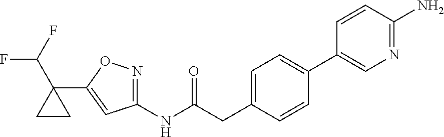 [SMILES: Nc1ccc(-c2ccc(CC(=O)Nc3cc(C4(C(F)F)CC4)on3)cc2)cn1]

Step 1: To a solution of ethyl 1-formylcyclopropanecarboxylate (Sun, D. et al. Bioorganic and Medicinal Chemistry Letters, 2009, vol. 19, p. 1522-1527) (1.1 g, 7.75 mmol) in DCM (20 mL) was added diethylaminofulfur trifluoride (2.5 g, 15.5 mmol) at 0° C. The reaction mixture was allowed to warm to rt and stirred at rt for overnight. The reaction mixture was cooled to 0° C. and carefully quenched with ice chips (30 g). The aqueous layer was seperated and extracted with DCM (2×50 mL). The combined organic layers were washed sequentially with sat aq NaHCO₃ and brine, dried over MgSO₄, filtered, and concentrated under reduced pressure. The residue was purified by silica gel chromatography eluting with 1:2 DCM/hexanes to afford ethyl 1-(difluoromethyl)cyclopropanecarboxylate (700 mg, 55%) as a colorless oil. ¹H NMR (300 MHz, CHLOROFORM-d) δ 6.21-6.77 (m, 1H), 4.22 (q, J=7.1 Hz, 2H), 1.15-1.48 (m, 7H).

Step 2: A stirred suspension of NaH (266 mg, 60% dispersion in mineral oil, 6.6 mmol) in dry THF (20 mL) was heated to 75° C. To this suspension was added a mixture of ethyl 1-(difluoromethyl)cyclopropanecarboxylate (700 mg, 4.27 mmol) from Step 1 of this example and dry acetonitrile (257 mg, 6.6 mmol) dropwise over the course of 15 min. The resulting suspension was heated at 70° C. for 2 h. After cooled to rt, the reaction mixture was poured into water (60 mL) and the resulting mixture was extracted with diethyl ether (2×30 mL). The aqueous layer was separated, acidified to pH˜2 with aq 2N hydrochloric acid and extracted with diethyl ether (2×40 mL). The combined organic layers were dried over MgSO₄, filtered, and concentrated under reduced pressure to afford 3-(1-(difluoromethyl)cyclopropyl)-3-oxopropanenitrileas (430 mg, 63%) as an yellow oil. ¹H NMR (300 MHz, CHLOROFORM-d) δ 6.18-6.73 (m, 1H), 5.46-5.99 (m, 1H), 3.87 (s, 1H), 1.50-1.63 (m, 1H), 1.14-1.46 (m, 3H).

Step 3: 5-(1-(Difluoromethyl)cyclopropyl)isoxazol-3-amine (200 mg, 42%) was synthesized as an yellow oil using a procedure analogous to that described in Step 2 of Example 100, substituting 3-(1-(difluoromethyl)cyclopropyl)-3-oxopropanenitrile from Step 2 of this example for 3-(1-methylcyclopropyl)-3-oxopropanenitrile used in Example 100. ¹H NMR (300 MHz, CHLOROFORM-d) δ 5.69-6.23 (m, 1H), 5.11 (s, 1H), 4.71 (br s, 2H), 0.91-1.51 (m, 4H). LC-MS (ESI) m/z 175 (M+H)⁺.

Step 4: N-(5-(1-(Difluoromethyl)cyclopropyl)isoxazol-3-yl)-2-(4-(4,4,5,5-tetramethyl-1,3,2-dioxaborolan-2-yl)phenyl)acetamide (170 mg, 35%) was synthesized as a light yellow solid using the procedure analogous to that described in Step 1 of Example 85, substituting 5-(1-(difluoromethyl)cyclopropyl)isoxazol-3-amine from Step 3 of this example for 5-tert-butylisoxazol-3-amine used in Example 85. LC-MS (ESI) m/z 419 (M+H)⁺.

Step 5: 2-(4-(6-Aminopyridin-3-yl)phenyl)-N-(5-(1-(difluoromethyl)cyclopropyl)isoxazol-3-yl)acetamide (18 mg, 14%) was synthesized as a solid according to the procedure described in Step 2 of Example 85, substituting N-(5-(1-(difluoromethyl)cyclopropyl)isoxazol-3-yl)-2-(4-(4,4,5,5-tetramethyl-1,3,2-dioxaborolan-2-yl)phenyl)acetamide from Step 4 of this example for N-(5-tert-butylisoxazol-3-yl)-2-(4-(4,4,5,5-tetramethyl-1,3,2-dioxaborolan-2-yl)phenyl)acetamide, and 5-bromopyridin-2-amine for 5-bromo-3-methylpyridin-2-amine used in Example 85. ¹H NMR (300 MHz, DMSO-d₆) δ 11.92 (s, 1H), 8.22 (d, J=1.9 Hz, 1H), 7.68 (dd, J=2.4, 8.6 Hz, 1H), 7.52 (d, J=8.3 Hz, 2H), 7.33 (d, J=7.9 Hz, 2H), 6.51 (d, J=8.5 Hz, 1H), 6.28 (s, 2H), 5.91-6.17 (m, 2H), 3.71 (s, 4H), 1.11-1.36 (m, 4H). LC-MS (ESI) m/z 385 (M+H)⁺.

### Example 150

### Preparation of 5-(4-(2-(5-tert-butylisoxazol-3-ylamino)-2-oxoethyl)phenyl)-4-chloropyridin-2-aminium methanesulfonate

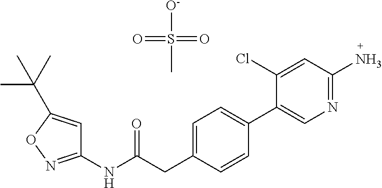

Step 1: 5-Bromo-4-chloropyridin-2-amine (1.97 g, 61%) was obtained as a faint yellow solid using a procedure analogous to that described in Step 1 of Example 94, substituting 4-chloropyridin-2-amine for 3-fluoropyridin-2-amine used in Example 94. ¹H NMR (300 MHz, CHLOROFORM-d) δ 8.19 (s, 1H), 6.65 (s, 1H), 4.56 (br s, 2H). LC-MS (ESI) m/z 207, 209, and 211 (M+H)⁺.

Step 2: 5-(4-(2-(5-tert-Butylisoxazol-3-ylamino)-2-oxoethyl)phenyl)-4-chloropyridin-2-aminium methanesulfonate (89 mg, 48%) was obtained as a pink solid using procedures analogous to those described in Steps 1-2 of Example 110, substituting 5-bromo-4-chloropyridin-2-amine for 5-bromo-N-methylpyridin-2-amine used in Example 110. ¹H NMR (300 MHz, DMSO-d₆) δ 11.25 (s, 1H), 8.03 (s, 1H), 7.63-7.95 (m, 2H), 7.34-7.50 (m, 4H), 7.08 (s, 1H), 6.57 (s, 1H), 3.73 (s, 2H), 2.33 (s, 3H), 1.27 (s, 9H). LC-MS (ESI) m/z 385 (M+H)⁺.

### Example 151

### Preparation of 5-(4-(2-(5-tert-butylisoxazol-3-ylamino)-2-oxoethyl)phenyl)-4-fluoropyridin-2-aminium methanesulfonate

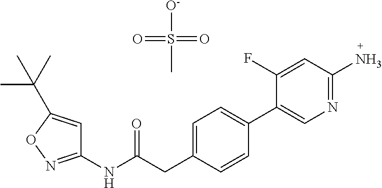

Step 1: 5-Bromo-4-fluoropyridin-2-amine (317 mg, 37%) was obtained as a faint orange solid using a procedure analogous to that described in Step 1 of Example 94, substituting 4-fluoropyridin-2-amine for 3-fluoropyridin-2-amine used in Example 94. ¹H NMR (300 MHz, CHLOROFORM-d) δ 7.90 (br s, 1H), 7.83 (d, J=6.8 Hz, 1H), 6.05-7.08 (m, 2H). LC-MS (ESI) m/z 191 and 193 (M+H)⁺.

Step 2: 5-(4-(2-(5-tert-Butylisoxazol-3-ylamino)-2-oxoethyl)phenyl)-4-fluoropyridin-2-aminium methanesulfonate (47 mg, 26%) was obtained as a pink solid using procedures analogous to those described in Steps 1-2 of Example 110, substituting 5-bromo-4-fluoropyridin-2-amine for 5-bromo-N-methylpyridin-2-amine used in Example 110. ¹H NMR (300 MHz, DMSO-d₆) δ 11.27 (s, 1H), 8.20 (t, J=2.5 Hz, 1H), 7.98 (dd, J=2.7, 8.6 Hz, 1H), 7.41-7.55 (m, 4H), 6.57 (s, 1H), 3.75 (s, 2H), 2.37 (s, 3H), 1.28 (s, 9H). LC-MS (ESI) m/z 369 (M+H)⁺.

### Example 152

### Preparation of 5-(4-(2-(5-tert-butylisoxazol-3-ylamino)-2-oxoethyl)phenyl)-3,6-difluoropyridin-2-aminium methanesulfonate

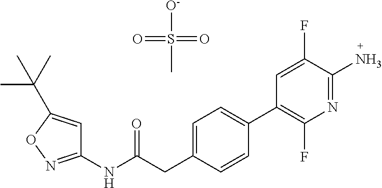

Step 1: 5-Bromo-3,6-difluoropyridin-2-amine (550 mg, 86%) was obtained as a faint orange solid using a procedure analogous to that described in Step 1 of Example 94, substituting 3,6-difluoropyridin-2-amine (Gudmundsson, Kristjan, et al. WO2007/87549; 2007) for 3-fluoropyridin-2-amine used in Example 94. ¹H NMR (300 MHz, CHLOROFORM-d) δ 7.45-7.53 (m, 1H), 4.74 (br s, 2H). LC-MS (ESI) m/z 209 and 211 (M+H)⁺.

Step 2: 5-(4-(2-(5-tert-Butylisoxazol-3-ylamino)-2-oxoethyl)phenyl)-3,6-difluoropyridin-2-aminium methanesulfonate (70 mg, 37%) was obtained as an off-white solid using procedures analogous to those described in Steps 1-2 of Example 110, substituting 5-bromo-3,6-difluoropyridin-2-amine for 5-bromo-N-methylpyridin-2-amine used in Example 110. ¹H NMR (300 MHz, DMSO-d₆) δ 11.21 (s, 1H), 7.73 (dd, J=7.9, 10.7 Hz, 1H), 7.44-7.49 (m, 2H), 7.32-7.38 (m, 2H), 6.57 (s, 1H), 3.68 (s, 2H), 2.33 (s, 3H), 1.27 (s, 9H). LC-MS (ESI) m/z 387 (M+H)⁺.

### Examples 153-191

The following additional examples were prepared by methods analogous to those described in the foregoing procedures. HPLC retention times were recorded on a Shimadzu LCMS-2010 EV equipped with an Agilent Zorbax Eclipse Plus C18 column, 150×2.0 mm I.D., 3.5 μm, operating at room temperature at a flow rate of 0.5 mL/min. The buffer systems are (A) 0.05% aq HOAc and (B) 0.05% HOAc/CH₃CN with elution according to the following gradient:
t=0 min, 10% B
t=12 min, 95% B
t=14 min, 95% B
t=15 min, 10% B

TABLE 1

| | | HPLC | |
|---|---|---|---|
| | | retention | |
| Example | Structure | time | m/z |
| 153 | 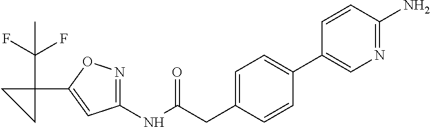 [SMILES: CC(F)(F)C1(c2cc(NC(=O)Cc3ccc(-c4ccc(N)nc4)cc3)no2)CC1] | 4.42 | 399 (M + H)+ |
| 154 | 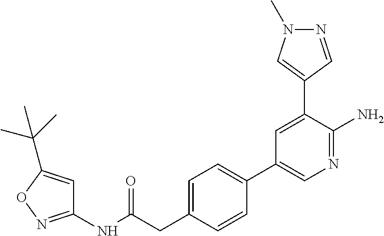 [SMILES: Cn1cc(-c2cc(-c3ccc(CC(=O)Nc4cc(C(C)(C)C)on4)cc3)cnc2N)cn1] | 4.61 | 431 (M + H)+ |
| 155 | 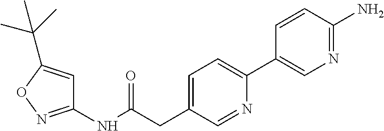 [SMILES: CC(C)(C)c1cc(NC(=O)Cc2ccc(-c3ccc(N)nc3)nc2)no1] | 3.48 | 352 (M + H)+ |
| 156 | 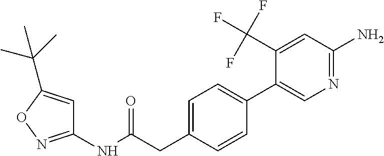 [SMILES: CC(C)(C)c1cc(NC(=O)Cc2ccc(-c3cnc(N)cc3C(F)(F)F)cc2)no1] | 7.66 | 419 (M + H)+ |
| 157 | 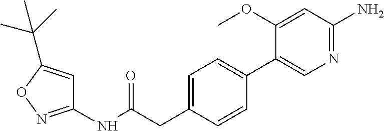 [SMILES: COc1cc(N)ncc1-c1ccc(CC(=O)Nc2cc(C(C)(C)C)on2)cc1] | 3.89 | 381 (M + H)+ |
| 158 | 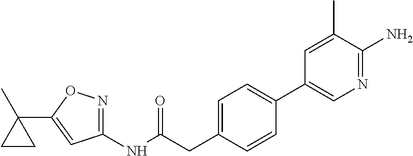 [SMILES: Cc1cc(-c2ccc(CC(=O)Nc3cc(C4(C)CC4)on3)cc2)cnc1N] | 3.78 | 363 (M + H)+ |
| 159 | 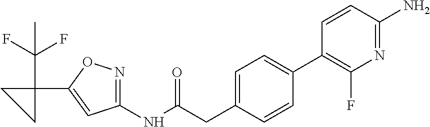 [SMILES: CC(F)(F)C1(c2cc(NC(=O)Cc3ccc(-c4ccc(N)nc4F)cc3)no2)CC1] | 7.38 | 417 (M + H)+ |
| 160 | 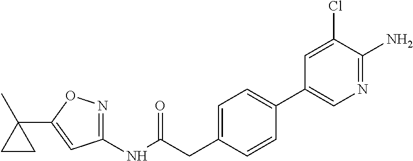 [SMILES: CC1(c2cc(NC(=O)Cc3ccc(-c4cnc(N)c(Cl)c4)cc3)no2)CC1] | 6.88 | 383 (M + H)+ |
| 161 | 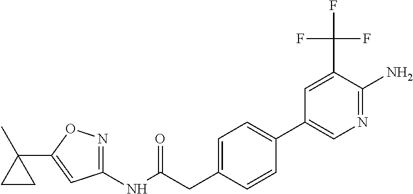 [SMILES: CC1(c2cc(NC(=O)Cc3ccc(-c4cnc(N)c(C(F)(F)F)c4)cc3)no2)CC1] | 7.65 | 417 (M + H)+ |
| 162 | 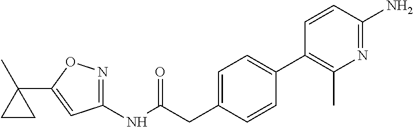 [SMILES: Cc1nc(N)ccc1-c1ccc(CC(=O)Nc2cc(C3(C)CC3)on2)cc1] | 3.68 | 363 (M + H)+ |
| 163 | 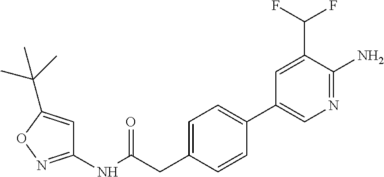 [SMILES: CC(C)(C)c1cc(NC(=O)Cc2ccc(-c3cnc(N)c(C(F)F)c3)cc2)no1] | 7.06 | 401 (M + H)+ |
| 164 | 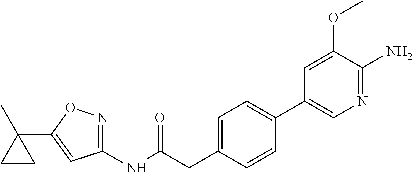 [SMILES: COc1cc(-c2ccc(CC(=O)Nc3cc(C4(C)CC4)on3)cc2)cnc1N] | 4.13 | 379 (M + H)+ |
| 165 | 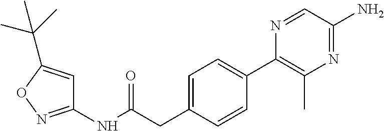 [SMILES: Cc1nc(N)cnc1-c1ccc(CC(=O)Nc2cc(C(C)(C)C)on2)cc1] | 6.08 | 366 (M + H)+ |
| 166 | 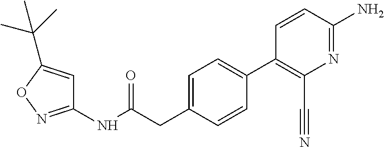 [SMILES: CC(C)(C)c1cc(NC(=O)Cc2ccc(-c3ccc(N)nc3C#N)cc2)no1] | 7.22 | 376 (M + H)+ |
| 167 | 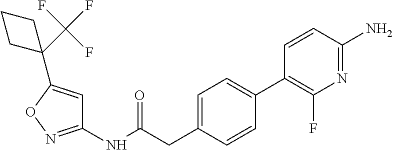 [SMILES: Nc1ccc(-c2ccc(CC(=O)Nc3cc(C4(C(F)(F)F)CCC4)on3)cc2)c(F)n1] | 8.06 | 435 (M + H)+ |
| 169 | 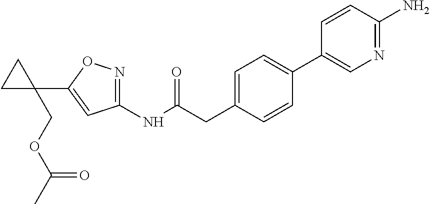 [SMILES: CC(=O)OCC1(c2cc(NC(=O)Cc3ccc(-c4ccc(N)nc4)cc3)no2)CC1] | 3.5 | 407 (M + H)+ |
| 170 | 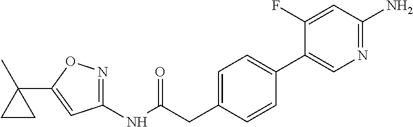 [SMILES: CC1(c2cc(NC(=O)Cc3ccc(-c4cnc(N)cc4F)cc3)no2)CC1] | 6.44 | 367 (M + H)+ |
| 171 | 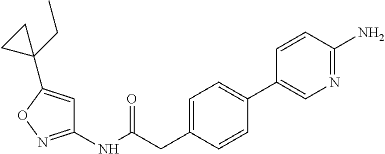 [SMILES: CCC1(c2cc(NC(=O)Cc3ccc(-c4ccc(N)nc4)cc3)no2)CC1] | 4.36 | 363 (M + H)+ |
| 172 | 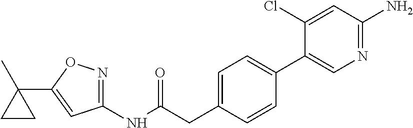 [SMILES: CC1(c2cc(NC(=O)Cc3ccc(-c4cnc(N)cc4Cl)cc3)no2)CC1] | 5.54 | 383 (M + H)+ |
| 173 | 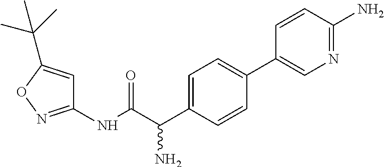 [SMILES: CC(C)(C)c1cc(NC(=O)C(N)c2ccc(-c3ccc(N)nc3)cc2)no1] | 1.69 | 366 (M + H)+ |
| 174 | 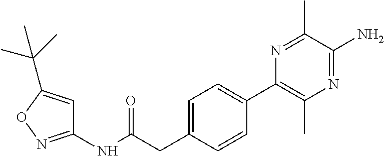 [SMILES: Cc1nc(-c2ccc(CC(=O)Nc3cc(C(C)(C)C)on3)cc2)c(C)nc1N] | 6.19 | 380 (M + H)+ |
| 175 | 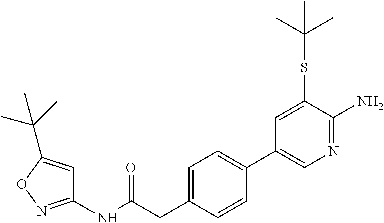 [SMILES: CC(C)(C)Sc1cc(-c2ccc(CC(=O)Nc3cc(C(C)(C)C)on3)cc2)cnc1N] | 8.09 | 439 (M + H)+ |
| 176 | 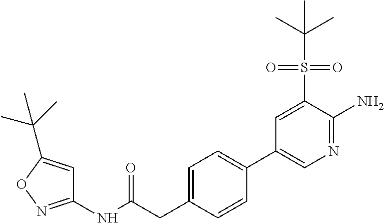 [SMILES: CC(C)(C)c1cc(NC(=O)Cc2ccc(-c3cnc(N)c(S(=O)(=O)C(C)(C)C)c3)cc2)no1] | 8.07 | 471 (M + H)+ |
| 177 | 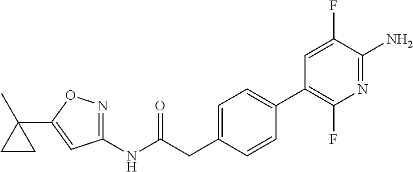 [SMILES: CC1(c2cc(NC(=O)Cc3ccc(-c4cc(F)c(N)nc4F)cc3)no2)CC1] | 7.4 | 385 (M + H)+ |
| 178 | 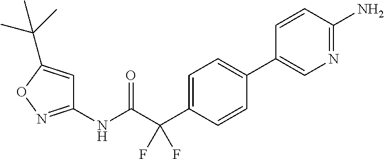 [SMILES: CC(C)(C)c1cc(NC(=O)C(F)(F)c2ccc(-c3ccc(N)nc3)cc2)no1] | 5.1 | 387 (M + H)+ |
| 179 | 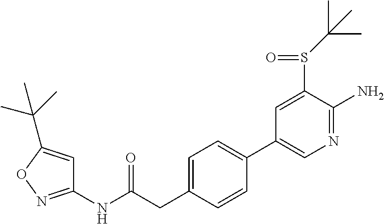 [SMILES: CC(C)(C)c1cc(NC(=O)Cc2ccc(-c3cnc(N)c(S(=O)C(C)(C)C)c3)cc2)no1] | 7.1 | 455 (M + H)+ |
| 180 | 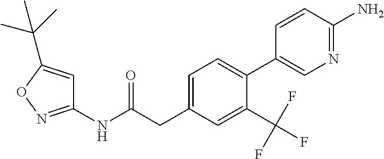 [SMILES: CC(C)(C)c1cc(NC(=O)Cc2ccc(-c3ccc(N)nc3)c(C(F)(F)F)c2)no1] | 4.9 | 419 (M + H)+ |
| 181 | 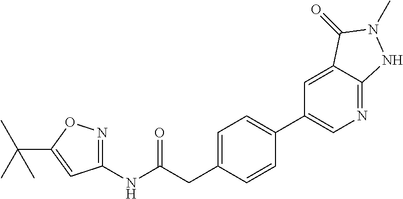 [SMILES: Cn1[nH]c2ncc(-c3ccc(CC(=O)Nc4cc(C(C)(C)C)on4)cc3)cc2c1=O] | 5.5 | 406 (M + H)+ |
| 182 | 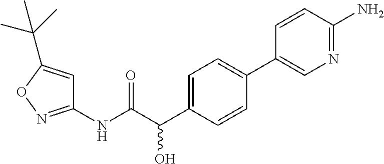 [SMILES: CC(C)(C)c1cc(NC(=O)C(O)c2ccc(-c3ccc(N)nc3)cc2)no1] | 3.3 | 367 (M + H)+ |
| 183 | 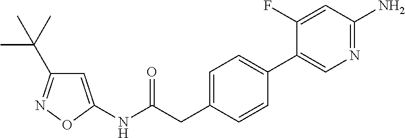 [SMILES: CC(C)(C)c1cc(NC(=O)Cc2ccc(-c3cnc(N)cc3F)cc2)on1] | 6.8 | 369 (M + H)+ |
| 184 | 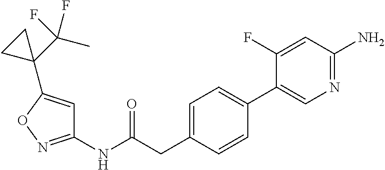 [SMILES: CC(F)(F)C1(c2cc(NC(=O)Cc3ccc(-c4cnc(N)cc4F)cc3)no2)CC1] | 7.1 | 417 (M + H)+ |
| 185 | 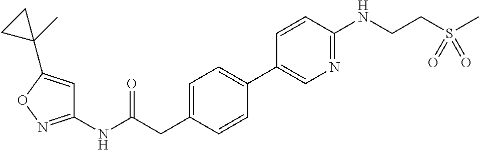 [SMILES: CC1(c2cc(NC(=O)Cc3ccc(-c4ccc(NCCS(C)(=O)=O)nc4)cc3)no2)CC1] | 4.9 | 455 (M + H)+ |
| 186 | 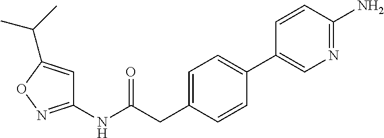 [SMILES: CC(C)c1cc(NC(=O)Cc2ccc(-c3ccc(N)nc3)cc2)no1] | 3.8 | 337 (M + H)+ |
| 187 | 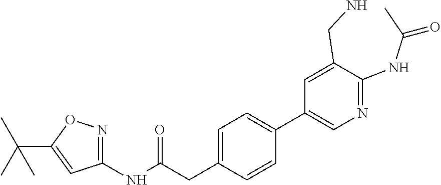 [SMILES: CC(=O)Nc1ncc(-c2ccc(CC(=O)Nc3cc(C(C)(C)C)on3)cc2)cc1CN] | 4.2 | 420 (M + H)+ |
| 188 | 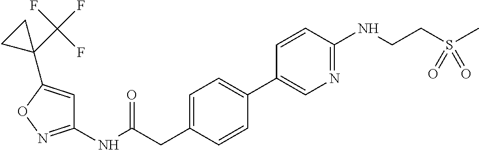 [SMILES: CS(=O)(=O)CCNc1ccc(-c2ccc(CC(=O)Nc3cc(C4(C(F)(F)F)CC4)on3)cc2)cn1] | 5.6 | 509 (M + H)+ |
| 189 | 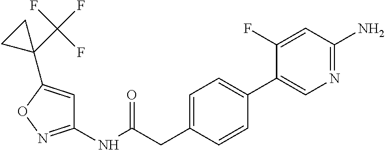 [SMILES: Nc1cc(F)c(-c2ccc(CC(=O)Nc3cc(C4(C(F)(F)F)CC4)on3)cc2)cn1] | 7.15 | 421 (M + H)+ |
| 190 | 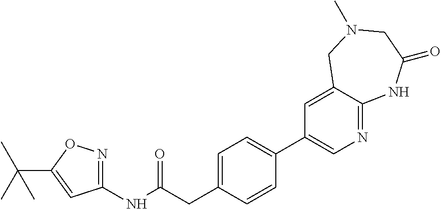 [SMILES: CN1CC(=O)Nc2ncc(-c3ccc(CC(=O)Nc4cc(C(C)(C)C)on4)cc3)cc2C1] | 4.8 | 434 (M + H)+ |
| 191 | 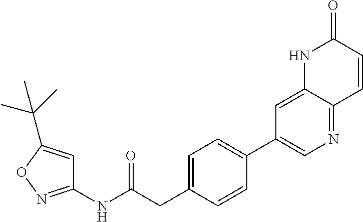 [SMILES: CC(C)(C)c1cc(NC(=O)Cc2ccc(-c3cnc4ccc(=O)[nH]c4c3)cc2)no1] | 5.97 | 403 (M + H)+ |

### Example 192

### M-NFS-60 Cell Proliferation Assay

The compounds disclosed herein were tested in an M-NFS-60 cell proliferation assay to determine their cellular potency against CSF1R. M-NFS-60s are mouse monocytic cells that depend on the binding of the ligand M-CSF to its receptor, CSF1R, to proliferate. Inhibition of CSF1R kinase activity will cause reduced growth and/or cell death. This assay assesses the potency of compounds as CSF1R inhibitors by measuring the reduction of Alamar Blue reagent by viable cells.

On day one of the experiment, M-NFS-60 cells were maintained in RPMI complete medium (Omega Scientific) plus 10% FBS supplemented with 20 ng/mL of M-CSF (R&D Systems). 96-well TC-treated, flat bottom plates were seeded at 10,000 cell/well at a volume of 100 μL per well. The cells were cultured overnight at 37° C. under 5% CO₂.

On day two, compounds were added to the cells at 9 different concentrations, with half-log intervals alongside a control reference compound serving as a positive control. Final DMSO concentration was kept at 0.5% for a final volume of 200 μL. The compounds were allowed to incubate with the cells for 72 hours at 37° C. under 5% CO₂.

On day five of the experiment, 40 μl of Alamar Blue reagent was added to each well and allowed to incubate for 3 hours. Alamar Blue fluorescence was read using SoftMax Pro software at 560 nm (excitation) and 590 nm (emission). IC₅₀s were generated as an average of duplicates and represents the concentration of test compound that achieves 50% inhibition of cellular proliferation compared to control.

In one embodiment, the compounds provided herein were found to have IC₅₀ of about or less than about 20, 15, 10, 5, 1, Or 0.5 μM. In another embodiment, the compounds provided herein were found to have IC₅₀ of about or less than about 1000, 500, 300, 100, 50, 40, 30 or 20 nM. In another embodiment, the compounds provided herein were found to have IC₅₀ of less than about 200 nM.

### Example 193

### MV4-11 Cell Proliferation Assay

The compounds disclosed herein were tested in an MV4-11 cell proliferation assay to determine their cellular potency against Flt3. MV4-11 cells carry ITD mutation within juxtamembrane domain of Flt3 kinase which renders the kinase constitutively active. The growth and/or survival of MV4-11 cells are greatly reduced in the presence of Flt3 inhibitors. This assay measures the potency of compounds as Flt3 inhibitors by measuring the reduction of Alamar Blue reagent by viable cells.

MV4-11 cells were grown in an incubator at 37° C. under 5% CO₂ in Iscove's media (Celgro) with 10% FBS. The cell density was kept between 1e5 and 8e5 cells/mL.

On day one of the experiment, cells were harvested and spun at 500 g for 5 min at 4° C., the supernatant aspirated and the cells resuspended in Iscove's media with 0.5% FBS. Cell density was maintained at 7.5e5 to achieve maximum viability of the cells. The resuspended cells were incubated at 37° C. in 5% CO₂ overnight.

On day two of the experiment, cells were diluted to 6.4e5/mL with Iscove's media with 0.5% FBS. 100 μL of the cell suspension (64,000 cells) were aliquoted into each well of a 96-well TC-treated plate. Compounds were added at 9 different concentrations, with half-log intervals alongside a control reference compound serving as a positive control. Final DMSO concentration was kept at 0.5% and final volume at 200 μL. The cells were then incubated at 37° C. under 5% CO₂ for 3 days.

On day five of the experiment, 40 μL of Alamar Blue reagent was added to each well and the mixture was allowed to incubate for 3 hours. Alamar Blue fluorescence was measured using SoftMax Pro software at 560 nm (excitation) and 590 nm (emission). IC₅₀s were generated as an average of duplicates and represents the concentration of test compound that achieves 50% inhibition of cellular proliferation compared to negative control.

In one embodiment, the compounds provided herein were found to have IC₅₀ of about or less than about 20, 15, 10, 5, 1, Or 0.5 μM. In another embodiment, the compounds provided herein were found to have activity IC₅₀ of about or less than about 1000, 500, 300, 100, 50, 40, 30 or 20 nM. In another embodiment, the compounds provided herein were found to have activity IC₅₀ of less than about 200 nM. In another embodiment, the compounds provided herein have IC₅₀ as indicated in Tables 2 and 3.

### Example 194

### Competition Binding Assay to Determine Selectivity Scores and Binding Constants (Kₐ) of the Compounds a Against a Panel of Kinases

Competition binding assays used herein were developed, validated and performed as described in Fabian et al., Nature Biotechnology 2005, 23,329-336. Kinases were produced as fusions to T7 phage (See, Fabian et al. or WO04/015142) or alternatively, the kinases were expressed in HEK-293 cells and subsequently tagged with DNA for PCR detection (See, WO08/005,310). For the binding assays, streptavidin-coated magnetic beads were treated with biotinylated affinity ligands for 30 min at room temperature to generate affinity resins. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinase, liganded affinity beads, and test compounds in 1× binding buffer (20% SeaBlock, 0.17×PBS, 0.05% Tween 20, 6 mM DTT). Test compounds were prepared as 100× stocks in DMSO and diluted into the aqueous environment. Kds were determined using an eleven point threefold serial dilutions. DMSO or control compounds were was added to control assays lacking a test compound. Primary screen assays were performed in polypropylene 384-well plates in a final volume of 20-40 μL, while Kd determinations were performed in polystyrene 96-well plates in a final volume of 135 μL. The assay plates were incubated at room temperature with shaking for 1 hour to allow the binding reactions to reach equilibrium, and the affinity beads were washed extensively with wash buffer (1×PBS, 0.05% Tween 20) to remove unbound protein. The beads were then resuspended in elution buffer (1×PBS, 0.05% Tween 20, 0.5 μM non-biotinylated affinity ligand) and incubated at room temperature with shaking for 30 min. The kinase concentration in the eluates was measured by quantitative PCR.

A selectivity score (S10) is a quantitative measure of selectivity of a compound against a panel of kinases. An S10 was calculated for a compound by dividing the number of kinases found to have a percent of control (DMSO) less than 10 by the total number of distinct kinases tested (excluding mutant variants). Percent of control (POC) is calculated by subtracting the signal of the control compound (POC=0) from the signal of the test compound and dividing the outcome by the signal of DMSO (POC=100) minus the signal of the control compound. For the compounds disclosed herein, S10 scores were obtained by testing the compounds at 10 μM concentration in a kinase panel containing either 359 or 386 distinct kinases.

In one embodiment, the compounds provided herein were found to have Kds of about or less than about 20 μM against CSF-1R kinase. In one embodiment, the compounds provided herein were found to have Kds of less than about 10, 5, 3, 1, 0.5, 0.1 or 0.01 μM against CSF-1R kinase. In one embodiment, the compounds provided herein were found to have Kds of less than about 100, 50, 10, 5, 4, 3, 2, or 1 nM against CSF-1R kinase. In another embodiment, the compounds provided herein were found to have Kds of about or less than about 1 nM against CSF-1R kinase. In another embodiment, the compounds provided herein have Kds against CSF-1R kinase as indicated in Tables 2 and 3.

In one embodiment, the compounds provided herein were found to have Kds of less than about 1000, 500, 100, 50, 10, 5, 4, 3, 2, or 1 nM against FLT3 kinase. In another embodiment, the compounds provided herein were found to have Kds of about or less than about 5, 1, or 0.5 nM against FLT3 kinase. In another embodiment, the compounds provided herein have Kds against FLT3 kinase as indicated in Tables 2 and 3.

In one embodiment, the compounds provided herein were found to have Kds of less than about 1000, 500, 100, 50, 10, 5, 4, 3, 2, or 1 nM against KIT kinase. In another embodiment, the compounds provided herein were found to have Kds of about or less than about 5, 1, or 0.5 nM against KIT kinase. In another embodiment, the compounds provided herein have Kds against KIT kinase as indicated in Tables 2 and 3.

In one embodiment, the compounds provided herein were found to have S10 score of less than about 0.3, 0.2, 0.1, 0.05, 0.01, or 0.005. In another embodiment, the compounds provided herein have S10 scores as indicated in Tables 2 and 3.

In Tables 2 and 3,
FLT3 kd (nM): A≦1 nM; 1<B≦10 nM; 10<C≦100 nM
KIT kd (nM): A≦1 nM; 1<B≦10 nM; 10<C≦100 nM
CSF-1R Kd (nM): A≦10 nM; 10<B≦100 nM; 100<C≦500 nM;
ND=no data; and NA=no activity
FLT3 Cell Proliferation Assay (MV4-11) IC₅₀ (nM): A≦1 nM;
1<B≦10 nM; 10<C≦100 nM; 100<D≦500 nM; ND=no data, and NA=no activity
S Score 10 μM (359 or 386 panel): A≦0.1; 0.1<B≦0.2 nM; 0.2<B≦0.5 nM; and ND=no data.

TABLE 2

| | Binding Assay: | | | Cell Assay: | | |
|---|---|---|---|---|---|---|
| | Binding | | Binding | MV4-11 | Kinase | Kinase |
| | Assay: | Binding | Assay: | Cell | specificty: | specificty: |
| | FLT3 | Assay: | CSF-1R | proliferation | S(10)-359 | S(10)-386 |
| Ex. | Kd | KIT Kd | Kd | assay: IC₅₀ | panel | panel |
| 44 | A | B | A | A | B | ND |
| 45 | A | A | A | NA | B | ND |
| 45 | A | A | A | B | B | ND |
| 46 | B | A | B | C | A | ND |
| 47 | B | B | B | C | A | ND |
| 48 | B | B | A | — | A | ND |
| 49 | A | A | A | A | B | ND |
| 50 | B | A | A | A | B | ND |
| 1 | A | A | A | A | A | A |
| 2 | A | B | A | A | B | ND |
| 51 | C | B | C | — | A | ND |
| 3 | A | A | A | A | A | ND |
| 52 | A | A | A | B | B | ND |
| 53 | A | A | A | B | B | ND |
| 54 | A | A | A | C | A | ND |
| 55 | B | A | A | D | A | ND |
| 4 | B | B | A | B | B | ND |
| 5 | A | B | A | A | B | ND |
| 56 | A | B | A | C | A | ND |
| 6 | B | B | B | B | A | ND |
| 57 | A | A | A | C | A | ND |
| 58 | A | A | A | B | B | ND |
| 7 | A | A | A | B | A | ND |
| 8 | B | B | A | NA | A | ND |
| 9 | B | B | A | C | A | ND |
| 59 | A | A | A | B | B | ND |
| 10 | B | B | A | D | A | ND |
| 11 | A | B | A | B | A | ND |
| 12 | B | B | A | B | A | ND |
| 13 | B | B | B | C | B | ND |
| 60 | A | B | A | C | B | ND |
| 14 | A | A | A | A | B | ND |
| 15 | B | B | B | C | B | ND |
| 16 | A | A | B | B | B | ND |
| 17 | A | A | A | B | A | ND |
| 18 | A | B | A | A | | A |
| 18 | B | B | A | A | A | ND |
| 19 | B | B | B | NA | A | ND |
| 20 | A | B | A | B | B | ND |
| 21 | A | A | A | B | B | ND |
| 22 | A | A | A | A | B | ND |
| 23 | A | B | A | C | A | ND |
| 24 | A | B | A | B | A | ND |
| 61 | A | A | A | C | A | ND |
| 62 | B | B | B | D | A | ND |
| 63 | A | A | A | C | B | ND |
| 64 | A | A | A | C | B | ND |
| 65 | A | A | A | C | B | ND |
| 25 | A | A | B | D | A | ND |
| 26 | A | A | A | D | A | ND |
| 66 | B | A | A | D | A | ND |
| 67 | B | A | B | C | A | ND |
| 27 | A | A | A | C | ND | ND |
| 28 | A | A | A | A | ND | ND |
| 29 | A | A | A | B | ND | ND |
| 30 | B | A | B | C | ND | ND |
| 31 | A | A | A | C | ND | ND |
| 32 | A | A | A | B | ND | ND |
| 33 | A | A | A | A | ND | ND |
| 34 | A | A | A | A | ND | ND |
| 35 | A | B | A | A | ND | ND |
| 36 | A | A | A | B | ND | ND |
| 37 | A | A | A | B | ND | ND |
| 38 | B | A | A | C | ND | ND |
| 39 | A | B | A | B | ND | ND |
| 40 | A | A | A | B | ND | ND |
| 41 | A | A | A | B | ND | ND |
| 42 | B | A | A | C | ND | ND |
| 43 | A | A | A | B | ND | ND |
| 68 | B | B | A | C | ND | ND |
| 69 | B | A | A | C | ND | ND |
| 70 | B | B | A | A | ND | ND |
| 71 | B | C | B | C | ND | ND |
| 72 | A | B | A | A | ND | ND |
| 73 | A | B | A | A | ND | A |
| 73 | A | B | A | A | ND | ND |
| 74 | A | B | A | A | ND | ND |
| 75 | A | B | A | A | ND | ND |
| 76 | A | A | A | B | ND | ND |
| 77 | A | B | A | B | ND | ND |
| 78 | A | B | A | A | ND | ND |
| 78 | A | B | A | — | ND | B |
| 79 | A | B | A | A | ND | ND |
| 80 | A | A | A | B | ND | ND |
| 81 | B | B | A | C | ND | ND |
| 82 | B | B | B | D | ND | ND |
| 83 | A | B | A | C | ND | ND |
| 84 | A | B | A | A | ND | ND |
| 85 | A | B | A | A | ND | ND |
| 86 | A | B | B | C | ND | ND |
| 87 | A | B | B | C | ND | ND |
| 88 | A | B | B | B | ND | ND |
| 89 | A | B | A | B | ND | ND |
| 90 | B | B | B | D | ND | ND |
| 91 | A | B | B | B | ND | A |
| 92 | A | B | A | B | ND | B |
| 93 | B | B | A | A | ND | C |
| 94 | B | B | A | A | ND | B |
| 95 | A | B | A | A | ND | A |
| 96 | A | A | A | B | ND | B |
| 97 | B | B | A | B | ND | A |
| 98 | B | B | A | C | ND | A |
| 99 | A | B | A | B | ND | A |
| 100 | A | A | A | B | ND | A |
| 101 | A | A | A | B | ND | A |
| 102 | A | B | A | A | ND | B |
| 103 | A | A | A | A | ND | A |
| 104 | A | A | A | C | ND | A |
| 105 | A | A | A | B | ND | B |
| 106 | A | A | A | B | ND | A |
| 107 | A | B | A | B | ND | A |
| 108 | A | B | A | B | ND | A |
| 109 | A | B | A | A | ND | B |
| 110 | A | B | A | A | ND | B |
| 111 | A | A | A | A | ND | A |
| 112 | A | B | A | B | ND | A |
| 113 | A | B | A | A | ND | B |
| 114 | A | A | A | A | ND | C |
| 115 | B | B | A | A | ND | B |
| 116 | A | B | A | A | ND | B |
| 117 | A | B | A | A | ND | B |
| 118 | A | A | A | C | ND | A |
| 119 | A | B | A | A | ND | A |
| 120 | A | B | A | B | ND | B |
| 121 | B | B | B | A | ND | A |
| 122 | B | C | C | C | ND | A |
| 123 | A | B | A | A | ND | B |
| 124 | A | B | A | A | ND | B |
| 125 | A | A | A | A | ND | A |
| 126 | A | B | B | C | ND | A |
| 127 | A | B | A | A | ND | B |
| 128 | A | B | A | B | ND | B |
| 129 | C | C | NA | D | ND | A |
| 130 | A | B | A | A | ND | A |
| 131 | B | C | NA | D | ND | A |
| 132 | A | B | A | B | ND | A |
| 133 | A | B | A | A | ND | B |
| 134 | A | A | A | B | ND | A |
| 135 | A | B | A | B | ND | B |
| 136 | A | B | A | A | ND | B |
| 137 | A | B | B | A | ND | A |
| 138 | A | A | A | A | ND | B |
| 139 | A | B | A | A | ND | B |
| 140 | A | B | A | C | ND | A |
| 141 | B | B | B | B | ND | B |
| 142 | A | B | A | A | ND | A |
| 143 | A | A | A | B | ND | A |
| 144 | A | A | A | A | ND | B |
| 145 | A | B | A | C | ND | A |
| 146 | A | A | A | B | ND | A |
| 147 | B | B | A | A | ND | B |
| 148 | A | A | A | B | ND | A |
| 149 | A | A | A | B | ND | B |
| 150 | A | B | A | B | ND | A |
| 151 | A | C | A | B | ND | B |
| 152 | A | B | A | A | ND | A |
| 153 | A | B | A | A | ND | B |
| 154 | A | B | B | B | ND | C |
| 155 | A | A | C | C | ND | A |
| 156 | A | B | B | C | ND | A |
| 157 | A | B | B | B | ND | A |
| 158 | A | A | A | B | ND | A |
| 159 | B | B | A | A | ND | A |
| 160 | A | A | A | A | ND | B |
| 161 | A | B | A | A | ND | B |
| 162 | A | A | A | C | ND | A |
| 163 | A | B | A | A | ND | B |
| 164 | A | A | A | B | ND | B |
| 165 | A | B | B | C | ND | A |
| 166 | A | B | B | C | ND | A |
| 167 | A | B | A | A | ND | A |
| 169 | A | B | A | C | ND | ND |
| 170 | A | B | A | B | ND | ND |
| 171 | A | B | A | A | ND | ND |
| 172 | A | A | A | B | ND | ND |
| 173 | B | C | NA | D | ND | ND |
| 174 | A | B | C | C | ND | ND |
| 175 | B | C | C | C | ND | ND |
| 176 | A | B | NA | C | ND | ND |

TABLE 3

| | | | | Cell Assay: |
|---|---|---|---|---|
| | Binding | Binding | Binding | MV4-11 Cell |
| | Assay: FLT3 | Assay: KIT | Assay: CSF1R | proliferation |
| Ex. | Kd | Kd | Kd | assay: IC₅₀ |
| 177 | A | B | A | A |
| 178 | A | B | A | B |
| 179 | A | B | C | B |
| 180 | C | NA | NA | NA |
| 181 | B | C | B | B |
| 182 | B | B | B | B |
| 183 | A | B | A | A |
| 184 | A | B | A | A |
| 185 | A | A | A | B |
| 186 | A | A | A | A |
| 187 | A | B | B | C |
| 188 | ND | B | B | A |
| 189 | ND | B | A | A |
| 190 | B | B | C | C |
| 191 | ND | ND | ND | B |

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

Since modifications will be apparent to those of skill in the art, it is intended that the claimed subject matter be limited only by the scope of the appended claims.

1. A compound having formula I: 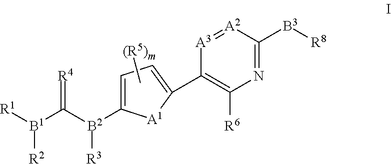
or a pharmaceutically acceptable salt thereof, wherein:
R¹ is optionally substituted aryl, optionally substituted heteroaryl or optionally substituted heterocyclyl; where the substituents when present are selected from one, two or three R⁹ groups, wherein each R⁹ is independently selected from halo, alkyl, alkenyl, alkynyl, alkoxy, hydroxyl, haloalkoxy, cycloalkyl, cycloalkylalkyl, hydroxyalkyl, haloalkyl, aryl, arylalkyl, heterocyclyl, heterocyclylalkyl, heteroaryl, and heteroarylalkyl, where the alkyl, alkenyl, alkynyl, alkoxy, haloalkoxy, cycloalkyl, cycloalkylalkyl, hydroxyalkyl, haloalkyl, aryl, heterocyclyl, and heteroaryl groups are optionally substituted with 1 to 5 groups selected from halo, alkyl, alkenyl, alkynyl, aryl, hydroxy, alkoxy, cycloalkyl, cyano, —R^{u}N(R^{y})(R^{z}), —R^{u}S(O)ₙR^{x}, —R^{u}C(O)OR^{x} and —R^{u}OC(O)R^{x};
R² and R³ are each independently hydrogen, halo, haloalkyl, hydroxy, alkyl, alkenyl, alkynyl, alkoxy or amino;
R⁴ is O, S, N—CN, or N—NO₂;
B¹ is N or CR^{2a};
B² is N or CR^{3a};
R^{2a} and R^{3a} are each independently hydrogen, halo, haloalkyl, hydroxy, alkyl, alkenyl, alkynyl or amino;
R⁵ is halo, alkyl, alkenyl, alkynyl, cycloalkyl, haloalkyl, hydroxyalkyl, alkoxyalkyl, cycloalkylalkyl, cyano, amino, hydroxy, alkoxy, —R^{u}N(R^{y})(R^{z}), aryl, heterocyclyl, or heteroaryl;
R⁶ is hydrogen, halo, alkyl, alkenyl, alkynyl, cycloalkyl, haloalkyl, hydroxyalkyl, alkoxyalkyl, cycloalkylalkyl, cyano, amino, hydroxy, alkoxy, hydroxyalkoxyalkyl, —R^{u}N(R^{y})(R^{z}), aryl, heterocyclyl, or heteroaryl;
B³ is O, NR⁷ or CR^{7a}R^{7a};
R⁷ is hydrogen, alkyl, alkenyl or alkynyl;
each R^{7a} is independently hydrogen, alkyl, alkenyl or alkynyl;
A² and R⁸ are selected as follows:
a) R⁸ is hydrogen, alkyl, alkenyl, alkynyl, cycloalkyl, —R^{u}OR^{x}, —R^{u}N(R^{y})(R^{z}), —R^{u}S(O)ₙN(R^{y})(R^{z}), —R^{u}S(O)ₙR^{x}, heterocyclyl, aryl, or heteroaryl; and A² is N, CH or CR¹⁰; or
b) A² is C; and R⁸ together with A² forms a 5-7 membered substituted or unsubstituted heterocycle, where the substituents when present are one, two or three Q groups, each independently selected from oxo, halo, hydroxyl, alkoxy, alkyl, alkenyl, alkynyl, cycloalkyl, —R^{u}N(R^{y})(R^{z}), —R^{u}S(O)ₙR^{x}, aryl, heterocyclyl, heteroaryl, hydroxyalkyl, haloalkyl and alkoxyalkyl;
R⁷ and R⁸ are each optionally substituted with 1-6, 1-3, one, two or three Q¹ groups, each independently selected from halo, hydroxyl, alkoxy, cycloalkyl, alkyl, alkenyl, alkynyl, haloalkyl, —R^{u}N(R^{y})(R^{z}), —R^{u}S(O)ₙR^{x}, aryl, heterocyclyl and heteroaryl;
Q and Q¹ groups are each optionally substituted with 1-8, 1-6, 1-5, 1-3, one, two or three Q² groups each independently selected from halo, alkyl, alkenyl, alkynyl, cycloalkyl, haloalkyl, aryl, amino, hydroxyl and alkoxy;
each R^{u} is independently alkylene, alkenylene or alkynylene or a direct bond;
each R^{x} is independently hydrogen, haloalkyl, alkyl, alkenyl or alkynyl;
each R^{y} and R^{z} is independently selected from (i) or (ii) below:
(i) R^{y} and R^{z} are each independently hydrogen, alkyl, alkenyl, alkynyl, cycloalkyl, alkoxyalkyl, or haloalkyl; or
(ii) R^{y} and R^{z}, together with the nitrogen atom to which they are attached, form a heterocyclyl or heteroaryl, optionally substituted with one, two, three, four or five halo, haloalkyl, alkyl, alkenyl or alkynyl groups;
A¹ is N═CR^{9a}, NR^{9a}, S, O, CR^{9a}═CR^{9a}, CR^{9a}═N; or N═N;
A³ is N, CH or CR¹⁰;
each R^{9a} is independently hydrogen, halo, alkyl, alkenyl, alkynyl, cycloalkyl, haloalkyl, hydroxyalkyl, alkoxyalkyl, cycloalkylalkyl, cyano, amino, hydroxyl, aryl, —R^{u}N(R^{y})(R^{z}), —R^{u}S(O)ₙR^{x} or alkoxy;
R¹⁰ is halo, alkyl, alkenyl, alkynyl, cycloalkyl, haloalkyl, hydroxyalkyl, alkoxyalkyl, cycloalkylalkyl, cyano, amino, hydroxyl, alkoxy, —R^{u}N(R^{a})(R^{b}), —R^{u}OR^{x}, —R^{u}OR^{x}OR^{x}, —C(O)N(R^{y})(R^{z}), —R^{u}S(O)ₙR^{x}, aryl, heterocyclyl, or non-azole heteroaryl;
R^{a} and R^{b} are each independently hydrogen, alkyl, alkenyl, alkynyl; or R^{a} and R^{b}, together with the nitrogen atom to which they are attached, form an optionally substituted heterocyclyl or heteroaryl, wherein the substituents when present are selected from halo, alkyl, hydroxy and haloalkyl;
R^{9a} and R¹⁰ are each optionally substituted with 1-8, 1-6, 1-5, one, two or three Q¹ groups, each independently selected from halo, hydroxyl, alkoxy, cycloalkyl, alkyl, alkenyl, alkynyl, haloalkyl, aryl, heterocyclyl and heteroaryl;
n is 0-2;
m is 0-2; and
wherein the compound is selected such that:
a) when A² is N, B³ is NH, R¹ is phenyl, A¹ is CH═CH and R⁸ is H, then R⁶ is not amino;
b) when R¹ thienyl, B¹ is CH, A² is N, B³ is NH, A¹ is CH═CH and R⁸ is H, then R⁶ is not amino; and
c) when R¹ is pyrazol-3-yl; 1,4,5,6-tetrahydrocyclopenta[c]pyrazol-3-yl; or pyridinyl, then B² is not CH, and
d) when R¹ is piperazinyl, then B¹ is not CH.

2. The compound of claim 1 having formula II: 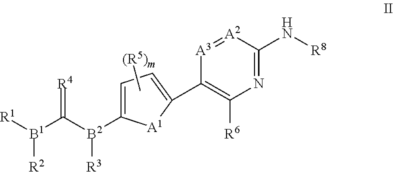
or a pharmaceutically acceptable salt thereof, wherein:
R¹ is optionally substituted aryl, optionally substituted heteroaryl or optionally substituted heterocyclyl; where the substituents when present are selected from one, two or three R⁹ groups, wherein each R⁹ is independently selected from halo, alkyl, alkenyl, alkynyl, alkoxy, hydroxyl, haloalkoxy, heterocyclyl and cycloalkyl, where the alkyl, alkenyl, alkynyl, alkoxy, haloalkoxy, heterocyclyl and cycloalkyl groups are optionally substituted with 1 to 5 groups selected from halo, alkyl, haloalkyl, alkoxyalkyl, hydroxy, alkoxy, cycloalkyl and —R^{u}OC(O)R^{x};
R² and R³ are each independently hydrogen, halo, hydroxy, haloalkyl or alkyl;
R⁴ is O or S;
B¹ is N or CR^{2a};
B² is N or CR^{3a};
R^{2a} and R^{3a} are each independently hydrogen, halo, or alkyl;
A¹ is N═CR^{9a}, S or CR^{9a}═CR^{9a};
R⁵ is halo, alkyl, alkenyl, alkynyl, cycloalkyl, haloalkyl, hydroxyalkyl, alkoxyalkyl, cycloalkylalkyl, cyano, amino, hydroxyl or alkoxy;
R⁶ is hydrogen, halo, alkyl, alkenyl, alkynyl, cycloalkyl, haloalkyl, hydroxyalkyl, alkoxyalkyl, hydroxyalkoxyalkyl, heterocyclylalkyl, cycloalkylalkyl, cyano, amino, hydroxyl or alkoxy;
A² and R⁸ are selected as follows:
a) R⁸ is hydrogen, alkyl, alkenyl, alkynyl, cycloalkyl, —R^{u}OR^{x}, —R^{u}N(R^{y})(R^{z}), —R^{u}S(O)ₙR^{x}, heterocyclyl, aryl, or heteroaryl; and A² is N, CH or CR¹⁰; or
b) A² is C; and R⁸ together with A² forms a 5-7 membered substituted or unsubstituted heterocyclyl, where the substituents when present are one, two or three Q groups, each independently selected from oxo, halo, hydroxyl, alkoxy, alkyl, alkenyl, alkynyl, cycloalkyl, heterocyclyl, heteroaryl, hydroxyalkyl, haloalkyl and alkoxyalkyl;
R⁸ is optionally substituted with one, two or three Q¹ groups, each independently selected from halo, hydroxyl, alkoxy, cycloalkyl, alkyl, alkenyl, alkynyl, haloalkyl, heterocyclyl and heteroaryl;
Q and Q¹ groups are each optionally substituted with 1-6, 1-5, one, two or three Q² groups each independently selected from halo, alkyl, alkenyl, alkynyl, cycloalkyl, haloalkyl, amino, hydroxyl and alkoxy;
each R^{u} is independently alkylene or a direct bond;
each R^{x} is independently hydrogen or alkyl;
each R^{y} and R^{z} is independently selected from (i) or (ii) below:
(i) R^{y} and R^{z} are each independently hydrogen, alkyl, alkenyl, alkynyl, or cycloalkyl; or
(ii) R^{y} and R^{z}, together with the nitrogen atom to which they are attached, form a heterocyclyl or heteroaryl, optionally substituted with one, two, three, four or five alkyl groups;
A³ is N, CH or CR¹⁰;
R^{9a} is hydrogen, halo or alkyl;
each R¹⁰ is independently alkyl, hydroxyalkyl, cyano, —R^{u}N(R^{a})(R^{b}), —R^{u}S(O)ₙR^{x} or —C(O)N(R^{y})(R^{z});
n is 0-2;
m is 0-2.

3. The compound of claim 1 having formula III: 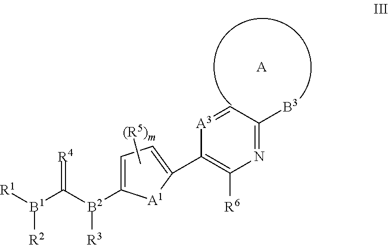
or a pharmaceutically acceptable salt thereof, wherein:
R¹ is optionally substituted aryl, heteroaryl or heterocyclyl; where the substituents when present are selected from one, two or three R⁹ groups, wherein each R⁹ is independently selected from halo, alkyl, alkenyl, alkynyl, alkoxy, hydroxyl, haloalkoxy, heterocyclyl and cycloalkyl, where the alkyl, alkenyl, alkynyl, alkoxy, haloalkoxy, and cycloalkyl groups are optionally substituted with 1 to 5 groups selected from halo, haloalkyl, alkoxyalkyl, hydroxy, alkoxy and cycloalkyl;
R² and R³ are each independently hydrogen, halo, hydroxy, amino or alkyl;
B¹ is N or CR^{2a};
B² is N or CR^{3a};
R^{2a} and R^{3a} are each independently hydrogen, halo, or alkyl;
R⁴ is O or S;
A¹ is N═CR^{9a}, S CR^{9a}═CR^{9a} or CR^{9a}═N;
R⁵ is halo, alkyl, alkenyl, alkynyl, cycloalkyl, haloalkyl, hydroxyalkyl, alkoxyalkyl, cycloalkylalkyl, cyano, amino, hydroxyl or alkoxy;
R⁶ is hydrogen, halo, alkyl, alkenyl, alkynyl, cycloalkyl, haloalkyl, hydroxyalkyl, alkoxyalkyl, hydroxyalkoxyalkyl, heterocyclylalkyl, cycloalkylalkyl, cyano, amino, hydroxyl or alkoxy;
B³ is NR⁷;
R⁷ is hydrogen or alkyl;
ring A is a 5-7 membered heterocyclyl optionally substituted with one, two or three Q groups, each independently selected from oxo, halo, hydroxyl, alkoxy, alkyl, alkenyl, alkynyl, cycloalkyl, heterocyclyl, heteroaryl, hydroxyalkyl, haloalkyl and alkoxyalkyl;
each Q is optionally substituted with one, two or three Q² groups each independently selected from halo, alkyl, alkenyl, alkynyl, cycloalkyl, haloalkyl, hydroxyl and alkoxy;
A³ is N, CH or CR¹⁰;
R^{9a} is hydrogen, halo or alkyl;
R¹⁰ is alkyl, hydroxyalkyl, cyano, —R^{u}N(R^{a})(R^{b}), —R^{u}OR^{x}, —R^{u}OR^{x}OR^{x}, —R^{u}S(O)ₙR^{x}, or —C(O)N(R^{y})(R^{z}) where R^{u} is direct bond or alkylene, and R^{a} and R^{b} are each hydrogen;
each R^{x} is independently hydrogen, alkyl, alkenyl or alkynyl;
each R^{y} and R^{z} is independently selected from (i) or (ii) below:
(i) R^{y} and R^{z} are each independently hydrogen, alkyl, alkenyl, alkynyl, cycloalkyl, alkoxyalkyl, or haloalkyl; or
(ii) R^{y} and R^{z}, together with the nitrogen atom to which they are attached, form a heterocyclyl or heteroaryl, optionally substituted with one, two, three, four or five halo, alkyl, haloalkyl, alkenyl or alkynyl groups;
n is 0-2; and
m is 0-2.

4. The compound of claim 1, where R¹ is: 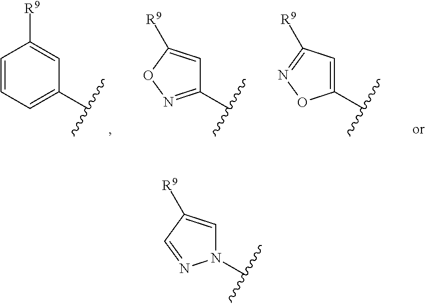
where R⁹ is alkyl, cycloalkyl or haloalkyl where the alkyl, cycloalkyl and haloalkyl is optionally substituted with 1 to 5 groups selected from halo, haloalkyl, alkoxyalkyl, hydroxy, alkoxy and cycloalkyl.

5. The compound of claim 1, where R¹ is: 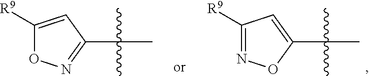
R⁹ is alkyl, cycloalkyl or haloalkyl where the alkyl, cycloalkyl and haloalkyl is optionally substituted with 1 to 5 groups selected from halo, haloalkyl, alkoxyalkyl, hydroxyl, alkoxy and cycloalkyl.

6. The compound of claim 1, where R¹ is optionally substituted phenyl, where substituents when present are selected from one, or two R⁹ groups, R⁹ is halo or alkyl, where alkyl is optionally substituted with 1 to 5 groups selected from halo and cycloalkyl.

7. The compound of claim 1, where R⁴ is O.

8. The compound of claim 1, where R⁵ is halo.

9. The compound of claim 1, where R⁶ is hydrogen, halo, alkyl or alkoxy.

10. The compound of claim 1, where R⁸ is hydrogen, alkyl, cycloalkyl, heterocyclyl, heterocyclylalkyl or heterocyclylalkenyl, where the alkyl, cycloalkyl, heterocyclyl, heterocyclylalkyl and heterocyclylalkenyl are optionally substituted with 1-5 or one or two alkyl, haloalkyl, hydroxy, alkoxy, amino or halo groups.

11. The compound of claim 1, where B³ is NH and R⁸ is hydrogen.

12. The compound of claim 1, where R⁸ together with A² forms a 5-7 membered heterocyclyl, optionally substituted with alkyl, hydroxyalkyl or oxo.

13. The compound of claim 1, where A¹ is N═CH, CH═CH or CH═N.

14. The compound of claim 1, where the compound has formula VA, VB, VC or VD: 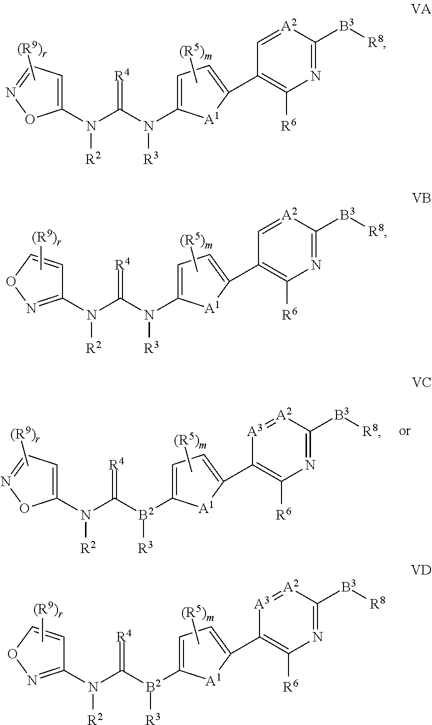
or a pharmaceutically acceptable salt thereof, wherein A¹ is N═CR^{9a}, S, CR^{9a}═N or CR^{9a}═CR^{9a};
R² is hydrogen or alkyl;
B² is N or CR^{3a};
R^{3a} is hydrogen, halo or alkyl;
R³ is hydrogen, halo, hydroxy, amino or alkyl;
R⁴ is O or S;
R⁵ is halo, alkyl, haloalkyl or alkoxy;
R⁶ is hydrogen, halo, alkyl, or alkoxy;
B³ is O, NH, or CH₂;
A² and R⁸ are selected as follows:
a) R⁸ is hydrogen, alkyl, alkenyl, alkynyl, cycloalkylalkyl, cycloalkyl, heterocyclyl, heterocyclylalkyl or heterocyclylalkenyl, where the alkyl, alkenyl, alkynyl, cycloalkyl, heterocyclyl, heterocyclylalkyl and heterocyclylalkenyl are optionally substituted with 1-6, 1-5, one or two alkyl, hydroxy, amino, alkylsulfonyl, or halo groups; and A² is N, CH or CR¹⁰; or
b) A² is C; and R⁸ together with A² forms a 5-7 membered substituted or unsubstituted heterocyclyl, optionally substituted with alkyl, hydroxyalkyl or oxo;
R⁹ is alkyl, where alkyl is optionally substituted with 1 to 5 groups selected from halo, hydroxy and cycloalkyl;
R^{9a} is hydrogen, halo, alkyl, or alkoxy;
R¹⁰ is alkyl, hydroxyalkyl, amido, cyano, —R^{u}S(O)ₙR^{x}, —C(O)N(R^{y})(R^{z}), —R^{u}N(R^{a})(R^{b}), —R^{u}OR^{x}, or —R^{u}OR^{x}OR^{x},
R^{u} is alkylene,
R^{a} and R^{b} are each independently hydrogen or alkyl
R^{y} and R^{z} are each independently hydrogen, alkyl, heterocyclyl or heteroaryl, alkyl, heterocyclyl or heteroaryl is each optionally substituted with one, two, three, four or five halo or alkyl;
A³ is N, CH or CR^{10a};
R^{10a} is halo, alkyl, or alkoxy;
n is 0-2;
m is 0 or 1; and
r is 1 or 2.

15. The compound of claim 1, where the compound has formula XII: 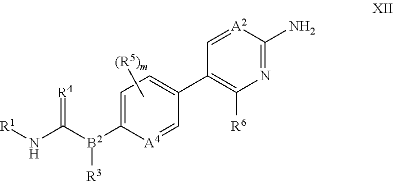
or a pharmaceutically acceptable salt thereof, wherein R¹ is substituted isoxazolyl where the substituents are selected from one or two R⁹ groups, wherein at least one R⁹ is a branched alkyl, heterocyclyl or cycloalkyl, and wherein the second optional R⁹ group is selected from halo, alkyl, haloalkyl, cycloalkyl and cycloalkylalkyl, where the alkyl, branched alkyl, haloalkyl, cycloalkyl or cycloalkylalkyl groups are each optionally substituted with one or two groups selected from halo, hydroxy, alkyl haloalkyl, alkoxyalkyl, alkoxy and cycloalkyl;
B² is N or CR^{3a};
R^{3a} is hydrogen, halo or alkyl;
R³ is hydrogen, halo, hydroxy, amino or alkyl;
A⁴ is N, or CR^{9a};
R⁵ is halo, alkyl, haloalkyl or alkoxy;
A² is N, CH or CR¹⁰;
R^{9a} is hydrogen, halo, alkyl, or alkoxy;
m is 0 or 1;
R¹⁰ is alkyl, hydroxyalkyl, cyano, or amido.

16. The compound of claim 1, wherein the compound is selected from:
1-(4-(6-aminopyridin-3-yl)phenyl)-3-(5-tert-butylisoxazol-3-yl)urea,
1-[4-(6-amino-5-cyanopyridin-3-yl)-phenyl]-3-(5-tert-butylisoxazol-3-yl)urea,
1-(5-tert-butylisoxazol-3-yl)-3-(4-(3-oxo-3,4-dihydro-2H-pyrido[3,2-b][1,4]oxazin-7-yl)phenyl)urea,
1-(5-tert-butylisoxazol-3-yl)-3-(4-(3,4-dihydro-2H-pyrido[3,2-b][1,4]oxazin-7-yl)phenyl)urea,
1-(5-tert-butylisoxazol-3-yl)-3-(4-(3-(2-hydroxyethyl)-3,4-dihydro-2H-pyrido[3,2-b][1,4]oxazin-7-yl)phenyl)urea,
1-(5-tert-butylisoxazol-3-yl)-3-(4-(5-cyano-6-(2-morpholinoethylamino)pyridin-3-yl)phenyl)urea,
1-(5-tert-butyl-isoxazol-3-yl)-3-{4-[6-(2-morpholin-4-yl-ethylamino)-pyridin-3-yl]-phenyl}urea,
1-(4-(6-amino-5-(hydroxymethyl)pyridin-3-yl)phenyl)-3-(5-tert-butylisoxazol-3-yl)urea,
1-(5-tert-butylisoxazol-3-yl)-3-(4-(7-oxo-5,6,7,8-tetrahydro-1,8-naphthyridin-3-yl)phenyl)urea,
1-(4-(6-amino-2,4-dimethylpyridin-3-yl)phenyl)-3-(5-tert-butylisoxazol-3-yl)urea,
1-(4-(6-aminopyridin-3-yl)-3-fluorophenyl)-3-(5-tert-butylisoxazol-3-yl)urea,
1-(5-tert-butylisoxazol-3-yl)-3-(4-(5,6,7,8-tetrahydro-1,8-naphthyridin-3-yl)phenyl)urea,
1-(4-(6-amino-5-(morpholinomethyl)pyridin-3-yl)phenyl)-3-(5-tert-butylisoxazol-3-yl)urea,
1-[4-(6-aminopyridin-3-yl)-2-fluorophenyl]-3-(5-tert-butylisoxazol-3-yl)-urea,
1-(4-(6-aminopyridin-3-yl)-2-chlorophenyl)-3-(5-tert-butylisoxazol-3-yl)urea,
1-(4-(6-aminopyridin-3-yl)-3-chlorophenyl)-3-(5-tert-butylisoxazol-3-yl)urea,
1-(6′-amino-[3,3′]bipyridinyl-6-yl)-3-(5-tert-butylisoxazol-3-yl)urea,
2-(4-(6-aminopyridin-3-yl)phenyl)-N-(5-tert-butylisoxazol-3-yl)acetamide,
1-(5-(6-aminopyridin-3-yl)thiophen-2-yl)-3-(5-tert-butylisoxazol-3-yl)urea,
1-(4-(6-aminopyridin-3-yl)-2,5-difluorophenyl)-3-(5-tert-butylisoxazol-3-yl)urea,
1-(5-tert-butylisoxazol-3-yl)-3-(4-(1,2,3,5-tetrahydropyrido[2,3-e][1,4]oxazepin-7-yl)phenyl)urea,
1-(4-(6-amino-5-((2-hydroxyethoxy)methyl)pyridin-3-yl)phenyl)-3-(5-tert-butylisoxazol-3-yl)urea,
1-(4-(6-amino-2-methylpyridin-3-yl)phenyl)-3-(5-tert-butylisoxazol-3-yl)urea,
1-(4-(6-amino-4-methylpyridin-3-yl)phenyl)-3-(5-tert-butylisoxazol-3-yl)urea,
1-(6′-amino-2′-methyl-3,3′-bipyridin-6-yl)-3-(5-tert-butylisoxazol-3-yl)urea,
1-(6′-amino-4′-methyl-3,3′-bipyridin-6-yl)-3-(5-tert-butylisoxazol-3-yl)urea,
1-(5-tert-butylisoxazol-3-yl)-3-(2-fluoro-4-(6-(2-(piperidin-1-yl)ethylamino)pyridin-3-yl)phenyl)urea,
1-(5-tert-butylisoxazol-3-yl)-3-(4-(6-(3-morpholinopropylamino)pyridin-3-yl)phenyl)urea,
1-(5-tert-butylisoxazol-3-yl)-3-(4-(6-(2-(1-methylpyrrolidin-2-yl)ethylamino)pyridin-3-yl)phenyl)urea,
1-(5-tert-butylisoxazol-3-yl)-3-(4-(6-((1-ethylpyrrolidin-2-yl)methylamino)pyridin-3-yl)phenyl)urea,
1-(4-(6-aminopyridin-3-yl)phenyl)-3-(5-tert-butylisoxazol-3-yl)-1-methylurea
5-(4-(3-(3-(2-fluoropropan-2-yl)isoxazol-5-yl)ureido)phenyl)pyridin-2-aminium methanesulfonate,
1-(4-(6-aminopyridin-3-yl)phenyl)-3-(3-(2-fluoropropan-2-yl)isoxazol-5-yl)urea
5-(4-(3-(5-(1-(trifluoromethyl)cyclopropyl)isoxazol-3-yl)ureido)phenyl)pyridin-2-aminium methanesulfonate,
1-(4-(6-aminopyridin-3-yl)phenyl)-3-(5-(1-(trifluoromethyl)cyclopropyl)isoxazol-3-yl)urea,
5-(4-(3-(5-(1,3-difluoro-2-methylpropan-2-yl)isoxazol-3-yl)ureido)phenyl)pyridin-2-aminium methanesulfonate,
1-(4-(6-aminopyridin-3-yl)phenyl)-3-(5-(1,3-difluoro-2-methylpropan-2-yl)isoxazol-3-yl)urea,
5-(4-(3-(3-(2-fluoropropan-2-yl)isoxazol-5-yl)ureido)phenyl)pyridin-2-aminium methanesulfonate,
1-(4-(6-aminopyridin-3-yl)phenyl)-3-(5-(1,1,1-trifluoro-2-methylpropan-2-yl)isoxazol-3-yl)urea,
4-(2-(5-(4-(3-(5-(1-(trifluoromethyl)cyclopropyl)isoxazol-3-yl)ureido)phenyl)pyridin-2-ylamino)ethyl)morpholin-4-ium methanesulfonate,
1-(4-(6-(2-morpholinoethylamino)pyridin-3-yl)phenyl)-3-(5-(1-(trifluoromethyl)cyclopropyl)isoxazol-3-yl)urea,
4-(2-(5-(4-(3-(5-(1,3-difluoro-2-methylpropan-2-yl)isoxazol-3-yl)ureido)phenyl)pyridin-2-ylamino)ethyl)morpholin-4-ium methanesulfonate,
1-(5-(1,3-difluoro-2-methylpropan-2-yl)isoxazol-3-yl)-3-(4-(6-(2-morpholinoethylamino)pyridin-3-yl)phenyl)urea,
4,4-(2-(5-(4-(3-(3-(2-fluoropropan-2-yl)isoxazol-5-yl)ureido)phenyl)pyridin-2-ylamino)ethyl)morpholin-4-ium methanesulfonate,
1-(3-(2-fluoropropan-2-yl)isoxazol-5-yl)-3-(4-(6-(2-morpholinoethylamino)pyridin-3-yl)phenyl)urea,
4-(2-(5-(4-(3-(5-(1,1,1-trifluoro-2-methylpropan-2-yl)isoxazol-3-yl)ureido)phenyl)pyridin-2-ylamino)ethyl)morpholin-4-ium methanesulfonate,
1-(4-(6-(2-morpholinoethylamino)pyridin-3-yl)phenyl)-3-(5-(1,1,1-trifluoro-2-methylpropan-2-yl)isoxazol-3-yl)urea,
1-(6′-amino-3,3′-bipyridin-6-yl)-3-(5-(1-(trifluoromethyl)cyclopropyl)isoxazol-3-yl)urea,
1-(6′-amino-3,3′-bipyridin-6-yl)-3-(5-(1,3-difluoro-2-methylpropan-2-yl)isoxazol-3-yl)urea,
1-(6′-amino-3,3′-bipyridin-6-yl)-3-(3-(2-fluoropropan-2-yl)isoxazol-5-yl)urea,
1-(6′-amino-3,3′-bipyridin-6-yl)-3-(5-(1,1,1-trifluoro-2-methylpropan-2-yl)isoxazol-3-yl)urea,
1-(4-(2-aminopyrimidin-5-yl)phenyl)-3-(5-tert-butylisoxazol-3-yl)urea,
1-(5-tert-butylisoxazol-3-yl)-3-{4-[2-(2-morpholin-4-yl-ethylamino)-pyrimidin-5-yl]-phenyl}urea,
N-(4-(2-aminopyrimidin-5-yl)phenyl)-2-(3-(trifluoromethyl)phenyl)acetamide,
1-(5-tert-butyl-isoxazol-3-yl)-3-{4-[2-(2-morpholin-4-yl-ethoxy)-pyrimidin-5-yl]-phenyl}-urea,
1-[4-(2-aminopyrimidin-5-yl)-2-methoxy-phenyl]-3-(5-tert-butylisoxazol-3-yl)-urea,
1-(4-(2-amino-4-methylpyrimidin-5-yl)phenyl)-3-(5-tert-butylisoxazol-3-yl)urea,
1-[4-(2-amino-4-methoxypyrimidin-5-yl)-phenyl]-3-(5-tert-butylisoxazol-3-yl)-urea,
1-(5-tert-butylisoxazol-3-yl)-3-(4-(2-(morpholinomethyl)pyrimidin-5-yl)phenyl)urea,
1-[5-(2-fluoro-1-fluoromethyl-1-methylethyl)isoxazol-3-yl]-3-{4-[2-(2-morpholin-4-yl-ethylamino)pyrimidin-5-yl]phenyl}urea,
1-{4-[2-(2-morpholin-4-yl-ethylamino)-pyrimidin-5-yl]-phenyl}-3-[5-(1-trifluoromethyl-cyclopropyl)-isoxazol-3-yl]urea,
1-(4-(2-(2-morpholinoethylamino)pyrimidin-5-yl)phenyl)-3-(3-(trifluoromethyl)phenyl)urea,
1-(2-fluoro-5-methylphenyl)-3-(4-(2-(2-morpholinoethylamino)pyrimidin-5-yl)phenyl)urea,
1-(5-tert-butylisoxazol-3-yl)-3-(4-(2-(3-morpholinopropyl)pyrimidin-5-yl)phenyl)urea,
1-(5-tert-butylisoxazol-3-yl)-3-(4-(2-(2-(dimethylamino)ethylamino)pyrimidin-5-yl)phenyl)urea,
1-(5-tert-butylisoxazol-3-yl)-3-{4-[2-(2-methoxyethylamino)pyrimidin-5-yl]-phenyl}urea,
1-[4-(6-aminopyridin-3-yl)-2-fluorophenyl]-3-(5-tert-butylisoxazol-3-yl)-urea,
1-(5-tert-butylisoxazol-3-yl)-3-(4-(2-(2-(piperidin-1-yl)ethylamino)pyrimidin-5-yl)phenyl)urea,
1-(5-tert-butylisoxazol-3-yl)-3-{5-[2-(2-morpholin-4-yl-ethylamino)pyrimidin-5-yl]-pyridin-2-yl}urea,
1-(5-(2-(tert-butylamino)pyrimidin-5-yl)pyridin-2-yl)-3-(5-tert-butylisoxazol-3-yl)urea,
1-(5-tert-butylisoxazol-3-yl)-3-(5-(2-(tetrahydro-2H-pyran-4-ylamino)pyrimidin-5-yl)pyridin-2-yl)urea,
1-(5-tert-butylisoxazol-3-yl)-3-[5-(2-cyclopropylaminopyrimidin-5-yl)-pyridin-2-yl]-urea,
1-(5-tert-butylisoxazol-3-yl)-3-(5-(2-(isopropylamino)pyrimidin-5-yl)pyridin-2-yl)urea,
N-(5-(2-(cyclopropylamino)pyrimidin-5-yl)pyridine-2-yl)-2-(3-(trifluoromethyl)phenyl)acetamide,
N-(5-(2-(isopropylamino)pyrimidin-5-yl)pyridin-2-yl)-2-(3-(trifluoromethyl)phenyl)acetamide,
1-(4-(6-aminopyridin-3-yl)-2-methoxyphenyl)-3-(5-(1,3-difluoro-2-methylpropan-2-yl)isoxazol-3-yl)urea,
1-(4-(6-aminopyridin-3-yl)-2-methoxyphenyl)-3-(5-(tert-butyl)isoxazol-3-yl)urea,
1-(4-(6-aminopyridin-3-yl)phenyl)-3-(3-(tert-butyl)isoxazol-5-yl)urea,
2-(4-(6-aminopyridin-3-yl)phenyl)-N-(5-(tert-butyl)isoxazol-3-yl)propanamide,
2-(4-(6-aminopyridin-3-yl)phenyl)-N-(5-(1,1,1-trifluoro-2-methylpropan-2-yl)isoxazol-3-yl)acetamide,
2-(4-(6-aminopyridin-3-yl)phenyl)-N-(5-(1-(trifluoromethyl)cyclopropyl)isoxazol-3-yl)acetamide,
2-(4-(6-aminopyridin-3-yl)phenyl)-N-(5-(1-(trifluoromethyl)cyclopropyl)isoxazol-3-yl)acetamide,
2-(4-(2-aminopyrimidin-5-yl)phenyl)-N-(5-(tert-butyl)isoxazol-3-yl)acetamide,
N-(5-(tert-butyl)isoxazol-3-yl)-2-(4-(2-((2-morpholinoethyl)amino)pyrimidin-5-yl)phenyl)acetamide,
2-(6′-amino-[3,3′-bipyridin]-6-yl)-N-(5-(tert-butyl)isoxazol-3-yl)acetamide,
2-(5-(2-aminopyrimidin-5-yl)pyridin-2-yl)-N-(5-(tert-butyl)isoxazol-3-yl)acetamide,
2-(4-(6-aminopyridin-3-yl)-2-fluorophenyl)-N-(5-(tert-butyl)isoxazol-3-yl)acetamide,
2-(4-(6-aminopyridin-3-yl)-2-fluorophenyl)-N-(5-(tert-butyl)isoxazol-3-yl)acetamide,
2-(4-(2-aminopyrimidin-5-yl)-2-fluorophenyl)-N-(5-(tert-butyl)isoxazol-3-yl)acetamide,
1-(4-(6-aminopyridin-3-yl)phenyl)-3-(5-(1-hydroxy-2-methylpropan-2-yl)isoxazol-3-yl)urea,
1-(4-(6-aminopyridin-3-yl)phenyl)-3-(3-(1-hydroxy-2-methylpropan-2-yl)isoxazol-5-yl)urea,
1-(4-(6-aminopyridin-3-yl)phenyl)-3-(3-(1-hydroxy-2-methylpropan-2-yl)-1-methyl-1H-pyrazol-5-yl)urea,
2-(4-(6-aminopyridin-3-yl)phenyl)-N-(3-(1-hydroxy-2-methylpropan-2-yl)isoxazol-5-yl)acetamide,
2-(4-(6-aminopyridin-3-yl)phenyl)-N-(3-(tert-butyl)isoxazol-5-yl)acetamide,
2-(4-(6-amino-5-methylpyridin-3-yl)phenyl)-N-(5-(tert-butyl)isoxazol-3-yl)acetamide,
2-(4-(6-amino-4-methylpyridin-3-yl)phenyl)-N-(5-(tert-butyl)isoxazol-3-yl)acetamide,
2-(4-(6-amino-2-methylpyridin-3-yl)phenyl)-N-(5-(tert-butyl)isoxazol-3-yl)acetamide,
2-(4-(6-aminopyridin-3-yl)phenyl)-N-(5-(1-hydroxy-2-methylpropan-2-yl)isoxazol-3-yl)acetamide,
N-(5-(tert-butyl)isoxazol-3-yl)-2-(4-(6-((2-methoxyethyl)amino)pyridin-3-yl)phenyl)acetamide,
1-(4-(6-aminopyridin-3-yl)phenyl)-3-(3-(1-fluoro-2-methylpropan-2-yl)-1-methyl-1H-pyrazol-5-yl)urea,
2-(4-(6-aminopyridin-3-yl)phenyl)-N-(3-(tert-butyl)-1H-pyrazol-1-yl)acetamide compound with 2-(4-(6-aminopyridin-3-yl)phenyl)-N-(5-(tert-butyl)-1H-pyrazol-1-yl)acetamide (1:1),
N-(5-(tert-butyl)isoxazol-3-yl)-2-(4-(6-((2-(methylsulfonyl)ethyl)amino)pyridin-3-yl)phenyl)acetamide,
2-(4-(6-amino-5-cyanopyridin-3-yl)phenyl)-N-(5-(tert-butyl)isoxazol-3-yl)acetamide,
2-(4-(6-amino-5-fluoropyridin-3-yl)phenyl)-N-(5-(tert-butyl)isoxazol-3-yl)acetamide,
2-(4-(6-amino-2-fluoropyridin-3-yl)phenyl)-N-(5-(tert-butyl)isoxazol-3-yl)acetamide,
N-(3-(tert-butyl)isoxazol-5-yl)-2-(4-(6-((2-morpholinoethyl)amino)pyridin-3-yl)phenyl)acetamide,
1-(4-(6-aminopyridin-3-yl)phenyl)-3-(3-(1-fluoro-2-methylpropan-2-yl)isoxazol-5-yl)urea,
1-(4-(6-aminopyridin-3-yl)phenyl)-3-(5-(3-methyloxetan-3-yl)isoxazol-3-yl)urea,
2-(4-(6-aminopyridin-3-yl)phenyl)-N-(5-(3-methyloxetan-3-yl)isoxazol-3-yl)acetamide,
2-(4-(6-aminopyridin-3-yl)phenyl)-N-(5-(1-methylcyclopropyl)isoxazol-3-yl)acetamide,
2-(4-(6-aminopyridin-3-yl)phenyl)-N-(3-(2-fluoropropan-2-yl)isoxazol-5-yl)acetamide,
2-(4-(6-aminopyridin-3-yl)phenyl)-N-(5-(2,2-difluoro-1-methylcyclopropyl)isoxazol-3-yl)acetamide,
2-(4-(6-aminopyridin-3-yl)phenyl)-N-(5-(1-(fluoromethyl)cyclopropyl)isoxazol-3-yl)acetamide,
1-(6′-amino-[3,3′-bipyridin]-6-yl)-3-(5-(1-methylcyclopropyl)isoxazol-3-yl)urea,
N-(5-(tert-butyl)isoxazol-3-yl)-2-(4-(6-((2-morpholinoethyl)amino)pyridin-3-yl)phenyl)acetamide,
1-(4-(6-aminopyridin-3-yl)phenyl)-3-(5-(1-methylcyclopropyl)isoxazol-3-yl)urea,
1-(5-(tert-butyl)isoxazol-3-yl)-3-(4-(6-((2-(4,4-difluoropiperidin-1-yl)ethyl)amino)pyridin-3-yl)phenyl)urea,
N-(5-(tert-butyl)isoxazol-3-yl)-2-(4-(6-((2-(4,4-difluoropiperidin-1-yl)ethyl)amino)pyridin-3-yl)phenyl)acetamide,
2-(4-(6-((2-morpholinoethyl)amino)pyridin-3-yl)phenyl)-N-(5-(1,1,1-trifluoro-2-methylpropan-2-yl)isoxazol-3-yl)acetamide,
N-(5-(tert-butyl)isoxazol-3-yl)-2-(4-(6-(methylamino)pyridin-3-yl)phenyl)acetamide,
N-(5-(tert-butyl)isoxazol-3-yl)-2-(4-(6-(ethylamino)pyridin-3-yl)phenyl)acetamide,
1-(4-(6-aminopyridin-3-yl)phenyl)-3-(5-(2,2-difluoro-1-methylcyclopropyl)isoxazol-3-yl)urea,
2-(4-(5-amino-6-methylpyrazin-2-yl)phenyl)-N-(5-(tert-butyl)isoxazol-3-yl)acetamide,
3-amino-6-(4-(2-((5-(tert-butyl)isoxazol-3-yl)amino)-2-oxoethyl)phenyl)pyrazine-2-carboxamide,
2-(4-(6-amino-5-chloropyridin-3-yl)phenyl)-N-(5-(tert-butyl)isoxazol-3-yl)acetamide,
2-(4-(6-amino-5-(trifluoromethyl)pyridin-3-yl)phenyl)-N-(5-(tert-butyl)isoxazol-3-yl)acetamide,
N-(5-(tert-butyl)isoxazol-3-yl)-2-(4-(6-((2-(1,2,2,6,6-pentamethylpiperidin-4-ylidene)ethyl)amino)pyridin-3-yl)phenyl)acetamide,
N-(3-(2-fluoropropan-2-yl)isoxazol-5-yl)-2-(4-(6-((2-morpholinoethyl)amino)pyridin-3-yl)phenyl)acetamide,
2-(4-(6-amino-2-fluoropyridin-3-yl)phenyl)-N-(3-(tert-butyl)isoxazol-5-yl)acetamide,
N-(5-(tert-butyl)isoxazol-3-yl)-2-(4-(5,6,7,8-tetrahydro-1,8-naphthyridin-3-yl)phenyl)acetamide,
1-(4-(6-aminopyridin-3-yl)phenyl)-3-(5-(1-(trifluoromethyl)cyclobutyl)isoxazol-3-yl)urea,
N-(5-(tert-butyl)isoxazol-3-yl)-2-(4-(6-((2-(3-methyloxetan-3-yl)ethyl)amino)pyridin-3-yl)phenyl)acetamide,
2-(4-(6-aminopyridin-3-yl)-2,6-difluorophenyl)-N-(5-(tert-butyl)isoxazol-3-yl)acetamide,
2-(4-(6-aminopyridin-3-yl)phenyl)-N-(5-(1-(trifluoromethyl)cyclobutyl)isoxazol-3-yl)acetamide,
2-(4-(6-aminopyridin-3-yl)-3-fluorophenyl)-N-(5-(tert-butyl)isoxazol-3-yl)acetamide,
2-(4-(6-aminopyridin-3-yl)phenyl)-N-(4-(trifluoromethyl)-1H-pyrazol-1-yl)acetamide,
2-(4-(6-amino-5-fluoropyridin-3-yl)phenyl)-N-(5-(1-(trifluoromethyl)cyclopropyl)isoxazol-3-yl)acetamide,
2-(4-(6-amino-5-chloropyridin-3-yl)phenyl)-N-(5-(3-methyloxetan-3-yl)isoxazol-3-yl)acetamide,
2-(4-(6-amino-2-(trifluoromethyl)pyridin-3-yl)phenyl)-N-(5-(tert-butyl)isoxazol-3-yl)acetamide,
2-(4-(6-amino-5-methylpyridin-3-yl)phenyl)-N-(5-(1-(trifluoromethyl)cyclopropyl)isoxazol-3-yl)acetamide,
2-(4-(6-amino-2-methoxypyridin-3-yl)phenyl)-N-(5-(tert-butyl)isoxazol-3-yl)acetamide,
2-(4-(6-amino-2-chloropyridin-3-yl)phenyl)-N-(5-(tert-butyl)isoxazol-3-yl)acetamide,
2-(4-(6-amino-5-chloropyridin-3-yl)phenyl)-N-(5-(1-(trifluoromethyl)cyclopropyl)isoxazol-3-yl)acetamide,
2-(4-(6-amino-2-fluoropyridin-3-yl)phenyl)-N-(5-(1-methylcyclopropyl)isoxazol-3-yl)acetamide,
2-(4-(6-amino-2-fluoropyridin-3-yl)phenyl)-N-(5-(1-methylcyclopropyl)isoxazol-3-yl)acetamide,
2-(4-(6-amino-5-(trifluoromethyl)pyridin-3-yl)phenyl)-N-(5-(3-methyloxetan-3-yl)isoxazol-3-yl)acetamide,
2-(4-(6-amino-5-methoxypyridin-3-yl)phenyl)-N-(5-(tert-butyl)isoxazol-3-yl)acetamide,
2-(4-(6-amino-2-fluoropyridin-3-yl)phenyl)-N-(5-(1-(trifluoromethyl)cyclopropyl)isoxazol-3-yl)acetamide,
2-(4-(6-amino-5-fluoropyridin-3-yl)phenyl)-N-(5-(1-methylcyclopropyl)isoxazol-3-yl)acetamide,
2-(4-(6-aminopyridin-3-yl)-2-fluorophenyl)-N-(5-(1-(trifluoromethyl)cyclopropyl)isoxazol-3-yl)acetamide,
2-(4-(6-amino-2-fluoropyridin-3-yl)phenyl)-N-(5-(3-methyloxetan-3-yl)isoxazol-3-yl)acetamide,
N-(5-(tert-butyl)isoxazol-3-yl)-2-(4-(2-oxo-2,3-dihydrooxazolo[4,5-b]pyridin-6-yl)phenyl)acetamide,
2-(4-(6-amino-2-fluoropyridin-3-yl)phenyl)-N-(5-(1,1,1-trifluoro-2-methylpropan-2-yl)isoxazol-3-yl)acetamide,
2-(4-(6-aminopyridin-3-yl)phenyl)-N-(3-cyclobutylisoxazol-5-yl)acetamide,
2-(4-(6-aminopyridin-3-yl)phenyl)-N-(5-(1-methylcyclobutyl)isoxazol-3-yl)acetamide,
2-(4-(6-amino-5-methylpyridin-3-yl)phenyl)-N-(5-(3-methyloxetan-3-yl)isoxazol-3-yl)acetamide,
2-(4-(6-amino-5-fluoropyridin-3-yl)phenyl)-N-(5-(3-methyloxetan-3-yl)isoxazol-3-yl)acetamide,
N-(5-(tert-butyl)isoxazol-3-yl)-2-(4-(2-oxo-2,3-dihydro-1H-imidazo[4,5-b]pyridin-6-yl)phenyl)acetamide,
2-(6′-amino-5-fluoro-[3,3′-bipyridin]-6-yl)-N-(5-(tert-butyl)isoxazol-3-yl)acetamide,
2-(4-(6-aminopyridin-3-yl)phenyl)-N-(5-(1-(difluoromethyl)cyclopropyl)isoxazol-3-yl)acetamide,
2-(4-(6-amino-4-chloropyridin-3-yl)phenyl)-N-(5-(tert-butyl)isoxazol-3-yl)acetamide,
2-(4-(6-amino-4-fluoropyridin-3-yl)phenyl)-N-(5-(tert-butyl)isoxazol-3-yl)acetamide,
2-(4-(6-amino-2,5-difluoropyridin-3-yl)phenyl)-N-(5-(tert-butyl)isoxazol-3-yl)acetamide,
2-(4-(6-aminopyridin-3-yl)phenyl)-N-(5-(1-(1,1-difluoroethyl)cyclopropyl)isoxazol-3-yl)acetamide,
2-(4-(6-amino-5-(1-methyl-1H-pyrazol-4-yl)pyridin-3-yl)phenyl)-N-(5-(tert-butyl)isoxazol-3-yl)acetamide,
2-(6′-amino-[2,3′-bipyridin]-5-yl)-N-(5-(tert-butyl)isoxazol-3-yl)acetamide,
2-(4-(6-amino-4-(trifluoromethyl)pyridin-3-yl)phenyl)-N-(5-(tert-butyl)isoxazol-3-yl)acetamide,
2-(4-(6-amino-4-methoxypyridin-3-yl)phenyl)-N-(5-(tert-butyl)isoxazol-3-yl)acetamide,
2-(4-(6-amino-5-methylpyridin-3-yl)phenyl)-N-(5-(1-methylcyclopropyl)isoxazol-3-yl)acetamide,
2-(4-(6-amino-2-fluoropyridin-3-yl)phenyl)-N-(5-(1-(1,1-difluoroethyl)cyclopropyl)isoxazol-3-yl)acetamide,
2-(4-(6-amino-5-chloropyridin-3-yl)phenyl)-N-(5-(1-methylcyclopropyl)isoxazol-3-yl)acetamide,
2-(4-(6-amino-5-(trifluoromethyl)pyridin-3-yl)phenyl)-N-(5-(1-methylcyclopropyl)isoxazol-3-yl)acetamide,
2-(4-(6-amino-2-methylpyridin-3-yl)phenyl)-N-(5-(1-methylcyclopropyl)isoxazol-3-yl)acetamide,
2-(4-(6-amino-5-(difluoromethyl)pyridin-3-yl)phenyl)-N-(5-(tert-butyl)isoxazol-3-yl)acetamide,
2-(4-(6-amino-5-methoxypyridin-3-yl)phenyl)-N-(5-(1-methylcyclopropyl)isoxazol-3-yl)acetamide,
2-(4-(5-amino-3-methylpyrazin-2-yl)phenyl)-N-(5-(tert-butyl)isoxazol-3-yl)acetamide,
2-(4-(6-amino-2-cyanopyridin-3-yl)phenyl)-N-(5-(tert-butyl)isoxazol-3-yl)acetamide,
2-(4-(6-amino-2-fluoropyridin-3-yl)phenyl)-N-(5-(1-(trifluoromethyl)cyclobutyl)isoxazol-3-yl)acetamide,
(1-(3-(2-(4-(6-aminopyridin-3-yl)phenyl)acetamido)isoxazol-5-yl)cyclopropyl)methyl acetate,
2-(4-(6-amino-4-fluoropyridin-3-yl)phenyl)-N-(5-(1-methylcyclopropyl)isoxazol-3-yl)acetamide,
2-(4-(6-aminopyridin-3-yl)phenyl)-N-(5-(1-ethylcyclopropyl)isoxazol-3-yl)acetamide,
2-(4-(6-amino-4-chloropyridin-3-yl)phenyl)-N-(5-(1-methylcyclopropyl)isoxazol-3-yl)acetamide,
2-amino-2-(4-(6-aminopyridin-3-yl)phenyl)-N-(5-(tert-butyl)isoxazol-3-yl)acetamide,
2-(4-(5-amino-3,6-dimethylpyrazin-2-yl)phenyl)-N-(5-(tert-butyl)isoxazol-3-yl)acetamide,
2-(4-(6-amino-5-(tert-butylthio)pyridin-3-yl)phenyl)-N-(5-(tert-butyl)isoxazol-3-yl)acetamide,
2-(4-(6-amino-5-(tert-butylsulfonyl)pyridin-3-yl)phenyl)-N-(5-(tert-butyl)isoxazol-3-yl)acetamide,
2-(4-(6-amino-2,5-difluoropyridin-3-yl)phenyl)-N-(5-(1-methylcyclopropyl)isoxazol-3-yl)acetamide,
2-(4-(6-aminopyridin-3-yl)phenyl)-N-(5-(tert-butyl)isoxazol-3-yl)-2,2-difluoroacetamide,
2-(4-(6-amino-5-(tert-butylsulfinyl)pyridin-3-yl)phenyl)-N-(5-(tert-butyl)isoxazol-3-yl)acetamide,
2-(4-(6-aminopyridin-3-yl)-3-(trifluoromethyl)phenyl)-N-(5-(tert-butyl)isoxazol-3-yl)acetamide,
N-(5-(tert-butyl)isoxazol-3-yl)-2-(4-(2-methyl-3-oxo-2,3-dihydro-1H-pyrazolo[3,4-b]pyridin-5-yl)phenyl)acetamide,
2-(4-(6-aminopyridin-3-yl)phenyl)-N-(5-(tert-butyl)isoxazol-3-yl)-2-hydroxyacetamide,
2-(4-(6-amino-4-fluoropyridin-3-yl)phenyl)-N-(3-(tert-butyl)isoxazol-5-yl)acetamide,
2-(4-(6-amino-4-fluoropyridin-3-yl)phenyl)-N-(5-(1-(1,1-difluoroethyl)cyclopropyl)isoxazol-3-yl)acetamide,
N-(5-(1-methylcyclopropyl)isoxazol-3-yl)-2-(4-(6-((2-(methylsulfonyl)ethyl)amino)pyridin-3-yl)phenyl)acetamide,
2-(4-(6-aminopyridin-3-yl)phenyl)-N-(5-isopropylisoxazol-3-yl)acetamide,
N-(5-(tert-butyl)isoxazol-3-yl)-2-(4-(2-oxo-2,3,4,5-tetrahydro-1H-pyrido[2,3-e][1,4]diazepin-7-yl)phenyl)acetamide,
2-(4-(6-((2-(methylsulfonyl)ethyl)amino)pyridin-3-yl)phenyl)-N-(5-(1-(trifluoromethyl)cyclopropyl)isoxazol-3-yl)acetamide,
N-(5-(tert-butyl)isoxazol-3-yl)-2-(4-(6-oxo-5,6-dihydro-1,5-naphthyridin-3-yl)phenyl)acetamide,
2-(4-(6-amino-4-fluoropyridin-3-yl)phenyl)-N-(5-(1-(trifluoromethyl)cyclopropyl)isoxazol-3-yl)acetamide, and
N-(5-(tert-butyl)isoxazol-3-yl)-2-(4-(4-methyl-2-oxo-2,3,4,5-tetrahydro-1H-pyrido[2,3-e][1,4]diazepin-7-yl)phenyl)acetamide, or a pharmaceutically acceptable salt thereof.

17. A pharmaceutical composition comprising a compound of claim 1 and a pharmaceutically acceptable carrier.

18. A method for treatment of a disease selected from an inflammatory disease, an inflammatory condition, an autoimmune disease and cancer, comprising administering a therapeutically effective amount of a compound of claim 1.

19. The method of claim 18, wherein the disease is modulated by KIT, CSF-1R and/or FLT3 kinase.

20. The method of claim 18, wherein the disease is modulated by wild type or mutant KIT, CSF-1R and/or FLT3 kinase.

21. A method for the treatment of a disease, comprising administering a therapeutically effective amount of a compound of claim 1, wherein the disease is selected from myeloproliferative disorder, polycythemia vera, essential thrombocythemia, primary myelofibrosis, chronic eosinophilic leukemia, chronic myelomonocytic leukemia, systemic mastocytosis, idiopathic myelofibrosis, myeloid leukemia, chronic myeloid leukemia, imatinib-resistant CML, acute myeloid leukemia, acute megakaryoblastic leukemia, myeloma, cancer of the head and neck, prostate cancer, breast cancer, ovarian cancer, melanoma, lung cancer, brain cancer, pancreatic cancer, renal cancer, immunodeficiency, autoimmune diseases, tissue transplant rejection, graft-versus-host disease, wound, kidney disease, multiple sclerosis, thyroiditis, type 1 diabetes, sarcoidosis, psoriasis, allergic rhinitis, inflammatory bowel disease including Crohn's disease and ulcerative colitis, systemic lupus erythematosis, arthritis, osteoarthritis, rheumatoid arthritis, osteoporosis, asthma, chronic obstructive pulmonary disease, mast cell leukemia, myeloid dysplastic syndrome, seminomas, dysgerminomas, gastrointestinal stromal tumor and sepsis.

22. The method of claim 18, further comprising administering a second pharmaceutical agent selected from an anti-proliferative agent, an anti-inflammatory agent, an immunomodulatory agent and an immunosuppressive agent.

23. A method of modulating CSF-1R and/or FLT3 kinase, comprising administering a compound of claim 1.

24-25. (canceled)

26. The compound of claim 1 selected from
5-(4-(3-(3-(3-methyloxetan-3-yl)isoxazol-5-yl)ureido)phenyl)pyridin-2-aminium,
5-(4-(2-((3-(3-methyloxetan-3-yl)isoxazol-5-yl)amino)-2-oxoethyl)phenyl)pyridin-2-aminium methanesulfonate,
5-(4-(2-((3-(1-methylcyclopropyl)isoxazol-5-yl)amino)-2-oxoethyl)phenyl)pyridin-2-aminium methanesulfonate,
5-(4-(2-((3-(2,2-difluoro-1-methylcyclopropyl)isoxazol-5-yl)amino)-2-oxoethyl)phenyl)pyridin-2-aminium acetate,
5-(4-(2-((3-(1-fluorocyclobutyl)isoxazol-5-yl)amino)-2-oxoethyl)phenyl)pyridin-2-aminium acetate,
6′-(3-(3-(1-methylcyclopropyl)isoxazol-5-yl)ureido)-[3,3′-bipyridin]-6-aminium methanesulfonate,
5-(4-(3-(3-(1-methylcyclopropyl)isoxazol-5-yl)ureido)phenyl)pyridin-2-aminium methanesulfonate,
5-(4-(3-(3-(2,2-difluoro-1-methylcyclopropyl)isoxazol-5-yl)ureido)phenyl)pyridin-2-aminium methanesulfonate,
5-(4-(3-(3-(1-(trifluoromethyl)cyclobutyl)isoxazol-5-yl)ureido)phenyl)pyridin-2-aminium methanesulfonate,
2-(4-(6-aminopyridin-3-yl)phenyl)-N-(3-(1-(trifluoromethyl)cyclobutyl)isoxazol-5-yl)acetamide,
3-chloro-5-(4-(2-((3-(3-methyloxetan-3-yl)isoxazol-5-yl)amino)-2-oxoethyl)phenyl)pyridin-2-aminium methanesulfonate,
2-(4-(6-amino-2-fluoropyridin-3-yl)phenyl)-N-(3-(1-methylcyclopropyl)isoxazol-5-yl)acetamide,
5-(4-(2-((3-(3-methyloxetan-3-yl)isoxazol-5-yl)amino)-2-oxoethyl)phenyl)-3-(trifluoromethyl)pyridin-2-aminium methanesulfonate,
3-fluoro-5-(4-(2-((3-(1-methylcyclopropyl)isoxazol-5-yl)amino)-2-oxoethyl)phenyl)pyridin-2-aminium methanesulfonate,
2-(4-(6-amino-2-fluoropyridin-3-yl)phenyl)-N-(3-(3-methyloxetan-3-yl)isoxazol-5-yl)acetamide,
5-(4-(2-((3-(1-methylcyclobutyl)isoxazol-5-yl)amino)-2-oxoethyl)phenyl)pyridin-2-aminium methanesulfonate,
3-methyl-5-(4-(2-((3-(3-methyloxetan-3-yl)isoxazol-5-yl)amino)-2-oxoethyl)phenyl)pyridin-2-aminium methanesulfonate,
3-fluoro-5-(4-(2-((3-(3-methyloxetan-3-yl)isoxazol-5-yl)amino)-2-oxoethyl)phenyl)pyridin-2-aminium methanesulfonate,
2-(4-(6-aminopyridin-3-yl)phenyl)-N-(3-(1-(difluoromethyl)cyclopropyl)isoxazol-5-yl)acetamide,
2-(4-(6-aminopyridin-3-yl)phenyl)-N-(3-(1-(1,1-difluoroethyl)cyclopropyl)isoxazol-5-yl)acetamide,
2-(4-(6-amino-5-methylpyridin-3-yl)phenyl)-N-(3-(1-methylcyclopropyl)isoxazol-5-yl)acetamide,
2-(4-(6-amino-2-fluoropyridin-3-yl)phenyl)-N-(3-(1-(1,1-difluoroethyl)cyclopropyl)isoxazol-5-yl)acetamide,
2-(4-(6-amino-5-chloropyridin-3-yl)phenyl)-N-(3-(1-methylcyclopropyl)isoxazol-5-yl)acetamide,
2-(4-(6-amino-5-(trifluoromethyl)pyridin-3-yl)phenyl)-N-(3-(1-methylcyclopropyl)isoxazol-5-yl)acetamide,
2-(4-(6-amino-2-methylpyridin-3-yl)phenyl)-N-(3-(1-methylcyclopropyl)isoxazol-5-yl)acetamide,
2-(4-(6-amino-5-methoxypyridin-3-yl)phenyl)-N-(3-(1-methylcyclopropyl)isoxazol-5-yl)acetamide,
2-(4-(6-amino-2-fluoropyridin-3-yl)phenyl)-N-(3-(1-(trifluoromethyl)cyclobutyl)isoxazol-5-yl)acetamide,
(1-(5-(2-(4-(6-aminopyridin-3-yl)phenyl)acetamido)isoxazol-3-yl)cyclopropyl)methyl acetate,
2-(4-(6-amino-4-fluoropyridin-3-yl)phenyl)-N-(3-(1-methylcyclopropyl)isoxazol-5-yl)acetamide,
2-(4-(6-aminopyridin-3-yl)phenyl)-N-(3-(1-ethylcyclopropyl)isoxazol-5-yl)acetamide,
2-(4-(6-amino-4-chloropyridin-3-yl)phenyl)-N-(3-(1-methylcyclopropyl)isoxazol-5-yl)acetamide,
2-(4-(6-amino-2,5-difluoropyridin-3-yl)phenyl)-N-(3-(1-methylcyclopropyl)isoxazol-5-yl)acetamide,
2-(4-(6-amino-4-fluoropyridin-3-yl)phenyl)-N-(3-(1-(1,1-difluoroethyl)cyclopropyl)isoxazol-5-yl)acetamide, and
N-(3-(1-methylcyclopropyl)isoxazol-5-yl)-2-(4-(6-((2-(methylsulfonyl)ethyl)amino)pyridin-3-yl)phenyl)acetamide.
